SEQUENCE LISTING (847 sequences)

SEQ ID NO: 1 - protein, 1368 aa, Streptococcus pyogenes [sequence omitted]

SEQ ID NO: 2 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 3 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSIMIQVKILHTTKSPAV

SEQ ID NO: 5 - protein, 145 aa, Homo sapiens
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKAPV

SEQ ID NO: 6 - protein, 198 aa, Homo sapiens [sequence omitted]

SEQ ID NO: 7 - protein, 198 aa, Mus sp.
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 8 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 9 - protein, 199 aa, Bos sp.
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 10 - protein, 239 aa, Rattus sp.
MAVGSKPKAALVGPHWERERIWCFLCSTGLGTQQTGQTSRWLRPAATQDPVSPPRSLLMKQRKFLYHFKNVRWAKGRHETYLCYVVKRRDSATSFSLDFGYLRNKSGCHVELLFLRYISDWDLDPGRCYRVTWFTSWSPCYDCARHVADFLRGNPNLSLRIFTARLTGWGALPAGLMSPARPSDYFYCWNTFVENHERTFKAWEGLHENSVRLSRRLRRILLPLYEVDDLRDAFRTLGL

SEQ ID NO: 11 - protein, 198 aa, Canis lupus; identical to SEQ ID NO: 8

SEQ ID NO: 12 - protein, 199 aa, Bos taurus; identical to SEQ ID NO: 9

SEQ ID NO: 13 - protein, 198 aa, Mus musculus [sequence omitted]

SEQ ID NO: 14 - protein, 229 aa, Rattus norvegicus [sequence omitted]

SEQ ID NO: 15 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 16 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 17 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 18 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 19 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 20 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 21 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 22 - protein, 429 aa, Mus sp.
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYIPVPSSSSSTLSNICLTKGLPETRFCVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 23 - protein, 430 aa, Rattus sp.
MGPFCLGCSHRKCYSPIRNLISQETFKFHFKNRLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 24 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 25 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 26 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 27 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 28 - protein, 377 aa, Chlorocebus sp.
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 29 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 30 - protein, 373 aa, Homo sapiens; identical to SEQ ID NO: 25

SEQ ID NO: 31 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 32 - protein, 395 aa, Rattus sp.
MQPQGLGPNAGMGPVCLGCSHRRPYSPIRNPLKKLYQQTFYFHFKNVRYAWGRKNNFLCYEVNGMDCALPVPLRQGVFRKQGHIHAELCFIYWFHDKVLRVLSPMEEFKVTWYMSWSPCSKCAEQVARFLAAHRNLSLAIFSSRLYYYLRNPNYQQKLCRLIQEGVHVAAMDLPEFKKCWNKFVDNDGQPFRPWMRLRINFSFYDCKLQEIFSRMNLLREDVFYLQFNNSHRVKPVQNRYYRRKSYLCYQLERANGQEPLKGYLLYKKGEQHVEILFLEKMRSMELSQVRITCYLTWSPCPNCARQLAAFKKDHPDLILRIYTSRLYFWRKKFQKGLCTLWRSGIHVDVMDLPQFADCWTNFVNPQRPFRPWNELEKNSWRIQRRLRRIKESWGL

SEQ ID NO: 33 - protein, 227 aa, Bos sp.
MDGWEVAFRSGTVLKAGVLGVSMTEGWAGSGHPGQGACVWTPGTRNTMNLLREVLFKQQFGNQPRVPAPYYRRKTYLCYQLKQRNDLTLDRGCFRNKKQRHAERFIDKINSLDLNPSQSYKIICYITWSPCPNCANELVNFITRNNHLKLEIFASRLYFHWIKSFKMGLQDLQNAGISVAVMTHTEFEDCWEQFVDNQSRPFQPWDKLEQYSASIRRRLQRILTAPI

SEQ ID NO: 34 - protein, 490 aa, Pan troglodytes
MNPQIRNPMEWMYQRTFYYNFENEPILYGRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRLSQAGARVKIMDDEEFAYCWENFVYNEGQPFMPWYKFDDNYAFLHRTLKEIIRHLMDPDTFTFNFNNDPLVLRRHQTYLCYEVERLDNGTWVLMDQHMGFLCNEAKNLLCGFYGRHAELRFLDLVPSLQLDPAQIYRVTWFISWSPCFSWGCAGQVRAFLQENTHVRLRIFAARIYDYDPLYKEALQMLRDAGAQVSIMTYDEFEYCWDTFVYRQGCPFQPWDGLEEHSQALSGRLRAILQVRASSLCMVPHRPPPPPQSPGPCLPLCSEPPLGSLLPTGRPAPSLPFLLTASFSFPPPASLPPLPSLSLSPGHLPVPSFHSLTSCSIQPPCSSRIRETEGWASVSKEGRDLG

SEQ ID NO: 35 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 36 - protein, 190 aa, Gorilla gorilla
MNPQIRNPMKAMYPGTFYFQFKNLWEANDRNETWLCFTVEGIKRRSVVSWKTGVFRNQVDSETHCHAERCFLSWECDDILSPNTNYQVTWYTSWSPCPECAGEVAEFLARHSNVNLTIFTARLYYFQDTDYQEGLRSLSQEGVAVKIMDYKDFKYCWENFVYNDDEPFKPWKGLKYNFRFLKRRLQEILE

SEQ ID NO: 37 - protein, 199 aa, Homo sapiens; identical to SEQ ID NO: 24

SEQ ID NO: 38 - protein, 202 aa, Macaca mulatta
MDGSPASRPRHLMDPNTFTFNFNNDLSVRGRHQTYLCYEVERLDNGTWVPMDERRGFLCNKAKNVPCGDYGCHVELRFLCEVPSWQLDPAQTYRVTWFISWSPCFRRGCAGQVRVFLQENKHVRLRIFAARIYDYDPLYQEALRTLRDAGAQVSIMTYEEFKHCWDTFVDRQGRPFQPWDGLDEHSQALSGRLRAILQNQGN

SEQ ID NO: 39 - protein, 185 aa, Bos sp.
MDEYTFTENFNNQGWPSKTYLCYEMERLDGDATIPLDEYKGFVRNKGLDQPEKPCHAELYFLGKIHSWNLDRNQHYRLTCFISWSPCYDCAQKLTTFLKENHHISLHILASRIYTHNRFGCHQSGLCELQAAGARITIMTFEDFKHCWETFVDHKGKPFQPWEGLNVKSQALCTELQAILKTQQN

SEQ ID NO: 40 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPKFADCWENFVDHEKPLSFNPYKMLEELDKNSRAIKRRLERIKIPGVRAQGRYMDILCDAEV

SEQ ID NO: 41 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 42 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 43 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 44 - protein, 229 aa, Mus sp.
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 45 - protein, 229 aa, Rattus sp. [sequence omitted]

SEQ ID NO: 46 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 47 - protein, 224 aa, Mus sp.
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 48 - protein, 224 aa, Rattus sp.
MAQKEEAAEAAAPASQNGDDLENLEDPEKLKELIDLPPFEIVTGVRLPVNFFKFQFRNVEYSSGRNKTFLCYVVEAQSKGGQVQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYLWQNFVEQEEGESKAFEPWEDIQENFLYYEEKLADILK

SEQ ID NO: 49 - protein, 224 aa, Bos sp.; identical to SEQ ID NO: 20

SEQ ID NO: 50 - protein, 208 aa, Petromyzon marinus
MTDAEYVRIHEKLDIYTFKKQFFNNKKSVSHRCYVLFELKRRGERRACFWGYAVNKPQSGTERGIHAEIFSIRKVEEYLRDNPGQFTINWYSSWSPCADCAEKILEWYNQELRGNGHTLKIWACKLYYEKNARNQIGLWNLRDNGVGLNVMVSEHYQCCRKIFIQSSHNQLNENRWLEKTLKRAEKRRSELSFMIQVKILHTTKSPAV

SEQ ID NO: 51 - protein, 381 aa, synthetic construct
  REGION 1..381: note = Description of Artificial Sequence: Syntheticpolypeptide
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKFNYDEFQHCWSKFVYSQRELFEPWNNLPKYYILLHFMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKKHVSLCIFTARIYRRQGRCQEGLRTLAEAGAKISFTYSEFKHCWDTFVDHQGCPFQPWDGLDEHSQDLSGRLRAILQNQEN

SEQ ID NO: 52 - protein, 182 aa, Homo sapiens
MDPPTFTFNFNNEPWWGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISFTYSEFKHCWDTFVDHQGCPFQPWDGLDEHSQDLSGRLRAILQ

SEQ ID NO: 53 - protein, 184 aa, synthetic construct
  REGION 1..184: note = Description of Artificial Sequence: Syntheticpolypeptide
MDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYRRQGRCQEGLRTLAEAGAKISFMTYSEFKHCWDTFVDHQGCPFQPWDGLDEHSQDLSGRLRAILQ

SEQ ID NO: 54 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 55 - protein, 198 aa, Mus musculus; identical to SEQ ID NO: 7

SEQ ID NO: 56 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 57 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 58 - protein, 224 aa, Petromyzon marinus
MAGYECVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLTMHFSRIYDRDREGDHRGLRGLKHVSNSFRMGVVGRAEVKECLAEYVEASRRTLTWLDTTESMAAKMRRKLFCILVRCAGMRESGIPLHLFTLQTPLLSGRVVWWRV

SEQ ID NO: 59 - protein, 331 aa, Petromyzon marinus
MELREVVDCALASCVRHEPLSRVAFLRCFAAPSQKPRGTVILFYVEGAGRGVTGGHAVNYNKQGTSIHAEVLLLSAVRAALLRRRRCEDGEEATRGCTLHCYSTYSPCRDCVEYIQEFGASTGVRVVIHCCRLYELDVNRRRSEAEGVLRSLSRLGRDFRLMGPRDAIALLLGGRLANTADGESGASGNAWVTETNVVEPLVDMTGFGDEDLHAQVQRNKQIREAYANYASAVSLMLGELHVDPDKFPFLAEFLAQTSVEPSGTPRETRGRPRGASSRGPEIGRQRPADFERALGAYGLFLHPRIVSREADREEIKRDLIVVMRKHNYQGP

SEQ ID NO: 60 - protein, 209 aa, Petromyzon marinus
MAGDENVRVSEKLDFDTFEFQFENLHYATERHRTYVIFDVKPQSAGGRSRRLWGYIINNPNVCHAELILMSMIDRHLESNPGVYAMTWYMSWSPCANCSSKLNPWLKNLLEEQGHTLMMHFSRIYDRDREGDHRGLRGLKHVSNSFRMGVVGRAEVKECLAEYVEASRRTLTWLDTTESMAAKMRRKLFCILVRCAGMRESGMPLHLFT

SEQ ID NO: 61 - protein, 158 aa, Saccharomyces cerevisiae
MVTGGMASKWDQKGMDIAYEEAALGYKEGGVPIGGCLINNKDGSVLGRGHNMRFQKGSATLHGEISTLENCGRLEGKVYKDTTLYTTLSPCDMCTGAIIMYGIPRCVVGENVNFKSKGEKYLQTRGHEVVVVDDERCKKIMKQFIDERPQDWFEDIGE

SEQ ID NO: 62 - protein, 218 aa, synthetic construct
  REGION 1..218: note = Description of Artificial Sequence: Syntheticpolypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRGLPPCLNILRRKQPQLTFFTIALQSCHYQRLPPHILWATGLK

SEQ ID NO: 63 - protein, 218 aa, synthetic construct
  REGION 1..218: note = Description of Artificial Sequence: Syntheticpolypeptide
MSSETGPVAVDPTLRRRIEPHEFEVFFDPRELRKETCLLYEINWGGRHSIWRHTSQNTNKHVEVNFIEKFTTERYFCPNTRCSITWFLSWSPCGECSRAITEFLSRYPHVTLFIYIARLYHHADPRNRQGLRDLISSGVTIQIMTEQESGYCWRNFVNYSPSNEAHWPRYPHLWVRLYVLELYCIILGLPPCLNILRRKQPQHYQRLPPHILWATGLK

SEQ ID NO: 64 - protein, 429 aa, Mus sp.; identical to SEQ ID NO: 22

SEQ ID NO: 65 - protein, 167 aa, synthetic construct
  REGION 1..167: note = Description of Artificial Sequence: Syntheticpolypeptide
MSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNAKTGAAGSLMDVLHYPGMNHRVEITEGILADECAALLCYFFRMPRQVFNAQKKAQSSTD

SEQ ID NO: 66 - protein, 1147 aa, unidentified
MAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYGERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMSGGSKRTADGSEFESPKKKRKVE

SEQ ID NO: 67 - protein, 1326 aa, synthetic construct
  REGION 1..1326: note = Description of Artificial Sequence: Syntheticpolypeptide
MSEVEFSHEYWMRHALTLAKRARDEREVPVGAVLVLNNRVIGEGWNRAIGLHDPTAHAEIMALRQGGLVMQNYRLYDATLYVTFEPCVMCAGAMIHSRIGRVVFGVRNAKTGAAGSLMDVLHHPGMNHRVEITEGILADECAALLCRFFRMPRRVFNAQKKAQSSTDGSSGSETPGTSESATPESSGAPKKKRKVGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIALGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYKERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQSMKRPAATKKAGQAKKKK

SEQ ID NO: 68 - protein, 1368 aa, Streptococcus pyogenes
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 69 - DNA, 20 bp, synthetic construct
  misc_feature 1..20: note = Description of Artificial Sequence: Syntheticoligonucleotide
  source 1..20: mol_type = other DNA
actcacgctggatagcctcc

SEQ ID NO: 70 - DNA, 20 bp, synthetic construct
  misc_feature 1..20: note = Description of Artificial Sequence: Syntheticoligonucleotide
  source 1..20: mol_type = other DNA
cttaccccacttaactatct

SEQ ID NO: 71 - DNA, 20 bp, synthetic construct
  misc_feature 1..20: note = Description of Artificial Sequence: Syntheticoligonucleotide
  source 1..20: mol_type = other DNA
cactcaccttagcctgagca

SEQ ID NO: 72 - DNA, 20 bp, synthetic construct
  misc_feature 1..20: note = Description of Artificial Sequence: Syntheticoligonucleotide
  source 1..20: mol_type = other DNA
cactcaccttagcctgagca

SEQ ID NO: 73 - DNA, 26 bp, synthetic construct
  misc_feature 1..26: note = Description of Artificial Sequence: Syntheticoligonucleotide
  source 1..26: mol_type = other DNA
tcctcaggtactccaaagattcaggt

SEQ ID NO: 74 - DNA, 23 bp, synthetic construct
  misc_feature 1..23: note = Description of Artificial Sequence: Syntheticoligonucleotide
  source 1..23: mol_type = other DNA
cgatctatgaaaaagacagtgga

SEQ ID NO: 75 - DNA, 23 bp, synthetic construct
  misc_feature 1..23: note = Description of Artificial Sequence: Syntheticoligonucleotide
  source 1..23: mol_type = other DNA
aaagaccagtccttgctgaaaga

SEQ ID NO: 76 - DNA, 23 bp, synthetic construct
  misc_feature 1..23: note = Description of Artificial Sequence: Syntheticoligonucleotide
  source 1..23: mol_type = other DNA
ggagtacctgaggaatatcggga

SEQ ID NO: 77 - DNA, 23 bp, synthetic construct
  misc_feature 1..23: note = Description of Artificial Sequence: Syntheticoligonucleotide
  source 1..23: mol_type = other DNA
ttcatagatcgagacatgtaagc

SEQ ID NO: 78 - DNA, 23 bp, synthetic construct
  misc_feature 1..23: note = Description of Artificial Sequence: Syntheticoligonucleotide
  source 1..23: mol_type = other DNA
ctcacgctggatagcctccaggc

SEQ ID NO: 79 - DNA, 23 bp, synthetic construct
  misc_feature 1..23: note = Description of Artificial Sequence: Syntheticoligonucleotide
  source 1..23: mol_type = other DNA
ttcatagatcgagacatgtaagc

SEQ ID NO: 80 - DNA, 23 bp, synthetic construct
  misc_feature 1..23: note = Description of Artificial Sequence: Syntheticoligonucleotide
  source 1..23: mol_type = other DNA
aggagagactcacgctggatagc

SEQ ID NO: 81 - DNA, 23 bp, synthetic construct
  misc_feature 1..23: note = Description of Artificial Sequence: Syntheticoligonucleotide
  source 1..23: mol_type = other DNA
ctcacgctggatagcctccaggc

SEQ ID NO: 82 - DNA, 23 bp, synthetic construct
  misc_feature 1..23: note = Description of Artificial Sequence: Syntheticoligonucleotide
  source 1..23: mol_type = other DNA
atagatcgagacatgtaagcagc

SEQ ID NO: 83 - DNA, 23 bp, synthetic construct
  misc_feature 1..23: note = Description of Artificial Sequence: Syntheticoligonucleotide
  source 1..23: mol_type = other DNA
taccccacttaactatcttgggc

SEQ ID NO: 84 - DNA, 23 bp, synthetic construct
  misc_feature 1..23: note = Description of Artificial Sequence: Syntheticoligonucleotide
  source 1..23: mol_type = other DNA
ctcacgctggatagcctccaggc

SEQ ID NO: 85 - DNA, 23 bp, synthetic construct
  misc_feature 1..23: note = Description of Artificial Sequence: Syntheticoligonucleotide
  source 1..23: mol_type = other DNA
ctcaggtactccaaagattcagg

SEQ ID NO: 86 - DNA, 23 bp, synthetic construct
  misc_feature 1..23: note = Description of Artificial Sequence: Syntheticoligonucleotide
  source 1..23: mol_type = other DNA
cttaccccacttaactatcttgg

SEQ ID NO: 87 - DNA, 23 bp, synthetic construct
  misc_feature 1..23: note = Description of Artificial Sequence: Syntheticoligonucleotide
  source 1..23: mol_type = other DNA
actcacgctggatagcctccagg

SEQ ID NO: 88 - DNA, 23 bp, synthetic construct
  misc_feature 1..23: note = Description of Artificial Sequence: Syntheticoligonucleotide
  source 1..23: mol_type = other DNA
ctcaggtactccaaagattcagg

SEQ ID NO: 89 - DNA, 23 bp, synthetic construct
  misc_feature 1..23: note = Description of Artificial Sequence: Syntheticoligonucleotide
  source 1..23: mol_type = other DNA
cttaccccacttaactatcttgg

SEQ ID NO: 90 - DNA, 23 bp, synthetic construct
  misc_feature 1..23: note = Description of Artificial Sequence: Syntheticoligonucleotide
  source 1..23: mol_type = other DNA
actcacgctggatagcctccagg

SEQ ID NO: 91 - DNA, 23 bp, synthetic construct
  misc_feature 1..23: note = Description of Artificial Sequence: Syntheticoligonucleotide
  source 1..23: mol_type = other DNA
tcgatctatgaaaaagacagtgg

SEQ ID NO: 92 - DNA, 23 bp, synthetic construct
  misc_feature 1..23: note = Description of Artificial Sequence: Syntheticoligonucleotide
  source 1..23: mol_type = other DNA
cttaccccacttaactatcttgg

SEQ ID NO: 93 - DNA, 23 bp, synthetic construct
  misc_feature 1..23: note = Description of Artificial Sequence: Syntheticoligonucleotide
  source 1..23: mol_type = other DNA
cttaccccacttaactatcttgg

SEQ ID NO: 94 - DNA, 23 bp, synthetic construct
  misc_feature 1..23: note = Description of Artificial Sequence: Syntheticoligonucleotide
  source 1..23: mol_type = other DNA
ttaccccacttaactatcttggg

SEQ ID NO: 95 - DNA, 23 bp, synthetic construct
  misc_feature 1..23: note = Description of Artificial Sequence: Syntheticoligonucleotide
  source 1..23: mol_type = other DNA
ttaccccacttaactatcttggg

SEQ ID NO: 96 - DNA, 23 bp, synthetic construct
  misc_feature 1..23: note = Description of Artificial Sequence: Syntheticoligonucleotide
  source 1..23: mol_type = other DNA
actcacgctggatagcctccagg

SEQ ID NO: 97 - RNA, 20 nt, synthetic construct
  misc_feature 1..20: note = Description of Artificial Sequence: Syntheticoligonucleotide
  source 1..20: mol_type = other RNA
tcctcaggtactccaaagat

SEQ ID NO: 98 - RNA, 20 nt, synthetic construct
  misc_feature 1..20: note = Description of Artificial Sequence: Syntheticoligonucleotide
  source 1..20: mol_type = other RNA
cgatctatgaaaaagacagt

SEQ ID NO: 99 - RNA, 20 nt, synthetic construct
  misc_feature 1..20: note = Description of Artificial Sequence: Syntheticoligonucleotide
  source 1..20: mol_type = other RNA
aaagaccagtccttgctgaa

SEQ ID NO: 100 - RNA, 20 nt, synthetic construct
  misc_feature 1..20: note = Description of Artificial Sequence: Syntheticoligonucleotide
  source 1..20: mol_type = other RNA
ggagtacctgaggaatatcg

SEQ ID NO: 101 - RNA, 20 nt, synthetic construct
  misc_feature 1..20: note = Description of Artificial Sequence: Syntheticoligonucleotide
  source 1..20: mol_type = other RNA
ttcatagatcgagacatgta

SEQ ID NO: 102 - RNA, 20 nt, synthetic construct
  misc_feature 1..20: note = Description of Artificial Sequence: Syntheticoligonucleotide
  source 1..20: mol_type = other RNA
ctcacgctggatagcctcca

SEQ ID NO: 103 - RNA, 20 nt, synthetic construct
  misc_feature 1..20: note = Description of Artificial Sequence: Syntheticoligonucleotide
  source 1..20: mol_type = other RNA
ttcatagatcgagacatgta

SEQ ID NO: 104 - RNA, 20 nt, synthetic construct
  misc_feature 1..20: note = Description of Artificial Sequence: Syntheticoligonucleotide
  source 1..20: mol_type = other RNA
aggagagactcacgctggat

SEQ ID NO: 105 - RNA, 20 nt, synthetic construct
  misc_feature 1..20: note = Description of Artificial Sequence: Syntheticoligonucleotide
  source 1..20: mol_type = other RNA
ctcacgctggatagcctcca

SEQ ID NO: 106 - RNA, 20 nt, synthetic construct
  misc_feature 1..20: note = Description of Artificial Sequence: Syntheticoligonucleotide
  source 1..20: mol_type = other RNA
atagatcgagacatgtaagc

SEQ ID NO: 107 - RNA, 20 nt, synthetic construct
  misc_feature 1..20: note = Description of Artificial Sequence: Syntheticoligonucleotide
  source 1..20: mol_type = other RNA
taccccacttaactatcttg

SEQ ID NO: 108 - RNA, 20 nt, synthetic construct
  misc_feature 1..20: note = Description of Artificial Sequence: Syntheticoligonucleotide
  source 1..20: mol_type = other RNA
ctcacgctggatagcctcca

SEQ ID NO: 109 - RNA, 20 nt, synthetic construct
  misc_feature 1..20: note = Description of Artificial Sequence: Syntheticoligonucleotide
  source 1..20: mol_type = other RNA
ctcaggtactccaaagattc

SEQ ID NO: 110 - RNA, 20 nt, synthetic construct
  misc_feature 1..20: note = Description of Artificial Sequence: Syntheticoligonucleotide
  source 1..20: mol_type = other RNA
cttaccccacttaactatct

SEQ ID NO: 111 - RNA, 20 nt, synthetic construct
  misc_feature 1..20: note = Description of Artificial Sequence: Syntheticoligonucleotide
  source 1..20: mol_type = other RNA
actcacgctggatagcctcc

SEQ ID NO: 112 - RNA, 20 nt, synthetic construct
  misc_feature 1..20: note = Description of Artificial Sequence: Syntheticoligonucleotide
  source 1..20: mol_type = other RNA
ctcaggtactccaaagattc

SEQ ID NO: 113 - RNA, 20 nt, synthetic construct
  misc_feature 1..20: note = Description of Artificial Sequence: Syntheticoligonucleotide
  source 1..20: mol_type = other RNA
cttaccccacttaactatct

SEQ ID NO: 114 - RNA, 20 nt, synthetic construct
  misc_feature 1..20: note = Description of Artificial Sequence: Syntheticoligonucleotide
  source 1..20: mol_type = other RNA
actcacgctggatagcctcc

SEQ ID NO: 115 - RNA, 20 nt, synthetic construct
  misc_feature 1..20: note = Description of Artificial Sequence: Syntheticoligonucleotide
  source 1..20: mol_type = other RNA
tcgatctatgaaaaagacag

SEQ ID NO: 116 - RNA, 20 nt, synthetic construct
  misc_feature 1..20: note = Description of Artificial Sequence: Syntheticoligonucleotide
  source 1..20: mol_type = other RNA
cttaccccacttaactatct

SEQ ID NO: 117 - RNA, 20 nt, synthetic construct
  misc_feature 1..20: note = Description of Artificial Sequence: Syntheticoligonucleotide
  source 1..20: mol_type = other RNA
cttaccccacttaactatct

SEQ ID NO: 118 - RNA, 20 nt, synthetic construct
  misc_feature 1..20: note = Description of Artificial Sequence: Syntheticoligonucleotide
  source 1..20: mol_type = other RNA
ttaccccacttaactatctt

SEQ ID NO: 119 - RNA, 20 nt, synthetic construct
  misc_feature 1..20: note = Description of Artificial Sequence: Syntheticoligonucleotide
  source 1..20: mol_type = other RNA
ttaccccacttaactatctt

SEQ ID NO: 120 - RNA, 20 nt, synthetic construct
  misc_feature 1..20: note = Description of Artificial Sequence: Syntheticoligonucleotide
  source 1..20: mol_type = other RNA
actcacgctggatagcctcc

SEQ ID NO: 121 - DNA, 24 bp, synthetic construct
  misc_feature 1..24: note = Description of Artificial Sequence: Syntheticoligonucleotide
  source 1..24: mol_type = other DNA
gttgtttaaaggcactacaaatac

SEQ ID NO: 122 - DNA, 26 bp, synthetic construct
  misc_feature 1..26: note = Description of Artificial Sequence: Syntheticoligonucleotide
  source 1..26: mol_type = other DNA
tgctcacctttcctgaacttgaagat

SEQ ID NO: 123 - DNA, 26 bp, synthetic construct
  misc_feature 1..26: note = Description of Artificial Sequence: Syntheticoligonucleotide
  source 1..26: mol_type = other DNA
ctttctttagcactaagtcaggagat

SEQ ID NO: 124 - DNA, 23 bp, synthetic construct
  misc_feature 1..23: note = Description of Artificial Sequence: Syntheticoligonucleotide
  source 1..23: mol_type = other DNA
atgctcacctttcctgaacttga

SEQ ID NO: 125 - DNA, 23 bp, synthetic construct
  misc_feature 1..23: note = Description of Artificial Sequence: Syntheticoligonucleotide
  source 1..23: mol_type = other DNA
ctcacctttcctgaacttgaaga

SEQ ID NO: 126 - DNA, 23 bp, synthetic construct
  misc_feature 1..23: note = Description of Artificial Sequence: Syntheticoligonucleotide
  source 1..23: mol_type = other DNA
cttactctccaggtaaggtgtga

SEQ ID NO: 127 - DNA, 23 bp, synthetic construct
  misc_feature 1..23: note = Description of Artificial Sequence: Syntheticoligonucleotide
  source 1..23: mol_type = other DNA
ttctttagcactaagtcaggaga

SEQ ID NO: 128 - DNA, 23 bp, synthetic construct
  misc_feature 1..23: note = Description of Artificial Sequence: Syntheticoligonucleotide
  source 1..23: mol_type = other DNA
atgctcacctttcctgaacttga

SEQ ID NO: 129 - DNA, 23 bp, synthetic construct
  misc_feature 1..23: note = Description of Artificial Sequence: Syntheticoligonucleotide
  source 1..23: mol_type = other DNA
ctcacctttcctgaacttgaaga

SEQ ID NO: 130 - DNA, 23 bp, synthetic construct
  misc_feature 1..23: note = Description of Artificial Sequence: Syntheticoligonucleotide
  source 1..23: mol_type = other DNA
gtttgctgaaacaaaggaaatga

SEQ ID NO: 131 - DNA, 23 bp, synthetic construct
  misc_feature 1..23: note = Description of Artificial Sequence: Syntheticoligonucleotide
  source 1..23: mol_type = other DNA
cttactctccaggtaaggtgtga

SEQ ID NO: 132 - DNA, 23 bp, synthetic construct
  misc_feature 1..23: note = Description of Artificial Sequence: Syntheticoligonucleotide
  source 1..23: mol_type = other DNA
cttagtgctaaagaaagaaaaga

SEQ ID NO: 133 - DNA, 23 bp, synthetic construct
  misc_feature 1..23: note = Description of Artificial Sequence: Syntheticoligonucleotide
  source 1..23: mol_type = other DNA
gtgctaaagaaagaaaagaagga

SEQ ID NO: 134 - DNA, 23 bp, synthetic construct
  misc_feature 1..23: note = Description of Artificial Sequence: Syntheticoligonucleotide
  source 1..23: mol_type = other DNA
ttaccttatttgaacagtgtaga

SEQ ID NO: 135 - DNA, 23 bp, synthetic construct
  misc_feature 1..23: note = Description of Artificial Sequence: Syntheticoligonucleotide
  source 1..23: mol_type = other DNA
ttcccactccaaaattgtcttga

SEQ ID NO: 136 - DNA, 23 bp, synthetic construct
  misc_feature 1..23: note = Description of Artificial Sequence: Syntheticoligonucleotide
  source 1..23: mol_type = other DNA
gtagtgcctttaaacaacagaga

SEQ ID NO: 137 - DNA, 23 bp, synthetic construct
  misc_feature 1..23: note = Description of Artificial Sequence: Syntheticoligonucleotide
  source 1..23: mol_type = other DNA
cctcggacagccaacaattcaga

SEQ ID NO: 138 - DNA, 23 bp, synthetic construct
  misc_feature 1..23: note = Description of Artificial Sequence: Syntheticoligonucleotide
  source 1..23: mol_type = other DNA
tgaacaggtgggaacaaaagtga

SEQ ID NO: 139 - DNA, 23 bp, synthetic construct
  misc_feature 1..23: note = Description of Artificial Sequence: Syntheticoligonucleotide
  source 1..23: mol_type = other DNA
tccctcccgaacagttaaccgga

SEQ ID NO: 140 - DNA, 23 bp, synthetic construct
  misc_feature 1..23: note = Description of Artificial Sequence: Syntheticoligonucleotide
  source 1..23: mol_type = other DNA
ctcccgaacagttaaccggaaga

SEQ ID NO: 141 - DNA, 23 bp, synthetic construct
  misc_feature 1..23: note = Description of Artificial Sequence: Syntheticoligonucleotide
  source 1..23: mol_type = other DNA
agtggggcagagctggaaggaga

SEQ ID NO: 142 - DNA, 23 bp, synthetic construct
  misc_feature 1..23: note = Description of Artificial Sequence: Syntheticoligonucleotide
  source 1..23: mol_type = other DNA
tgtttcagcaaacctcggacagc

SEQ ID NO: 143 - DNA, 23 bp, synthetic construct
  misc_feature 1..23: note = Description of Artificial Sequence: Syntheticoligonucleotide
  source 1..23: mol_type = other DNA
tgtttcagcaaacctcggacagc

SEQ ID NO: 144 - DNA, 23 bp, synthetic construct
  misc_feature 1..23: note = Description of Artificial Sequence: Syntheticoligonucleotide
  source 1..23: mol_type = other DNA
tgccactcacctgaagcgccggc

SEQ ID NO: 145 - DNA, 23 bp, synthetic construct
  misc_feature 1..23: note = Description of Artificial Sequence: Syntheticoligonucleotide
  source 1..23: mol_type = other DNA
tgtttcagcaaacctcggacagc

SEQ ID NO: 146 - DNA, 23 bp, synthetic construct
  misc_feature 1..23: note = Description of Artificial Sequence: Syntheticoligonucleotide
  source 1..23: mol_type = other DNA
ttccagctctgccccactcctgc

SEQ ID NO: 147 - DNA, 23 bp, synthetic construct
  misc_feature 1..23: note = Description of Artificial Sequence: Syntheticoligonucleotide
  source 1..23: mol_type = other DNA
aatatttctgaaaaataaagggc

SEQ ID NO: 148 - DNA, 23 bp, synthetic construct
  misc_feature 1..23: note = Description of Artificial Sequence: Syntheticoligonucleotide
  source 1..23: mol_type = other DNA
ttgctgaaacaaaggaaatgagc

SEQ ID NO: 149 - DNA, 23 bp, synthetic construct
  misc_feature 1..23: note = Description of Artificial Sequence: Syntheticoligonucleotide
  source 1..23: mol_type = other DNA
tttccaatctcctgacttagtgc

SEQ ID NO: 150 - DNA, 23 bp, synthetic construct
  misc_feature 1..23: note = Description of Artificial Sequence: Syntheticoligonucleotide
  source 1..23: mol_type = other DNA
gatttccaagttaaattatatgc

SEQ ID NO: 151 - DNA, 23 bp, synthetic construct
  misc_feature 1..23: note = Description of Artificial Sequence: Syntheticoligonucleotide
  source 1..23: mol_type = other DNA
ttccaagttaaattatatgctgc

SEQ ID NO: 152 - DNA, 23 bp, synthetic construct
  misc_feature 1..23: note = Description of Artificial Sequence: Syntheticoligonucleotide
  source 1..23: mol_type = other DNA
cgaagacccagccgagcaggagc

SEQ ID NO: 153 - DNA, 23 bp, synthetic construct
  misc_feature 1..23: note = Description of Artificial Sequence: Syntheticoligonucleotide
  source 1..23: mol_type = other DNA
taaaggcactacaaatactgtgg

SEQ ID NO: 154 - DNA, 23 bp, synthetic construct
  misc_feature 1..23: note = Description of Artificial Sequence: Syntheticoligonucleotide
  source 1..23: mol_type = other DNA
agccacttactctccaggtaagg

SEQ ID NO: 155 - DNA, 23 bp, synthetic construct
  misc_feature 1..23: note = Description of Artificial Sequence: Syntheticoligonucleotide
  source 1..23: mol_type = other DNA
gtgccactcacctgaagcgccgg

SEQ ID NO: 156 - DNA, 23 bp, synthetic construct
  misc_feature 1..23: note = Description of Artificial Sequence: Syntheticoligonucleotide
  source 1..23: mol_type = other DNA
taaaggcactacaaatactgtgg

SEQ ID NO: 157 - DNA, 23 bp, synthetic construct
  misc_feature 1..23: note = Description of Artificial Sequence: Syntheticoligonucleotide
  source 1..23: mol_type = other DNA
tctttctttagcactaagtcagg

SEQ ID NO: 158 - DNA, 23 bp, synthetic construct
  misc_feature 1..23: note = Description of Artificial Sequence: Syntheticoligonucleotide
  source 1..23: mol_type = other DNA
tcctttgtttcagcaaacctcgg

SEQ ID NO: 159 - DNA, 23 bp, synthetic construct
  misc_feature 1..23: note = Description of Artificial Sequence: Syntheticoligonucleotide
  source 1..23: mol_type = other DNA
agtgctaaagaaagaaaagaagg

SEQ ID NO: 160 - DNA, 23 bp, synthetic construct
  misc_feature 1..23: note = Description of Artificial Sequence: Syntheticoligonucleotide
  source 1..23: mol_type = other DNA
gcccaggtggccggcgcttcagg

SEQ ID NO: 161 - DNA, 23 bp, synthetic construct
  misc_feature 1..23: note = Description of Artificial Sequence: Syntheticoligonucleotide
  source 1..23: mol_type = other DNA
actgttcaaataaggtaagctgg

SEQ ID NO: 162 - DNA, 23 bp, synthetic construct
  misc_feature 1..23: note = Description of Artificial Sequence: Syntheticoligonucleotide
  source 1..23: mol_type = other DNA
ctgttcaaataaggtaagctggg

SEQ ID NO: 163 - DNA, 23 bp, synthetic construct
  misc_feature 1..23: note = Description of Artificial Sequence: Syntheticoligonucleotide
  source 1..23: mol_type = other DNA
tgaagcagacgtacttggcacgg

SEQ ID NO: 164 - DNA, 23 bp, synthetic construct
  misc_feature 1..23: note = Description of Artificial Sequence: Syntheticoligonucleotide
  source 1..23: mol_type = other DNA
gaagcagacgtacttggcacggg

SEQ ID NO: 165 - DNA, 23 bp, synthetic construct
  misc_feature 1..23: note = Description of Artificial Sequence: Syntheticoligonucleotide
  source 1..23: mol_type = other DNA
aacaattcagagttttgaacagg

SEQ ID NO: 166 - DNA, 23 bp, synthetic construct
  misc_feature 1..23: note = Description of Artificial Sequence: Syntheticoligonucleotide
  source 1..23: mol_type = other DNA
aattcagagttttgaacaggtgg

SEQ ID NO: 167 - DNA, 23 bp, synthetic construct
  misc_feature 1..23: note = Description of Artificial Sequence: Syntheticoligonucleotide
  source 1..23: mol_type = other DNA
attcagagttttgaacaggtggg

SEQ ID NO: 168 - RNA, 20 nt, synthetic construct
  misc_feature 1..20: note = Description of Artificial Sequence: Syntheticoligonucleotide
  source 1..20: mol_type = other RNA
tttaaaggcactacaaatac

SEQ ID NO: 169 - RNA, 20 nt, synthetic construct
  misc_feature 1..20: note = Description of Artificial Sequence: Syntheticoligonucleotide
  source 1..20: mol_type = other RNA
tgctcacctttcctgaactt

SEQ ID NO: 170 - RNA, 20 nt, synthetic construct
  misc_feature 1..20: note = Description of Artificial Sequence: Syntheticoligonucleotide
  source 1..20: mol_type = other RNA
ctttctttagcactaagtca

SEQ ID NO: 171 - RNA, 20 nt, synthetic construct
  misc_feature 1..20: note = Description of Artificial Sequence: Syntheticoligonucleotide
  source 1..20: mol_type = other RNA
atgctcacctttcctgaact

SEQ ID NO: 172 - RNA, 20 nt, synthetic construct
  misc_feature 1..20: note = Description of Artificial Sequence: Syntheticoligonucleotide
  source 1..20: mol_type = other RNA
ctcacctttcctgaacttga

SEQ ID NO: 173 - RNA, 20 nt, synthetic construct
  misc_feature 1..20: note = Description of Artificial Sequence: Syntheticoligonucleotide
  source 1..20: mol_type = other RNA
cttactctccaggtaaggtg

SEQ ID NO: 174 - RNA, 20 nt, synthetic construct
  misc_feature 1..20: note = Description of Artificial Sequence: Syntheticoligonucleotide
  source 1..20: mol_type = other RNA
ttctttagcactaagtcagg

SEQ ID NO: 175 - RNA, 20 nt, synthetic construct
  misc_feature 1..20: note = Description of Artificial Sequence: Syntheticoligonucleotide
  source 1..20: mol_type = other RNA
atgctcacctttcctgaact

SEQ ID NO: 176 - RNA, 20 nt, synthetic construct
  misc_feature 1..20: note = Description of Artificial Sequence: Syntheticoligonucleotide
  source 1..20: mol_type = other RNA
ctcacctttcctgaacttga

SEQ ID NO: 177 - RNA, 20 nt, synthetic construct
  misc_feature 1..20: note = Description of Artificial Sequence: Syntheticoligonucleotide
  source 1..20: mol_type = other RNA
gtttgctgaaacaaaggaaa

SEQ ID NO: 178 - RNA, 20 nt, synthetic construct
  misc_feature 1..20: note = Description of Artificial Sequence: Syntheticoligonucleotide
  source 1..20: mol_type = other RNA
cttactctccaggtaaggtg

SEQ ID NO: 179 - RNA, 20 nt, synthetic construct
  misc_feature 1..20: note = Description of Artificial Sequence: Syntheticoligonucleotide
  source 1..20: mol_type = other RNA
cttagtgctaaagaaagaaa

SEQ ID NO: 180 - RNA, 20 nt, synthetic construct
  misc_feature 1..20: note = Description of Artificial Sequence: Syntheticoligonucleotide
  source 1..20: mol_type = other RNA
gtgctaaagaaagaaaagaa

SEQ ID NO: 181 - RNA, 20 nt, synthetic construct
  misc_feature 1..20: note = Description of Artificial Sequence: Syntheticoligonucleotide
  source 1..20: mol_type = other RNA
ttaccttatttgaacagtgt

SEQ ID NO: 182 - RNA, 20 nt, synthetic construct
  misc_feature 1..20: note = Description of Artificial Sequence: Syntheticoligonucleotide
  source 1..20: mol_type = other RNA
ttcccactccaaaattgtct

SEQ ID NO: 183 - RNA, 20 nt, synthetic construct
  misc_feature 1..20: note = Description of Artificial Sequence: Syntheticoligonucleotide
  source 1..20: mol_type = other RNA
gtagtgcctttaaacaacag

SEQ ID NO: 184 - RNA, 20 nt, synthetic construct
  misc_feature 1..20: note = Description of Artificial Sequence: Syntheticoligonucleotide
  source 1..20: mol_type = other RNA
cctcggacagccaacaattc

SEQ ID NO: 185 - RNA, 20 nt, synthetic construct
  misc_feature 1..20: note = Description of Artificial Sequence: Syntheticoligonucleotide
  source 1..20: mol_type = other RNA
tgaacaggtgggaacaaaag

SEQ ID NO: 186 - RNA, 20 nt, synthetic construct
  misc_feature 1..20: note = Description of Artificial Sequence: Syntheticoligonucleotide
  source 1..20: mol_type = other RNA
tccctcccgaacagttaacc

SEQ ID NO: 187 - RNA, 20 nt, synthetic construct
  misc_feature 1..20: note = Description of Artificial Sequence: Syntheticoligonucleotide
  source 1..20: mol_type = other RNA
ctcccgaacagttaaccgga

SEQ ID NO: 188 - RNA, 20 nt, synthetic construct
  misc_feature 1..20: note = Description of Artificial Sequence: Syntheticoligonucleotide
  source 1..20: mol_type = other RNA
agtggggcagagctggaagg

SEQ ID NO: 189 - RNA, 20 nt, synthetic construct
  misc_feature 1..20: note = Description of Artificial Sequence: Syntheticoligonucleotide
  source 1..20: mol_type = other RNA
tgtttcagcaaacctcggac

SEQ ID NO: 190 - RNA, 20 nt, synthetic construct
  misc_feature 1..20: note = Description of Artificial Sequence: Syntheticoligonucleotide
  source 1..20: mol_type = other RNA
tgccactcacctgaagcgcc

SEQ ID NO: 191 - RNA, 20 nt, synthetic construct
  misc_feature 1..20: note = Description of Artificial Sequence: Syntheticoligonucleotide
  source 1..20: mol_type = other RNA
tgtttcagcaaacctcggac

SEQ ID NO: 192 - RNA, 20 nt, synthetic construct
  misc_feature 1..20: note = Description of Artificial Sequence: Syntheticoligonucleotide
  source 1..20: mol_type = other RNA
ttccagctctgccccactcc

SEQ ID NO: 193 - RNA, 20 nt, synthetic construct
  misc_feature 1..20: note = Description of Artificial Sequence: Syntheticoligonucleotide
  source 1..20: mol_type = other RNA
aatatttctgaaaaataaag

SEQ ID NO: 194 - RNA, 20 nt, synthetic construct
  misc_feature 1..20: note = Description of Artificial Sequence: Syntheticoligonucleotide
  source 1..20: mol_type = other RNA
ttgctgaaacaaaggaaatg

SEQ ID NO: 195 - RNA, 20 nt, synthetic construct
  misc_feature 1..20: note = Description of Artificial Sequence: Syntheticoligonucleotide
  source 1..20: mol_type = other RNA
tttttaatctcctgacttag

SEQ ID NO: 196 - skipped

SEQ ID NO: 197 - RNA, 20 nt, synthetic construct
  misc_feature 1..20: note = Description of Artificial Sequence: Syntheticoligonucleotide
  source 1..20: mol_type = other RNA
gatttccaagttaaattata

SEQ ID NO: 198 - RNA, 20 nt, synthetic construct
  misc_feature 1..20: note = Description of Artificial Sequence: Syntheticoligonucleotide
  source 1..20: mol_type = other RNA
ttccaagttaaattatatgc

SEQ ID NO: 199 - RNA, 20 nt, synthetic construct
  misc_feature 1..20: note = Description of Artificial Sequence: Syntheticoligonucleotide
  source 1..20: mol_type = other RNA
cgaagacccagccgagcagg

SEQ ID NO: 200 - RNA, 20 nt, synthetic construct
  misc_feature 1..20: note = Description of Artificial Sequence: Syntheticoligonucleotide
  source 1..20: mol_type = other RNA
taaaggcactacaaatactg

SEQ ID NO: 201 - RNA, 20 nt, synthetic construct
  misc_feature 1..20: note = Description of Artificial Sequence: Syntheticoligonucleotide
  source 1..20: mol_type = other RNA
agccacttactctccaggta

SEQ ID NO: 202 - RNA, 20 nt, synthetic construct
  misc_feature 1..20: note = Description of Artificial Sequence: Syntheticoligonucleotide
  source 1..20: mol_type = other RNA
gtgccactcacctgaagcgc

SEQ ID NO: 203 - RNA, 20 nt, synthetic construct
  misc_feature 1..20: note = Description of Artificial Sequence: Syntheticoligonucleotide
  source 1..20: mol_type = other RNA
taaaggcactacaaatactg

SEQ ID NO: 204 - RNA, 20 nt, synthetic construct
  misc_feature 1..20: note = Description of Artificial Sequence: Syntheticoligonucleotide
  source 1..20: mol_type = other RNA
tctttctttagcactaagtc

SEQ ID NO: 205 - RNA, 20 nt, synthetic construct
  misc_feature 1..20: note = Description of Artificial Sequence: Syntheticoligonucleotide
  source 1..20: mol_type = other RNA
tcctttgtttcagcaaacct

SEQ ID NO: 206 - RNA, 20 nt, synthetic construct
  misc_feature 1..20: note = Description of Artificial Sequence: Syntheticoligonucleotide
  source 1..20: mol_type = other RNA
agtgctaaagaaagaaaaga

SEQ ID NO: 207 - RNA, 20 nt, synthetic construct
  misc_feature 1..20: note = Description of Artificial Sequence: Syntheticoligonucleotide
  source 1..20: mol_type = other RNA
gcccaggtggccggcgcttc

SEQ ID NO: 208 - RNA, 20 nt, synthetic construct
  misc_feature 1..20: note = Description of Artificial Sequence: Syntheticoligonucleotide
  source 1..20: mol_type = other RNA
actgttcaaataaggtaagc

SEQ ID NO: 209 - RNA, 20 nt, synthetic construct
  misc_feature 1..20: note = Description of Artificial Sequence: Syntheticoligonucleotide
  source 1..20: mol_type = other RNA
ctgttcaaataaggtaagct

SEQ ID NO: 210 - RNA, 20 nt, synthetic construct
  misc_feature 1..20: note = Description of Artificial Sequence: Syntheticoligonucleotide
  source 1..20: mol_type = other RNA
tgaagcagacgtacttggca

SEQ ID NO: 211 - RNA, 20 nt, synthetic construct
  misc_feature 1..20: note = Description of Artificial Sequence: Syntheticoligonucleotide
  source 1..20: mol_type = other RNA
gaagcagacgtacttggcac

SEQ ID NO: 212 - RNA, 20 nt, synthetic construct
  misc_feature 1..20: note = Description of Artificial Sequence: Syntheticoligonucleotide
  source 1..20: mol_type = other RNA
aacaattcagagttttgaac

SEQ ID NO: 213 - RNA, 20 nt, synthetic construct
  misc_feature 1..20: note = Description of Artificial Sequence: Syntheticoligonucleotide
  source 1..20: mol_type = other RNA
aattcagagttttgaacagg

SEQ ID NO: 214 - RNA, 20 nt, synthetic construct
  misc_feature 1..20: note = Description of Artificial Sequence: Syntheticoligonucleotide
  source 1..20: mol_type = other RNA
attcagagttttgaacaggt

SEQ ID NO: 215 - DNA, 24 bp, synthetic construct
  misc_feature 1..24: note = Description of Artificial Sequence: Syntheticoligonucleotide
  source 1..24: mol_type = other DNA
gttttctctgcagccttcccagag

SEQ ID NO: 216 - DNA, 24 bp, synthetic construct
  misc_feature 1..24: note = Description of Artificial Sequence: Syntheticoligonucleotide
  source 1..24: mol_type = other DNA
gttctctccaggacgagaagttcc

SEQ ID NO: 217 - DNA, 24 bp, synthetic construct
  misc_feature 1..24: note = Description of Artificial Sequence: Syntheticoligonucleotide
  source 1..24: mol_type = other DNA
attccacctgcagcctggatgcgc

SEQ ID NO: 218 - DNA, 24 bp, synthetic construct
  misc_feature 1..24: note = Description of Artificial Sequence: Syntheticoligonucleotide
  source 1..24: mol_type = other DNA
gtttccgacagcttgtacaataac

SEQ ID NO: 219 - DNA, 24 bp, synthetic construct
  misc_feature 1..24: note = Description of Artificial Sequence: Syntheticoligonucleotide
  source 1..24: mol_type = other DNA
gtttctcttgccagcgtccagtac

SEQ ID NO: 220 - DNA, 26 bp, synthetic construct
  misc_feature 1..26: note = Description of Artificial Sequence: Syntheticoligonucleotide
  source 1..26: mol_type = other DNA
tgtctgggcagcggaactggaccagt

SEQ ID NO: 221 - DNA, 26 bp, synthetic construct
  misc_feature 1..26: note = Description of Artificial Sequence: Syntheticoligonucleotide
  source 1..26: mol_type = other DNA
tcaaagtagagcacataggaccagat

SEQ ID NO: 222 - DNA, 26 bp, synthetic construct
  misc_feature 1..26: note = Description of Artificial Sequence: Syntheticoligonucleotide
  source 1..26: mol_type = other DNA
ctcacagctgagccccccactgtggt

SEQ ID NO: 223 - DNA, 26 bp, synthetic construct
  misc_feature 1..26: note = Description of Artificial Sequence: Syntheticoligonucleotide
  source 1..26: mol_type = other DNA
ggcctttgcagagccggtggagcagt

SEQ ID NO: 224 - DNA, 26 bp, synthetic construct
  misc_feature 1..26: note = Description of Artificial Sequence: Syntheticoligonucleotide
  source 1..26: mol_type = other DNA
ccacctgcagcctggatgcgctgagt

SEQ ID NO: 225 - DNA, 26 bp, synthetic construct
  misc_feature 1..26: note = Description of Artificial Sequence: Syntheticoligonucleotide
  source 1..26: mol_type = other DNA
tcttgccagcgtccagtacaacaagt

SEQ ID NO: 226 - DNA, 26 bp, synthetic construct
  misc_feature 1..26: note = Description of Artificial Sequence: Syntheticoligonucleotide
  source 1..26: mol_type = other DNA
ccactcaccttagcctgagcagggat

SEQ ID NO: 227 - DNA, 26 bp, synthetic construct
  misc_feature 1..26: note = Description of Artificial Sequence: Syntheticoligonucleotide
  source 1..26: mol_type = other DNA
cctaacatactgggaatctggtcggt

SEQ ID NO: 228 - DNA, 26 bp, synthetic construct
  misc_feature 1..26: note = Description of Artificial Sequence: Syntheticoligonucleotide
  source 1..26: mol_type = other DNA
gagggcccacctgagtagagctcaat

SEQ ID NO: 229 - DNA, 26 bp, synthetic construct
  misc_feature 1..26: note = Description of Artificial Sequence: Syntheticoligonucleotide
  source 1..26: mol_type = other DNA
ctttttaccttggggctctgacaggt

SEQ ID NO: 230 - DNA, 23 bp, synthetic construct
  misc_feature 1..23: note = Description of Artificial Sequence: Syntheticoligonucleotide
  source 1..23: mol_type = other DNA
aaagtagagcacataggaccaga

SEQ ID NO: 231 - DNA, 23 bp, synthetic construct
  misc_feature 1..23: note = Description of Artificial Sequence: Syntheticoligonucleotide
  source 1..23: mol_type = other DNA
ctccacagggctgccttgagcga

SEQ ID NO: 232 - DNA, 23 bp, synthetic construct
  misc_feature 1..23: note = Description of Artificial Sequence: Syntheticoligonucleotide
  source 1..23: mol_type = other DNA
tccaggacgagaagttcctcgga

SEQ ID NO: 233 - DNA, 23 bp, synthetic construct
  misc_feature 1..23: note = Description of Artificial Sequence: Syntheticoligonucleotide
  source 1..23: mol_type = other DNA
cacctgcagcctggatgcgctga

SEQ ID NO: 234 - DNA, 23 bp, synthetic construct
  misc_feature 1..23: note = Description of Artificial Sequence: Syntheticoligonucleotide
  source 1..23: mol_type = other DNA
tgcagcctggatgcgctgagtga

SEQ ID NO: 235 - DNA, 23 bp, synthetic construct
  misc_feature 1..23: note = Description of Artificial Sequence: Syntheticoligonucleotide
  source 1..23: mol_type = other DNA
cttacgccagcgtctccacatga

SEQ ID NO: 236 - DNA, 23 bp, synthetic construct
  misc_feature 1..23: note = Description of Artificial Sequence: Syntheticoligonucleotide
  source 1..23: mol_type = other DNA
actcactccatcacccggaggga

SEQ ID NO: 237 - DNA, 23 bp, synthetic construct
  misc_feature 1..23: note = Description of Artificial Sequence: Syntheticoligonucleotide
  source 1..23: mol_type = other DNA
actcaccttagcctgagcaggga

SEQ ID NO: 238 - DNA, 23 bp, synthetic construct
  misc_feature 1..23: note = Description of Artificial Sequence: Syntheticoligonucleotide
  source 1..23: mol_type = other DNA
actcacttgagggtttccaagga

SEQ ID NO: 239 - DNA, 23 bp, synthetic construct
  misc_feature 1..23: note = Description of Artificial Sequence: Syntheticoligonucleotide
  source 1..23: mol_type = other DNA
actcactccagatgctgcaggga

SEQ ID NO: 240 - DNA, 23 bp, synthetic construct
  misc_feature 1..23: note = Description of Artificial Sequence: Syntheticoligonucleotide
  source 1..23: mol_type = other DNA
atcactcaccaggccattttgga

SEQ ID NO: 241 - DNA, 23 bp, synthetic construct
  misc_feature 1..23: note = Description of Artificial Sequence: Syntheticoligonucleotide
  source 1..23: mol_type = other DNA
gccccaaggtaaaaaggccggga

SEQ ID NO: 242 - DNA, 23 bp, synthetic construct
  misc_feature 1..23: note = Description of Artificial Sequence: Syntheticoligonucleotide
  source 1..23: mol_type = other DNA
agctcacagtgtgccaccatgga

SEQ ID NO: 243 - DNA, 23 bp, synthetic construct
  misc_feature 1..23: note = Description of Artificial Sequence: Syntheticoligonucleotide
  source 1..23: mol_type = other DNA
atgaccagatggacctggctgga

SEQ ID NO: 244 - DNA, 23 bp, synthetic construct
  misc_feature 1..23: note = Description of Artificial Sequence: Syntheticoligonucleotide
  source 1..23: mol_type = other DNA
gaccagatggacctggctggaga

SEQ ID NO: 245 - DNA, 23 bp, synthetic construct
  misc_feature 1..23: note = Description of Artificial Sequence: Syntheticoligonucleotide
  source 1..23: mol_type = other DNA
ctggaccagtatgtcttccagga

SEQ ID NO: 246 - DNA, 23 bp, synthetic construct
  misc_feature 1..23: note = Description of Artificial Sequence: Syntheticoligonucleotide
  source 1..23: mol_type = other DNA
gtcttccaggactcccagctgga

SEQ ID NO: 247 - DNA, 23 bp, synthetic construct
  misc_feature 1..23: note = Description of Artificial Sequence: Syntheticoligonucleotide
  source 1..23: mol_type = other DNA
ggactcccagctggagggcctga

SEQ ID NO: 248 - DNA, 23 bp, synthetic construct
  misc_feature 1..23: note = Description of Artificial Sequence: Syntheticoligonucleotide
  source 1..23: mol_type = other DNA
ttgggcagaaaagtcagaaaaga

SEQ ID NO: 249 - DNA, 23 bp, synthetic construct
  misc_feature 1..23: note = Description of Artificial Sequence: Syntheticoligonucleotide
  source 1..23: mol_type = other DNA
aaagtcagaaaagacgtgagtga

SEQ ID NO: 250 - DNA, 23 bp, synthetic construct
  misc_feature 1..23: note = Description of Artificial Sequence: Syntheticoligonucleotide
  source 1..23: mol_type = other DNA
ctccggccagatgcgcctggaga

SEQ ID NO: 251 - DNA, 23 bp, synthetic construct
  misc_feature 1..23: note = Description of Artificial Sequence: Syntheticoligonucleotide
  source 1..23: mol_type = other DNA
tctggcaaatctctgaggctgga

SEQ ID NO: 252 - DNA, 23 bp, synthetic construct
  misc_feature 1..23: note = Description of Artificial Sequence: Syntheticoligonucleotide
  source 1..23: mol_type = other DNA
ccacccaatgcccggcagctgga

SEQ ID NO: 253 - DNA, 23 bp, synthetic construct
  misc_feature 1..23: note = Description of Artificial Sequence: Syntheticoligonucleotide
  source 1..23: mol_type = other DNA
acccaatgcccggcagctggaga

SEQ ID NO: 254 - DNA, 23 bp, synthetic construct
  misc_feature 1..23: note = Description of Artificial Sequence: Syntheticoligonucleotide
  source 1..23: mol_type = other DNA
ctgcaggacacgtatggtgccga

SEQ ID NO: 255 - DNA, 23 bp, synthetic construct
  misc_feature 1..23: note = Description of Artificial Sequence: Syntheticoligonucleotide
  source 1..23: mol_type = other DNA
tctggtgcaggccaggctggaga

SEQ ID NO: 256 - DNA, 23 bp, synthetic construct
  misc_feature 1..23: note = Description of Artificial Sequence: Syntheticoligonucleotide
  source 1..23: mol_type = other DNA
ggtgcaggccaggctggagagga

SEQ ID NO: 257 - DNA, 23 bp, synthetic construct
  misc_feature 1..23: note = Description of Artificial Sequence: Syntheticoligonucleotide
  source 1..23: mol_type = other DNA
ctggcccaaggaggcctggctga

SEQ ID NO: 258 - DNA, 23 bp, synthetic construct
  misc_feature 1..23: note = Description of Artificial Sequence: Syntheticoligonucleotide
  source 1..23: mol_type = other DNA
ccacagccactcgtggcggccga

SEQ ID NO: 259 - DNA, 23 bp, synthetic construct
  misc_feature 1..23: note = Description of Artificial Sequence: Syntheticoligonucleotide
  source 1..23: mol_type = other DNA
ttttccagaagaagctgctccga

SEQ ID NO: 260 - DNA, 23 bp, synthetic construct
  misc_feature 1..23: note = Description of Artificial Sequence: Syntheticoligonucleotide
  source 1..23: mol_type = other DNA
gtccagagcctgagcaaggccga

SEQ ID NO: 261 - DNA, 23 bp, synthetic construct
  misc_feature 1..23: note = Description of Artificial Sequence: Syntheticoligonucleotide
  source 1..23: mol_type = other DNA
ggagcaggcccaggcatacgtga

SEQ ID NO: 262 - DNA, 23 bp, synthetic construct
  misc_feature 1..23: note = Description of Artificial Sequence: Syntheticoligonucleotide
  source 1..23: mol_type = other DNA
agagcaccaagacagagccctga

SEQ ID NO: 263 - DNA, 23 bp, synthetic construct
  misc_feature 1..23: note = Description of Artificial Sequence: Syntheticoligonucleotide
  source 1..23: mol_type = other DNA
agacatcaaagtaccctacagga

SEQ ID NO: 264 - DNA, 23 bp, synthetic construct
  misc_feature 1..23: note = Description of Artificial Sequence: Syntheticoligonucleotide
  source 1..23: mol_type = other DNA
catcaaagtaccctacaggagga

SEQ ID NO: 265 - DNA, 23 bp, synthetic construct
  misc_feature 1..23: note = Description of Artificial Sequence: Syntheticoligonucleotide
  source 1..23: mol_type = other DNA
gaggaccagttcccatccgcaga

SEQ ID NO: 266 - DNA, 23 bp, synthetic construct
  misc_feature 1..23: note = Description of Artificial Sequence: Syntheticoligonucleotide
  source 1..23: mol_type = other DNA
gctcccgcagtacctagcattga

SEQ ID NO: 267 - DNA, 23 bp, synthetic construct
  misc_feature 1..23: note = Description of Artificial Sequence: Syntheticoligonucleotide
  source 1..23: mol_type = other DNA
caggaagcagaaggtgcttgcga

SEQ ID NO: 268 - DNA, 23 bp, synthetic construct
  misc_feature 1..23: note = Description of Artificial Sequence: Syntheticoligonucleotide
  source 1..23: mol_type = other DNA
atttggcagcacgtggtacagga

SEQ ID NO: 269 - DNA, 23 bp, synthetic construct
  misc_feature 1..23: note = Description of Artificial Sequence: Syntheticoligonucleotide
  source 1..23: mol_type = other DNA
gcgggccaagacttctccctgga

SEQ ID NO: 270 - DNA, 23 bp, synthetic construct
  misc_feature 1..23: note = Description of Artificial Sequence: Syntheticoligonucleotide
  source 1..23: mol_type = other DNA
gtccctgcagcagcatggggaga

SEQ ID NO: 271 - DNA, 23 bp, synthetic construct
  misc_feature 1..23: note = Description of Artificial Sequence: Syntheticoligonucleotide
  source 1..23: mol_type = other DNA
gctacttcaggcagcagaggaga

SEQ ID NO: 272 - DNA, 23 bp, synthetic construct
  misc_feature 1..23: note = Description of Artificial Sequence: Syntheticoligonucleotide
  source 1..23: mol_type = other DNA
cttgtgcagactcagaggtgaga

SEQ ID NO: 273 - DNA, 23 bp, synthetic construct
  misc_feature 1..23: note = Description of Artificial Sequence: Syntheticoligonucleotide
  source 1..23: mol_type = other DNA
gtgcagactcagaggtgagagga

SEQ ID NO: 274 - DNA, 23 bp, synthetic construct
  misc_feature 1..23: note = Description of Artificial Sequence: Syntheticoligonucleotide
  source 1..23: mol_type = other DNA
gcagactcagaggtgagaggaga

SEQ ID NO: 275 - DNA, 23 bp, synthetic construct
  misc_feature 1..23: note = Description of Artificial Sequence: Syntheticoligonucleotide
  source 1..23: mol_type = other DNA
ttcccccagctgaagtccttgga

SEQ ID NO: 276 - DNA, 23 bp, synthetic construct
  misc_feature 1..23: note = Description of Artificial Sequence: Syntheticoligonucleotide
  source 1..23: mol_type = other DNA
ctgtcccagaacaacatcactga

SEQ ID NO: 277 - DNA, 23 bp, synthetic construct
  misc_feature 1..23: note = Description of Artificial Sequence: Syntheticoligonucleotide
  source 1..23: mol_type = other DNA
cctgcaacaacaggattcacgga

SEQ ID NO: 278 - DNA, 23 bp, synthetic construct
  misc_feature 1..23: note = Description of Artificial Sequence: Syntheticoligonucleotide
  source 1..23: mol_type = other DNA
cgtccacatcctgcaagggggga

SEQ ID NO: 279 - DNA, 23 bp, synthetic construct
  misc_feature 1..23: note = Description of Artificial Sequence: Syntheticoligonucleotide
  source 1..23: mol_type = other DNA
acatcctgcaaggggggatggga

SEQ ID NO: 280 - DNA, 23 bp, synthetic construct
  misc_feature 1..23: note = Description of Artificial Sequence: Syntheticoligonucleotide
  source 1..23: mol_type = other DNA
cttacgccagcgtctccacatga

SEQ ID NO: 281 - DNA, 23 bp, synthetic construct
  misc_feature 1..23: note = Description of Artificial Sequence: Syntheticoligonucleotide
  source 1..23: mol_type = other DNA
actcactccatcacccggaggga

SEQ ID NO: 282 - DNA, 23 bp, synthetic construct
  misc_feature 1..23: note = Description of Artificial Sequence: Syntheticoligonucleotide
  source 1..23: mol_type = other DNA
actcaccttagcctgagcaggga

SEQ ID NO: 283 - DNA, 23 bp, synthetic construct
  misc_feature 1..23: note = Description of Artificial Sequence: Syntheticoligonucleotide
  source 1..23: mol_type = other DNA
gacagactgcggggacacagtga

SEQ ID NO: 284 - DNA, 23 bp, synthetic construct
  misc_feature 1..23: note = Description of Artificial Sequence: Syntheticoligonucleotide
  source 1..23: mol_type = other DNA
actcacttgagggtttccaagga

SEQ ID NO: 285 - DNA, 23 bp, synthetic construct
  misc_feature 1..23: note = Description of Artificial Sequence: Syntheticoligonucleotide
  source 1..23: mol_type = other DNA
tccaggctgcaggtggaatcaga

SEQ ID NO: 286 - DNA, 23 bp, synthetic construct
  misc_feature 1..23: note = Description of Artificial Sequence: Syntheticoligonucleotide
  source 1..23: mol_type = other DNA
actcactccagatgctgcaggga

SEQ ID NO: 287 - DNA, 23 bp, synthetic construct
  misc_feature 1..23: note = Description of Artificial Sequence: Syntheticoligonucleotide
  source 1..23: mol_type = other DNA
agccagccacagggcccccagga

SEQ ID NO: 288 - DNA, 23 bp, synthetic construct
  misc_feature 1..23: note = Description of Artificial Sequence: Syntheticoligonucleotide
  source 1..23: mol_type = other DNA
gcccaggtcctcacgtctgcgga

SEQ ID NO: 289 - DNA, 23 bp, synthetic construct
  misc_feature 1..23: note = Description of Artificial Sequence: Syntheticoligonucleotide
  source 1..23: mol_type = other DNA
cagcccaatagctcttgccctga

SEQ ID NO: 290 - DNA, 23 bp, synthetic construct
  misc_feature 1..23: note = Description of Artificial Sequence: Syntheticoligonucleotide
  source 1..23: mol_type = other DNA
ggccattttggaagcttgttgga

SEQ ID NO: 291 - DNA, 23 bp, synthetic construct
  misc_feature 1..23: note = Description of Artificial Sequence: Syntheticoligonucleotide
  source 1..23: mol_type = other DNA
ttacctgtcatgtttgctcggga

SEQ ID NO: 292 - DNA, 23 bp, synthetic construct
  misc_feature 1..23: note = Description of Artificial Sequence: Syntheticoligonucleotide
  source 1..23: mol_type = other DNA
acctcacctacattgggggtgga

SEQ ID NO: 293 - DNA, 23 bp, synthetic construct
  misc_feature 1..23: note = Description of Artificial Sequence: Syntheticoligonucleotide
  source 1..23: mol_type = other DNA
ctcacctacattgggggtggaga

SEQ ID NO: 294 - DNA, 23 bp, synthetic construct
  misc_feature 1..23: note = Description of Artificial Sequence: Syntheticoligonucleotide
  source 1..23: mol_type = other DNA
tttgccagagcccatggggcaga

SEQ ID NO: 295 - DNA, 23 bp, synthetic construct
  misc_feature 1..23: note = Description of Artificial Sequence: Syntheticoligonucleotide
  source 1..23: mol_type = other DNA
aaggctgcagagaaaacatgtga

SEQ ID NO: 296 - DNA, 23 bp, synthetic construct
  misc_feature 1..23: note = Description of Artificial Sequence: Syntheticoligonucleotide
  source 1..23: mol_type = other DNA
gctctactttgagaaaaaccaga

SEQ ID NO: 297 - DNA, 23 bp, synthetic construct
  misc_feature 1..23: note = Description of Artificial Sequence: Syntheticoligonucleotide
  source 1..23: mol_type = other DNA
ctcccaggcagctcacagtgtgc

SEQ ID NO: 298 - DNA, 23 bp, synthetic construct
  misc_feature 1..23: note = Description of Artificial Sequence: Syntheticoligonucleotide
  source 1..23: mol_type = other DNA
ctctgcagccttcccagaggagc

SEQ ID NO: 299 - DNA, 23 bp, synthetic construct
  misc_feature 1..23: note = Description of Artificial Sequence: Syntheticoligonucleotide
  source 1..23: mol_type = other DNA
aatgtaggtgaggtgccccaggc

SEQ ID NO: 300 - DNA, 23 bp, synthetic construct
  misc_feature 1..23: note = Description of Artificial Sequence: Syntheticoligonucleotide
  source 1..23: mol_type = other DNA
ccgacagcttgtacaataactgc

SEQ ID NO: 301 - DNA, 23 bp, synthetic construct
  misc_feature 1..23: note = Description of Artificial Sequence: Syntheticoligonucleotide
  source 1..23: mol_type = other DNA
ctcacctctgagtctgcacaagc

SEQ ID NO: 302 - DNA, 23 bp, synthetic construct
  misc_feature 1..23: note = Description of Artificial Sequence: Syntheticoligonucleotide
  source 1..23: mol_type = other DNA
actcaccaggccattttggaagc

SEQ ID NO: 303 - DNA, 23 bp, synthetic construct
  misc_feature 1..23: note = Description of Artificial Sequence: Syntheticoligonucleotide
  source 1..23: mol_type = other DNA
cctgcacacctggcttccagtgc

SEQ ID NO: 304 - DNA, 23 bp, synthetic construct
  misc_feature 1..23: note = Description of Artificial Sequence: Syntheticoligonucleotide
  source 1..23: mol_type = other DNA
aaacttactgaaaatgtccttgc

SEQ ID NO: 305 - DNA, 23 bp, synthetic construct
  misc_feature 1..23: note = Description of Artificial Sequence: Syntheticoligonucleotide
  source 1..23: mol_type = other DNA
tcctcaccgatattggcataagc

SEQ ID NO: 306 - DNA, 23 bp, synthetic construct
  misc_feature 1..23: note = Description of Artificial Sequence: Syntheticoligonucleotide
  source 1..23: mol_type = other DNA
ctcccaggcagctcacagtgtgc

SEQ ID NO: 307 - DNA, 23 bp, synthetic construct
  misc_feature 1..23: note = Description of Artificial Sequence: Syntheticoligonucleotide
  source 1..23: mol_type = other DNA
ctctgcagccttcccagaggagc

SEQ ID NO: 308 - DNA, 23 bp, synthetic construct
  misc_feature 1..23: note = Description of Artificial Sequence: Syntheticoligonucleotide
  source 1..23: mol_type = other DNA
cacccccaatgtaggtgaggtgc

SEQ ID NO: 309 - DNA, 23 bp, synthetic construct
  misc_feature 1..23: note = Description of Artificial Sequence: Syntheticoligonucleotide
  source 1..23: mol_type = other DNA
aatgtaggtgaggtgccccaggc

SEQ ID NO: 310 - DNA, 23 bp, synthetic construct
  misc_feature 1..23: note = Description of Artificial Sequence: Syntheticoligonucleotide
  source 1..23: mol_type = other DNA
ggcctttgcagagccggtggagc

SEQ ID NO: 311 - DNA, 23 bp, synthetic construct
  misc_feature 1..23: note = Description of Artificial Sequence: Syntheticoligonucleotide
  source 1..23: mol_type = other DNA
ccctccacagggctgccttgagc

SEQ ID NO: 312 - DNA, 23 bp, synthetic construct
  misc_feature 1..23: note = Description of Artificial Sequence: Syntheticoligonucleotide
  source 1..23: mol_type = other DNA
gattccacctgcagcctggatgc

SEQ ID NO: 313 - DNA, 23 bp, synthetic construct
  misc_feature 1..23: note = Description of Artificial Sequence: Syntheticoligonucleotide
  source 1..23: mol_type = other DNA
ttccacctgcagcctggatgcgc

SEQ ID NO: 314 - DNA, 23 bp, synthetic construct
  misc_feature 1..23: note = Description of Artificial Sequence: Syntheticoligonucleotide
  source 1..23: mol_type = other DNA
ccgacagcttgtacaataactgc

SEQ ID NO: 315 - DNA, 23 bp, synthetic construct
  misc_feature 1..23: note = Description of Artificial Sequence: Syntheticoligonucleotide
  source 1..23: mol_type = other DNA
ccccccttgcaggatgtggacgc

SEQ ID NO: 316 - DNA, 23 bp, synthetic construct
  misc_feature 1..23: note = Description of Artificial Sequence: Syntheticoligonucleotide
  source 1..23: mol_type = other DNA
gccccactcaccttagcctgagc

SEQ ID NO: 317 - DNA, 23 bp, synthetic construct
  misc_feature 1..23: note = Description of Artificial Sequence: Syntheticoligonucleotide
  source 1..23: mol_type = other DNA
gagtctatactcactccagatgc

SEQ ID NO: 318 - DNA, 23 bp, synthetic construct
  misc_feature 1..23: note = Description of Artificial Sequence: Syntheticoligonucleotide
  source 1..23: mol_type = other DNA
tctatactcactccagatgctgc

SEQ ID NO: 319 - DNA, 23 bp, synthetic construct
  misc_feature 1..23: note = Description of Artificial Sequence: Syntheticoligonucleotide
  source 1..23: mol_type = other DNA
tctcctctcacctctgagtctgc

SEQ ID NO: 320 - DNA, 23 bp, synthetic construct
  misc_feature 1..23: note = Description of Artificial Sequence: Syntheticoligonucleotide
  source 1..23: mol_type = other DNA
ctcacctctgagtctgcacaagc

SEQ ID NO: 321 - DNA, 23 bp, synthetic construct
  misc_feature 1..23: note = Description of Artificial Sequence: Syntheticoligonucleotide
  source 1..23: mol_type = other DNA
actcaccaggccattttggaagc

SEQ ID NO: 322 - DNA, 23 bp, synthetic construct
  misc_feature 1..23: note = Description of Artificial Sequence: Syntheticoligonucleotide
  source 1..23: mol_type = other DNA
gggtccttacctgtcatgtttgc

SEQ ID NO: 323 - DNA, 23 bp, synthetic construct
  misc_feature 1..23: note = Description of Artificial Sequence: Syntheticoligonucleotide
  source 1..23: mol_type = other DNA
cctgcacacctggcttccagtgc

SEQ ID NO: 324 - DNA, 23 bp, synthetic construct
  misc_feature 1..23: note = Description of Artificial Sequence: Syntheticoligonucleotide
  source 1..23: mol_type = other DNA
aaacttactgaaaatgtccttgc

SEQ ID NO: 325 - DNA, 23 bp, synthetic construct
  misc_feature 1..23: note = Description of Artificial Sequence: Syntheticoligonucleotide
  source 1..23: mol_type = other DNA
ggtgcttcctcaccgatattggc

SEQ ID NO: 326 - DNA, 23 bp, synthetic construct
  misc_feature 1..23: note = Description of Artificial Sequence: Syntheticoligonucleotide
  source 1..23: mol_type = other DNA
tcctcaccgatattggcataagc

SEQ ID NO: 327 - DNA, 23 bp, synthetic construct
  misc_feature 1..23: note = Description of Artificial Sequence: Syntheticoligonucleotide
  source 1..23: mol_type = other DNA
aggagggcccacctgagtagagc

SEQ ID NO: 328 - DNA, 23 bp, synthetic construct
  misc_feature 1..23: note = Description of Artificial Sequence: Syntheticoligonucleotide
  source 1..23: mol_type = other DNA
actcccagctggagggcctgagc

SEQ ID NO: 329 - DNA, 23 bp, synthetic construct
  misc_feature 1..23: note = Description of Artificial Sequence: Syntheticoligonucleotide
  source 1..23: mol_type = other DNA
agtcagaaaagacgtgagtgagc

SEQ ID NO: 330 - DNA, 23 bp, synthetic construct
  misc_feature 1..23: note = Description of Artificial Sequence: Syntheticoligonucleotide
  source 1..23: mol_type = other DNA
tcaaccaggagccagcctccggc

SEQ ID NO: 331 - DNA, 23 bp, synthetic construct
  misc_feature 1..23: note = Description of Artificial Sequence: Syntheticoligonucleotide
  source 1..23: mol_type = other DNA
ggagcagttctaccgctcactgc

SEQ ID NO: 332 - DNA, 23 bp, synthetic construct
  misc_feature 1..23: note = Description of Artificial Sequence: Syntheticoligonucleotide
  source 1..23: mol_type = other DNA
tcactgcaggacacgtatggtgc

SEQ ID NO: 333 - DNA, 23 bp, synthetic construct
  misc_feature 1..23: note = Description of Artificial Sequence: Syntheticoligonucleotide
  source 1..23: mol_type = other DNA
aacggcagctggcccaaggaggc

SEQ ID NO: 334 - DNA, 23 bp, synthetic construct
  misc_feature 1..23: note = Description of Artificial Sequence: Syntheticoligonucleotide
  source 1..23: mol_type = other DNA
tgagacacgagtgattgctgtgc

SEQ ID NO: 335 - DNA, 23 bp, synthetic construct
  misc_feature 1..23: note = Description of Artificial Sequence: Syntheticoligonucleotide
  source 1..23: mol_type = other DNA
ggtcagggcaagagctattgggc

SEQ ID NO: 336 - DNA, 23 bp, synthetic construct
  misc_feature 1..23: note = Description of Artificial Sequence: Syntheticoligonucleotide
  source 1..23: mol_type = other DNA
ggcccacagccactcgtggcggc

SEQ ID NO: 337 - DNA, 23 bp, synthetic construct
  misc_feature 1..23: note = Description of Artificial Sequence: Syntheticoligonucleotide
  source 1..23: mol_type = other DNA
ggaagcgcaagatggcttcctgc

SEQ ID NO: 338 - DNA, 23 bp, synthetic construct
  misc_feature 1..23: note = Description of Artificial Sequence: Syntheticoligonucleotide
  source 1..23: mol_type = other DNA
ctggtccagagcctgagcaaggc

SEQ ID NO: 339 - DNA, 23 bp, synthetic construct
  misc_feature 1..23: note = Description of Artificial Sequence: Syntheticoligonucleotide
  source 1..23: mol_type = other DNA
gcaggcccaggcatacgtgatgc

SEQ ID NO: 340 - DNA, 23 bp, synthetic construct
  misc_feature 1..23: note = Description of Artificial Sequence: Syntheticoligonucleotide
  source 1..23: mol_type = other DNA
aggcccaggcatacgtgatgcgc

SEQ ID NO: 341 - DNA, 23 bp, synthetic construct
  misc_feature 1..23: note = Description of Artificial Sequence: Syntheticoligonucleotide
  source 1..23: mol_type = other DNA
gcaccaagacagagccctgacgc

SEQ ID NO: 342 - DNA, 23 bp, synthetic construct
  misc_feature 1..23: note = Description of Artificial Sequence: Syntheticoligonucleotide
  source 1..23: mol_type = other DNA
gtgccagctctcagaggccctgc

SEQ ID NO: 343 - DNA, 23 bp, synthetic construct
  misc_feature 1..23: note = Description of Artificial Sequence: Syntheticoligonucleotide
  source 1..23: mol_type = other DNA
ttagtccaacacccaccgcgggc

SEQ ID NO: 344 - DNA, 23 bp, synthetic construct
  misc_feature 1..23: note = Description of Artificial Sequence: Syntheticoligonucleotide
  source 1..23: mol_type = other DNA
gtccaacacccaccgcgggccgc

SEQ ID NO: 345 - DNA, 23 bp, synthetic construct
  misc_feature 1..23: note = Description of Artificial Sequence: Syntheticoligonucleotide
  source 1..23: mol_type = other DNA
ctcctgcaatgcttcctgggggc

SEQ ID NO: 346 - DNA, 23 bp, synthetic construct
  misc_feature 1..23: note = Description of Artificial Sequence: Syntheticoligonucleotide
  source 1..23: mol_type = other DNA
tcttccagcctcccgcccgctgc

SEQ ID NO: 347 - DNA, 23 bp, synthetic construct
  misc_feature 1..23: note = Description of Artificial Sequence: Syntheticoligonucleotide
  source 1..23: mol_type = other DNA
gcggctgcagccggggacactgc

SEQ ID NO: 348 - DNA, 23 bp, synthetic construct
  misc_feature 1..23: note = Description of Artificial Sequence: Syntheticoligonucleotide
  source 1..23: mol_type = other DNA
ctgcagccggggacactgcgggc

SEQ ID NO: 349 - DNA, 23 bp, synthetic construct
  misc_feature 1..23: note = Description of Artificial Sequence: Syntheticoligonucleotide
  source 1..23: mol_type = other DNA
ggcgcggcagctgctggagctgc

SEQ ID NO: 350 - DNA, 23 bp, synthetic construct
  misc_feature 1..23: note = Description of Artificial Sequence: Syntheticoligonucleotide
  source 1..23: mol_type = other DNA
gcggcagctgctggagctgctgc

SEQ ID NO: 351 - DNA, 23 bp, synthetic construct
  misc_feature 1..23: note = Description of Artificial Sequence: Syntheticoligonucleotide
  source 1..23: mol_type = other DNA
ttggcagcacgtggtacaggagc

SEQ ID NO: 352 - DNA, 23 bp, synthetic construct
  misc_feature 1..23: note = Description of Artificial Sequence: Syntheticoligonucleotide
  source 1..23: mol_type = other DNA
tggtacaggagctccccggccgc

SEQ ID NO: 353 - DNA, 23 bp, synthetic construct
  misc_feature 1..23: note = Description of Artificial Sequence: Syntheticoligonucleotide
  source 1..23: mol_type = other DNA
gcagcagcatggggagaccaagc

SEQ ID NO: 354 - DNA, 23 bp, synthetic construct
  misc_feature 1..23: note = Description of Artificial Sequence: Syntheticoligonucleotide
  source 1..23: mol_type = other DNA
gactcagaggtgagaggagaggc

SEQ ID NO: 355 - DNA, 23 bp, synthetic construct
  misc_feature 1..23: note = Description of Artificial Sequence: Syntheticoligonucleotide
  source 1..23: mol_type = other DNA
gtccagtacaacaagttcacggc

SEQ ID NO: 356 - DNA, 23 bp, synthetic construct
  misc_feature 1..23: note = Description of Artificial Sequence: Syntheticoligonucleotide
  source 1..23: mol_type = other DNA
gggcccagcagctcgctgccagc

SEQ ID NO: 357 - DNA, 23 bp, synthetic construct
  misc_feature 1..23: note = Description of Artificial Sequence: Syntheticoligonucleotide
  source 1..23: mol_type = other DNA
aacaacaggattcacggatcagc

SEQ ID NO: 358 - DNA, 23 bp, synthetic construct
  misc_feature 1..23: note = Description of Artificial Sequence: Syntheticoligonucleotide
  source 1..23: mol_type = other DNA
tacaagctgtcggaaacagaggc

SEQ ID NO: 359 - DNA, 23 bp, synthetic construct
  misc_feature 1..23: note = Description of Artificial Sequence: Syntheticoligonucleotide
  source 1..23: mol_type = other DNA
gcccagcctaggaggcaaagagc

SEQ ID NO: 360 - DNA, 23 bp, synthetic construct
  misc_feature 1..23: note = Description of Artificial Sequence: Syntheticoligonucleotide
  source 1..23: mol_type = other DNA
ctcacctctgagtctgcacaagc

SEQ ID NO: 361 - DNA, 23 bp, synthetic construct
  misc_feature 1..23: note = Description of Artificial Sequence: Syntheticoligonucleotide
  source 1..23: mol_type = other DNA
ctcccacagcgccaccgtgtcgc

SEQ ID NO: 362 - DNA, 23 bp, synthetic construct
  misc_feature 1..23: note = Description of Artificial Sequence: Syntheticoligonucleotide
  source 1..23: mol_type = other DNA
ccacctgaaacgggtgacacagc

SEQ ID NO: 363 - DNA, 23 bp, synthetic construct
  misc_feature 1..23: note = Description of Artificial Sequence: Syntheticoligonucleotide
  source 1..23: mol_type = other DNA
tgccaaattccagcctcctcggc

SEQ ID NO: 364 - DNA, 23 bp, synthetic construct
  misc_feature 1..23: note = Description of Artificial Sequence: Syntheticoligonucleotide
  source 1..23: mol_type = other DNA
cctccagccagttgtcatagggc

SEQ ID NO: 365 - DNA, 23 bp, synthetic construct
  misc_feature 1..23: note = Description of Artificial Sequence: Syntheticoligonucleotide
  source 1..23: mol_type = other DNA
cagccacagggcccccaggaagc

SEQ ID NO: 366 - DNA, 23 bp, synthetic construct
  misc_feature 1..23: note = Description of Artificial Sequence: Syntheticoligonucleotide
  source 1..23: mol_type = other DNA
atcgcccaggtcctcacgtctgc

SEQ ID NO: 367 - DNA, 23 bp, synthetic construct
  misc_feature 1..23: note = Description of Artificial Sequence: Syntheticoligonucleotide
  source 1..23: mol_type = other DNA
gctcccaggccagcttggccagc

SEQ ID NO: 368 - DNA, 23 bp, synthetic construct
  misc_feature 1..23: note = Description of Artificial Sequence: Syntheticoligonucleotide
  source 1..23: mol_type = other DNA
caagcccaggcccggctcactgc

SEQ ID NO: 369 - DNA, 23 bp, synthetic construct
  misc_feature 1..23: note = Description of Artificial Sequence: Syntheticoligonucleotide
  source 1..23: mol_type = other DNA
ccggctctgcaaaggccaggggc

SEQ ID NO: 370 - DNA, 23 bp, synthetic construct
  misc_feature 1..23: note = Description of Artificial Sequence: Syntheticoligonucleotide
  source 1..23: mol_type = other DNA
actcaccaggccattttggaagc

SEQ ID NO: 371 - DNA, 23 bp, synthetic construct
  misc_feature 1..23: note = Description of Artificial Sequence: Syntheticoligonucleotide
  source 1..23: mol_type = other DNA
ttgtgctctggagatggagaagc

SEQ ID NO: 372 - DNA, 23 bp, synthetic construct
  misc_feature 1..23: note = Description of Artificial Sequence: Syntheticoligonucleotide
  source 1..23: mol_type = other DNA
agatttgccagagcccatggggc

SEQ ID NO: 373 - DNA, 23 bp, synthetic construct
  misc_feature 1..23: note = Description of Artificial Sequence: Syntheticoligonucleotide
  source 1..23: mol_type = other DNA
aaggcactgcaagagacaaaggc

SEQ ID NO: 374 - DNA, 23 bp, synthetic construct
  misc_feature 1..23: note = Description of Artificial Sequence: Syntheticoligonucleotide
  source 1..23: mol_type = other DNA
ggctcagctgtgaggaagtgggc

SEQ ID NO: 375 - DNA, 23 bp, synthetic construct
  misc_feature 1..23: note = Description of Artificial Sequence: Syntheticoligonucleotide
  source 1..23: mol_type = other DNA
ggcttccagtgcttcaggtctgc

SEQ ID NO: 376 - DNA, 23 bp, synthetic construct
  misc_feature 1..23: note = Description of Artificial Sequence: Syntheticoligonucleotide
  source 1..23: mol_type = other DNA
aaacttactgaaaatgtccttgc

SEQ ID NO: 377 - DNA, 23 bp, synthetic construct
  misc_feature 1..23: note = Description of Artificial Sequence: Syntheticoligonucleotide
  source 1..23: mol_type = other DNA
ccgctgcccagacaaggaaaagc

SEQ ID NO: 378 - DNA, 23 bp, synthetic construct
  misc_feature 1..23: note = Description of Artificial Sequence: Syntheticoligonucleotide
  source 1..23: mol_type = other DNA
tcctcaccgatattggcataagc

SEQ ID NO: 379 - DNA, 23 bp, synthetic construct
  misc_feature 1..23: note = Description of Artificial Sequence: Syntheticoligonucleotide
  source 1..23: mol_type = other DNA
ctgcctgggagggaagacaatgc

SEQ ID NO: 380 - DNA, 26 bp, synthetic construct
  misc_feature 1..26: note = Description of Artificial Sequence: Syntheticoligonucleotide
  source 1..26: mol_type = other DNA
ccacctgcagcctggatgcgctgagt

SEQ ID NO: 381 - DNA, 26 bp, synthetic construct
  misc_feature 1..26: note = Description of Artificial Sequence: Syntheticoligonucleotide
  source 1..26: mol_type = other DNA
ccactcaccttagcctgagcagggat

SEQ ID NO: 382 - DNA, 23 bp, synthetic construct
  misc_feature 1..23: note = Description of Artificial Sequence: Syntheticoligonucleotide
  source 1..23: mol_type = other DNA
cacagctgagccccccactgtgg

SEQ ID NO: 383 - DNA, 23 bp, synthetic construct
  misc_feature 1..23: note = Description of Artificial Sequence: Syntheticoligonucleotide
  source 1..23: mol_type = other DNA
caatgtaggtgaggtgccccagg

SEQ ID NO: 384 - DNA, 23 bp, synthetic construct
  misc_feature 1..23: note = Description of Artificial Sequence: Syntheticoligonucleotide
  source 1..23: mol_type = other DNA
ctccaggacgagaagttcctcgg

SEQ ID NO: 385 - DNA, 23 bp, synthetic construct
  misc_feature 1..23: note = Description of Artificial Sequence: Syntheticoligonucleotide
  source 1..23: mol_type = other DNA
cctaggctgggccctgtctcagg

SEQ ID NO: 386 - DNA, 23 bp, synthetic construct
  misc_feature 1..23: note = Description of Artificial Sequence: Syntheticoligonucleotide
  source 1..23: mol_type = other DNA
acactcactccatcacccggagg

SEQ ID NO: 387 - DNA, 23 bp, synthetic construct
  misc_feature 1..23: note = Description of Artificial Sequence: Syntheticoligonucleotide
  source 1..23: mol_type = other DNA
cactcactccatcacccggaggg

SEQ ID NO: 388 - DNA, 23 bp, synthetic construct
  misc_feature 1..23: note = Description of Artificial Sequence: Syntheticoligonucleotide
  source 1..23: mol_type = other DNA
ccactcaccttagcctgagcagg

SEQ ID NO: 389 - DNA, 23 bp, synthetic construct
  misc_feature 1..23: note = Description of Artificial Sequence: Syntheticoligonucleotide
  source 1..23: mol_type = other DNA
cactcaccttagcctgagcaggg

SEQ ID NO: 390 - DNA, 23 bp, synthetic construct
  misc_feature 1..23: note = Description of Artificial Sequence: Syntheticoligonucleotide
  source 1..23: mol_type = other DNA
cactcacttgagggtttccaagg

SEQ ID NO: 391 - DNA, 23 bp, synthetic construct
  misc_feature 1..23: note = Description of Artificial Sequence: Syntheticoligonucleotide
  source 1..23: mol_type = other DNA
atactcactccagatgctgcagg

SEQ ID NO: 392 - DNA, 23 bp, synthetic construct
  misc_feature 1..23: note = Description of Artificial Sequence: Syntheticoligonucleotide
  source 1..23: mol_type = other DNA
tactcactccagatgctgcaggg

SEQ ID NO: 393 - DNA, 23 bp, synthetic construct
  misc_feature 1..23: note = Description of Artificial Sequence: Syntheticoligonucleotide
  source 1..23: mol_type = other DNA
ccttacctgtcatgtttgctcgg

SEQ ID NO: 394 - DNA, 23 bp, synthetic construct
  misc_feature 1..23: note = Description of Artificial Sequence: Syntheticoligonucleotide
  source 1..23: mol_type = other DNA
cttacctgtcatgtttgctcggg

SEQ ID NO: 395 - DNA, 23 bp, synthetic construct
  misc_feature 1..23: note = Description of Artificial Sequence: Syntheticoligonucleotide
  source 1..23: mol_type = other DNA
taacatactgggaatctggtcgg

SEQ ID NO: 396 - DNA, 23 bp, synthetic construct
  misc_feature 1..23: note = Description of Artificial Sequence: Syntheticoligonucleotide
  source 1..23: mol_type = other DNA
ttttaccttggggctctgacagg

SEQ ID NO: 397 - DNA, 23 bp, synthetic construct
  misc_feature 1..23: note = Description of Artificial Sequence: Syntheticoligonucleotide
  source 1..23: mol_type = other DNA
ccttgtctgggcagcggaactgg

SEQ ID NO: 398 - DNA, 23 bp, synthetic construct
  misc_feature 1..23: note = Description of Artificial Sequence: Syntheticoligonucleotide
  source 1..23: mol_type = other DNA
tttctcaaagtagagcacatagg

SEQ ID NO: 399 - DNA, 23 bp, synthetic construct
  misc_feature 1..23: note = Description of Artificial Sequence: Syntheticoligonucleotide
  source 1..23: mol_type = other DNA
tttctctgcagccttcccagagg

SEQ ID NO: 400 - DNA, 23 bp, synthetic construct
  misc_feature 1..23: note = Description of Artificial Sequence: Syntheticoligonucleotide
  source 1..23: mol_type = other DNA
cacagctgagccccccactgtgg

SEQ ID NO: 401 - DNA, 23 bp, synthetic construct
  misc_feature 1..23: note = Description of Artificial Sequence: Syntheticoligonucleotide
  source 1..23: mol_type = other DNA
caatgtaggtgaggtgccccagg

SEQ ID NO: 402 - DNA, 23 bp, synthetic construct
  misc_feature 1..23: note = Description of Artificial Sequence: Syntheticoligonucleotide
  source 1..23: mol_type = other DNA
cctggcctttgcagagccggtgg

SEQ ID NO: 403 - DNA, 23 bp, synthetic construct
  misc_feature 1..23: note = Description of Artificial Sequence: Syntheticoligonucleotide
  source 1..23: mol_type = other DNA
ctccaggacgagaagttcctcgg

SEQ ID NO: 404 - DNA, 23 bp, synthetic construct
  misc_feature 1..23: note = Description of Artificial Sequence: Syntheticoligonucleotide
  source 1..23: mol_type = other DNA
cctaggctgggccctgtctcagg

SEQ ID NO: 405 - DNA, 23 bp, synthetic construct
  misc_feature 1..23: note = Description of Artificial Sequence: Syntheticoligonucleotide
  source 1..23: mol_type = other DNA
cccacactcactccatcacccgg

SEQ ID NO: 406 - DNA, 23 bp, synthetic construct
  misc_feature 1..23: note = Description of Artificial Sequence: Syntheticoligonucleotide
  source 1..23: mol_type = other DNA
acactcactccatcacccggagg

SEQ ID NO: 407 - DNA, 23 bp, synthetic construct
  misc_feature 1..23: note = Description of Artificial Sequence: Syntheticoligonucleotide
  source 1..23: mol_type = other DNA
cactcactccatcacccggaggg

SEQ ID NO: 408 - DNA, 23 bp, synthetic construct
  misc_feature 1..23: note = Description of Artificial Sequence: Syntheticoligonucleotide
  source 1..23: mol_type = other DNA
ccactcaccttagcctgagcagg

SEQ ID NO: 409 - DNA, 23 bp, synthetic construct
  misc_feature 1..23: note = Description of Artificial Sequence: Syntheticoligonucleotide
  source 1..23: mol_type = other DNA
cactcaccttagcctgagcaggg

SEQ ID NO: 410 - DNA, 23 bp, synthetic construct
  misc_feature 1..23: note = Description of Artificial Sequence: Syntheticoligonucleotide
  source 1..23: mol_type = other DNA
cactcacttgagggtttccaagg

SEQ ID NO: 411 - DNA, 23 bp, synthetic construct
  misc_feature 1..23: note = Description of Artificial Sequence: Syntheticoligonucleotide
  source 1..23: mol_type = other DNA
atactcactccagatgctgcagg

SEQ ID NO: 412 - DNA, 23 bp, synthetic construct
  misc_feature 1..23: note = Description of Artificial Sequence: Syntheticoligonucleotide
  source 1..23: mol_type = other DNA
tactcactccagatgctgcaggg

SEQ ID NO: 413 - DNA, 23 bp, synthetic construct
  misc_feature 1..23: note = Description of Artificial Sequence: Syntheticoligonucleotide
  source 1..23: mol_type = other DNA
gcccctcaccccacctgaaacgg

SEQ ID NO: 414 - DNA, 23 bp, synthetic construct
  misc_feature 1..23: note = Description of Artificial Sequence: Syntheticoligonucleotide
  source 1..23: mol_type = other DNA
cccctcaccccacctgaaacggg

SEQ ID NO: 415 - DNA, 23 bp, synthetic construct
  misc_feature 1..23: note = Description of Artificial Sequence: Syntheticoligonucleotide
  source 1..23: mol_type = other DNA
catcactcaccaggccattttgg

SEQ ID NO: 416 - DNA, 23 bp, synthetic construct
  misc_feature 1..23: note = Description of Artificial Sequence: Syntheticoligonucleotide
  source 1..23: mol_type = other DNA
ccttacctgtcatgtttgctcgg

SEQ ID NO: 417 - DNA, 23 bp, synthetic construct
  misc_feature 1..23: note = Description of Artificial Sequence: Syntheticoligonucleotide
  source 1..23: mol_type = other DNA
cttacctgtcatgtttgctcggg

SEQ ID NO: 418 - DNA, 23 bp, synthetic construct
  misc_feature 1..23: note = Description of Artificial Sequence: Syntheticoligonucleotide
  source 1..23: mol_type = other DNA
cccctaacatactgggaatctgg

SEQ ID NO: 419 - DNA, 23 bp, synthetic construct
  misc_feature 1..23: note = Description of Artificial Sequence: Syntheticoligonucleotide
  source 1..23: mol_type = other DNA
taacatactgggaatctggtcgg

SEQ ID NO: 420 - DNA, 23 bp, synthetic construct
  misc_feature 1..23: note = Description of Artificial Sequence: Syntheticoligonucleotide
  source 1..23: mol_type = other DNA
aggtgcttcctcaccgatattgg

SEQ ID NO: 421 - DNA, 23 bp, synthetic construct
  misc_feature 1..23: note = Description of Artificial Sequence: Syntheticoligonucleotide
  source 1..23: mol_type = other DNA
ttttaccttggggctctgacagg

SEQ ID NO: 422 - DNA, 23 bp, synthetic construct
  misc_feature 1..23: note = Description of Artificial Sequence: Syntheticoligonucleotide
  source 1..23: mol_type = other DNA
gagccccaaggtaaaaaggccgg

SEQ ID NO: 423 - DNA, 23 bp, synthetic construct
  misc_feature 1..23: note = Description of Artificial Sequence: Syntheticoligonucleotide
  source 1..23: mol_type = other DNA
agccccaaggtaaaaaggccggg

SEQ ID NO: 424 - DNA, 23 bp, synthetic construct
  misc_feature 1..23: note = Description of Artificial Sequence: Syntheticoligonucleotide
  source 1..23: mol_type = other DNA
cagctcacagtgtgccaccatgg

SEQ ID NO: 425 - DNA, 23 bp, synthetic construct
  misc_feature 1..23: note = Description of Artificial Sequence: Syntheticoligonucleotide
  source 1..23: mol_type = other DNA
tatgaccagatggacctggctgg

SEQ ID NO: 426 - DNA, 23 bp, synthetic construct
  misc_feature 1..23: note = Description of Artificial Sequence: Syntheticoligonucleotide
  source 1..23: mol_type = other DNA
actggaccagtatgtcttccagg

SEQ ID NO: 427 - DNA, 23 bp, synthetic construct
  misc_feature 1..23: note = Description of Artificial Sequence: Syntheticoligonucleotide
  source 1..23: mol_type = other DNA
tgtcttccaggactcccagctgg

SEQ ID NO: 428 - DNA, 23 bp, synthetic construct
  misc_feature 1..23: note = Description of Artificial Sequence: Syntheticoligonucleotide
  source 1..23: mol_type = other DNA
cttccaggactcccagctggagg

SEQ ID NO: 429 - DNA, 23 bp, synthetic construct
  misc_feature 1..23: note = Description of Artificial Sequence: Syntheticoligonucleotide
  source 1..23: mol_type = other DNA
ttccaggactcccagctggaggg

SEQ ID NO: 430 - DNA, 23 bp, synthetic construct
  misc_feature 1..23: note = Description of Artificial Sequence: Syntheticoligonucleotide
  source 1..23: mol_type = other DNA
ttcaaccaggagccagcctccgg

SEQ ID NO: 431 - DNA, 23 bp, synthetic construct
  misc_feature 1..23: note = Description of Artificial Sequence: Syntheticoligonucleotide
  source 1..23: mol_type = other DNA
gaccagattcccagtatgttagg

SEQ ID NO: 432 - DNA, 23 bp, synthetic construct
  misc_feature 1..23: note = Description of Artificial Sequence: Syntheticoligonucleotide
  source 1..23: mol_type = other DNA
ctctggcaaatctctgaggctgg

SEQ ID NO: 433 - DNA, 23 bp, synthetic construct
  misc_feature 1..23: note = Description of Artificial Sequence: Syntheticoligonucleotide
  source 1..23: mol_type = other DNA
agccaagtaccccctcccagtgg

SEQ ID NO: 434 - DNA, 23 bp, synthetic construct
  misc_feature 1..23: note = Description of Artificial Sequence: Syntheticoligonucleotide
  source 1..23: mol_type = other DNA
acctcccgagcaaacatgacagg

SEQ ID NO: 435 - DNA, 23 bp, synthetic construct
  misc_feature 1..23: note = Description of Artificial Sequence: Syntheticoligonucleotide
  source 1..23: mol_type = other DNA
cccacccaatgcccggcagctgg

SEQ ID NO: 436 - DNA, 23 bp, synthetic construct
  misc_feature 1..23: note = Description of Artificial Sequence: Syntheticoligonucleotide
  source 1..23: mol_type = other DNA
tggtgcaggccaggctggagagg

SEQ ID NO: 437 - DNA, 23 bp, synthetic construct
  misc_feature 1..23: note = Description of Artificial Sequence: Syntheticoligonucleotide
  source 1..23: mol_type = other DNA
gaacggcagctggcccaaggagg

SEQ ID NO: 438 - DNA, 23 bp, synthetic construct
  misc_feature 1..23: note = Description of Artificial Sequence: Syntheticoligonucleotide
  source 1..23: mol_type = other DNA
ggcccaaggaggcctggctgagg

SEQ ID NO: 439 - DNA, 23 bp, synthetic construct
  misc_feature 1..23: note = Description of Artificial Sequence: Syntheticoligonucleotide
  source 1..23: mol_type = other DNA
gacacgagtgattgctgtgctgg

SEQ ID NO: 440 - DNA, 23 bp, synthetic construct
  misc_feature 1..23: note = Description of Artificial Sequence: Syntheticoligonucleotide
  source 1..23: mol_type = other DNA
acacgagtgattgctgtgctggg

SEQ ID NO: 441 - DNA, 23 bp, synthetic construct
  misc_feature 1..23: note = Description of Artificial Sequence: Syntheticoligonucleotide
  source 1..23: mol_type = other DNA
ctggtcagggcaagagctattgg

SEQ ID NO: 442 - DNA, 23 bp, synthetic construct
  misc_feature 1..23: note = Description of Artificial Sequence: Syntheticoligonucleotide
  source 1..23: mol_type = other DNA
tggtcagggcaagagctattggg

SEQ ID NO: 443 - DNA, 23 bp, synthetic construct
  misc_feature 1..23: note = Description of Artificial Sequence: Syntheticoligonucleotide
  source 1..23: mol_type = other DNA
gggcccacagccactcgtggcgg

SEQ ID NO: 444 - DNA, 23 bp, synthetic construct
  misc_feature 1..23: note = Description of Artificial Sequence: Syntheticoligonucleotide
  source 1..23: mol_type = other DNA
ttccagaagaagctgctccgagg

SEQ ID NO: 445 - DNA, 23 bp, synthetic construct
  misc_feature 1..23: note = Description of Artificial Sequence: Syntheticoligonucleotide
  source 1..23: mol_type = other DNA
cctggtccagagcctgagcaagg

SEQ ID NO: 446 - DNA, 23 bp, synthetic construct
  misc_feature 1..23: note = Description of Artificial Sequence: Syntheticoligonucleotide
  source 1..23: mol_type = other DNA
cagacatcaaagtaccctacagg

SEQ ID NO: 447 - DNA, 23 bp, synthetic construct
  misc_feature 1..23: note = Description of Artificial Sequence: Syntheticoligonucleotide
  source 1..23: mol_type = other DNA
acatcaaagtaccctacaggagg

SEQ ID NO: 448 - DNA, 23 bp, synthetic construct
  misc_feature 1..23: note = Description of Artificial Sequence: Syntheticoligonucleotide
  source 1..23: mol_type = other DNA
cttagtccaacacccaccgcggg

SEQ ID NO: 449 - DNA, 23 bp, synthetic construct
  misc_feature 1..23: note = Description of Artificial Sequence: Syntheticoligonucleotide
  source 1..23: mol_type = other DNA
cctcctgcaatgcttcctggggg

SEQ ID NO: 450 - DNA, 23 bp, synthetic construct
  misc_feature 1..23: note = Description of Artificial Sequence: Syntheticoligonucleotide
  source 1..23: mol_type = other DNA
ggaagcagaaggtgcttgcgagg

SEQ ID NO: 451 - DNA, 23 bp, synthetic construct
  misc_feature 1..23: note = Description of Artificial Sequence: Syntheticoligonucleotide
  source 1..23: mol_type = other DNA
ggctgcagccggggacactgcgg

SEQ ID NO: 452 - DNA, 23 bp, synthetic construct
  misc_feature 1..23: note = Description of Artificial Sequence: Syntheticoligonucleotide
  source 1..23: mol_type = other DNA
gctgcagccggggacactgcggg

SEQ ID NO: 453 - DNA, 23 bp, synthetic construct
  misc_feature 1..23: note = Description of Artificial Sequence: Syntheticoligonucleotide
  source 1..23: mol_type = other DNA
aatttggcagcacgtggtacagg

SEQ ID NO: 454 - DNA, 23 bp, synthetic construct
  misc_feature 1..23: note = Description of Artificial Sequence: Syntheticoligonucleotide
  source 1..23: mol_type = other DNA
ggcgggccaagacttctccctgg

SEQ ID NO: 455 - DNA, 23 bp, synthetic construct
  misc_feature 1..23: note = Description of Artificial Sequence: Syntheticoligonucleotide
  source 1..23: mol_type = other DNA
tgtgcagactcagaggtgagagg

SEQ ID NO: 456 - DNA, 23 bp, synthetic construct
  misc_feature 1..23: note = Description of Artificial Sequence: Syntheticoligonucleotide
  source 1..23: mol_type = other DNA
agactcagaggtgagaggagagg

SEQ ID NO: 457 - DNA, 23 bp, synthetic construct
  misc_feature 1..23: note = Description of Artificial Sequence: Syntheticoligonucleotide
  source 1..23: mol_type = other DNA
cccccaggctttccccaaactgg

SEQ ID NO: 458 - DNA, 23 bp, synthetic construct
  misc_feature 1..23: note = Description of Artificial Sequence: Syntheticoligonucleotide
  source 1..23: mol_type = other DNA
cttcccccagctgaagtccttgg

SEQ ID NO: 459 - DNA, 23 bp, synthetic construct
  misc_feature 1..23: note = Description of Artificial Sequence: Syntheticoligonucleotide
  source 1..23: mol_type = other DNA
cgtccagtacaacaagttcacgg

SEQ ID NO: 460 - DNA, 23 bp, synthetic construct
  misc_feature 1..23: note = Description of Artificial Sequence: Syntheticoligonucleotide
  source 1..23: mol_type = other DNA
acctgcaacaacaggattcacgg

SEQ ID NO: 461 - DNA, 23 bp, synthetic construct
  misc_feature 1..23: note = Description of Artificial Sequence: Syntheticoligonucleotide
  source 1..23: mol_type = other DNA
tgggcgtccacatcctgcaaggg

SEQ ID NO: 462 - DNA, 23 bp, synthetic construct
  misc_feature 1..23: note = Description of Artificial Sequence: Syntheticoligonucleotide
  source 1..23: mol_type = other DNA
gggcgtccacatcctgcaagggg

SEQ ID NO: 463 - DNA, 23 bp, synthetic construct
  misc_feature 1..23: note = Description of Artificial Sequence: Syntheticoligonucleotide
  source 1..23: mol_type = other DNA
ggcgtccacatcctgcaaggggg

SEQ ID NO: 464 - DNA, 23 bp, synthetic construct
  misc_feature 1..23: note = Description of Artificial Sequence: Syntheticoligonucleotide
  source 1..23: mol_type = other DNA
gcgtccacatcctgcaagggggg

SEQ ID NO: 465 - DNA, 23 bp, synthetic construct
  misc_feature 1..23: note = Description of Artificial Sequence: Syntheticoligonucleotide
  source 1..23: mol_type = other DNA
ccacatcctgcaaggggggatgg

SEQ ID NO: 466 - DNA, 23 bp, synthetic construct
  misc_feature 1..23: note = Description of Artificial Sequence: Syntheticoligonucleotide
  source 1..23: mol_type = other DNA
cacatcctgcaaggggggatggg

SEQ ID NO: 467 - DNA, 23 bp, synthetic construct
  misc_feature 1..23: note = Description of Artificial Sequence: Syntheticoligonucleotide
  source 1..23: mol_type = other DNA
acactcactccatcacccggagg

SEQ ID NO: 468 - DNA, 23 bp, synthetic construct
  misc_feature 1..23: note = Description of Artificial Sequence: Syntheticoligonucleotide
  source 1..23: mol_type = other DNA
cactcactccatcacccggaggg

SEQ ID NO: 469 - DNA, 23 bp, synthetic construct
  misc_feature 1..23: note = Description of Artificial Sequence: Syntheticoligonucleotide
  source 1..23: mol_type = other DNA
gtacaagctgtcggaaacagagg

SEQ ID NO: 470 - DNA, 23 bp, synthetic construct
  misc_feature 1..23: note = Description of Artificial Sequence: Syntheticoligonucleotide
  source 1..23: mol_type = other DNA
ccactcaccttagcctgagcagg

SEQ ID NO: 471 - DNA, 23 bp, synthetic construct
  misc_feature 1..23: note = Description of Artificial Sequence: Syntheticoligonucleotide
  source 1..23: mol_type = other DNA
cactcaccttagcctgagcaggg

SEQ ID NO: 472 - DNA, 23 bp, synthetic construct
  misc_feature 1..23: note = Description of Artificial Sequence: Syntheticoligonucleotide
  source 1..23: mol_type = other DNA
cagactgcggggacacagtgagg

SEQ ID NO: 473 - DNA, 23 bp, synthetic construct
  misc_feature 1..23: note = Description of Artificial Sequence: Syntheticoligonucleotide
  source 1..23: mol_type = other DNA
agactgcggggacacagtgaggg

SEQ ID NO: 474 - DNA, 23 bp, synthetic construct
  misc_feature 1..23: note = Description of Artificial Sequence: Syntheticoligonucleotide
  source 1..23: mol_type = other DNA
cactcacttgagggtttccaagg

SEQ ID NO: 475 - DNA, 23 bp, synthetic construct
  misc_feature 1..23: note = Description of Artificial Sequence: Syntheticoligonucleotide
  source 1..23: mol_type = other DNA
aggctgcaggtggaatcagatgg

SEQ ID NO: 476 - DNA, 23 bp, synthetic construct
  misc_feature 1..23: note = Description of Artificial Sequence: Syntheticoligonucleotide
  source 1..23: mol_type = other DNA
atactcactccagatgctgcagg

SEQ ID NO: 477 - DNA, 23 bp, synthetic construct
  misc_feature 1..23: note = Description of Artificial Sequence: Syntheticoligonucleotide
  source 1..23: mol_type = other DNA
tactcactccagatgctgcaggg

SEQ ID NO: 478 - DNA, 23 bp, synthetic construct
  misc_feature 1..23: note = Description of Artificial Sequence: Syntheticoligonucleotide
  source 1..23: mol_type = other DNA
tcactccagatgctgcagggagg

SEQ ID NO: 479 - DNA, 23 bp, synthetic construct
  misc_feature 1..23: note = Description of Artificial Sequence: Syntheticoligonucleotide
  source 1..23: mol_type = other DNA
agcctaggaggcaaagagcaagg

SEQ ID NO: 480 - DNA, 23 bp, synthetic construct
  misc_feature 1..23: note = Description of Artificial Sequence: Syntheticoligonucleotide
  source 1..23: mol_type = other DNA
ctgccaaattccagcctcctcgg

SEQ ID NO: 481 - DNA, 23 bp, synthetic construct
  misc_feature 1..23: note = Description of Artificial Sequence: Syntheticoligonucleotide
  source 1..23: mol_type = other DNA
gagccagccacagggcccccagg

SEQ ID NO: 482 - DNA, 23 bp, synthetic construct
  misc_feature 1..23: note = Description of Artificial Sequence: Syntheticoligonucleotide
  source 1..23: mol_type = other DNA
cgcccaggtcctcacgtctgcgg

SEQ ID NO: 483 - DNA, 23 bp, synthetic construct
  misc_feature 1..23: note = Description of Artificial Sequence: Syntheticoligonucleotide
  source 1..23: mol_type = other DNA
accggctctgcaaaggccagggg

SEQ ID NO: 484 - DNA, 23 bp, synthetic construct
  misc_feature 1..23: note = Description of Artificial Sequence: Syntheticoligonucleotide
  source 1..23: mol_type = other DNA
aggccattttggaagcttgttgg

SEQ ID NO: 485 - DNA, 23 bp, synthetic construct
  misc_feature 1..23: note = Description of Artificial Sequence: Syntheticoligonucleotide
  source 1..23: mol_type = other DNA
tgctctggagatggagaagcagg

SEQ ID NO: 486 - DNA, 23 bp, synthetic construct
  misc_feature 1..23: note = Description of Artificial Sequence: Syntheticoligonucleotide
  source 1..23: mol_type = other DNA
ccttacctgtcatgtttgctcgg

SEQ ID NO: 487 - DNA, 23 bp, synthetic construct
  misc_feature 1..23: note = Description of Artificial Sequence: Syntheticoligonucleotide
  source 1..23: mol_type = other DNA
cttacctgtcatgtttgctcggg

SEQ ID NO: 488 - DNA, 23 bp, synthetic construct
  misc_feature 1..23: note = Description of Artificial Sequence: Syntheticoligonucleotide
  source 1..23: mol_type = other DNA
aaaggcactgcaagagacaaagg

SEQ ID NO: 489 - DNA, 23 bp, synthetic construct
  misc_feature 1..23: note = Description of Artificial Sequence: Syntheticoligonucleotide
  source 1..23: mol_type = other DNA
taacatactgggaatctggtcgg

SEQ ID NO: 490 - DNA, 23 bp, synthetic construct
  misc_feature 1..23: note = Description of Artificial Sequence: Syntheticoligonucleotide
  source 1..23: mol_type = other DNA
ttccagtgcttcaggtctgccgg

SEQ ID NO: 491 - DNA, 23 bp, synthetic construct
  misc_feature 1..23: note = Description of Artificial Sequence: Syntheticoligonucleotide
  source 1..23: mol_type = other DNA
ttttaccttggggctctgacagg

SEQ ID NO: 492 - RNA, 20 nt, synthetic construct
  misc_feature 1..20: note = Description of Artificial Sequence: Syntheticoligonucleotide
  source 1..20: mol_type = other RNA
tctctgcagccttcccagag

SEQ ID NO: 493 - RNA, 20 nt, synthetic construct
  misc_feature 1..20: note = Description of Artificial Sequence: Syntheticoligonucleotide
  source 1..20: mol_type = other RNA
tctccaggacgagaagttcc

SEQ ID NO: 494 - RNA, 20 nt, synthetic construct
  misc_feature 1..20: note = Description of Artificial Sequence: Syntheticoligonucleotide
  source 1..20: mol_type = other RNA
cacctgcagcctggatgcgc

SEQ ID NO: 495 - RNA, 20 nt, synthetic construct
  misc_feature 1..20: note = Description of Artificial Sequence: Syntheticoligonucleotide
  source 1..20: mol_type = other RNA
ccgacagcttgtacaataac

SEQ ID NO: 496 - RNA, 20 nt, synthetic construct
  misc_feature 1..20: note = Description of Artificial Sequence: Syntheticoligonucleotide
  source 1..20: mol_type = other RNA
ctcttgccagcgtccagtac

SEQ ID NO: 497 - RNA, 20 nt, synthetic construct
  misc_feature 1..20: note = Description of Artificial Sequence: Syntheticoligonucleotide
  source 1..20: mol_type = other RNA
tgtctgggcagcggaactgg

SEQ ID NO: 498 - RNA, 20 nt, synthetic construct
  misc_feature 1..20: note = Description of Artificial Sequence: Syntheticoligonucleotide
  source 1..20: mol_type = other RNA
tcaaagtagagcacatagga

SEQ ID NO: 499 - RNA, 20 nt, synthetic construct
  misc_feature 1..20: note = Description of Artificial Sequence: Syntheticoligonucleotide
  source 1..20: mol_type = other RNA
ctcacagctgagccccccac

SEQ ID NO: 500 - RNA, 20 nt, synthetic construct
  misc_feature 1..20: note = Description of Artificial Sequence: Syntheticoligonucleotide
  source 1..20: mol_type = other RNA
ggcctttgcagagccggtgg

SEQ ID NO: 501 - RNA, 20 nt, synthetic construct
  misc_feature 1..20: note = Description of Artificial Sequence: Syntheticoligonucleotide
  source 1..20: mol_type = other RNA
ccacctgcagcctggatgcg

SEQ ID NO: 502 - RNA, 20 nt, synthetic construct
  misc_feature 1..20: note = Description of Artificial Sequence: Syntheticoligonucleotide
  source 1..20: mol_type = other RNA
tcttgccagcgtccagtaca

SEQ ID NO: 503 - RNA, 20 nt, synthetic construct
  misc_feature 1..20: note = Description of Artificial Sequence: Syntheticoligonucleotide
  source 1..20: mol_type = other RNA
ccactcaccttagcctgagc

SEQ ID NO: 504 - RNA, 20 nt, synthetic construct
  misc_feature 1..20: note = Description of Artificial Sequence: Syntheticoligonucleotide
  source 1..20: mol_type = other RNA
cctaacatactgggaatctg

SEQ ID NO: 505 - RNA, 20 nt, synthetic construct
  misc_feature 1..20: note = Description of Artificial Sequence: Syntheticoligonucleotide
  source 1..20: mol_type = other RNA
gagggcccacctgagtagag

SEQ ID NO: 506 - RNA, 20 nt, synthetic construct
  misc_feature 1..20: note = Description of Artificial Sequence: Syntheticoligonucleotide
  source 1..20: mol_type = other RNA
ctttttaccttggggctctg

SEQ ID NO: 507 - RNA, 20 nt, synthetic construct
  misc_feature 1..20: note = Description of Artificial Sequence: Syntheticoligonucleotide
  source 1..20: mol_type = other RNA
aaagtagagcacataggacc

SEQ ID NO: 508 - RNA, 20 nt, synthetic construct
  misc_feature 1..20: note = Description of Artificial Sequence: Syntheticoligonucleotide
  source 1..20: mol_type = other RNA
ctccacagggctgccttgag

SEQ ID NO: 509 - RNA, 20 nt, synthetic construct
  misc_feature 1..20: note = Description of Artificial Sequence: Syntheticoligonucleotide
  source 1..20: mol_type = other RNA
tccaggacgagaagttcctc

SEQ ID NO: 510 - RNA, 20 nt, synthetic construct
  misc_feature 1..20: note = Description of Artificial Sequence: Syntheticoligonucleotide
  source 1..20: mol_type = other RNA
cacctgcagcctggatgcgc

SEQ ID NO: 511 - RNA, 20 nt, synthetic construct
  misc_feature 1..20: note = Description of Artificial Sequence: Syntheticoligonucleotide
  source 1..20: mol_type = other RNA
tgcagcctggatgcgctgag

SEQ ID NO: 512 - RNA, 20 nt, synthetic construct
  misc_feature 1..20: note = Description of Artificial Sequence: Syntheticoligonucleotide
  source 1..20: mol_type = other RNA
cttacgccagcgtctccaca

SEQ ID NO: 513 - RNA, 20 nt, synthetic construct
  misc_feature 1..20: note = Description of Artificial Sequence: Syntheticoligonucleotide
  source 1..20: mol_type = other RNA
actcactccatcacccggag

SEQ ID NO: 514 - RNA, 20 nt, synthetic construct
  misc_feature 1..20: note = Description of Artificial Sequence: Syntheticoligonucleotide
  source 1..20: mol_type = other RNA
actcaccttagcctgagcag

SEQ ID NO: 515 - RNA, 20 nt, synthetic construct
  misc_feature 1..20: note = Description of Artificial Sequence: Syntheticoligonucleotide
  source 1..20: mol_type = other RNA
actcacttgagggtttccaa

SEQ ID NO: 516 - RNA, 20 nt, synthetic construct
  misc_feature 1..20: note = Description of Artificial Sequence: Syntheticoligonucleotide
  source 1..20: mol_type = other RNA
actcactccagatgctgcag

SEQ ID NO: 517 - RNA, 20 nt, synthetic construct
  misc_feature 1..20: note = Description of Artificial Sequence: Syntheticoligonucleotide
  source 1..20: mol_type = other RNA
atcactcaccaggccatttt

SEQ ID NO: 518 - RNA, 20 nt, synthetic construct
  misc_feature 1..20: note = Description of Artificial Sequence: Syntheticoligonucleotide
  source 1..20: mol_type = other RNA
gccccaaggtaaaaaggccg

SEQ ID NO: 519 - RNA, 20 nt, synthetic construct
  misc_feature 1..20: note = Description of Artificial Sequence: Syntheticoligonucleotide
  source 1..20: mol_type = other RNA
agctcacagtgtgccaccat

SEQ ID NO: 520 - RNA, 20 nt, synthetic construct
  misc_feature 1..20: note = Description of Artificial Sequence: Syntheticoligonucleotide
  source 1..20: mol_type = other RNA
atgaccagatggacctggct

SEQ ID NO: 521 - RNA, 20 nt, synthetic construct
  misc_feature 1..20: note = Description of Artificial Sequence: Syntheticoligonucleotide
  source 1..20: mol_type = other RNA
gaccagatggacctggctgg

SEQ ID NO: 522 - RNA, 20 nt, synthetic construct
  misc_feature 1..20: note = Description of Artificial Sequence: Syntheticoligonucleotide
  source 1..20: mol_type = other RNA
ctggaccagtatgtcttcca

SEQ ID NO: 523 - RNA, 20 nt, synthetic construct
  misc_feature 1..20: note = Description of Artificial Sequence: Syntheticoligonucleotide
  source 1..20: mol_type = other RNA
gtcttccaggactcccagct

SEQ ID NO: 524 - RNA, 20 nt, synthetic construct
  misc_feature 1..20: note = Description of Artificial Sequence: Syntheticoligonucleotide
  source 1..20: mol_type = other RNA
ggactcccagctggagggcc

SEQ ID NO: 525 - RNA, 20 nt, synthetic construct
  misc_feature 1..20: note = Description of Artificial Sequence: Syntheticoligonucleotide
  source 1..20: mol_type = other RNA
ttgggcagaaaagtcagaaa

SEQ ID NO: 526 - RNA, 20 nt, synthetic construct
  misc_feature 1..20: note = Description of Artificial Sequence: Syntheticoligonucleotide
  source 1..20: mol_type = other RNA
aaagtcagaaaagacgtgag

SEQ ID NO: 527 - RNA, 20 nt, synthetic construct
  misc_feature 1..20: note = Description of Artificial Sequence: Syntheticoligonucleotide
  source 1..20: mol_type = other RNA
ctccggccagatgcgcctgg

SEQ ID NO: 528 - RNA, 20 nt, synthetic construct
  misc_feature 1..20: note = Description of Artificial Sequence: Syntheticoligonucleotide
  source 1..20: mol_type = other RNA
tctggcaaatctctgaggct

SEQ ID NO: 529 - RNA, 20 nt, synthetic construct
  misc_feature 1..20: note = Description of Artificial Sequence: Syntheticoligonucleotide
  source 1..20: mol_type = other RNA
ccacccaatgcccggcagct

SEQ ID NO: 530 - RNA, 20 nt, synthetic construct
  misc_feature 1..20: note = Description of Artificial Sequence: Syntheticoligonucleotide
  source 1..20: mol_type = other RNA
acccaatgcccggcagctgg

SEQ ID NO: 531 - RNA, 20 nt, synthetic construct
  misc_feature 1..20: note = Description of Artificial Sequence: Syntheticoligonucleotide
  source 1..20: mol_type = other RNA
ctgcaggacacgtatggtgc

SEQ ID NO: 532 - RNA, 20 nt, synthetic construct
  misc_feature 1..20: note = Description of Artificial Sequence: Syntheticoligonucleotide
  source 1..20: mol_type = other RNA
tctggtgcaggccaggctgg

SEQ ID NO: 533 - RNA, 20 nt, synthetic construct
  misc_feature 1..20: note = Description of Artificial Sequence: Syntheticoligonucleotide
  source 1..20: mol_type = other RNA
ggtgcaggccaggctggaga

SEQ ID NO: 534 - RNA, 20 nt, synthetic construct
  misc_feature 1..20: note = Description of Artificial Sequence: Syntheticoligonucleotide
  source 1..20: mol_type = other RNA
ctggcccaaggaggcctggc

SEQ ID NO: 535 - RNA, 20 nt, synthetic construct
  misc_feature 1..20: note = Description of Artificial Sequence: Syntheticoligonucleotide
  source 1..20: mol_type = other RNA
ccacagccactcgtggcggc

SEQ ID NO: 536 - RNA, 20 nt, synthetic construct
  misc_feature 1..20: note = Description of Artificial Sequence: Syntheticoligonucleotide
  source 1..20: mol_type = other RNA
ttttccagaagaagctgctc

SEQ ID NO: 537 - RNA, 20 nt, synthetic construct
  misc_feature 1..20: note = Description of Artificial Sequence: Syntheticoligonucleotide
  source 1..20: mol_type = other RNA
gtccagagcctgagcaaggc

SEQ ID NO: 538 - RNA, 20 nt, synthetic construct
  misc_feature 1..20: note = Description of Artificial Sequence: Syntheticoligonucleotide
  source 1..20: mol_type = other RNA
ggagcaggcccaggcatacg

SEQ ID NO: 539 - RNA, 20 nt, synthetic construct
  misc_feature 1..20: note = Description of Artificial Sequence: Syntheticoligonucleotide
  source 1..20: mol_type = other RNA
agagcaccaagacagagccc

SEQ ID NO: 540 - RNA, 20 nt, synthetic construct
  misc_feature 1..20: note = Description of Artificial Sequence: Syntheticoligonucleotide
  source 1..20: mol_type = other RNA
agacatcaaagtaccctaca

SEQ ID NO: 541 - RNA, 20 nt, synthetic construct
  misc_feature 1..20: note = Description of Artificial Sequence: Syntheticoligonucleotide
  source 1..20: mol_type = other RNA
catcaaagtaccctacagga

SEQ ID NO: 542 - RNA, 20 nt, synthetic construct
  misc_feature 1..20: note = Description of Artificial Sequence: Syntheticoligonucleotide
  source 1..20: mol_type = other RNA
gaggaccagttcccatccgc

SEQ ID NO: 543 - RNA, 20 nt, synthetic construct
  misc_feature 1..20: note = Description of Artificial Sequence: Syntheticoligonucleotide
  source 1..20: mol_type = other RNA
gctcccgcagtacctagcat

SEQ ID NO: 544 - RNA, 20 nt, synthetic construct
  misc_feature 1..20: note = Description of Artificial Sequence: Syntheticoligonucleotide
  source 1..20: mol_type = other RNA
caggaagcagaaggtgcttg

SEQ ID NO: 545 - RNA, 20 nt, synthetic construct
  misc_feature 1..20: note = Description of Artificial Sequence: Syntheticoligonucleotide
  source 1..20: mol_type = other RNA
atttggcagcacgtggtaca

SEQ ID NO: 546 - RNA, 20 nt, synthetic construct
  misc_feature 1..20: note = Description of Artificial Sequence: Syntheticoligonucleotide
  source 1..20: mol_type = other RNA
gcgggccaagacttctccct

SEQ ID NO: 547 - RNA, 20 nt, synthetic construct
  misc_feature 1..20: note = Description of Artificial Sequence: Syntheticoligonucleotide
  source 1..20: mol_type = other RNA
gtccctgcagcagcatgggg

SEQ ID NO: 548 - RNA, 20 nt, synthetic construct
  misc_feature 1..20: note = Description of Artificial Sequence: Syntheticoligonucleotide
  source 1..20: mol_type = other RNA
gctacttcaggcagcagagg

SEQ ID NO: 549 - RNA, 20 nt, synthetic construct
  misc_feature 1..20: note = Description of Artificial Sequence: Syntheticoligonucleotide
  source 1..20: mol_type = other RNA
cttgtgcagactcagaggtg

SEQ ID NO: 550 - RNA, 20 nt, synthetic construct
  misc_feature 1..20: note = Description of Artificial Sequence: Syntheticoligonucleotide
  source 1..20: mol_type = other RNA
gtgcagactcagaggtgaga

SEQ ID NO: 551 - RNA, 20 nt, synthetic construct
  misc_feature 1..20: note = Description of Artificial Sequence: Syntheticoligonucleotide
  source 1..20: mol_type = other RNA
gcagactcagaggtgagagg

SEQ ID NO: 552 - RNA, 20 nt, synthetic construct
  misc_feature 1..20: note = Description of Artificial Sequence: Syntheticoligonucleotide
  source 1..20: mol_type = other RNA
ttcccccagctgaagtcctt

SEQ ID NO: 553 - RNA, 20 nt, synthetic construct
  misc_feature 1..20: note = Description of Artificial Sequence: Syntheticoligonucleotide
  source 1..20: mol_type = other RNA
ctgtcccagaacaacatcac

SEQ ID NO: 554 - RNA, 20 nt, synthetic construct
  misc_feature 1..20: note = Description of Artificial Sequence: Syntheticoligonucleotide
  source 1..20: mol_type = other RNA
cctgcaacaacaggattcac

SEQ ID NO: 555 - RNA, 20 nt, synthetic construct
  misc_feature 1..20: note = Description of Artificial Sequence: Syntheticoligonucleotide
  source 1..20: mol_type = other RNA
cgtccacatcctgcaagggg

SEQ ID NO: 556 - RNA, 20 nt, synthetic construct
  misc_feature 1..20: note = Description of Artificial Sequence: Syntheticoligonucleotide
  source 1..20: mol_type = other RNA
acatcctgcaaggggggatg

SEQ ID NO: 557 - RNA, 20 nt, synthetic construct
  misc_feature 1..20: note = Description of Artificial Sequence: Syntheticoligonucleotide
  source 1..20: mol_type = other RNA
cttacgccagcgtctccaca

SEQ ID NO: 558 - RNA, 20 nt, synthetic construct
  misc_feature 1..20: note = Description of Artificial Sequence: Syntheticoligonucleotide
  source 1..20: mol_type = other RNA
actcactccatcacccggag

SEQ ID NO: 559 - RNA, 20 nt, synthetic construct
  misc_feature 1..20: note = Description of Artificial Sequence: Syntheticoligonucleotide
  source 1..20: mol_type = other RNA
actcaccttagcctgagcag

SEQ ID NO: 560 - RNA, 20 nt, synthetic construct
  misc_feature 1..20: note = Description of Artificial Sequence: Syntheticoligonucleotide
  source 1..20: mol_type = other RNA
gacagactgcggggacacag

SEQ ID NO: 561 - RNA, 20 nt, synthetic construct
  misc_feature 1..20: note = Description of Artificial Sequence: Syntheticoligonucleotide
  source 1..20: mol_type = other RNA
actcacttgagggtttccaa

SEQ ID NO: 562 - RNA, 20 nt, synthetic construct
  misc_feature 1..20: note = Description of Artificial Sequence: Syntheticoligonucleotide
  source 1..20: mol_type = other RNA
tccaggctgcaggtggaatc

SEQ ID NO: 563 - RNA, 20 nt, synthetic construct
  misc_feature 1..20: note = Description of Artificial Sequence: Syntheticoligonucleotide
  source 1..20: mol_type = other RNA
actcactccagatgctgcag

SEQ ID NO: 564 - RNA, 20 nt, synthetic construct
  misc_feature 1..20: note = Description of Artificial Sequence: Syntheticoligonucleotide
  source 1..20: mol_type = other RNA
agccagccacagggccccca

SEQ ID NO: 565 - RNA, 20 nt, synthetic construct
  misc_feature 1..20: note = Description of Artificial Sequence: Syntheticoligonucleotide
  source 1..20: mol_type = other RNA
gcccaggtcctcacgtctgc

SEQ ID NO: 566 - RNA, 20 nt, synthetic construct
  misc_feature 1..20: note = Description of Artificial Sequence: Syntheticoligonucleotide
  source 1..20: mol_type = other RNA
cagcccaatagctcttgccc

SEQ ID NO: 567 - RNA, 20 nt, synthetic construct
  misc_feature 1..20: note = Description of Artificial Sequence: Syntheticoligonucleotide
  source 1..20: mol_type = other RNA
ggccattttggaagcttgtt

SEQ ID NO: 568 - RNA, 20 nt, synthetic construct
  misc_feature 1..20: note = Description of Artificial Sequence: Syntheticoligonucleotide
  source 1..20: mol_type = other RNA
ttacctgtcatgtttgctcg

SEQ ID NO: 569 - RNA, 20 nt, synthetic construct
  misc_feature 1..20: note = Description of Artificial Sequence: Syntheticoligonucleotide
  source 1..20: mol_type = other RNA
acctcacctacattgggggt

SEQ ID NO: 570 - RNA, 20 nt, synthetic construct
  misc_feature 1..20: note = Description of Artificial Sequence: Syntheticoligonucleotide
  source 1..20: mol_type = other RNA
ctcacctacattgggggtgg

SEQ ID NO: 571 - RNA, 20 nt, synthetic construct
  misc_feature 1..20: note = Description of Artificial Sequence: Syntheticoligonucleotide
  source 1..20: mol_type = other RNA
tttgccagagcccatggggc

SEQ ID NO: 572 - RNA, 20 nt, synthetic construct
  misc_feature 1..20: note = Description of Artificial Sequence: Syntheticoligonucleotide
  source 1..20: mol_type = other RNA
aaggctgcagagaaaacatg

SEQ ID NO: 573 - RNA, 20 nt, synthetic construct
  misc_feature 1..20: note = Description of Artificial Sequence: Syntheticoligonucleotide
  source 1..20: mol_type = other RNA
gctctactttgagaaaaacc

SEQ ID NO: 574 - RNA, 20 nt, synthetic construct
  misc_feature 1..20: note = Description of Artificial Sequence: Syntheticoligonucleotide
  source 1..20: mol_type = other RNA
ctcccaggcagctcacagtg

SEQ ID NO: 575 - RNA, 20 nt, synthetic construct
  misc_feature 1..20: note = Description of Artificial Sequence: Syntheticoligonucleotide
  source 1..20: mol_type = other RNA
ctctgcagccttcccagagg

SEQ ID NO: 576 - RNA, 20 nt, synthetic construct
  misc_feature 1..20: note = Description of Artificial Sequence: Syntheticoligonucleotide
  source 1..20: mol_type = other RNA
aatgtaggtgaggtgcccca

SEQ ID NO: 577 - RNA, 20 nt, synthetic construct
  misc_feature 1..20: note = Description of Artificial Sequence: Syntheticoligonucleotide
  source 1..20: mol_type = other RNA
ccgacagcttgtacaataac

SEQ ID NO: 578 - RNA, 20 nt, synthetic construct
  misc_feature 1..20: note = Description of Artificial Sequence: Syntheticoligonucleotide
  source 1..20: mol_type = other RNA
ctcacctctgagtctgcaca

SEQ ID NO: 579 - RNA, 20 nt, synthetic construct
  misc_feature 1..20: note = Description of Artificial Sequence: Syntheticoligonucleotide
  source 1..20: mol_type = other RNA
actcaccaggccattttgga

SEQ ID NO: 580 - RNA, 20 nt, synthetic construct
  misc_feature 1..20: note = Description of Artificial Sequence: Syntheticoligonucleotide
  source 1..20: mol_type = other RNA
cctgcacacctggcttccag

SEQ ID NO: 581 - RNA, 20 nt, synthetic construct
  misc_feature 1..20: note = Description of Artificial Sequence: Syntheticoligonucleotide
  source 1..20: mol_type = other RNA
aaacttactgaaaatgtcct

SEQ ID NO: 582 - RNA, 20 nt, synthetic construct
  misc_feature 1..20: note = Description of Artificial Sequence: Syntheticoligonucleotide
  source 1..20: mol_type = other RNA
tcctcaccgatattggcata

SEQ ID NO: 583 - RNA, 20 nt, synthetic construct
  misc_feature 1..20: note = Description of Artificial Sequence: Syntheticoligonucleotide
  source 1..20: mol_type = other RNA
ctcccaggcagctcacagtg

SEQ ID NO: 584 - RNA, 20 nt, synthetic construct
  misc_feature 1..20: note = Description of Artificial Sequence: Syntheticoligonucleotide
  source 1..20: mol_type = other RNA
ctctgcagccttcccagagg

SEQ ID NO: 585 - RNA, 20 nt, synthetic construct
  misc_feature 1..20: note = Description of Artificial Sequence: Syntheticoligonucleotide
  source 1..20: mol_type = other RNA
cacccccaatgtaggtgagg

SEQ ID NO: 586 - RNA, 20 nt, synthetic construct
  misc_feature 1..20: note = Description of Artificial Sequence: Syntheticoligonucleotide
  source 1..20: mol_type = other RNA
aatgtaggtgaggtgcccca

SEQ ID NO: 587 - RNA, 20 nt, synthetic construct
  misc_feature 1..20: note = Description of Artificial Sequence: Syntheticoligonucleotide
  source 1..20: mol_type = other RNA
ggcctttgcagagccggtgg

SEQ ID NO: 588 - RNA, 20 nt, synthetic construct
  misc_feature 1..20: note = Description of Artificial Sequence: Syntheticoligonucleotide
  source 1..20: mol_type = other RNA
ccctccacagggctgccttg

SEQ ID NO: 589 - RNA, 20 nt, synthetic construct
  misc_feature 1..20: note = Description of Artificial Sequence: Syntheticoligonucleotide
  source 1..20: mol_type = other RNA
gattccacctgcagcctgga

SEQ ID NO: 590 - RNA, 20 nt, synthetic construct
  misc_feature 1..20: note = Description of Artificial Sequence: Syntheticoligonucleotide
  source 1..20: mol_type = other RNA
ttccacctgcagcctggatg

SEQ ID NO: 591 - RNA, 20 nt, synthetic construct
  misc_feature 1..20: note = Description of Artificial Sequence: Syntheticoligonucleotide
  source 1..20: mol_type = other RNA
ccgacagcttgtacaataac

SEQ ID NO: 592 - RNA, 20 nt, synthetic construct
  misc_feature 1..20: note = Description of Artificial Sequence: Syntheticoligonucleotide
  source 1..20: mol_type = other RNA
ccccccttgcaggatgtgga

SEQ ID NO: 593 - RNA, 20 nt, synthetic construct
  misc_feature 1..20: note = Description of Artificial Sequence: Syntheticoligonucleotide
  source 1..20: mol_type = other RNA
gccccactcaccttagcctg

SEQ ID NO: 594 - RNA, 20 nt, synthetic construct
  misc_feature 1..20: note = Description of Artificial Sequence: Syntheticoligonucleotide
  source 1..20: mol_type = other RNA
gagtctatactcactccaga

SEQ ID NO: 595 - RNA, 20 nt, synthetic construct
  misc_feature 1..20: note = Description of Artificial Sequence: Syntheticoligonucleotide
  source 1..20: mol_type = other RNA
tctatactcactccagatgc

SEQ ID NO: 596 - RNA, 20 nt, synthetic construct
  misc_feature 1..20: note = Description of Artificial Sequence: Syntheticoligonucleotide
  source 1..20: mol_type = other RNA
tctcctctcacctctgagtc

SEQ ID NO: 597 - RNA, 20 nt, synthetic construct
  misc_feature 1..20: note = Description of Artificial Sequence: Syntheticoligonucleotide
  source 1..20: mol_type = other RNA
ctcacctctgagtctgcaca

SEQ ID NO: 598 - RNA, 20 nt, synthetic construct
  misc_feature 1..20: note = Description of Artificial Sequence: Syntheticoligonucleotide
  source 1..20: mol_type = other RNA
actcaccaggccattttgga

SEQ ID NO: 599 - RNA, 20 nt, synthetic construct
  misc_feature 1..20: note = Description of Artificial Sequence: Syntheticoligonucleotide
  source 1..20: mol_type = other RNA
gggtccttacctgtcatgtt

SEQ ID NO: 600 - RNA, 20 nt, synthetic construct
  misc_feature 1..20: note = Description of Artificial Sequence: Syntheticoligonucleotide
  source 1..20: mol_type = other RNA
cctgcacacctggcttccag

SEQ ID NO: 601 - RNA, 20 nt, synthetic construct
  misc_feature 1..20: note = Description of Artificial Sequence: Syntheticoligonucleotide
  source 1..20: mol_type = other RNA
aaacttactgaaaatgtcct

SEQ ID NO: 602 - RNA, 20 nt, synthetic construct
  misc_feature 1..20: note = Description of Artificial Sequence: Syntheticoligonucleotide
  source 1..20: mol_type = other RNA
ggtgcttcctcaccgatatt

SEQ ID NO: 603 - RNA, 20 nt, synthetic construct
  misc_feature 1..20: note = Description of Artificial Sequence: Syntheticoligonucleotide
  source 1..20: mol_type = other RNA
tcctcaccgatattggcata

SEQ ID NO: 604 - RNA, 20 nt, synthetic construct
  misc_feature 1..20: note = Description of Artificial Sequence: Syntheticoligonucleotide
  source 1..20: mol_type = other RNA
aggagggcccacctgagtag

SEQ ID NO: 605 - RNA, 20 nt, synthetic construct
  misc_feature 1..20: note = Description of Artificial Sequence: Syntheticoligonucleotide
  source 1..20: mol_type = other RNA
actcccagctggagggcctg

SEQ ID NO: 606 - RNA, 20 nt, synthetic construct
  misc_feature 1..20: note = Description of Artificial Sequence: Syntheticoligonucleotide
  source 1..20: mol_type = other RNA
agtcagaaaagacgtgagtg

SEQ ID NO: 607 - RNA, 20 nt, synthetic construct
  misc_feature 1..20: note = Description of Artificial Sequence: Syntheticoligonucleotide
  source 1..20: mol_type = other RNA
tcaaccaggagccagcctcc

SEQ ID NO: 608 - RNA, 20 nt, synthetic construct
  misc_feature 1..20: note = Description of Artificial Sequence: Syntheticoligonucleotide
  source 1..20: mol_type = other RNA
ggagcagttctaccgctcac

SEQ ID NO: 609 - RNA, 20 nt, synthetic construct
  misc_feature 1..20: note = Description of Artificial Sequence: Syntheticoligonucleotide
  source 1..20: mol_type = other RNA
tcactgcaggacacgtatgg

SEQ ID NO: 610 - RNA, 20 nt, synthetic construct
  misc_feature 1..20: note = Description of Artificial Sequence: Syntheticoligonucleotide
  source 1..20: mol_type = other RNA
aacggcagctggcccaagga

SEQ ID NO: 611 - RNA, 20 nt, synthetic construct
  misc_feature 1..20: note = Description of Artificial Sequence: Syntheticoligonucleotide
  source 1..20: mol_type = other RNA
tgagacacgagtgattgctg

SEQ ID NO: 612 - RNA, 20 nt, synthetic construct
  misc_feature 1..20: note = Description of Artificial Sequence: Syntheticoligonucleotide
  source 1..20: mol_type = other RNA
ggtcagggcaagagctattg

SEQ ID NO: 613 - RNA, 20 nt, synthetic construct
  misc_feature 1..20: note = Description of Artificial Sequence: Syntheticoligonucleotide
  source 1..20: mol_type = other RNA
ggcccacagccactcgtggc

SEQ ID NO: 614 - RNA, 20 nt, synthetic construct
  misc_feature 1..20: note = Description of Artificial Sequence: Syntheticoligonucleotide
  source 1..20: mol_type = other RNA
ggaagcgcaagatggcttcc

SEQ ID NO: 615 - RNA, 20 nt, synthetic construct
  misc_feature 1..20: note = Description of Artificial Sequence: Syntheticoligonucleotide
  source 1..20: mol_type = other RNA
ctggtccagagcctgagcaa

SEQ ID NO: 616 - RNA, 20 nt, synthetic construct
  misc_feature 1..20: note = Description of Artificial Sequence: Syntheticoligonucleotide
  source 1..20: mol_type = other RNA
gcaggcccaggcatacgtga

SEQ ID NO: 617 - RNA, 20 nt, synthetic construct
  misc_feature 1..20: note = Description of Artificial Sequence: Syntheticoligonucleotide
  source 1..20: mol_type = other RNA
aggcccaggcatacgtgatg

SEQ ID NO: 618 - RNA, 20 nt, synthetic construct
  misc_feature 1..20: note = Description of Artificial Sequence: Syntheticoligonucleotide
  source 1..20: mol_type = other RNA
gcaccaagacagagccctga

SEQ ID NO: 619 - RNA, 20 nt, synthetic construct
  misc_feature 1..20: note = Description of Artificial Sequence: Syntheticoligonucleotide
  source 1..20: mol_type = other RNA
gtgccagctctcagaggccc

SEQ ID NO: 620 - RNA, 20 nt, synthetic construct
  misc_feature 1..20: note = Description of Artificial Sequence: Syntheticoligonucleotide
  source 1..20: mol_type = other RNA
ttagtccaacacccaccgcg

SEQ ID NO: 621 - RNA, 20 nt, synthetic construct
  misc_feature 1..20: note = Description of Artificial Sequence: Syntheticoligonucleotide
  source 1..20: mol_type = other RNA
gtccaacacccaccgcgggc

SEQ ID NO: 622 - RNA, 20 nt, synthetic construct
  misc_feature 1..20: note = Description of Artificial Sequence: Syntheticoligonucleotide
  source 1..20: mol_type = other RNA
ctcctgcaatgcttcctggg

SEQ ID NO: 623 - RNA, 20 nt, synthetic construct
  misc_feature 1..20: note = Description of Artificial Sequence: Syntheticoligonucleotide
  source 1..20: mol_type = other RNA
tcttccagcctcccgcccgc

SEQ ID NO: 624 - RNA, 20 nt, synthetic construct
  misc_feature 1..20: note = Description of Artificial Sequence: Syntheticoligonucleotide
  source 1..20: mol_type = other RNA
gcggctgcagccggggacac

SEQ ID NO: 625 - RNA, 20 nt, synthetic construct
  misc_feature 1..20: note = Description of Artificial Sequence: Syntheticoligonucleotide
  source 1..20: mol_type = other RNA
ctgcagccggggacactgcg

SEQ ID NO: 626 - RNA, 20 nt, synthetic construct
  misc_feature 1..20: note = Description of Artificial Sequence: Syntheticoligonucleotide
  source 1..20: mol_type = other RNA
ggcgcggcagctgctggagc

SEQ ID NO: 627 - RNA, 20 nt, synthetic construct
  misc_feature 1..20: note = Description of Artificial Sequence: Syntheticoligonucleotide
  source 1..20: mol_type = other RNA
gcggcagctgctggagctgc

SEQ ID NO: 628 - RNA, 20 nt, synthetic construct
  misc_feature 1..20: note = Description of Artificial Sequence: Syntheticoligonucleotide
  source 1..20: mol_type = other RNA
ttggcagcacgtggtacagg

SEQ ID NO: 629 - RNA, 20 nt, synthetic construct
  misc_feature 1..20: note = Description of Artificial Sequence: Syntheticoligonucleotide
  source 1..20: mol_type = other RNA
tggtacaggagctccccggc

SEQ ID NO: 630 - RNA, 20 nt, synthetic construct
  misc_feature 1..20: note = Description of Artificial Sequence: Syntheticoligonucleotide
  source 1..20: mol_type = other RNA
gcagcagcatggggagacca

SEQ ID NO: 631 - RNA, 20 nt, synthetic construct
  misc_feature 1..20: note = Description of Artificial Sequence: Syntheticoligonucleotide
  source 1..20: mol_type = other RNA
gactcagaggtgagaggaga

SEQ ID NO: 632 - RNA, 20 nt, synthetic construct
  misc_feature 1..20: note = Description of Artificial Sequence: Syntheticoligonucleotide
  source 1..20: mol_type = other RNA
gtccagtacaacaagttcac

SEQ ID NO: 633 - RNA, 20 nt, synthetic construct
  misc_feature 1..20: note = Description of Artificial Sequence: Syntheticoligonucleotide
  source 1..20: mol_type = other RNA
gggcccagcagctcgctgcc

SEQ ID NO: 634 - RNA, 20 nt, synthetic construct
  misc_feature 1..20: note = Description of Artificial Sequence: Syntheticoligonucleotide
  source 1..20: mol_type = other RNA
aacaacaggattcacggatc

SEQ ID NO: 635 - RNA, 20 nt, synthetic construct
  misc_feature 1..20: note = Description of Artificial Sequence: Syntheticoligonucleotide
  source 1..20: mol_type = other RNA
tacaagctgtcggaaacaga

SEQ ID NO: 636 - RNA, 20 nt, synthetic construct
  misc_feature 1..20: note = Description of Artificial Sequence: Syntheticoligonucleotide
  source 1..20: mol_type = other RNA
gcccagcctaggaggcaaag

SEQ ID NO: 637 - RNA, 20 nt, synthetic construct
  misc_feature 1..20: note = Description of Artificial Sequence: Syntheticoligonucleotide
  source 1..20: mol_type = other RNA
ctcacctctgagtctgcaca

SEQ ID NO: 638 - RNA, 20 nt, synthetic construct
  misc_feature 1..20: note = Description of Artificial Sequence: Syntheticoligonucleotide
  source 1..20: mol_type = other RNA
ctcccacagcgccaccgtgt

SEQ ID NO: 639 - RNA, 20 nt, synthetic construct
  misc_feature 1..20: note = Description of Artificial Sequence: Syntheticoligonucleotide
  source 1..20: mol_type = other RNA
ccacctgaaacgggtgacac

SEQ ID NO: 640 - RNA, 20 nt, synthetic construct
  misc_feature 1..20: note = Description of Artificial Sequence: Syntheticoligonucleotide
  source 1..20: mol_type = other RNA
tgccaaattccagcctcctc

SEQ ID NO: 641 - RNA, 20 nt, synthetic construct
  misc_feature 1..20: note = Description of Artificial Sequence: Syntheticoligonucleotide
  source 1..20: mol_type = other RNA
cctccagccagttgtcatag

SEQ ID NO: 642 - RNA, 20 nt, synthetic construct
  misc_feature 1..20: note = Description of Artificial Sequence: Syntheticoligonucleotide
  source 1..20: mol_type = other RNA
cagccacagggcccccagga

SEQ ID NO: 643 - RNA, 20 nt, synthetic construct
  misc_feature 1..20: note = Description of Artificial Sequence: Syntheticoligonucleotide
  source 1..20: mol_type = other RNA
atcgcccaggtcctcacgtc

SEQ ID NO: 644 - RNA, 20 nt, synthetic construct
  misc_feature 1..20: note = Description of Artificial Sequence: Syntheticoligonucleotide
  source 1..20: mol_type = other RNA
gctcccaggccagcttggcc

SEQ ID NO: 645 - RNA, 20 nt, synthetic construct
  misc_feature 1..20: note = Description of Artificial Sequence: Syntheticoligonucleotide
  source 1..20: mol_type = other RNA
caagcccaggcccggctcac

SEQ ID NO: 646 - RNA, 20 nt, synthetic construct
  misc_feature 1..20: note = Description of Artificial Sequence: Syntheticoligonucleotide
  source 1..20: mol_type = other RNA
ccggctctgcaaaggccagg

SEQ ID NO: 647 - RNA, 20 nt, synthetic construct
  misc_feature 1..20: note = Description of Artificial Sequence: Syntheticoligonucleotide
  source 1..20: mol_type = other RNA
actcaccaggccattttgga

SEQ ID NO: 648 - RNA, 20 nt, synthetic construct
  misc_feature 1..20: note = Description of Artificial Sequence: Syntheticoligonucleotide
  source 1..20: mol_type = other RNA
ttgtgctctggagatggaga

SEQ ID NO: 649 - RNA, 20 nt, synthetic construct
  misc_feature 1..20: note = Description of Artificial Sequence: Syntheticoligonucleotide
  source 1..20: mol_type = other RNA
agatttgccagagcccatgg

SEQ ID NO: 650 - RNA, 20 nt, synthetic construct
  misc_feature 1..20: note = Description of Artificial Sequence: Syntheticoligonucleotide
  source 1..20: mol_type = other RNA
aaggcactgcaagagacaaa

SEQ ID NO: 651 - RNA, 20 nt, synthetic construct
  misc_feature 1..20: note = Description of Artificial Sequence: Syntheticoligonucleotide
  source 1..20: mol_type = other RNA
ggctcagctgtgaggaagtg

SEQ ID NO: 652 - RNA, 20 nt, synthetic construct
  misc_feature 1..20: note = Description of Artificial Sequence: Syntheticoligonucleotide
  source 1..20: mol_type = other RNA
ggcttccagtgcttcaggtc

SEQ ID NO: 653 - RNA, 20 nt, synthetic construct
  misc_feature 1..20: note = Description of Artificial Sequence: Syntheticoligonucleotide
  source 1..20: mol_type = other RNA
aaacttactgaaaatgtcct

SEQ ID NO: 654 - RNA, 20 nt, synthetic construct
  misc_feature 1..20: note = Description of Artificial Sequence: Syntheticoligonucleotide
  source 1..20: mol_type = other RNA
ccgctgcccagacaaggaaa

SEQ ID NO: 655 - RNA, 20 nt, synthetic construct
  misc_feature 1..20: note = Description of Artificial Sequence: Syntheticoligonucleotide
  source 1..20: mol_type = other RNA
tcctcaccgatattggcata

SEQ ID NO: 656 - DNA, 20 bp, synthetic construct
  misc_feature 1..20: note = Description of Artificial Sequence: Syntheticoligonucleotide
  misc_feature 1..20: note = Description of Combined DNA/RNA Molecule: Syntheticoligonucleotide
  source 1..20: mol_type = other DNA
ctgcctgggagggaagacaa

SEQ ID NO: 657 - RNA, 20 nt, synthetic construct
  misc_feature 1..20: note = Description of Artificial Sequence: Syntheticoligonucleotide
  source 1..20: mol_type = other RNA
ccacctgcagcctggatgcg

SEQ ID NO: 658 - RNA, 20 nt, synthetic construct
  misc_feature 1..20: note = Description of Artificial Sequence: Syntheticoligonucleotide
  source 1..20: mol_type = other RNA
ccactcaccttagcctgagc

SEQ ID NO: 659 - RNA, 20 nt, synthetic construct
  misc_feature 1..20: note = Description of Artificial Sequence: Syntheticoligonucleotide
  source 1..20: mol_type = other RNA
cacagctgagccccccactg

SEQ ID NO: 660 - RNA, 20 nt, synthetic construct
  misc_feature 1..20: note = Description of Artificial Sequence: Syntheticoligonucleotide
  source 1..20: mol_type = other RNA
caatgtaggtgaggtgcccc

SEQ ID NO: 661 - RNA, 20 nt, synthetic construct
  misc_feature 1..20: note = Description of Artificial Sequence: Syntheticoligonucleotide
  source 1..20: mol_type = other RNA
ctccaggacgagaagttcct

SEQ ID NO: 662 - RNA, 20 nt, synthetic construct
  misc_feature 1..20: note = Description of Artificial Sequence: Syntheticoligonucleotide
  source 1..20: mol_type = other RNA
cctaggctgggccctgtctc

SEQ ID NO: 663 - RNA, 20 nt, synthetic construct
  misc_feature 1..20: note = Description of Artificial Sequence: Syntheticoligonucleotide
  source 1..20: mol_type = other RNA
acactcactccatcacccgg

SEQ ID NO: 664 - RNA, 20 nt, synthetic construct
  misc_feature 1..20: note = Description of Artificial Sequence: Syntheticoligonucleotide
  source 1..20: mol_type = other RNA
cactcactccatcacccgga

SEQ ID NO: 665 - RNA, 20 nt, synthetic construct
  misc_feature 1..20: note = Description of Artificial Sequence: Syntheticoligonucleotide
  source 1..20: mol_type = other RNA
ccactcaccttagcctgagc

SEQ ID NO: 666 - RNA, 20 nt, synthetic construct
  misc_feature 1..20: note = Description of Artificial Sequence: Syntheticoligonucleotide
  source 1..20: mol_type = other RNA
cactcaccttagcctgagca

SEQ ID NO: 667 - RNA, 20 nt, synthetic construct
  misc_feature 1..20: note = Description of Artificial Sequence: Syntheticoligonucleotide
  source 1..20: mol_type = other RNA
cactcacttgagggtttcca

SEQ ID NO: 668 - RNA, 20 nt, synthetic construct
  misc_feature 1..20: note = Description of Artificial Sequence: Syntheticoligonucleotide
  source 1..20: mol_type = other RNA
atactcactccagatgctgc

SEQ ID NO: 669 - RNA, 20 nt, synthetic construct
  misc_feature 1..20: note = Description of Artificial Sequence: Syntheticoligonucleotide
  source 1..20: mol_type = other RNA
tactcactccagatgctgca

SEQ ID NO: 670 - RNA, 20 nt, synthetic construct
  misc_feature 1..20: note = Description of Artificial Sequence: Syntheticoligonucleotide
  source 1..20: mol_type = other RNA
ccttacctgtcatgtttgct

SEQ ID NO: 671 - RNA, 20 nt, synthetic construct
  misc_feature 1..20: note = Description of Artificial Sequence: Syntheticoligonucleotide
  source 1..20: mol_type = other RNA
cttacctgtcatgtttgctc

SEQ ID NO: 672 - RNA, 20 nt, synthetic construct
  misc_feature 1..20: note = Description of Artificial Sequence: Syntheticoligonucleotide
  source 1..20: mol_type = other RNA
taacatactgggaatctggt

SEQ ID NO: 673 - RNA, 20 nt, synthetic construct
  misc_feature 1..20: note = Description of Artificial Sequence: Syntheticoligonucleotide
  source 1..20: mol_type = other RNA
ttttaccttggggctctgac

SEQ ID NO: 674 - RNA, 20 nt, synthetic construct
  misc_feature 1..20: note = Description of Artificial Sequence: Syntheticoligonucleotide
  source 1..20: mol_type = other RNA
ccttgtctgggcagcggaac

SEQ ID NO: 675 - RNA, 20 nt, synthetic construct
  misc_feature 1..20: note = Description of Artificial Sequence: Syntheticoligonucleotide
  source 1..20: mol_type = other RNA
tttctcaaagtagagcacat

SEQ ID NO: 676 - RNA, 20 nt, synthetic construct
  misc_feature 1..20: note = Description of Artificial Sequence: Syntheticoligonucleotide
  source 1..20: mol_type = other RNA
tttctctgcagccttcccag

SEQ ID NO: 677 - RNA, 20 nt, synthetic construct
  misc_feature 1..20: note = Description of Artificial Sequence: Syntheticoligonucleotide
  source 1..20: mol_type = other RNA
cacagctgagccccccactg

SEQ ID NO: 678 - RNA, 20 nt, synthetic construct
  misc_feature 1..20: note = Description of Artificial Sequence: Syntheticoligonucleotide
  source 1..20: mol_type = other RNA
caatgtaggtgaggtgcccc

SEQ ID NO: 679 - RNA, 20 nt, synthetic construct
  misc_feature 1..20: note = Description of Artificial Sequence: Syntheticoligonucleotide
  source 1..20: mol_type = other RNA
cctggcctttgcagagccgg

SEQ ID NO: 680 - RNA, 20 nt, synthetic construct
  misc_feature 1..20: note = Description of Artificial Sequence: Syntheticoligonucleotide
  source 1..20: mol_type = other RNA
ctccaggacgagaagttcct

SEQ ID NO: 681 - RNA, 20 nt, synthetic construct
  misc_feature 1..20: note = Description of Artificial Sequence: Syntheticoligonucleotide
  source 1..20: mol_type = other RNA
cctaggctgggccctgtctc

SEQ ID NO: 682 - RNA, 20 nt, synthetic construct
  misc_feature 1..20: note = Description of Artificial Sequence: Syntheticoligonucleotide
  source 1..20: mol_type = other RNA
cccacactcactccatcacc

SEQ ID NO: 683 - RNA, 20 nt, synthetic construct
  misc_feature 1..20: note = Description of Artificial Sequence: Syntheticoligonucleotide
  source 1..20: mol_type = other RNA
acactcactccatcacccgg

SEQ ID NO: 684 - RNA, 20 nt, synthetic construct
  misc_feature 1..20: note = Description of Artificial Sequence: Syntheticoligonucleotide
  source 1..20: mol_type = other RNA
cactcactccatcacccgga

SEQ ID NO: 685 - RNA, 20 nt, synthetic construct
  misc_feature 1..20: note = Description of Artificial Sequence: Syntheticoligonucleotide
  source 1..20: mol_type = other RNA
ccactcaccttagcctgagc

SEQ ID NO: 686 - RNA, 20 nt, synthetic construct
  misc_feature 1..20: note = Description of Artificial Sequence: Syntheticoligonucleotide
  source 1..20: mol_type = other RNA
cactcaccttagcctgagca

SEQ ID NO: 687 - RNA, 20 nt, synthetic construct
  misc_feature 1..20: note = Description of Artificial Sequence: Syntheticoligonucleotide
  source 1..20: mol_type = other RNA
cactcacttgagggtttcca

SEQ ID NO: 688 - RNA, 20 nt, synthetic construct
  misc_feature 1..20: note = Description of Artificial Sequence: Syntheticoligonucleotide
  source 1..20: mol_type = other RNA
atactcactccagatgctgc

SEQ ID NO: 689 - RNA, 20 nt, synthetic construct
  misc_feature 1..20: note = Description of Artificial Sequence: Syntheticoligonucleotide
  source 1..20: mol_type = other RNA
tactcactccagatgctgca

SEQ ID NO: 690 - RNA, 20 nt, synthetic construct
  misc_feature 1..20: note = Description of Artificial Sequence: Syntheticoligonucleotide
  source 1..20: mol_type = other RNA
gcccctcaccccacctgaaa

SEQ ID NO: 691 - RNA, 20 nt, synthetic construct
  misc_feature 1..20: note = Description of Artificial Sequence: Syntheticoligonucleotide
  source 1..20: mol_type = other RNA
cccctcaccccacctgaaac

SEQ ID NO: 692 - RNA, 20 nt, synthetic construct
  misc_feature 1..20: note = Description of Artificial Sequence: Syntheticoligonucleotide
  source 1..20: mol_type = other RNA
catcactcaccaggccattt

SEQ ID NO: 693 - RNA, 20 nt, synthetic construct
  misc_feature 1..20: note = Description of Artificial Sequence: Syntheticoligonucleotide
  source 1..20: mol_type = other RNA
ccttacctgtcatgtttgct

SEQ ID NO: 694 - RNA, 20 nt, synthetic construct
  misc_feature 1..20: note = Description of Artificial Sequence: Syntheticoligonucleotide
  source 1..20: mol_type = other RNA
cttacctgtcatgtttgctc

SEQ ID NO: 695 - RNA, 20 nt, synthetic construct
  misc_feature 1..20: note = Description of Artificial Sequence: Syntheticoligonucleotide
  source 1..20: mol_type = other RNA
cccctaacatactgggaatc

SEQ ID NO: 696 - RNA, 20 nt, synthetic construct
  misc_feature 1..20: note = Description of Artificial Sequence: Syntheticoligonucleotide
  source 1..20: mol_type = other RNA
taacatactgggaatctggt

SEQ ID NO: 697 - RNA, 20 nt, synthetic construct
  misc_feature 1..20: note = Description of Artificial Sequence: Syntheticoligonucleotide
  source 1..20: mol_type = other RNA
aggtgcttcctcaccgatat

SEQ ID NO: 698 - RNA, 20 nt, synthetic construct
  misc_feature 1..20: note = Description of Artificial Sequence: Syntheticoligonucleotide
  source 1..20: mol_type = other RNA
ttttaccttggggctctgac

SEQ ID NO: 699 - RNA, 20 nt, synthetic construct
  misc_feature 1..20: note = Description of Artificial Sequence: Syntheticoligonucleotide
  source 1..20: mol_type = other RNA
gagccccaaggtaaaaaggc

SEQ ID NO: 700 - RNA, 20 nt, synthetic construct
  misc_feature 1..20: note = Description of Artificial Sequence: Syntheticoligonucleotide
  source 1..20: mol_type = other RNA
agccccaaggtaaaaaggcc

SEQ ID NO: 701 - RNA, 20 nt, synthetic construct
  misc_feature 1..20: note = Description of Artificial Sequence: Syntheticoligonucleotide
  source 1..20: mol_type = other RNA
cagctcacagtgtgccacca

SEQ ID NO: 702 - RNA, 20 nt, synthetic construct
  misc_feature 1..20: note = Description of Artificial Sequence: Syntheticoligonucleotide
  source 1..20: mol_type = other RNA
tatgaccagatggacctggc

SEQ ID NO: 703 - DNA, 20 bp, synthetic construct
  misc_feature 1..20: note = Description of Artificial Sequence: Syntheticoligonucleotide
  misc_feature 1..20: note = Description of Combined DNA/RNA Molecule: Syntheticoligonucleotide
  source 1..20: mol_type = other DNA
actggaccagtatgtcttcc

SEQ ID NO: 704 - RNA, 20 nt, synthetic construct
  misc_feature 1..20: note = Description of Artificial Sequence: Syntheticoligonucleotide
  source 1..20: mol_type = other RNA
tgtcttccaggactcccagc

SEQ ID NO: 705 - RNA, 20 nt, synthetic construct
  misc_feature 1..20: note = Description of Artificial Sequence: Syntheticoligonucleotide
  source 1..20: mol_type = other RNA
cttccaggactcccagctgg

SEQ ID NO: 706 - RNA, 20 nt, synthetic construct
  misc_feature 1..20: note = Description of Artificial Sequence: Syntheticoligonucleotide
  source 1..20: mol_type = other RNA
ttccaggactcccagctgga

SEQ ID NO: 707 - RNA, 20 nt, synthetic construct
  misc_feature 1..20: note = Description of Artificial Sequence: Syntheticoligonucleotide
  source 1..20: mol_type = other RNA
ttcaaccaggagccagcctc

SEQ ID NO: 708 - RNA, 20 nt, synthetic construct
  misc_feature 1..20: note = Description of Artificial Sequence: Syntheticoligonucleotide
  source 1..20: mol_type = other RNA
gaccagattcccagtatgtt

SEQ ID NO: 709 - RNA, 20 nt, synthetic construct
  misc_feature 1..20: note = Description of Artificial Sequence: Syntheticoligonucleotide
  source 1..20: mol_type = other RNA
ctctggcaaatctctgaggc

SEQ ID NO: 710 - RNA, 20 nt, synthetic construct
  misc_feature 1..20: note = Description of Artificial Sequence: Syntheticoligonucleotide
  source 1..20: mol_type = other RNA
agccaagtaccccctcccag

SEQ ID NO: 711 - RNA, 20 nt, synthetic construct
  misc_feature 1..20: note = Description of Artificial Sequence: Syntheticoligonucleotide
  source 1..20: mol_type = other RNA
acctcccgagcaaacatgac

SEQ ID NO: 712 - RNA, 20 nt, synthetic construct
  misc_feature 1..20: note = Description of Artificial Sequence: Syntheticoligonucleotide
  source 1..20: mol_type = other RNA
cccacccaatgcccggcagc

SEQ ID NO: 713 - RNA, 20 nt, synthetic construct
  misc_feature 1..20: note = Description of Artificial Sequence: Syntheticoligonucleotide
  source 1..20: mol_type = other RNA
tggtgcaggccaggctggag

SEQ ID NO: 714 - RNA, 20 nt, synthetic construct
  misc_feature 1..20: note = Description of Artificial Sequence: Syntheticoligonucleotide
  source 1..20: mol_type = other RNA
gaacggcagctggcccaagg

SEQ ID NO: 715 - RNA, 20 nt, synthetic construct
  misc_feature 1..20: note = Description of Artificial Sequence: Syntheticoligonucleotide
  source 1..20: mol_type = other RNA
ggcccaaggaggcctggctg

SEQ ID NO: 716 - RNA, 20 nt, synthetic construct
  misc_feature 1..20: note = Description of Artificial Sequence: Syntheticoligonucleotide
  source 1..20: mol_type = other RNA
gacacgagtgattgctgtgc

SEQ ID NO: 717 - RNA, 20 nt, synthetic construct
  misc_feature 1..20: note = Description of Artificial Sequence: Syntheticoligonucleotide
  source 1..20: mol_type = other RNA
acacgagtgattgctgtgct

SEQ ID NO: 718 - RNA, 20 nt, synthetic construct
  misc_feature 1..20: note = Description of Artificial Sequence: Syntheticoligonucleotide
  source 1..20: mol_type = other RNA
ctggtcagggcaagagctat

SEQ ID NO: 719 - RNA, 20 nt, synthetic construct
  misc_feature 1..20: note = Description of Artificial Sequence: Syntheticoligonucleotide
  source 1..20: mol_type = other RNA
tggtcagggcaagagctatt

SEQ ID NO: 720 - RNA, 20 nt, synthetic construct
  misc_feature 1..20: note = Description of Artificial Sequence: Syntheticoligonucleotide
  source 1..20: mol_type = other RNA
gggcccacagccactcgtgg

SEQ ID NO: 721 - RNA, 20 nt, synthetic construct
  misc_feature 1..20: note = Description of Artificial Sequence: Syntheticoligonucleotide
  source 1..20: mol_type = other RNA
ttccagaagaagctgctccg

SEQ ID NO: 722 - RNA, 20 nt, synthetic construct
  misc_feature 1..20: note = Description of Artificial Sequence: Syntheticoligonucleotide
  source 1..20: mol_type = other RNA
cctggtccagagcctgagca

SEQ ID NO: 723 - RNA, 20 nt, synthetic construct
  misc_feature 1..20: note = Description of Artificial Sequence: Syntheticoligonucleotide
  source 1..20: mol_type = other RNA
cagacatcaaagtaccctac

SEQ ID NO: 724 - RNA, 20 nt, synthetic construct
  misc_feature 1..20: note = Description of Artificial Sequence: Syntheticoligonucleotide
  source 1..20: mol_type = other RNA
acatcaaagtaccctacagg

SEQ ID NO: 725 - RNA, 20 nt, synthetic construct
  misc_feature 1..20: note = Description of Artificial Sequence: Syntheticoligonucleotide
  source 1..20: mol_type = other RNA
cttagtccaacacccaccgc

SEQ ID NO: 726 - RNA, 20 nt, synthetic construct
  misc_feature 1..20: note = Description of Artificial Sequence: Syntheticoligonucleotide
  source 1..20: mol_type = other RNA
cctcctgcaatgcttcctgg

SEQ ID NO: 727 - RNA, 20 nt, synthetic construct
  misc_feature 1..20: note = Description of Artificial Sequence: Syntheticoligonucleotide
  source 1..20: mol_type = other RNA
ggaagcagaaggtgcttgcg

SEQ ID NO: 728 - RNA, 23 nt, synthetic construct
  misc_feature 1..23: note = Description of Artificial Sequence: Syntheticoligonucleotide
  source 1..23: mol_type = other RNA
ggctgcagccggggacactgcgg

SEQ ID NO: 729 - RNA, 20 nt, synthetic construct
  misc_feature 1..20: note = Description of Artificial Sequence: Syntheticoligonucleotide
  source 1..20: mol_type = other RNA
gctgcagccggggacactgc

SEQ ID NO: 730 - RNA, 20 nt, synthetic construct
  misc_feature 1..20: note = Description of Artificial Sequence: Syntheticoligonucleotide
  source 1..20: mol_type = other RNA
aatttggcagcacgtggtac

SEQ ID NO: 731 - RNA, 20 nt, synthetic construct
  misc_feature 1..20: note = Description of Artificial Sequence: Syntheticoligonucleotide
  source 1..20: mol_type = other RNA
ggcgggccaagacttctccc

SEQ ID NO: 732 - RNA, 20 nt, synthetic construct
  misc_feature 1..20: note = Description of Artificial Sequence: Syntheticoligonucleotide
  source 1..20: mol_type = other RNA
tgtgcagactcagaggtgag

SEQ ID NO: 733 - RNA, 20 nt, synthetic construct
  misc_feature 1..20: note = Description of Artificial Sequence: Syntheticoligonucleotide
  source 1..20: mol_type = other RNA
agactcagaggtgagaggag

SEQ ID NO: 734 - RNA, 20 nt, synthetic construct
  misc_feature 1..20: note = Description of Artificial Sequence: Syntheticoligonucleotide
  source 1..20: mol_type = other RNA
cccccaggctttccccaaac

SEQ ID NO: 735 - RNA, 20 nt, synthetic construct
  misc_feature 1..20: note = Description of Artificial Sequence: Syntheticoligonucleotide
  source 1..20: mol_type = other RNA
cttcccccagctgaagtcct

SEQ ID NO: 736 - RNA, 20 nt, synthetic construct
  misc_feature 1..20: note = Description of Artificial Sequence: Syntheticoligonucleotide
  source 1..20: mol_type = other RNA
cgtccagtacaacaagttca

SEQ ID NO: 737 - RNA, 20 nt, synthetic construct
  misc_feature 1..20: note = Description of Artificial Sequence: Syntheticoligonucleotide
  source 1..20: mol_type = other RNA
acctgcaacaacaggattca

SEQ ID NO: 738 - RNA, 20 nt, synthetic construct
  misc_feature 1..20: note = Description of Artificial Sequence: Syntheticoligonucleotide
  source 1..20: mol_type = other RNA
tgggcgtccacatcctgcaa

SEQ ID NO: 739 - RNA, 20 nt, synthetic construct
  misc_feature 1..20: note = Description of Artificial Sequence: Syntheticoligonucleotide
  source 1..20: mol_type = other RNA
gggcgtccacatcctgcaag

SEQ ID NO: 740 - RNA, 20 nt, synthetic construct
  misc_feature 1..20: note = Description of Artificial Sequence: Syntheticoligonucleotide
  source 1..20: mol_type = other RNA
ggcgtccacatcctgcaagg

SEQ ID NO: 741 - RNA, 20 nt, synthetic construct
  misc_feature 1..20: note = Description of Artificial Sequence: Syntheticoligonucleotide
  source 1..20: mol_type = other RNA
gcgtccacatcctgcaaggg

SEQ ID NO: 742 - RNA, 20 nt, synthetic construct
  misc_feature 1..20: note = Description of Artificial Sequence: Syntheticoligonucleotide
  source 1..20: mol_type = other RNA
ccacatcctgcaagggggga

SEQ ID NO: 743 - RNA, 20 nt, synthetic construct
  misc_feature 1..20: note = Description of Artificial Sequence: Syntheticoligonucleotide
  source 1..20: mol_type = other RNA
cacatcctgcaaggggggat

SEQ ID NO: 744 - RNA, 20 nt, synthetic construct
  misc_feature 1..20: note = Description of Artificial Sequence: Syntheticoligonucleotide
  source 1..20: mol_type = other RNA
acactcactccatcacccgg

SEQ ID NO: 745 - RNA, 20 nt, synthetic construct
  misc_feature 1..20: note = Description of Artificial Sequence: Syntheticoligonucleotide
  source 1..20: mol_type = other RNA
cactcactccatcacccgga

SEQ ID NO: 746 - RNA, 20 nt, synthetic construct
  misc_feature 1..20: note = Description of Artificial Sequence: Syntheticoligonucleotide
  source 1..20: mol_type = other RNA
gtacaagctgtcggaaacag

SEQ ID NO: 747 - RNA, 20 nt, synthetic construct
  misc_feature 1..20: note = Description of Artificial Sequence: Syntheticoligonucleotide
  source 1..20: mol_type = other RNA
ccactcaccttagcctgagc

SEQ ID NO: 748 - RNA, 20 nt, synthetic construct
  misc_feature 1..20: note = Description of Artificial Sequence: Syntheticoligonucleotide
  source 1..20: mol_type = other RNA
cactcaccttagcctgagca

SEQ ID NO: 749 - RNA, 20 nt, synthetic construct
  misc_feature 1..20: note = Description of Artificial Sequence: Syntheticoligonucleotide
  source 1..20: mol_type = other RNA
cagactgcggggacacagtg

SEQ ID NO: 750 - RNA, 20 nt, synthetic construct
  misc_feature 1..20: note = Description of Artificial Sequence: Syntheticoligonucleotide
  source 1..20: mol_type = other RNA
agactgcggggacacagtga

SEQ ID NO: 751 - RNA, 20 nt, synthetic construct
  misc_feature 1..20: note = Description of Artificial Sequence: Syntheticoligonucleotide
  source 1..20: mol_type = other RNA
cactcacttgagggtttcca

SEQ ID NO: 752 - RNA, 20 nt, synthetic construct
  misc_feature 1..20: note = Description of Artificial Sequence: Syntheticoligonucleotide
  source 1..20: mol_type = other RNA
aggctgcaggtggaatcaga

SEQ ID NO: 753 - RNA, 20 nt, synthetic construct
  misc_feature 1..20: note = Description of Artificial Sequence: Syntheticoligonucleotide
  source 1..20: mol_type = other RNA
atactcactccagatgctgc

SEQ ID NO: 754 - RNA, 20 nt, synthetic construct
  misc_feature 1..20: note = Description of Artificial Sequence: Syntheticoligonucleotide
  source 1..20: mol_type = other RNA
tactcactccagatgctgca

SEQ ID NO: 755 - RNA, 20 nt, synthetic construct
  misc_feature 1..20: note = Description of Artificial Sequence: Syntheticoligonucleotide
  source 1..20: mol_type = other RNA
tcactccagatgctgcaggg

SEQ ID NO: 756 - RNA, 20 nt, synthetic construct
  misc_feature 1..20: note = Description of Artificial Sequence: Syntheticoligonucleotide
  source 1..20: mol_type = other RNA
agcctaggaggcaaagagca

SEQ ID NO: 757 - RNA, 20 nt, synthetic construct
  misc_feature 1..20: note = Description of Artificial Sequence: Syntheticoligonucleotide
  source 1..20: mol_type = other RNA
ctgccaaattccagcctcct

SEQ ID NO: 758 - RNA, 20 nt, synthetic construct
  misc_feature 1..20: note = Description of Artificial Sequence: Syntheticoligonucleotide
  source 1..20: mol_type = other RNA
gagccagccacagggccccc

SEQ ID NO: 759 - RNA, 20 nt, synthetic construct
  misc_feature 1..20: note = Description of Artificial Sequence: Syntheticoligonucleotide
  source 1..20: mol_type = other RNA
cgcccaggtcctcacgtctg

SEQ ID NO: 760 - RNA, 20 nt, synthetic construct
  misc_feature 1..20: note = Description of Artificial Sequence: Syntheticoligonucleotide
  source 1..20: mol_type = other RNA
accggctctgcaaaggccag

SEQ ID NO: 761 - RNA, 20 nt, synthetic construct
  misc_feature 1..20: note = Description of Artificial Sequence: Syntheticoligonucleotide
  source 1..20: mol_type = other RNA
aggccattttggaagcttgt

SEQ ID NO: 762 - RNA, 20 nt, synthetic construct
  misc_feature 1..20: note = Description of Artificial Sequence: Syntheticoligonucleotide
  source 1..20: mol_type = other RNA
tgctctggagatggagaagc

SEQ ID NO: 763 - RNA, 20 nt, synthetic construct
  misc_feature 1..20: note = Description of Artificial Sequence: Syntheticoligonucleotide
  source 1..20: mol_type = other RNA
ccttacctgtcatgtttgct

SEQ ID NO: 764 - RNA, 20 nt, synthetic construct
  misc_feature 1..20: note = Description of Artificial Sequence: Syntheticoligonucleotide
  source 1..20: mol_type = other RNA
cttacctgtcatgtttgctc

SEQ ID NO: 765 - RNA, 20 nt, synthetic construct
  misc_feature 1..20: note = Description of Artificial Sequence: Syntheticoligonucleotide
  source 1..20: mol_type = other RNA
aaaggcactgcaagagacaa

SEQ ID NO: 766 - RNA, 20 nt, synthetic construct
  misc_feature 1..20: note = Description of Artificial Sequence: Syntheticoligonucleotide
  source 1..20: mol_type = other RNA
taacatactgggaatctggt

SEQ ID NO: 767 - RNA, 20 nt, synthetic construct
  misc_feature 1..20: note = Description of Artificial Sequence: Syntheticoligonucleotide
  source 1..20: mol_type = other RNA
ttccagtgcttcaggtctgc

SEQ ID NO: 768 - RNA, 20 nt, synthetic construct
  misc_feature 1..20: note = Description of Artificial Sequence: Syntheticoligonucleotide
  source 1..20: mol_type = other RNA
ttttaccttggggctctgac

SEQ ID NO: 769 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 770 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 771 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 772 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 773 - DNA, 23 bp, synthetic construct
  misc_feature 1..23: note = Description of Artificial Sequence: Syntheticoligonucleotide
  source 1..23: mol_type = other DNA
ttaccccatctcagggtgagggg

SEQ ID NO: 774 - DNA, 23 bp, synthetic construct
  misc_feature 1..23: note = Description of Artificial Sequence: Syntheticoligonucleotide
  source 1..23: mol_type = other DNA
ttaccccatctcagggtgagggg

SEQ ID NO: 775 - DNA, 23 bp, synthetic construct
  misc_feature 1..23: note = Description of Artificial Sequence: Syntheticoligonucleotide
  source 1..23: mol_type = other DNA
cagggcccagcacctcagggtgg

SEQ ID NO: 776 - DNA, 23 bp, synthetic construct
  misc_feature 1..23: note = Description of Artificial Sequence: Syntheticoligonucleotide
  source 1..23: mol_type = other DNA
ccccaggctcccactccatgagg

SEQ ID NO: 777 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 778 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 779 - DNA, 23 bp, synthetic construct
  misc_feature 1..23: note = Description of Artificial Sequence: Syntheticoligonucleotide
  source 1..23: mol_type = other DNA
gtcactcaccggcctcgctctgg

SEQ ID NO: 780 - DNA, 23 bp, synthetic construct
  misc_feature 1..23: note = Description of Artificial Sequence: Syntheticoligonucleotide
  source 1..23: mol_type = other DNA
ctccttaccccatctcagggtga

SEQ ID NO: 781 - DNA, 23 bp, synthetic construct
  misc_feature 1..23: note = Description of Artificial Sequence: Syntheticoligonucleotide
  source 1..23: mol_type = other DNA
ccccaggctcccactccatgagg

SEQ ID NO: 782 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 783 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 784 - DNA, 23 bp, synthetic construct
  misc_feature 1..23: note = Description of Artificial Sequence: Syntheticoligonucleotide
  source 1..23: mol_type = other DNA
gtcactcaccggcctcgctctgg

SEQ ID NO: 785 - DNA, 23 bp, synthetic construct
  misc_feature 1..23: note = Description of Artificial Sequence: Syntheticoligonucleotide
  source 1..23: mol_type = other DNA
ccggggtcactcaccggcctcgc

SEQ ID NO: 786 - DNA, 26 bp, synthetic construct
  misc_feature 1..26: note = Description of Artificial Sequence: Syntheticoligonucleotide
  source 1..26: mol_type = other DNA
ctacaaccagagcgaggccggtgagt

SEQ ID NO: 787 - DNA, 26 bp, synthetic construct
  misc_feature 1..26: note = Description of Artificial Sequence: Syntheticoligonucleotide
  source 1..26: mol_type = other DNA
gggtcactcaccggcctcgctctggt

SEQ ID NO: 788 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 789 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 790 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 791 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 792 - RNA, 20 nt, synthetic construct
  misc_feature 1..20: note = Description of Artificial Sequence: Syntheticoligonucleotide
  source 1..20: mol_type = other RNA
ttaccccatctcagggtgag

SEQ ID NO: 793 - RNA, 20 nt, synthetic construct
  misc_feature 1..20: note = Description of Artificial Sequence: Syntheticoligonucleotide
  source 1..20: mol_type = other RNA
ttaccccatctcagggtgag

SEQ ID NO: 794 - RNA, 20 nt, synthetic construct
  misc_feature 1..20: note = Description of Artificial Sequence: Syntheticoligonucleotide
  source 1..20: mol_type = other RNA
cagggcccagcacctcaggg

SEQ ID NO: 795 - RNA, 20 nt, synthetic construct
  misc_feature 1..20: note = Description of Artificial Sequence: Syntheticoligonucleotide
  source 1..20: mol_type = other RNA
ccccaggctcccactccatg

SEQ ID NO: 796 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 797 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 798 - RNA, 20 nt, synthetic construct
  misc_feature 1..20: note = Description of Artificial Sequence: Syntheticoligonucleotide
  source 1..20: mol_type = other RNA
gtcactcaccggcctcgctc

SEQ ID NO: 799 - RNA, 20 nt, synthetic construct
  misc_feature 1..20: note = Description of Artificial Sequence: Syntheticoligonucleotide
  source 1..20: mol_type = other RNA
ctccttaccccatctcaggg

SEQ ID NO: 800 - RNA, 20 nt, synthetic construct
  misc_feature 1..20: note = Description of Artificial Sequence: Syntheticoligonucleotide
  source 1..20: mol_type = other RNA
ccccaggctcccactccatg

SEQ ID NO: 801 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 802 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 803 - RNA, 20 nt, synthetic construct
  misc_feature 1..20: note = Description of Artificial Sequence: Syntheticoligonucleotide
  source 1..20: mol_type = other RNA
gtcactcaccggcctcgctc

SEQ ID NO: 804 - RNA, 20 nt, synthetic construct
  misc_feature 1..20: note = Description of Artificial Sequence: Syntheticoligonucleotide
  source 1..20: mol_type = other RNA
ccggggtcactcaccggcct

SEQ ID NO: 805 - RNA, 20 nt, synthetic construct
  misc_feature 1..20: note = Description of Artificial Sequence: Syntheticoligonucleotide
  source 1..20: mol_type = other RNA
ctacaaccagagcgaggccg

SEQ ID NO: 806 - RNA, 20 nt, synthetic construct
  misc_feature 1..20: note = Description of Artificial Sequence: Syntheticoligonucleotide
  source 1..20: mol_type = other RNA
gggtcactcaccggcctcgc

SEQ ID NO: 807 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 808 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 809 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 810 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 811 - DNA, 23 bp, synthetic construct
  misc_feature 1..23: note = Description of Artificial Sequence: Syntheticoligonucleotide
  source 1..23: mol_type = other DNA
ttaccccatctcagggtgagggg

SEQ ID NO: 812 - DNA, 23 bp, synthetic construct
  misc_feature 1..23: note = Description of Artificial Sequence: Syntheticoligonucleotide
  source 1..23: mol_type = other DNA
ttaccccatctcagggtgagggg

SEQ ID NO: 813 - DNA, 23 bp, synthetic construct
  misc_feature 1..23: note = Description of Artificial Sequence: Syntheticoligonucleotide
  source 1..23: mol_type = other DNA
cagggcccagcacctcagggtgg

SEQ ID NO: 814 - DNA, 23 bp, synthetic construct
  misc_feature 1..23: note = Description of Artificial Sequence: Syntheticoligonucleotide
  source 1..23: mol_type = other DNA
ccccaggctcccactccatgagg

SEQ ID NO: 815 - DNA, 23 bp, synthetic construct
  misc_feature 1..23: note = Description of Artificial Sequence: Syntheticoligonucleotide
  source 1..23: mol_type = other DNA
ccttaccccatctcagggtgagg

SEQ ID NO: 816 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 817 - DNA, 23 bp, synthetic construct
  misc_feature 1..23: note = Description of Artificial Sequence: Syntheticoligonucleotide
  source 1..23: mol_type = other DNA
gtcactcaccggcctcgctctgg

SEQ ID NO: 818 - DNA, 23 bp, synthetic construct
  misc_feature 1..23: note = Description of Artificial Sequence: Syntheticoligonucleotide
  source 1..23: mol_type = other DNA
ctccttaccccatctcagggtga

SEQ ID NO: 819 - DNA, 23 bp, synthetic construct
  misc_feature 1..23: note = Description of Artificial Sequence: Syntheticoligonucleotide
  source 1..23: mol_type = other DNA
ccccaggctcccactccatgagg

SEQ ID NO: 820 - DNA, 23 bp, synthetic construct
  misc_feature 1..23: note = Description of Artificial Sequence: Syntheticoligonucleotide
  source 1..23: mol_type = other DNA
cttaccccatctcagggtgaggg

SEQ ID NO: 821 - DNA, 23 bp, synthetic construct
  misc_feature 1..23: note = Description of Artificial Sequence: Syntheticoligonucleotide
  source 1..23: mol_type = other DNA
gtcactcaccggcctcgctctgg

SEQ ID NO: 822 - DNA, 23 bp, synthetic construct
  misc_feature 1..23: note = Description of Artificial Sequence: Syntheticoligonucleotide
  source 1..23: mol_type = other DNA
ccccaggctcccactccatgagg

SEQ ID NO: 823 - DNA, 23 bp, synthetic construct
  misc_feature 1..23: note = Description of Artificial Sequence: Syntheticoligonucleotide
  source 1..23: mol_type = other DNA
ccggggtcactcaccggcctcgc

SEQ ID NO: 824 - DNA, 26 bp, synthetic construct
  misc_feature 1..26: note = Description of Artificial Sequence: Syntheticoligonucleotide
  source 1..26: mol_type = other DNA
ctacaaccagagcgaggccggtgagt

SEQ ID NO: 825 - DNA, 26 bp, synthetic construct
  misc_feature 1..26: note = Description of Artificial Sequence: Syntheticoligonucleotide
  source 1..26: mol_type = other DNA
gggtcactcaccggcctcgctctggt

SEQ ID NO: 826 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 827 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 828 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 829 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 830 - RNA, 20 nt, synthetic construct
  misc_feature 1..20: note = Description of Artificial Sequence: Syntheticoligonucleotide
  source 1..20: mol_type = other RNA
ttaccccatctcagggtgag

SEQ ID NO: 831 - RNA, 20 nt, synthetic construct
  misc_feature 1..20: note = Description of Artificial Sequence: Syntheticoligonucleotide
  source 1..20: mol_type = other RNA
ttaccccatctcagggtgag

SEQ ID NO: 832 - RNA, 20 nt, synthetic construct
  misc_feature 1..20: note = Description of Artificial Sequence: Syntheticoligonucleotide
  source 1..20: mol_type = other RNA
cagggcccagcacctcaggg

SEQ ID NO: 833 - RNA, 20 nt, synthetic construct
  misc_feature 1..20: note = Description of Artificial Sequence: Syntheticoligonucleotide
  source 1..20: mol_type = other RNA
ccccaggctcccactccatg

SEQ ID NO: 834 - RNA, 20 nt, synthetic construct
  misc_feature 1..20: note = Description of Artificial Sequence: Syntheticoligonucleotide
  source 1..20: mol_type = other RNA
ccttaccccatctcagggtg

SEQ ID NO: 835 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 836 - RNA, 20 nt, synthetic construct
  misc_feature 1..20: note = Description of Artificial Sequence: Syntheticoligonucleotide
  source 1..20: mol_type = other RNA
gtcactcaccggcctcgctc

SEQ ID NO: 837 - RNA, 20 nt, synthetic construct
  misc_feature 1..20: note = Description of Artificial Sequence: Syntheticoligonucleotide
  source 1..20: mol_type = other RNA
ctccttaccccatctcaggg

SEQ ID NO: 838 - RNA, 20 nt, synthetic construct
  misc_feature 1..20: note = Description of Artificial Sequence: Syntheticoligonucleotide
  source 1..20: mol_type = other RNA
ccccaggctcccactccatg

SEQ ID NO: 839 - RNA, 20 nt, synthetic construct
  misc_feature 1..20: note = Description of Artificial Sequence: Syntheticoligonucleotide
  source 1..20: mol_type = other RNA
cttaccccatctcagggtga

SEQ ID NO: 840 - RNA, 20 nt, synthetic construct
  misc_feature 1..20: note = Description of Artificial Sequence: Syntheticoligonucleotide
  source 1..20: mol_type = other RNA
gtcactcaccggcctcgctc

SEQ ID NO: 841 - RNA, 20 nt, synthetic construct
  misc_feature 1..20: note = Description of Artificial Sequence: Syntheticoligonucleotide
  source 1..20: mol_type = other RNA
ccccaggctcccactccatg

SEQ ID NO: 842 - RNA, 20 nt, synthetic construct
  misc_feature 1..20: note = Description of Artificial Sequence: Syntheticoligonucleotide
  source 1..20: mol_type = other RNA
ccggggtcactcaccggcct

SEQ ID NO: 843 - RNA, 20 nt, synthetic construct
  misc_feature 1..20: note = Description of Artificial Sequence: Syntheticoligonucleotide
  source 1..20: mol_type = other RNA
ctacaaccagagcgaggccg

SEQ ID NO: 844 - RNA, 20 nt, synthetic construct
  misc_feature 1..20: note = Description of Artificial Sequence: Syntheticoligonucleotide
  source 1..20: mol_type = other RNA
gggtcactcaccggcctcgc

SEQ ID NO: 845 - DNA, 23 bp, unidentified
  misc_feature 1..23: note = Description of Unknown:Target sequence
  source 1..23: mol_type = other DNA
cctggaggctatccagcgtgagt

SEQ ID NO: 846 - DNA, 20 bp, unidentified
  misc_feature 1..20: note = Description of Unknown:Target sequence
  source 1..20: mol_type = other DNA
ccaagatagttaagtggggt

SEQ ID NO: 847 - DNA, 23 bp, unidentified
  misc_feature 1..23: note = Description of Unknown:Target sequence
  source 1..23: mol_type = other DNA
ccctgctcaggctaaggtgagtg

(19) United States
(12) Patent Application Publication
(10) Pub. No.: US 2024/0252550 A1
(43) Pub. Date: Aug. 1, 2024

(54) GENETIC MODIFICATION OF HEPATOCYTES

(71) Applicant: Beam Therapeutics Inc., Cambridge, MA (US)

(72) Inventors: Giuseppe CIARAMELLA, Cambridge, MA (US); Jason Michael GEHRKE, Cambridge, MA (US); Ryan MURRAY, Cambridge, MA (US)

(21) Appl. No.: 18/486,067

(22) Filed: Apr. 5, 2024

Related U.S. Application Data
(63) Continuation of application No. PCT/US2022/025078, filed on Apr. 15, 2022.
(60) Provisional application No. 63/176,104, filed on Apr. 16, 2021.

(51) Int. Cl. A61K 35/407; A61P 1/16; C12N 5/071; C12N 9/22; C12N 9/78; C12N 15/11; C12N 15/90

(52) U.S. Cl. CPC A61K 35/407; A61P 1/16; C12N 5/067; C12N 9/22; C12N 9/78; C12N 15/11; C12N 15/90; C12Y 305/04; C12N 2310/20; C12N 2510/00

(57) ABSTRACT

The present invention provides methods of producing genetically modified human hepatocytes suitable for hepatocyte transplantation comprising: disrupting one or more major histocompatibility complex (MHC) Class I or Class II genes in isolated human hepatocytes or in a hepatocyte progenitor cell by introducing a base editor and one or more gRNAs that hybridize with a target sequence in the one or more Class I or Class II genes, thereby producing genetically modified human hepatocytes.

### CROSS-REFERENCE TO RELATED APPLICATIONS

This application is a Continuation Application of International Application No. PCT/US2022/025078, filed on Apr. 15, 2022, which claims priority to U.S. Provisional Patent Application Ser. No. 63/176,104, filed on Apr. 16, 2021, each of which is incorporated by reference herein in its entirety for all purposes.

### INCORPORATION BY REFERENCE OF SEQUENCE LISTING

The instant application contains a Sequence Listing which has been submitted electronically in XML format and is hereby incorporated by reference in its entirety. Said XML copy, created on Mar. 29, 2024, is named BEM-009US1_ST26.xml and is 1,080 kilobytes in size.

### BACKGROUND

Orthrotopic Liver Transplant (OLT) is the gold standard treatment for end-stage liver disease, acute liver failure, and liver-based metabolic disorders. OLT has several major disadvantages which includes the scarcity of donor organs, the risks of complications related to surgery, the high cost of the procedure, and the need for lifelong immunosuppression.

Hepatocyte transplantation (HT) is a very attractive and clinically safe alternative to OLT as it is less invasive and less expensive, and it can be performed repeatedly if required. Limitations of HT relate to the limited supply of high-quality hepatocytes and to the insufficient engraftment/long-term acceptance of allografts. Although encouraging clinical improvements are seen in patients transplanted with allogeneic hepatocytes, long term efficacy is still hampered by the limited long-term acceptance of cellular allografts, despite immunosuppression.

### SUMMARY

Human primary hepatocytes are highly immunogenic and thus alternative strategies of immunomodulation prior to their transplantation are desirable to improve engraftment of the hepatocytes. Several impediments currently exist with regard to using hepatocytes for treating liver disease. These are generally: 1) limited human hepatocyte supply; and 2) insufficient engraftment of hepatocytes into subjects. The limited supply of high-quality hepatocytes is at least in part due to a limited supply of donor livers from which high-quality hepatocytes can be isolated. The production and use of humanized animal models that function as hepatocyte bioreactors have rendered the procurement and expansion of the human hepatocytes feasible for program scale development. The second impediment, referenced above, that of insufficient engraftment has to date limited long-term acceptance of cellular allografts despite immunosuppression. The inventors have surprisingly discovered a unique methodology to genetically modify hepatocytes which makes the genetically modified hepatocytes suitable for administration to subjects in need thereof.

In some aspects, a method of producing genetically modified human hepatocytes suitable for hepatocyte transplantation is provided, the method comprising: disrupting one or more major histocompatibility complex (MHC) Class I or Class II genes in isolated human hepatocytes or in a hepatocyte progenitor cell by introducing a base editor and one or more gRNAs that hybridize with a target sequence in the one or more Class I or Class II genes, thereby producing genetically modified human hepatocytes. In some embodiments, disrupting one or more MHC Class I or Class II genes occurs in isolated human hepatocytes. The isolated human hepatocytes can be freshly isolated or previously expanded. MHC Class I and Class II genes are known in the art.

For example, MHC Class I genes include HLA-A, HLA-B, HLA-C, HLA-E, HLA-F, HLA-G, HLA-K and HLA-L. For example, MHC Class II genes include HLA-DP, HLA-DM, HLA-DOA, HLA-DOB, HLA-DQ, HLA-DR.

In some embodiments, the base editor comprises a CRISPR protein fused to a deaminase.

In some embodiments, the genetically modified human hepatocytes have one or more nucleobases edits in a target sequence. For example, the genetically modified can have one, two, three, four, five, six, seven, eight, nine, ten or more than ten nucleobase edits.

In some embodiments, the genetically modified human hepatocytes have a disrupted target sequence. In some embodiments, the disrupted target sequence results in a decreased expression of a target gene. In some embodiments, the disrupted target sequence results in an increased expression of a target gene.

In some embodiments, the genetically modified human hepatocytes have reduced or abolished alloreactivity. Accordingly, in some embodiments, the genetically modified human hepatocytes have reduced alloreactivity. In some embodiments, the genetically modified human hepatocytes have abolished alloreactivity. By “abolished” is meant that no detectable alloreactivity is present by using methods known in the art.

In some embodiments, the Class I or Class II genes are selected from one or more of B2M, CD142, CIITA, HLA-A or HLA-B genes. Accordingly, in some embodiments, the Class I or Class II gene is B2M. In some embodiments, the Class I or Class II gene is CD142. In some embodiments, the Class I or Class II gene is CIITA. In some embodiments, the Class I or Class II gene is HLA-A. In some embodiments, the Class I or Class II gene is HLA-B.

In some embodiments, a stop codon or a splice site is introduced into one or more of the B2M, CD142, CIITA, HLA-A or HLA-B genes. Accordingly, in some embodiments, a stop codon is introduced into one or more of the B2M, CD142, CIITA, HLA-A or HLA-B genes. In some embodiments, a splice site is introduced into one or more of the B2M, CD142, CIITA, HLA-A or HLA-B genes.

In some embodiments, a splice site is introduced at nucleotide position 19 of the B2M gene.

In some embodiments, a stop codon is introduced at nucleotide position 5 of the B2M gene.

In some embodiments, a splice site is introduced at nucleotide position 28 of the CD142 gene.

In some embodiments, a stop codon is introduced at nucleotide position 19 of the CD142 gene.

In some embodiments, a splice site is introduced at nucleotide position 147 of the CIITA gene.

In some embodiments, a stop codon is introduced at nucleotide position 130 of the CIITA gene.

In some embodiments, the CRISPR protein is Cas9 or Cas12. Accordingly, in some embodiments, the CRISPR protein is a Cas9 protein. In some embodiments, the CRISPR protein is a Cas12 protein.

In some embodiments, the Cas9 is from Streptococcus pyogenes (SpCas9) or Staphylococcus aureus (SaCas9). Accordingly, in some embodiments, the Cas9 is from Streptococcus pyogenes (SpCas9). In some embodiments, the Cas9 is from Staphylococcus aureus (SaCas9). Various Cas9 proteins are described in the art obtained or modified from variety of bacteria, including Cas9 with mutations. Cas9, and mutants thereof, are described in various publications, including, for example WO 2013/176772, U.S. Pat. No. 10,266,850, WO 2014/093661, WO 2014/093655, WO 2014/093595, the contents of which are incorporated herein by reference.

Various Cas12 proteins are known in the art and include, for example, Class 2 Type V and Type VI proteins. For example, Class 2 Type V Cas12 include: Cas12a, Cas12b, Cas12c, among others. Various designations for Cas12 have been used and include Cpf1, C2c1, C2c1p, C2c3, C2cp3, C2c2p. In some embodiments of the methods disclosed herein, a Cas12 protein from Class2 Type V or Type VI proteins is used. For example, in some embodiments, a suitable Cas12 for the methods described herein includes a Cas12a protein. In some embodiments, a suitable Cas12 for the methods described herein includes a Cas12b protein. In some embodiments, a suitable Cas12 for the methods described herein includes a Cas12c protein. In some embodiments, a suitable Cas12 for the methods described herein includes a Cpf1 protein. In some embodiments, a suitable Cas12 for the methods described herein includes a C2c1 protein. In some embodiments, a suitable Cas12 for the methods described herein includes a C2c1p protein. In some embodiments, a suitable Cas12 for the methods described herein includes a C2c3 protein. In some embodiments, a suitable Cas12 for the methods described herein includes a C2cp3 protein. In some embodiments, a suitable Cas12 for the methods described herein includes a C2c2p protein. Various Cas12 are described in WO/2016/205711 and WO/2016/205749, the contents of which are incorporated by reference.

In some embodiments, the Cas9 protein is a hyper-accurate Cas9. In some embodiments, the Cas9 protein comprises mutations corresponding to N692A, M694A, Q695A and/or H698A with reference to SpyCas9 (SEQ ID NO: 68). In some embodiments, the Cas9 protein is a high-fidelity Cas9. In some embodiments, the Cas9 protein comprises mutations corresponding to N467A, R661A, Q695A and/or Q926A with reference to SpyCas9 (SEQ ID NO: 68). In some embodiments, the Cas9 protein is a SuperFi-Cas9. In some embodiments, the Cas9 protein comprises mutations wherein, Y1016, R1019, Y1010, Y1013, K1031, Q1027 and/or V1018 residues corresponding to SpyCas9 (SEQ ID NO: 68) are mutated to aspartic acid.

In some embodiments, the CRISPR protein is fused to an adenine or adenosine base editor (ABE), a cytidine or cytosine base editor (CBE), or an inosine base editor (IBE). Accordingly, in some embodiments, the CRISPR protein is fused to an ABE. In some embodiments, the CRIPSR protein is fused to a CBE. In some embodiments, the CRISPR protein is fused to an IBE. In some embodiments, the CRISPR is fused to a base editor comprising an adenine or adenosine deaminase domain or a cytidine or cytosine deaminase domain. In some embodiments, the CRISPR is fused to a base editor comprising an adenine or adenosine deaminase domain and a cytidine or cytosine deaminase domain.

In some embodiments, the CRISPR protein comprises a nuclear localization sequence (NLS) and/or a FLAG, HIS or HA tag. Accordingly, in some embodiments, the CRISPR protein comprises an NLS. In some embodiments, the CRISPR protein comprises a FLAG tag. In some embodiments, the CRISPR protein comprises a HIS tag. In some embodiments, the CRISPR protein comprises an HA tag.

In some embodiments, the CRISPR protein comprises at least one, at least two, at least three, at least four, at least five, at least six, at least seven, at least eight, at least nine, or at least 10 mutations in SEQ ID NO: 1 (SpCas9), SEQ ID NO: 2 (SaCas9), or SEQ ID NO: 3 (Cpf1 Cas12). Amino acid sequences for SpCas9, SaCas9, and Cpf1 Cas12 are presented in the table below.

Exemplary CRISPR protein sequences, modifications thereof, and base editor fusions Staphylococcus pyogenes (SpCas9)

Exemplary CRISPR protein sequences, modifications thereof,
and base editor fusions
Staphylococcus pyogenes (SpCas9)
(SEQ ID NO: 1; NCBI Accession Q99ZW2)
MDKKYSIGLDIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFG
NIVDEVAYHEKYPTIYHLRKKLVDSTDKADLRLIYLALAHMIKFRGHFLIEGDLNPDNSD
VDKLFIQLVQTYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLFLAAKNLSDAI
LLSDILRVNTEITKAPLSASMIKRYDEHHQDLILLKALVRQQLPEKYKEIFFDQSKNGYA
GYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEE
VVDKGASAQSFIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQLKRRRYTGWG
RLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRER
MKRIEEGIKELGSQILKEHPVENTQLQNEKLYLYYLQNGRDMYVDQELDINRLSDYDVDH
IVPQSFLKDDSIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLIREVKVITLKS
KLVSDFRKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDF
ATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPK
YSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGA
PAAFKYFDTTIDRKRYTSTKEVLDATLIHQSITGLYETRIDLSQLGGD
Staphylococcus aureus (SaCas9)
(SEQ ID NO: 2; NCBI Accession: J7RUA5)
MKRNYILGLDIGITSVGYGIIDYETRDVIDAGVRLFKEANVENNEGRRSKRGARRLKRRR
RHRIQRVKKLLFDYNLLTDHSELSGINPYEARVKGLSQKLSEEEFSAALLHLAKRRGVHN
VNEVEEDTGNELSTKEQISRNSKALEEKYVAELQLERLKKDGEVRGSINRFKTSDYVKEA
KQLLKVQKAYHQLDQSFIDTYIDLLETRRTYYEGPGEGSPFGWKDIKEWYEMLMGHCTYF
PEELRSVKYAYNADLYNALNDLNNLVITRDENEKLEYYEKFQIIENVFKQKKKPTLKQIA
KEILVNEEDIKGYRVTSTGKPEFTNLKVYHDIKDITARKEIIENAELLDQIAKILTIYQS
SEDIQEELTNLNSELTQEEIEQISNLKGYTGTHNLSLKAINLILDELWHTNDNQIAIFNR
LKLVPKKVDLSQQKEIPTTLVDDFILSPVVKRSFIQSIKVINAIIKKYGLPNDIIIELAR
EKNSKDAQKMINEMQKRNRQTNERIEEIIRTTGKENAKYLIEKIKLHDMQEGKCLYSLEA
IPLEDLLNNPFNYEVDHIIPRSVSFDNSFNNKVLVKQEENSKKGNRTPFQYLSSSDSKIS
YETFKKHILNLAKGKGRISKTKKEYLLEERDINRFSVQKDFINRNLVDTRYATRGLMNLL
RSYFRVNNLDVKVKSINGGFTSFLRRKWKFKKERNKGYKHHAEDALIIANADFIFKEWKK
LDKAKKVMENQMFEEKQAESMPEIETEQEYKEIFITPHQIKHIKDFKDYKYSHRVDKKPN
RELINDTLYSTRKDDKGNTLIVNNLNGLYDKDNDKLKKLINKSPEKLLMYHHDPQTYQKL
KLIMEQYGDEKNPLYKYYEETGNYLTKYSKKDNGPVIKKIKYYGNKLNAHLDITDDYPNS
RNKVVKLSLKPYRFDVYLDNGVYKFVTVKNLDVIKKENYYEVNSKCYEEAKKLKKISNQA
EFIASFYNNDLIKINGELYRVIGVNNDLLNRIEVNMIDITYREYLENMNDKRPPRIIKTI
ASKTQSIKKYSTDILGNLYEVKSKKHPQIIKKG
Francisella tularensis subsp. novicida (strain U112)
fnCPF1-Cas12
(SEQ ID NO: 3; NCBI Accession: A0Q7Q2)
MSIYQEFVNKYSLSKTLRFELIPQGKTLENIKARGLILDDEKRAKDYKKAKQIIDKYHQF
FIEEILSSVCISEDLLQNYSDVYFKLKKSDDDNLQKDFKSAKDTIKKQISEYIKDSEKFK
NLFNQNLIDAKKGQESDLILWLKQSKDNGIELFKANSDITDIDEALEIIKSFKGWTTYFK
GFHENRKNVYSSNDIPTSIIYRIVDDNLPKFLENKAKYESLKDKAPEAINYEQIKKDLAE
ELTFDIDYKTSEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVTTMQSFYEQIA
AFKTVEEKSIKETLSLLFDDLKAQKLDLSKIYFKNDKSLTDLSQQVFDDYSVIGTAVLEY
ITQQIAPKNLDNPSKKEQELIAKKTEKAKYLSLETIKLALEEFNKHRDIDKQCRFEEILA
NFAAIPMIFDEIAQNKDNLAQISIKYQNQGKKDLLQASAEDDVKAIKDLLDQTNNLLHKL
KIFHISQSEDKANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENKGEGYKKIVYK
LLPGANKMLPKVFFSAKSIKFYNPSEDILRIRNHSTHTKNGSPQKGYEKFEFNIEDCRKF
IDFYKQSISKHPEWKDFGFRFSDTQRYNSIDEFYREVENQGYKLTFENISESYIDSVVNQ
GKLYLFQIYNKDFSAYSKGRPNLHTLYWKALFDERNLQDVVYKLNGEAELFYRKQSIPKK
ITHPAKEAIANKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMKTNYHDKLAAI
EKDRDSARKDWKKINNIKEMKEGYLSQVVHEIAKLVIEYNAIVVFEDLNFGFKRGRFKVE
KQVYQKLEKMLIEKLNYLVFKDNEFDKTGGVLRAYQLTAPFETFKKMGKQTGIIYYVPAG
FTSKICPVTGFVNQLYPKYESVSKSQEFFSKFDKICYNLDKGYFEFSFDYKNFGDKAAKG
KWTIASFGSRLINFRNSDKNHNWDTREVYPTKELEKLLKDYSIEYGHGECIKAAICGESD
KKFFAKLISVLNTILQMRNSKIGTELDYLISPVADVNGNFFDSRQAPKNMPQDADANGAY
HIGLKGLMLLGRIKNNQEGKKLNLVIKNEEYFEFVQNRNN
Bacillus hisashii - bhCas12bv4 nuclease
(SEQ ID NO: 66)
MAPKKKRKVGIHGVPAAATRSFILKIEPNEEVKKGLWKTHEVLNHGIAYYMNILKLIRQEAIY
EHHEQDPKNPKKVSKAEIQAELWDFVLKMQKCNSFTHEVDKDEVFNILRELYEELVPSSVEKK
GEANQLSNKFLYPLVDPNSQSGKGTASSGRKPRWYNLKIAGDPSWEEEKKKWEEDKKKDPLAK
ILGKLAEYGLIPLFIPYTDSNEPIVKEIKWMEKSRNQSVRRLDKDMFIQALERFLSWESWNLK
VKEEYEKVEKEYKTLEERIKEDIQALKALEQYEKERQEQLLRDTLNTNEYRLSKRGLRGWREI
IQKWLKMDENEPSEKYLEVFKDYQRKHPREAGDYSVYEFLSKKENHFIWRNHPEYPYLYATFC
EIDKKKKDAKQQATFTLADPINHPLWVRFEERSGSNLNKYRILTEQLHTEKLKKKLTVQLDRL
IYPTESGGWEEKGKVDIVLLPSRQFYNQIFLDIEEKGKHAFTYKDESIKFPLKGTLGGARVQF
DRDHLRRYPHKVESGNVGRIYFNMTVNIEPTESPVSKSLKIHRDDFPKVVNFKPKELTEWIKD
SKGKKLKSGIESLEIGLRVMSIDLGQRQAAAASIFEVVDQKPDIEGKLFFPIKGTELYAVHRA
SFNIKLPGETLVKSREVLRKAREDNLKLMNQKLNFLRNVLHFQQFEDITEREKRVTKWISRQE
NSDVPLVYQDELIQIRELMYKPYKDWVAFLKQLHKRLEVEIGKEVKHWRKSLSDGRKGLYGIS
LKNIDEIDRTRKFLLRWSLRPTEPGEVRRLEPGQRFAIDQLNHLNALKEDRLKKMANTIIMHA
LGYCYDVRKKKWQAKNPACQIILFEDLSNYNPYGERSRFENSRLMKWSRREIPRQVALQGEIY
GLQVGEVGAQFSSRFHAKTGSPGIRCRVVTKEKLQDNRFFKNLQREGRLILDKIAVLKEGDLY
PDKGGEKFISLSKDRKCVTTHADINAAQNLQKRFWTRTHGFYKVYCKAYQVDGQTVYIPESKD
QKQKIIEEFGEGYFILKDGVYEWVNAGKLKIKKGSSKQSSSELVDSDILKDSFDLASELKGEK
LMLYRDPSGNVFPSDKWMAAGVFFGKLERILISKLTNQYSISTIEDDSSKQSMSGGSKRTADG
SEFESPKKKRKVE*
ABE8.13m-dead-bhCas12bv4
(SEQ ID NO: 67)
MSEVEFSHEYWMRHALTLAKRARDEREVPVGAVLVLNNRVIGEGWNRAIGLHDPTAHAEIMAL
RQGGLVMQNYRLYDATLYVTFEPCVMCAGAMIHSRIGRVVFGVRNAKTGAAGSLMDVLHHPGM
NHRVEITEGILADECAALLCRFFRMPRRVFNAQKKAQSSTDGSSGSETPGTSESATPESSGAP
KKKRKVGIHGVPAAATRSFILKIEPNEEVKKGLWKTHEVLNHGIAYYMNILKLIRQEAIYEHH
EQDPKNPKKVSKAEIQAELWDFVLKMQKCNSFTHEVDKDEVFNILRELYEELVPSSVEKKGEA
NQLSNKFLYPLVDPNSQSGKGTASSGRKPRWYNLKIAGDPSWEEEKKKWEEDKKKDPLAKILG
KLAEYGLIPLFIPYTDSNEPIVKEIKWMEKSRNQSVRRLDKDMFIQALERFLSWESWNLKVKE
EYEKVEKEYKILEERIKEDIQALKALEQYEKERQEQLLRDTLNTNEYRLSKRGLRGWREIIQK
WLKMDENEPSEKYLEVFKDYQRKHPREAGDYSVYEFLSKKENHFIWRNHPEYPYLYATFCEID
KKKKDAKQQATFTLADPINHPLWVRFEERSGSNLNKYRILTEQLHTEKLKKKLTVQLDRLIYP
TESGGWEEKGKVDIVLLPSRQFYNQIFLDIEEKGKHAFTYKDESIKFPLKGTLGGARVQFDRD
HLRRYPHKVESGNVGRIYFNMTVNIEPTESPVSKSLKIHRDDFPKVVNFKPKELTEWIKDSKG
KKLKSGIESLEIGLRVMSIALGQRQAAAASIFEVVDQKPDIEGKLFFPIKGTELYAVHRASFN
IKLPGETLVKSREVLRKAREDNLKLMNQKLNFLRNVLHFQQFEDITEREKRVTKWISRQENSD
VPLVYQDELIQIRELMYKPYKDWVAFLKQLHKRLEVEIGKEVKHWRKSLSDGRKGLYGISLKN
IDEIDRTRKFLLRWSLRPTEPGEVRRLEPGQRFAIDQLNHLNALKEDRLKKMANTIIMHALGY
CYDVRKKKWQAKNPACQIILFEDLSNYNPYKERSRFENSRLMKWSRREIPRQVALQGEIYGLQ
VGEVGAQFSSRFHAKTGSPGIRCRVVTKEKLQDNRFFKNLQREGRLILDKIAVLKEGDLYPDK
GGEKFISLSKDRKCVTTHADINAAQNLQKRFWTRTHGFYKVYCKAYQVDGQTVYIPESKDQKQ
KIIEEFGEGYFILKDGVYEWVNAGKLKIKKGSSKQSSSELVDSDILKDSFDLASELKGEKLML
YRDPSGNVFPSDKWMAAGVFFGKLERILISKLTNQYSISTIEDDSSKQSMKRPAATKKAGQAK
KKK*
Streptococcus pyogenes Cas9 (SpyCas9; GenBank: QSG91308.1)
(SEQ ID NO: 68)
MDKKYSIGLDIGTNSVGWAVITDDYKVPSKKFKVLGNTDRHSIKKNLIGALLFDSGETAEATR
LKRTARRRYTRRKNRICYLQEIFSNEMAKVDDSFFHRLEESFLVEEDKKHERHPIFGNIVDEV
AYHEKYPTIYHLRKKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFGNLIALSLGLTPNF
KSNFDLAEDAKLQLSKDTYDDDLDNLLAQIGDQYADLFLAAKNLSDAILLSDILRVNTEITKA
PLSASMIKRYDEHHQDLTLLKALVRQQLPEKYKEIFFDQSKNGYAGYIDGGASQEEFYKFIKP
ILEKMDGTEELLVKLNREDLLRKQRTFDNGSIPHQIHLGELHAILRRQEDFYPFLKDNREKIE
KILTFRIPYYVGPLARGNSRFAWMTRKSEETITPWNFEEVVDKGASAQSFIERMTNFDKNLPN
EKVLPKHSLLYEYFTVYNELTKVKYVTEGMRKPAFLSGEQKKAIVDLLFKTNRKVTVKQLKED
YFKKIECFDSVEISGVEDRFNASLGTYHDLLKIIKDKDFLDNEENEDILEDIVLTLTLFEDRE
MIEERLKTYAHLFDDKVMKQLKRRRYTGWGRLSRKLINGIRDKQSGKTILDFLKSDGFANRNF
MQLIHDDSLTFKEDIQKAQVSGQGDSLHEHIANLAGSPAIKKGILQTVKVVDELVKVMGRHKP
ENIVIEMARENQTTQKGQKNSRERMKRIEEGIKELGSQILKEHPVENTQLQNEKLYLYYLQNG
RDMYVDQELDINRLSDYDVDHIVPQSFLKDDSIDNKVLIRSDKNRGKSDNVPSEEVVKKMKNY
WRQLLNAKLITQRKFDNLTKAERGGLSELDKAGFIKRQLVETRQITKHVAQILDSRMNTKYDE
NDKLIREVKVITLKSKLVSDFRKDFQFYKVREINNYHHAHDAYLNAVVGTALIKKYPKLESEF
VYGDYKVYDVRKMIAKSEQEIGKATAKYFFYSNIMNFFKTEITLANGEIRKRPLIETNGETGE
IVWDKGRDFATVRKVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEAKGYKEVKKDLIIK
LPKYSLFELENGRKRMLASAGELQKGNELALPSKYVNFLYLASHYEKLKGSPEDNEQKQLFVE
QHKHYLDEIIEQISEFSKRVILADANLDKVLSAYNKHRDKPIREQAENIIHLFTLTNLGAPAA
FKYFDTTIDRKRYTSTKEVLDATLIHQSITGLYETRIDLSQLGGD.

In some embodiments, the mutation is an amino acid substitution.

In some embodiments, the at least one mutation results in an inactive Cas9 (dCas9).

In some embodiments, the at least one mutation is one or more amino acid substitutions in the PAM interacting domain, RuvC domain and/or the HNH domain of Cas9. Accordingly, in some embodiments, the at least one mutation is one or more amino acid substitutions in the PAM interacting domain. In some embodiments, the at least one mutation is one or more amino acid substitutions in the RuvC domain. In some embodiments, the at least one mutation is one or more amino acid substitutions in the HNH domain. In some embodiments, the at least one or more amino acid substitutions occurs in the PAM interacting domain, the RuvC domain and the HNH domain. In some embodiments, the at least one or more amino acid substitutions occurs in the PAM interacting domain and the RuvC domain. In some embodiments, the at least one or more amino acid substitutions occurs in the PAM interacting domain and the HNH domain. In some embodiments, the at least one or more amino acid substitutions occurs in the RuvC domain and the HNH domain.

In some embodiments, the at least one mutation is an aspartic acid-to-alanine substitution at amino acid 10 (D10A) of SpCas9, or a corresponding mutation thereof in a Cas9 protein.

In some embodiments, the at least one mutation is a histidine-to-alanine substitution at amino acid 840 (H840A) of SpCas9, or a corresponding mutation thereof in a Cas9 protein.

In some embodiments, the Cas9 protein has nickase activity. In some embodiments, the one or more mutations in the Cas9 protein renders the Cas9 catalytically inactive, otherwise referred to as a “dead Cas9” or “dCas9.”

In some embodiments, the CRISPR protein is fused to an adenosine deaminase and has an amino acid sequence at least 80% identical to SEQ ID NO: 65

In some embodiments, the CRISPR protein is fused to a cytosine deaminase and has an amino acid sequence at least 80% identical to SEQ ID NO: 4-64

In some embodiments, the SpCas9 protein recognizes a PAM sequence comprising 5′-NGG-3′,5′-NGA-3′, or 5′-NGC-3′. Accordingly, in some embodiments, the SpCas9 protein recognizes a PAM sequence comprising 5′-NGG-3′. In some embodiments, the SpCas9 protein recognizes a PAM sequence comprising 5′-NGA-3′. In some embodiments, the SpCas9 protein recognizes a PAM sequence comprising 5′-NGC-3′.

In some embodiments, the SaCas9 protein recognizes a PAM sequence comprising 5′-NNNRRT-3′, or 5′-NNGRRT-3′. In some embodiments, the SaCas9 protein recognizes a PAM sequence comprising 5′-NNNRRT-3′. In some embodiments, the SaCas9 protein recognizes a PAM sequence comprising 5′-NNGRRT-3′.

In some embodiments, the Cas12 protein recognizes a PAM sequence comprising 5′-RTTN-3′.

In some embodiments, the isolated human hepatocytes have been previously cryopreserved and subsequently thawed. In some embodiments, the isolated human hepatocytes are primary cultures. In some embodiments, the isolated human hepatocytes are freshly isolated.

In some embodiments, the genetically modified human hepatocytes overexpress CIITA in comparison to non-genetically modified human hepatocytes. In some embodiments, the genetically modified human hepatocytes overexpress B2M. In some embodiments, the genetically modified human hepatocytes overexpress B2M-HLA-E fusion protein. In some embodiments, the genetically modified human hepatocytes overexpress PDL1. In some embodiments, the genetically modified human hepatocytes overexpress PDL2.

In some embodiments, the genetically modified human hepatocytes are engrafted into a humanized animal model for expansion.

In some embodiments, the humanized animal model is an FRG pig, an FRG mouse, or an FRG rat. Accordingly, in some embodiments, the humanized animal model is an FRG pig. In some embodiments, the humanized animal model is an FRG mouse. In some embodiments, the humanized animal model is an FRG rat.

In some embodiments, the genetically modified human hepatocytes are first engrafted into an FRG mouse or FRG rat for an initial cell expansion. In some embodiments, the genetically modified human hepatocytes are first engrafted into an FRG mouse for an initial expansion. In some embodiments, the genetically modified human hepatocytes are first engrafted into an FRG rat for an initial expansion.

In some embodiments, following the initial cell expansion, the genetically modified cells are subsequently engrafted into the FRG pig for further cell expansion.

In some embodiments, the initially expanded cells or the further expanded cells are isolated from an animal.

In some embodiments, the initially expanded cells or the further expanded cells are isolated by fluorescence-activated cell sorting, immunomagnetic cell separation, density gradient centrifugation, and/or immunodensity cell separation. Any kind of isolation strategy which preserves cell viability can be used in the methods herein. In some embodiments, the cells are isolated by fluorescence-activated cell sorting. In some embodiments, the cells are isolated by immunomagnetic cell separation. In some embodiments, the cells are isolated by density gradient centrifugation. In some embodiments, the cells are isolated by immunodensity cell separation.

In some embodiments, the genetically modified human hepatocytes have one, two, three or more nucleobase edits. Accordingly, in some embodiments, the genetically modified human hepatocytes have one nucleobase edits. In some embodiments, the genetically modified human hepatocytes have two nucleobase edits. In some embodiments, the genetically modified human hepatocytes have three nucleobase edits. In some embodiments, the genetically modified human hepatocytes have four nucleobase edits. In some embodiments, the genetically modified human hepatocytes have five nucleobase edits. In some embodiments, the genetically modified human hepatocytes have six nucleobase edits. In some embodiments, the genetically modified human hepatocytes have seven nucleobase edits. In some embodiments, the genetically modified human hepatocytes have eight nucleobase edits. In some embodiments, the genetically modified human hepatocytes have nine nucleobase edits. In some embodiments, the genetically modified human hepatocytes have ten nucleobase edits. In some embodiments, the genetically modified human hepatocytes have more than ten nucleobase edits.

In some embodiments, a single base editor used in combination with more than one guide produces two, three or more nucleobase edits. Accordingly, in some embodiments, a single base editor used in combination with more than one guide produces two nucleobase edits. In some embodiments, a single base editor used in combination with more than one guide produces three nucleobase edits. In some embodiments, a single base editor used in combination with more than one guide produces more than three nucleobase edits. This method thus allows for multiplexing of the nucleobase edits.

In some embodiments, more than one base editor produces the one, two, three or more nucleobase edits.

In some aspects, a nucleic acid encoding the base editor and one or more gRNAs that hybridize with a target sequence as described herein is provided.

In some embodiments, the nucleic acid is codon-optimized for expression in mammalian cells.

In some embodiments, the nucleic acid is codon-optimized for expression in human cells.

In some aspects, a vector encoding the nucleic acids described herein is provided.

In some aspects, a eukaryotic cell comprising the base editor and one or more gRNAs that hybridize with a target sequence as described herein is provided.

In some embodiments, the cell is a mammalian cell. In some embodiments, the cell is a human cell. In some embodiments, the human cell is a hepatocyte.

In some aspects, a method of treating a liver disease is provided, the method comprising administering to a subject in need thereof, genetically modified human hepatocytes produced in accordance with the methods described herein.

In some embodiments, the genetically modified human hepatocytes are injected into the portal vein of a subject in need thereof.

In some embodiments, about 10-15 billion genetically modified human hepatocytes are administered to a subject in need thereof. In some embodiments, genetically modified human hepatocytes are injected into the portal vein of a subject in need thereof. In some embodiments, between about 5-20 billion genetically modified human hepatocytes are injected into the portal vein of a subject in need thereof. In some embodiments, between about 10-12 billion genetically modified human hepatocytes are injected into the portal vein of a subject in need thereof. In some embodiments, between about 12-15 billion genetically modified human hepatocytes are injected into the portal vein of a subject in need thereof.

In some aspects, a base editor and one or more guide RNAs that target the B2M gene is provided, wherein the base editor and corresponding one or more guide RNAs are selected from Table 2.

In some aspects, a base editor and one or more guide RNAs that target the CD142 gene is provided, wherein the base editor and corresponding one or more guide RNAs are selected from Table 3.

In some aspects, a base editor and one or more guide RNAs that target the CIITA gene is provided, wherein the base editor and corresponding one or more guide RNAs are selected from Table 4.

In some aspects, a base editor and one or more guide RNAs that target the HLA-A gene, wherein the base editor and corresponding one or more guide RNAs are selected from Table 5.

In some aspects, a base editor and one or more guide RNAs that target the HLA-B gene, wherein the base editor and corresponding one or more guide RNAs are selected from Table 6.

In some embodiments, the base editor and the one or more guide RNAs is provided, wherein one, two, three, or more than three edits are made to the target gene.

In some aspects, a cell comprising a base editor and one or more guide RNAs is provided.

In some aspects, a genetically modified human hepatocyte that has one or more edits in an MHC gene is provided as described herein.

In some embodiments, the MHC gene is selected from B2M, CD142, CIITA, HLA-A and/or HLA-B. Accordingly, in some embodiments, the MHC gene is the B2M gene. In some embodiments, the MHC gene is the CD142 gene. In some embodiments, the MHC gene is the CIITA gene. In some embodiments, the MHC gene is the HLA-A gene. In some embodiments, the MHC gene is the HLA-B gene.

In some embodiments, edits to one or more of B2M, CD142, CIITA, HLA-A and/or HLA-B genes results in increased expression of the B2M, CD142, CIITA, HLA-A and/or HLA-B genes in comparison to a non-genetically modified human hepatocyte. For example, in some embodiments, edits to the B2M gene results in increased expression of the B2M gene. In some embodiments, edits to the CD142 gene results in increased expression of the CD142 gene. In some embodiments, edits to the CIITA gene results in increased expression of the CIITA gene.

In some embodiments, edits to the HLA-A gene results in increased expression of the HLA-A gene. In some embodiments, edits to the HLA-B gene results in increased expression of the HLA-B gene.

### Definitions

In order for the present invention to be more readily understood, certain terms are first defined below. Additional definitions for the following terms and other terms are set forth throughout the specification.

A or An: The articles “a” and “an” are used herein to refer to one or to more than one (i.e., to at least one) of the grammatical object of the article. By way of example, “an element” means one element or more than one element.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Base Editor: By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is a cytosine base editor (CBE). In some embodiments, the base editor is an adenosine base editor (ABE). In some embodiments, the base editor is an adenine base editor (ABE). In some embodiments, the base editor is an adenosine or adenine base editor (ABE) and a cytosine or cytidine base editor (CBE). In some embodiments, the base editor is a nuclease-inactive Cas9 (dCas9) fused to an adenosine deaminase. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference. As used herein, the term “base editor” may also include a CRISPR protein, such as a Cas9 or a Cas12 protein.

Base Editing Activity: By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C. In another embodiment, the base editing activity is cytidine deaminase activity, e.g., converting target C·G to T·A and adenosine or adenine deaminase activity, e.g., converting A·T to G·C.

Base Editor System: The term “base editor system” refers to a system for editing a nucleobase of a target nucleotide sequence. In various embodiments, the base editor (BE) system comprises (1) a polynucleotide programmable nucleotide binding domain (e.g., Cas9 or Cas12), a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain.

In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

Biologically active: As used herein, the phrase “biologically active” refers to a characteristic of any agent that has activity in a biological system, and particularly in an organism. For instance, an agent that, when administered to an organism, has a biological effect on that organism, is considered to be biologically active. In particular embodiments, where a peptide is biologically active, a portion of that peptide that shares at least one biological activity of the peptide is typically referred to as a “biologically active” portion.

Cleavage: As used herein, cleavage refers to a break in a target nucleic acid created by a nuclease of a CRISPR system described herein. In some embodiments, the cleavage event is a double-stranded DNA break. In some embodiments, the cleavage event is a single-stranded DNA break. In some embodiments, the cleavage event is a single-stranded RNA break. In some embodiments, the cleavage event is a double-stranded RNA break.

Complementary: As used herein, complementary refers to a nucleic acid strand that forms Watson-Crick base pairing, such that A base pairs with T, and C base pairs with G, or non-traditional base pairing with bases on a second nucleic acid strand. In other words, it refers to nucleic acids that hybridize with each other under appropriate conditions.

Clustered Interspaced Short Palindromic Repeat (CRISPR)-associated (Cas) system: As used herein, CRISPR-Cas9 system refers to nucleic acids and/or proteins involved in the expression of, or directing the activity of, CRISPR-effectors, including sequences encoding CRISPR effectors, RNA guides, and other sequences and transcripts from a CRISPR locus. In some embodiments, the CRISPR system is an engineered, non-naturally occurring CRISPR system. In some embodiments, the components of a CRISPR system may include a nucleic acid(s) (e.g., a vector) encoding one or more components of the system, a component(s) in protein form, or a combination thereof.

CRISPR Array: The term “CRISPR array”, as used herein, refers to the nucleic acid (e.g., DNA) segment that includes CRISPR repeats and spacers, starting with the first nucleotide of the first CRISPR repeat and ending with the last nucleotide of the last (terminal) CRISPR repeat. Typically, each spacer in a CRISPR array is located between two repeats. The terms “CRISPR repeat” or “CRISPR direct repeat,” or “direct repeat,” as used herein, refer to multiple short direct repeating sequences, which show very little or no sequence variation within a CRISPR array.

CRISPR-associated protein (Cas): The term “CRISPR-associated protein,” “CRISPR effector,” “effector,” or “CRISPR enzyme” as used herein refers to a protein that carries out an enzymatic activity or that binds to a target site on a nucleic acid specified by a RNA guide. In different embodiments, a CRISPR effector has endonuclease activity, nickase activity, exonuclease activity, transposase activity, and/or excision activity. In some embodiments, the Cas is a high-accuracy Cas. In some embodiments, the Cas is a high-fidelity Cas. In some embodiments, the Cas is a SuperFi-Cas. In some embodiments, the high-accuracy, high-fidelity and SuperFi-Cas are as described in Bravo, J. et al. Structural basis for mismatch surveillance by CRISPR-Cas9 Nature, 603, March 2022.

crRNA: The term “CRISPR RNA” or “crRNA,” as used herein, refers to a RNA molecule including a guide sequence used by a CRISPR effector to target a specific nucleic acid sequence. Typically, crRNAs contains a sequence that mediates target recognition and a sequence that forms a duplex with a tracrRNA. In some embodiments, the crRNA: tracrRNA duplex binds to a CRISPR effector.

Ex Vivo: As used herein, the term “ex vivo” refers to events that occur in cells or tissues, grown outside rather than within a multi-cellular organism.

Functional equivalent or analog: As used herein, the term “functional equivalent” or “functional analog” denotes, in the context of a functional derivative of an amino acid sequence, a molecule that retains a biological activity (either function or structural) that is substantially similar to that of the original sequence. A functional derivative or equivalent may be a natural derivative or is prepared synthetically. Exemplary functional derivatives include amino acid sequences having substitutions, deletions, or additions of one or more amino acids, provided that the biological activity of the protein is conserved. The substituting amino acid desirably has physicochemical properties which are similar to that of the substituted amino acid. Desirable similar physicochemical properties include, similarities in charge, bulkiness, hydrophobicity, hydrophilicity, and the like.

Half-Life: As used herein, the term “half-life” is the time required for a quantity such as protein concentration or activity to fall to half of its value as measured at the beginning of a time period.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

Inhibition: As used herein, the terms “inhibition,” “inhibit” and “inhibiting” refer to processes or methods of decreasing or reducing activity and/or expression of a protein or a gene of interest. Typically, inhibiting a protein or a gene refers to reducing expression or a relevant activity of the protein or gene by at least 10% or more, for example, 20%, 30%, 40%, or 50%, 60%, 70%, 80%, 90% or more, or a decrease in expression or the relevant activity of greater than 1-fold, 2-fold, 3-fold, 4-fold, 5-fold, 10-fold, 50-fold, 100-fold or more as measured by one or more methods described herein or recognized in the art.

Hybridization: As used herein, the term “hybridization” refers to a reaction in which two or more nucleic acids bind with each other via hydrogen bonding by Watson-Crick pairing, Hoogstein binding or other sequence-specific binding between the bases of the two nucleic acids. A sequence capable of hybridizing with another sequence is termed the “complement” of the sequence, and is said to be “complementary” or show “complementarity”.

Indel: As used herein, the term “indel” refers to insertion or deletion of bases in a nucleic acid sequence. It commonly results in mutations and is a common form of genetic variation.

In Vitro: As used herein, the term “in vitro” refers to events that occur in an artificial environment, e.g., in a test tube or reaction vessel, in cell culture, etc., rather than within a multi-cellular organism.

In Vivo: As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

Mutation: As used herein, the term “mutation” has the ordinary meaning in the art, and includes, for example, point mutations, substitutions, insertions, deletions, inversions, and deletions.

Oligonucleotide: As used herein, the term “oligonucleotide” generally refers to polynucleotides of between about 5 and about 100 nucleotides of single- or double-stranded DNA. Oligonucleotides are also known as “oligomers” or “oligos” and may be isolated from genes, or chemically synthesized.

PAM: The term “PAM” or “Protospacer Adjacent Motif” refers to a short nucleic acid sequence (usually 2-6 base pairs in length) that follows the nucleic acid region targeted for cleavage by the CRISPR system, such as CRISPR-Cas9. The PAM is required for a Cas nuclease to cut and is generally found 3-4 nucleotides downstream from the cut site.

Polypeptide: The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably.

Prevent: As used herein, the term “prevent” or “prevention”, when used in connection with the occurrence of a disease, disorder, and/or condition, refers to reducing the risk of developing the disease, disorder and/or condition.

Protein: The term “protein” as used herein refers to one or more polypeptides that function as a discrete unit. If a single polypeptide is the discrete functioning unit and does not require permanent or temporary physical association with other polypeptides in order to form the discrete functioning unit, the terms “polypeptide” and “protein” may be used interchangeably. If the discrete functional unit is comprised of more than one polypeptide that physically associate with one another, the term “protein” refers to the multiple polypeptides that are physically coupled and function together as the discrete unit.

Reference: A “reference” entity, system, amount, set of conditions, etc., is one against which a test entity, system, amount, set of conditions, etc. is compared as described herein. For example, in some embodiments, a “reference” antibody is a control antibody that is not engineered as described herein.

RNA guide: The term RNA guide refers to an RNA molecule that facilitates the targeting of a protein described herein to a target nucleic acid. Exemplary “RNA guides” or “guide RNAs” include, but are not limited to, crRNAs or crRNAs in combination with cognate tracrRNAs. The latter may be independent RNAs or fused as a single RNA using a linker (sgRNAs). In some embodiments, the RNA guide is engineered to include a chemical or biochemical modification, in some embodiments, an RNA guide may include one or more nucleotides.

Subject: The term “subject”, as used herein, means any subject for whom diagnosis, prognosis, or therapy is desired. For example, a subject can be a mammal, e.g., a human or non-human primate (such as an ape, monkey, orangutan, or chimpanzee), a dog, cat, guinea pig, rabbit, rat, mouse, horse, cattle, or cow.

sgRNA: The term “sgRNA” or “single guide RNA” refers to a single guide RNA containing (i) a guide sequence (crRNA sequence) and (ii) a Cas9 nuclease-recruiting sequence (tracrRNA).

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLASTN for nucleotide sequences and BLASTP, gapped BLAST, and PSI-BLAST for amino acid sequences. Exemplary such programs are described in Altschul, et al., Basic local alignment search tool, J. Mol. Biol., 215(3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Target Nucleic Acid: The term “target nucleic acid” as used herein refers to nucleotides of any length (oligonucleotides or polynucleotides) to which the CRISPR-Cas9 system binds, either deoxyribonucleotides, ribonucleotides, or analogs thereof. Target nucleic acids may have three-dimensional structure, may including coding or non-coding regions, may include exons, introns, mRNA, tRNA, rRNA, siRNA, shRNA, miRNA, ribozymes, cDNA, plasmids, vectors, exogenous sequences, endogenous sequences. A target nucleic acid can comprise modified nucleotides, include methylated nucleotides, or nucleotide analogs. A target nucleic acid may be interspersed with non-nucleic acid components. A target nucleic acid is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

Therapeutically effective amount: As used herein, the term “therapeutically effective amount” refers to an amount of a therapeutic molecule (e.g., an engineered antibody described herein) which confers a therapeutic effect on a treated subject, at a reasonable benefit/risk ratio applicable to any medical treatment. The therapeutic effect may be objective (i.e., measurable by some test or marker) or subjective (i.e., subject gives an indication of or feels an effect). In particular, the “therapeutically effective amount” refers to an amount of a therapeutic molecule or composition effective to treat, ameliorate, or prevent a particular disease or condition, or to exhibit a detectable therapeutic or preventative effect, such as by ameliorating symptoms associated with the disease, preventing or delaying the onset of the disease, and/or also lessening the severity or frequency of symptoms of the disease. A therapeutically effective amount can be administered in a dosing regimen that may comprise multiple unit doses. For any particular therapeutic molecule, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular subject may depend upon a variety of factors including the disorder being treated and the severity of the disorder; the activity of the specific pharmaceutical agent employed; the specific composition employed; the age, body weight, general health, sex and diet of the subject; the time of administration, route of administration, and/or rate of excretion or metabolism of the specific therapeutic molecule employed; the duration of the treatment; and like factors as is well known in the medical arts.

tracrRNA: The term “tracrRNA” or “trans-activating crRNA” as used herein refers to an RNA including a sequence that forms a structure required for a CRISPR-associated protein to bind to a specified target nucleic acid.

Treatment: As used herein, the term “treatment” (also “treat” or “treating”) refers to any administration of a therapeutic molecule (e.g., a CRISPR-Cas therapeutic protein or system described herein) that partially or completely alleviates, ameliorates, relieves, inhibits, delays onset of, reduces severity of and/or reduces incidence of one or more symptoms or features of a particular disease, disorder, and/or condition. Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### BRIEF DESCRIPTION OF THE DRAWINGS

FIG. 1A is a schematic that shows a B2M gene BE4- and an ABE-compatible target sequence and associated PAM and protospacer region sites. FIG. 1B is a graph that shows base-editing of the B2M gene. The data from these studies show editing efficiency of the B2M gene using either an ABE editor (ABE7.10) or a BE4 editor.

FIG. 2A is a schematic that shows a CIITA BE4- and an ABE-compatible target sequence and associated PAM and protospacer region sites. FIG. 2B is a graph that shows base editing of the CIITA gene. THE data from these studies show editing efficiency of the CIITA gene using either an ABE editor (ABE8.2m) or a BE4 editor.

### DETAILED DESCRIPTION

Described herein is the production of genetically modified human hepatocytes that are suitable for use in the treatment of disease. Also described are suitable compositions comprising vectors, nucleic acids, and/or cells that achieve the genetically modified human hepatocytes. Furthermore, various methods of treating subjects in need thereof using the genetically modified hepatocytes are described.

### Methods of Producing Genetically Modified Human Hepatocytes

A method of producing genetically modified human hepatocytes suitable for hepatocyte transplantation is provided, the method comprising: disrupting one or more major histocompatibility complex (MHC) Class I or Class II genes in isolated human hepatocytes or in a hepatocyte progenitor cell by introducing a base editor and one or more gRNAs that hybridize with a target sequence in the one or more Class I or Class II genes, thereby producing genetically modified human hepatocytes. The genetically modified human hepatocytes can have one or more nucleobase edits that alter the expression of a corresponding MHC Class I or Class II gene. Alternatively, or complementarily, the genetically modified human hepatocytes have reduced or suppressed expression of one or more MHC Class I or Class II genes. In this manner, the genetically modified hepatocytes once transplanted into a subject in need thereof, will not cause a rejection that will lead to the selective death of the transplanted genetically modified hepatocytes. As such, the genetically modified human hepatocytes have reduced or abolished alloreactivity.

Any kind or MHC Class I or Class II gene can be targeted to either reduce, abolish or suppress gene expression. For example, MHC Class I genes include HLA-A, HLA-B, HLA-C, HLA-E, HLA-F, HLA-G, HLA-K and HLA-L. For example, MHC Class II genes include HLA-DP, HLA-DM, HLA-DOA, HLA-DOB, HLA-DQ, HLA-DR. In some embodiments, one or more MHC Class I or Class II gene are targeted to increase gene expression. In some embodiments, one or more MHC Class I or Class II gene are targeted to decrease gene expression. In some embodiments, the genetically modified human hepatocytes overexpress CD47 and/or CD142 in comparison to a non-genetically modified human hepatocyte.

The isolated human hepatocytes can be obtained from any suitable donor. In some embodiments, the donor does not have liver disease. In some embodiments, the donor has a liver disease. The method can be used with freshly isolated hepatocytes or once-frozen then thawed hepatocytes. In some embodiments, the method uses hepatocytes obtained from a progenitor or stem cell. For example, the progenitor or stem cell can be any suitable pluripotent cell, such as a induced pluripotent cell (iPS cell) or an embryonic stem (ES) cell.

In some embodiments, the method comprises a base editor and one or more guide RNAs that target the B2M gene, wherein the base editor and corresponding one or more guide RNAs comprising any one of the protospacer sequences listed in Table 2 are selected.

In some embodiments, the method comprises a base editor and one or more guide RNAs that target the CD142 gene, wherein the base editor and corresponding one or more guide RNAs comprising any one of the protospacer sequences listed in Table 3 are selected.

In some embodiments, the method comprises a base editor and one or more guide RNAs that target the CIITA gene, wherein the base editor and corresponding one or more guide RNAs comprising any one of the protospacer sequences listed in Table 4 are selected.

In some embodiments, the method comprises a base editor and one or more guide RNAs that target the HLA-A gene, wherein the base editor and corresponding one or more guide RNAs comprising any one of the protospacer sequences listed in Table 5 are selected.

In some embodiments, the method comprises a base editor and one or more guide RNAs that target the HLA-B gene, wherein the base editor and corresponding one or more guide RNAs comprising any one of the protospacer sequences listed in Table 6 are selected.

In some embodiments, provided herein is a guide RNA comprising any one of the protospacer sequences listed in Table 2. In some embodiments, a guide RNA comprises any one of the protospacer sequences listed in Table 3. In some embodiments, a guide RNA comprises any one of the protospacer sequences listed in Table 4. In some embodiments, a guide RNA comprises any one of the protospacer sequences listed in Table 5. In some embodiments, a guide RNA comprises any one of the protospacer sequences listed in Table 6.

In some embodiments, provided herein is a guide RNA comprising any one of the sequences listed in Table 2A. In some embodiments, a guide RNA comprises any one of the sequences listed in Table 3A. In some embodiments, a guide RNA comprises any one of the sequences listed in Table 4A. In some embodiments, a guide RNA comprises any one of the sequences listed in Table 5A. In some embodiments, a guide RNA comprises any one of the sequences listed in Table 6A.

In some embodiments, the method comprises a base editor and one or more guide RNAs that target the B2M gene, wherein the base editor and corresponding one or more guide RNAs comprising any one of the sequences listed in Table 2A are selected.

In some embodiments, the method comprises a base editor and one or more guide RNAs that target the CD142 gene, wherein the base editor and corresponding one or more guide RNAs comprising any one of the sequences listed in Table 3A are selected.

In some embodiments, the method comprises a base editor and one or more guide RNAs that target the CIITA gene, wherein the base editor and corresponding one or more guide RNAs comprising any one of the sequences listed in Table 4A are selected.

In some embodiments, the method comprises a base editor and one or more guide RNAs that target the HLA-A gene, wherein the base editor and corresponding one or more guide RNAs comprising any one of the sequences listed in Table 5A are selected.

In some embodiments, the method comprises a base editor and one or more guide RNAs that target the HLA-B gene, wherein the base editor and corresponding one or more guide RNAs comprising any one of the sequences listed in Table 6A are selected.

Various base editors can be used in the methods to make the genetically modified human hepatocytes.

Base editors comprising a CRISPR protein and any one or more of an adenine base editor (ABE), a cytidine base editor (CBE) or an inosine base editor (IBE) are suitable for the methods described herein. In some embodiments, the methods described herein can be achieved by using a CRISPR protein to achieve a targeted repression of a gene of interest, such as one or more of MHC Class I or Class II genes.

CRISPR proteins suitable for the methods described herein are described throughout, and include any Cas9 or Cas12 CRISPR proteins. For example, Cas9 can be selected from any suitable bacterium, including Cas9 described isolated from Streptococcus pyogenes (SpCas9) or Staphylococcus aureus (SaCas9). Cas12 CRISPR proteins suitable for the methods described herein include any Class 2 Type V or Type VI Cas12 protein, including, for example, Class 2 Type V Cas12 include: Cas12a, Cas12b, Cas12c, among others.

The CRISPR protein suitable for the methods described herein can have one or more mutations. The one or more mutations can result in a CRISPR protein that is a nickase or a catalytically inactive CRISPR protein. By “mutation” is meant any of, or any combination of, a point mutation, a substitution, a deletion, an inversion, or a fusion. The fusion can occur anywhere in the CRISPR protein, for example, at either the N-terminus, the C-terminus, or between the N- and C-termini. To achieve a nickase or a catalytically dead CRISPR protein, one or more mutations can be made in any of, or in any combination of, the PAM interacting domain, the RuvC domain, and/or the HNH domain. Various mutations are described in the art, and include for example those described in U.S. Pat. No. 9,790,490, the contents of which are incorporated herein.

In some embodiments, the Cas9 is a high-fidelity Cas9. In some embodiments, the high-fidelity Cas9 variant comprises enhanced specificity, which minimizes off-target cleavage. In some embodiments, the Cas9 is a hyper-accurate Cas9. In some embodiments, engineered variants, for example, ‘hyper-accurate Cas9’ (N692A, M694A, Q695A and/or H698A mutations corresponding to SpyCas9) and/or ‘high-fidelity Cas9’ (N467A, R661A, Q695A and/or Q926A mutations corresponding to SpyCas9) are used which comprise mutations mainly within the REC3 domain and achieve higher specificity and fidelity. High-fidelity variants reduce the capacity of Cas9 to stabilize mismatches and reduce off-target DNA cleavage. In some embodiments, the increase in specificity is accompanied by a loss in efficiency of on-target cleavage by about 100 fold. In some embodiments, a SuperFi-Cas9 is used, which is a high-fidelity variant that maintains on-target cleavage rates comparable to wild-type Cas9. In some embodiments, the SuperFi-Cas9 comprises mutations in the RuvC loop. In some embodiments, the mutations inhibit formation of a kinked conformation that facilitates subsequent cleavage of gRNA-TS duplex. In some embodiments, the Y1016, R1019, Y1010, Y1013, K1031, Q1027 and/or V1018 residues corresponding to SpyCas9 are mutated, for example, to aspartic acid. (Bravo, J. et al. Structural basis for mismatch surveillance by CRISPR-Cas9 Nature, 603, March 2022).

In some embodiments, the CRISPR protein is fused with a deaminase, such as an adenosine deaminase, a cytosine deaminase, or an inosine deaminase as described herein. Multiple configurations of base editors are possible to achieve a multiplexing-type of multiple base edits. For example, in some embodiments, a single base editor is used in combination with more than one guide to produce two, three or more nucleobase edits. Alternatively, in some embodiments, multiple base editors paired with a suitable guide are used to produce two, three, or more nucleobase edits. Multiple base editors and associated guides are shown in Tables 2, 3, 4, 5, and 6. Accordingly, in some embodiments, a base editor and a suitable guide is provided to target one or more specific genes, such as the B2M gene, the CD142 gene, the CIITA gene, the HLA-A gene, and the HLA-B gene.

In some embodiments, the base editing system is provided in one or more vectors. For example, the base editing system can be provided in a single vector or in a “split vector,” which is comprised of more than one vector which delivers the components of the base editing system. The corresponding nucleic acids can be codon-optimized. Such codon optimization is performed to optimize the nucleic acids for expression in human cells.

Following production of genetically modified hepatocytes, the genetically modified cells are expanded in a suitable humanized animal model. This expansion allows for the production of a suitable number of cells that sufficient for transplantation into a subject in need thereof. Various humanized animal models are known in the art, and include, for example the FRG pig, the FRG mouse, and the FRG rat animals. In some embodiments, the genetically modified hepatocytes under a first expansion within the FRG mouse and/or FRG mouse animal, followed by a second expansion in a larger humanized FRG animal, such as in the pig. As a general matter, about 0.5-1.0 million cells generate about 80-150 million hepatocytes per FRG mouse. As a general matter, about 0.5-1.0 million cells generate about 480-900 million hepatocytes per FRG rat. FRG pigs can generally generate about 100× more than the FRG rat in terms of cellular expansion.

Following the expansion phase, the genetically modified human hepatocytes are subsequently isolated from the FRG animals. Such isolation follows methods known in the art and include, for example, fluorescence-activated cell sorting, immunomagnetic cell separation, density gradient centrifugation, and/or immunodensity cell separation.

Method of Treating Liver Disease Described herein are methods of using the described genetically modified human hepatocytes for treating subjects who have liver disease. The genetically modified human hepatocytes can be used for treating various liver disease, including for example, alpha-1 antitrypsin deficiency, Crigler-Najjar syndrome type 1, familial hypercholesterolemia, congenital coagulation factor VII deficiency, hemophilia A, glycogen storage disease type I, infantile refusum disease, maple syrup urine disease, neonatal hemochromatosis, progressive familial intrahepatic cholestasis type 2 (PFIC2), urea cycle defects such as ornithine transcarbamylase (OTC) deficiency, arginosuccinate lyase deficiency, carbamoylphosphate synthase type 1 deficiency, citrullinemia, Wilson's disease, acute liver failure, fatty liver of pregnancy and acute-on-chronic liver failure. Accordingly, the methods described herein can be used to treat either congenital or acquired liver disease. Accordingly, in some embodiments, the genetically modified human hepatocytes are used for treating alpha-1 antitrypsin deficiency. In some embodiments, the genetically modified human hepatocytes are used for treating Crigler-Najjar syndrome type 1. In some embodiments, the genetically modified human hepatocytes are used for treating familial hypercholesterolemia. In some embodiments, the genetically modified human hepatocytes are used for treating congenital coagulation factor VII deficiency. In some embodiments, the genetically modified human hepatocytes are used treating for hemophilia A. In some embodiments, the genetically modified human hepatocytes are used for treating glycogen storage disease type I. In some embodiments, the genetically modified human hepatocytes are used for treating infantile refusum disease. In some embodiments, the genetically modified human hepatocytes are used for treating maple syrup urine disease. In some embodiments, the genetically modified human hepatocytes are used for treating neonatal hemochromatosis. In some embodiments, the genetically modified human hepatocytes are used for treating progressive familial intrahepatic cholestasis type 2 (PFIC2). In some embodiments, the genetically modified human hepatocytes are used for treating progressive familial intrahepatic cholestasis type 2 (PFIC2). In some embodiments, the genetically modified human hepatocytes are used for treating urea cycle defects such as ornithine transcarbamylase (OTC) deficiency, arginosuccinate lyase deficiency, carbamoylphosphate synthase type 1 deficiency, citrullinemia, Wilson's disease. In some embodiments, the genetically modified human hepatocytes are used for treating acute liver failure. In some embodiments, the genetically modified human hepatocytes are used for treating fatty liver of pregnancy. In some embodiments, the genetically modified human hepatocytes are used for treating acute-on-chronic liver failure.

The method of treating a subject in need thereof comprises the administration of the genetically modified human hepatocytes described herein. Various modes of administration are suitable for treating a subject in need thereof, such as for example, intraportal infusion or injection of the cells. In some embodiments, the genetically modified human hepatocytes are administered into the portal vein of a subject in need thereof. For dosing purposes, the amount of genetically modified human hepatocytes administered to a subject in need thereof is about 5-20 billion cells. In some embodiments, between about 5-20 billion genetically modified human hepatocytes are injected into the portal vein of a subject in need thereof. In some embodiments, between about 10-12 billion genetically modified human hepatocytes are injected into the portal vein of a subject in need thereof. In some embodiments, between about 12-15 billion genetically modified human hepatocytes are injected into the portal vein of a subject in need thereof.

In some embodiments, the genetically modified hepatocytes are administered to a subject in a quantity of about 2-15% of the total liver mass. In some embodiments, the genetically modified hepatocytes are administered to a subject in a quantity of about 5-10% of the total liver mass. Accordingly, in some embodiments, the genetically modified hepatocytes are administered to a subject in a quantity of about 2% of the total liver mass. In some embodiments, the genetically modified hepatocytes are administered to a subject in a quantity of about 3% of the total liver mass. In some embodiments, the genetically modified hepatocytes are administered to a subject in a quantity of about 4% of the total liver mass. In some embodiments, the genetically modified hepatocytes are administered to a subject in a quantity of about 5% of the total liver mass. In some embodiments, the genetically modified hepatocytes are administered to a subject in a quantity of about 6% of the total liver mass. In some embodiments, the genetically modified hepatocytes are administered to a subject in a quantity of about 7% of the total liver mass. In some embodiments, the genetically modified hepatocytes are administered to a subject in a quantity of about 8% of the total liver mass. In some embodiments, the genetically modified hepatocytes are administered to a subject in a quantity of about 9% of the total liver mass. In some embodiments, the genetically modified hepatocytes are administered to a subject in a quantity of about 10% of the total liver mass. In some embodiments, the genetically modified hepatocytes are administered to a subject in a quantity of about 11% of the total liver mass. In some embodiments, the genetically modified hepatocytes are administered to a subject in a quantity of about 12% of the total liver mass. In some embodiments, the genetically modified hepatocytes are administered to a subject in a quantity of about 13% of the total liver mass. In some embodiments, the genetically modified hepatocytes are administered to a subject in a quantity of about 14% of the total liver mass. In some embodiments, the genetically modified hepatocytes are administered to a subject in a quantity of about 15% of the total liver mass.

In some embodiments, the genetically modified hepatocytes are administered to a subject up to a dose of about 2×10⁸ cells per kg of body weight. In some embodiments, the genetically modified hepatocytes are administered to a subject up at about 1.5×10⁸ cells per kg of body weight. In some embodiments, the genetically modified hepatocytes are administered to a subject up at about 1.2×10⁸ cells per kg of body weight. In some embodiments, the genetically modified hepatocytes are administered to a subject up at about 1.0×10⁸ cells per kg of body weight. In some embodiments, the genetically modified hepatocytes are administered to a subject up at about 0.8×10⁸ cells per kg of body weight. In some embodiments, the genetically modified hepatocytes are administered to a subject up at about 0.5×10⁸ cells per kg of body weight.

### CRISPR Fusion Proteins

In some embodiments, a Cas9 or a Cas12 protein is fused to one or more heterologous protein domains. In some embodiments, the Cas9 or Cas12 enzyme is fused to more than about 1, 2, 3, 4, 5, 6, 7, 8, 9, 10 or more protein domains. In some embodiments, the heterologous protein domain is fused to the C-terminus of the Cas9 or Cas12 enzyme. In some embodiments, the heterologous protein domain is fused to the N-terminus of the Cas9 or Cas12 enzyme. In some embodiments, the heterologous protein domain is fused internally, between the C-terminus and the N-terminus of the Cas9 or Cas12 enzyme. In some embodiments, the internal fusion is made within the Cas9 RuvCI, RuvC II, RuvCIII, HNH, REC I, or PAM interacting domain.

A Cas9 or Cas12 protein may be directly or indirectly linked to another protein domain. In some embodiments, a suitable CRISPR system contains a linker or spacer that joins a Cas9 protein and a heterologous protein. An amino acid linker or spacer is generally designed to be flexible or to interpose a structure, such as an alpha-helix, between the two protein moieties. A linker or spacer can be relatively short, or can be longer. Typically, a linker or spacer contains for example 1-100 (e.g., 1-100, 5-100, 10-100, 20-100 30-100, 40-100, 50-100, 60-100, 70-100, 80-100, 90-100, 5-55, 10-50, 10-45, 10-40, 10-35, 10-30, 10-25, 10-20) amino acids in length. In some embodiments, a linker or spacer is equal to or longer than 1, 2, 3, 4, 5, 6, 7, 8, 9, 10, 15, 20, 25, 30, 35, 40, 45, 50, 55, 60, 65, 70, 75, 80, 85, 90, 95, or 100 amino acids in length. Typically, a longer linker may decrease steric hindrance. In some embodiments, a linker will comprise a mixture of glycine and serine residues. In some embodiments, the linker may additionally comprise threonine, proline and/or alanine residues.

In some embodiments, a Cas9 or Cas12 protein is fused to cellular localization signals, epitope tags, reporter genes, and protein domains with enzymatic activity, epigenetic modifying activity, RNA cleavage activity, nucleic acid binding activity, transcription modulation activity. In some embodiments, the Cas9 protein is fused to a nuclear localization sequence (NLS), a FLAG tag, a HIS tag, and/or a HA tag.

Suitable fusion partners include, but are not limited to, a polypeptide that provides for methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity, demyristoylation activity, integrase activity, transposase activity, recombinase activity, polymerase activity, ligase activity, helicase activity, or nuclease activity, any of which can modify DNA or a DNA-associated polypeptide (e.g., a histone or DNA binding protein). In some embodiments, the Cas9 protein is fused to a histone demethylase, a transcriptional activator or a deaminase.

Further suitable fusion partners include, but are not limited to boundary elements (e.g., CTCF), proteins and fragments thereof that provide periphery recruitment (e.g., Lamin A, Lamin B, etc.), and protein docking elements (e.g., FKBP/FRB, Pill/Abyl, etc.).

In particular embodiments, a Cas9 is fused to a cytidine or adenosine deaminase domain, e.g., for use in base editing. In some embodiments, Cas9 is fused to a adenine and cytosine base editor (ACBE or CABE), wherein ACBE or CABE is generated by fusing a heterodimer of TadA and an activation-induced cytidine deaminase (AID) to the N- and C-terminals of Cas9 nickase (nCas9). In some embodiments, the ACBE or CABE simultaneously induces C-to-T and A-to-G base editing at the same target site. Xie, J et al. ACBE, a new base editor for simultaneous C-to-T and A-to-G substitutions in mammalian systems. BMC Biology (18: 131), 2020)

In particular embodiments, a Cas9 or Cas12 is fused to a cytidine or adenosine deaminase domain, e.g., for use in base editing. In some embodiments, the terms “cytidine deaminase” and “cytosine deaminase” can be used interchangeably. In certain embodiments, the cytidine deaminase domain may have sequence identity of 70%, 75%, 80%, 85%, 90%, 91%, 92%, 93%, 94%, 95%, 96%, 97%, 98%, 99% or more to any cytidine deaminase described herein. In some embodiments, the cytidine deaminase domain has cytidine deaminase activity, (e.g., converting C to U). In certain embodiments, the adenosine deaminase domain may have sequence identity of 70%, 75%, 80%, 85%, 90%, 91%, 92%, 93%, 94%, 95%, 96%, 97%, 98%, 99% or more to any adenosine deaminase described herein. In some embodiments, the adenosine deaminase domain has adenosine deaminase activity, (e.g., converting A to I). In some embodiments, the terms “adenosine deaminase” and “adenine deaminase” can be used interchangeably.

In some embodiments, a cytidine deaminase can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a fusion protein comprises all or a portion of an APOBEC1 deaminase. In some embodiments, a deaminase incorporated into a fusion protein comprises all or a portion of APOBEC2 deaminase. In some embodiments, a deaminase incorporated into a fusion protein comprises all or a portion of is an APOBEC3 deaminase. In some embodiments, a deaminase incorporated into a fusion protein comprises all or a portion of an APOBEC3A deaminase. In some embodiments, a deaminase incorporated into a fusion protein comprises all or a portion of APOBEC3B deaminase. In some embodiments, a deaminase incorporated into a fusion protein comprises all or a portion of APOBEC3C deaminase. In some embodiments, a deaminase incorporated into a fusion protein comprises all or a portion of APOBEC3D deaminase. In some embodiments, a deaminase incorporated into a fusion protein comprises all or a portion of APOBEC3E deaminase. In some embodiments, a deaminase incorporated into a fusion protein comprises all or a portion of APOBEC3F deaminase. In some embodiments, a deaminase incorporated into a fusion protein comprises all or a portion of APOBEC3G deaminase. In some embodiments, a deaminase incorporated into a fusion protein comprises all or a portion of APOBEC3H deaminase. In some embodiments, a deaminase incorporated into a fusion protein comprises all or a portion of APOBEC4 deaminase. In some embodiments, a deaminase incorporated into a fusion protein comprises all or a portion of activation-induced deaminase (AID). In some embodiments a deaminase incorporated into a fusion protein comprises all or a portion of cytidine deaminase 1 (CDA1). It should be appreciated that a fusion protein can comprise a deaminase from any suitable organism (e.g., a human or a rat). In some embodiments, a deaminase domain of a fusion protein is from a human, chimpanzee, gorilla, monkey, cow, dog, rat, or mouse. In some embodiments, the deaminase domain of the fusion protein is derived from rat (e.g., rat APOBEC1). In some embodiments, the deaminase domain is human APOBEC1. In some embodiments, the deaminase domain is pmCDA1.

### Sequences of Exemplary Cytidine Deaminases are Provided Below.

pmCDA1 (Petromyzon marinus)
(SEQ ID NO: 4)
MTDAEYVRIHEKLDIYTFKKQFFNNKKSVSHRCYVLFELKRRGERRACFWGYAVNKPQ
SGTERGIHAEIFSIRKVEEYLRDNPGQFTINWYSSWSPCADCAEKILEWYNQELRGNGHT
LKIWACKLYYEKNARNQIGLWNLRDNGVGLNVMVSEHYQCCRKIFIQSSHNQLNENR
WLEKTLKRAEKRRSELSIMIQVKILHTTKSPAV
Human AID:
(SEQ ID NO: 5)
MDSLLMNRRKFLYQFKNVRWAKGRRETYLCYVVKRRDSATSFSLDFGYLRNKNGCHV
ELLFLRYISDWDLDPGRCYRVTWFTSWSPCYDCARHVADFLRGNPNLSLRIFTARLYFC
EDRKAEPEGLRRLHRAGVQIAIMTFKAPV
Human AID:
(SEQ ID NO: 6)
MDSLLMNRRKFLYQFKNVRWAKGRRETYLCYVVKRRDSATSFSLDFGYLRNKNGCHV
ELLFLRYISDWDLDPGRCYRVTWFTSWSPCYDCARHVADFLRGNPNLSLRIFTARLYFC
EDRKAEPEGLRRLHRAGVQIAIMTFKDYFYCWNTFVENHERTFKAWEGLHENSVRLSR
QLRRILLPLYEVDDLRDAFRTLGL
(underline: nuclear localization sequence; double
underline: nuclear export signal)
Mouse AID:
(SEQ ID NO: 7)
MDSLLMKQKKFLYHFKNVRWAKGRHETYLCYVVKRRDSATSCSLDFGHLRNKSGCHV
ELLFLRYISDWDLDPGRCYRVTWFTSWSPCYDCARHVAEFLRWNPNLSLRIFTARLYFC
EDRKAEPEGLRRLHRAGVQIGIMTFKDYFYCWNTFVENRERTFKAWEGLHENSVRLTR
QLRRILLPLYEVDDLRDAFRMLGF
(underline: nuclear localization sequence; double
underline: nuclear export signal)
Canine AID:
(SEQ ID NO: 8)
MDSLLMKQRKFLYHFKNVRWAKGRHETYLCYVVKRRDSATSFSLDFGHLRNKSGCHV
ELLFLRYISDWDLDPGRCYRVTWFTSWSPCYDCARHVADFLRGYPNLSLRIFAARLYFC
EDRKAEPEGLRRLHRAGVQIAIMTFKDYFYCWNTFVENREKTFKAWEGLHENSVRLSR
QLRRILLPLYEVDDLRDAFRTLGL
(underline: nuclear localization sequence; double
underline: nuclear export signal)
Bovine AID:
(SEQ ID NO: 9)
MDSLLKKQRQFLYQFKNVRWAKGRHETYLCYVVKRRDSPTSFSLDFGHLRNKAGCHV
ELLFLRYISDWDLDPGRCYRVTWFTSWSPCYDCARHVADFLRGYPNLSLRIFTARLYFC
DKERKAEPEGLRRLHRAGVQIAIMTFKDYFYCWNTFVENHERTFKAWEGLHENSVRLS
RQLRRILLPLYEVDDLRDAFRTLGL
(underline: nuclear localization sequence; double
underline: nuclear export signal)
Rat AID:
(SEQ ID NO: 10)
MAVGSKPKAALVGPHWERERIWCFLCSTGLGTQQTGQTSRWLRPAATQDPVSPPRSLL
MKQRKFLYHFKNVRWAKGRHETYLCYVVKRRDSATSFSLDFGYLRNKSGCHVELLFL
RYISDWDLDPGRCYRVTWFTSWSPCYDCARHVADFLRGNPNLSLRIFTARLTGWGALP
AGLMSPARPSDYFYCWNTFVENHERTFKAWEGLHENSVRLSRRLRRILLPLYEVDDLR
DAFRTLGL
(underline: nuclear localization sequence; double
underline: nuclear export signal)
clAID (Canis lupus familiaris):
(SEQ ID NO: 11)
MDSLLMKQRKFLYHFKNVRWAKGRHETYLCYVVKRRDSATSFSLDFGHLRNKSGCHV
ELLFLRYISDWDLDPGRCYRVTWFTSWSPCYDCARHVADFLRGYPNLSLRIFAARLYFC
EDRKAEPEGLRRLHRAGVQIAIMTFKDYFYCWNTFVENREKTFKAWEGLHENSVRLSR
QLRRILLPLYEVDDLRDAFRTLGL
btAID (Bos Taurus):
(SEQ ID NO: 12)
MDSLLKKQRQFLYQFKNVRWAKGRHETYLCYVVKRRDSPTSFSLDFGHLRNKAGCHV
ELLFLRYISDWDLDPGRCYRVTWFTSWSPCYDCARHVADFLRGYPNLSLRIFTARLYFC
DKERKAEPEGLRRLHRAGVQIAIMTFKDYFYCWNTFVENHERTFKAWEGLHENSVRLS
RQLRRILLPLYEVDDLRDAFRTLGL
mAID (Mus musculus):
(SEQ ID NO: 13)
MDSLLMNRRKFLYQFKNVRWAKGRRETYLCYVVKRRDSATSFSLDFGYLRNKNGCHV
ELLFLRYISDWDLDPGRCYRVTWFTSWSPCYDCARHVADFLRGNPNLSLRIFTARLYFC
EDRKAEPEGLRRLHRAGVQIAIMTFKDYFYCWNTFVENHERTFKAWEGLHENSVRLSR
QLRRILLPLYEVDDLRDAFRTLGL
rAPOBEC-1 (Rattus norvegicus):
(SEQ ID NO: 14)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVL
ELYCIILGLPPCLNILRRKQPQLTFFTIALQSCHYQRLPPHILWATGLK
maAPOBEC-1 (Mesocricetus auratus):
(SEQ ID NO: 15)
MSSETGPVVVDPTLRRRIEPHEFDAFFDQGELRKETCLLYEIRWGGRHNIWRHTGQNTS
RHVEINFIEKFTSERYFYPSTRCSIVWFLSWSPCGECSKAITEFLSGHPNVTLFIYAARLYH
HTDQRNRQGLRDLISRGVTIRIMTEQEYCYCWRNFVNYPPSNEVYWPRYPNLWMRLYA
LELYCIHLGLPPCLKIKRRHQYPLTFFRLNLQSCHYQRIPPHILWATGFI
ppAPOBEC-1 (Pongo pygmaeus):
(SEQ ID NO: 16)
MTSEKGPSTGDPTLRRRIESWEFDVFYDPRELRKETCLLYEIKWGMSRKIWRSSGKNTT
NHVEVNFIKKFTSERRFHSSISCSITWFLSWSPCWECSQAIREFLSQHPGVTLVIYVARLF
WHMDQRNRQGLRDLVNSGVTIQIMRASEYYHCWRNFVNYPPGDEAHWPQYPPLWMM
LYALELHCIILSLPPCLKISRRWQNHLAFFRLHLQNCHYQTIPPHILLATGLIHPSVTWR
ocAPOBEC1 (Oryctolagus cuniculus):
(SEQ ID NO: 17)
MASEKGPSNKDYTLRRRIEPWEFEVFFDPQELRKEACLLYEIKWGASSKTWRSSGKNTT
NHVEVNFLEKLTSEGRLGPSTCCSITWFLSWSPCWECSMAIREFLSQHPGVTLIIFVARLF
QHMDRRNRQGLKDLVTSGVTVRVMSVSEYCYCWENFVNYPPGKAAQWPRYPPRWML
MYALELYCIILGLPPCLKISRRHQKQLTFFSLTPQYCHYKMIPPYILLATGLLQPSVPWR
mdAPOBEC-1 (Monodelphis domestica):
(SEQ ID NO: 18)
MNSKTGPSVGDATLRRRIKPWEFVAFFNPQELRKETCLLYEIKWGNQNIWRHSNQNTSQ
HAEINFMEKFTAERHFNSSVRCSITWFLSWSPCWECSKAIRKFLDHYPNVTLAIFISRLY
WHMDQQHRQGLKELVHSGVTIQIMSYSEYHYCWRNFVDYPQGEEDYWPKYPYLWIM
LYVLELHCIILGLPPCLKISGSHSNQLALFSLDLQDCHYQKIPYNVLVATGLVQPFVTWR
ppAPOBEC-2 (Pongo pygmaeus):
(SEQ ID NO: 19)
MAQKEEAAAATEAASQNGEDLENLDDPEKLKELIELPPFEIVTGERLPANFFKFQFRNVE
YSSGRNKTFLCYVVEAQGKGGQVQASRGYLEDEHAAAHAEEAFFNTILPAFDPALRYN
VTWYVSSSPCAACADRIIKTLSKTKNLRLLILVGRLFMWEELEIQDALKKLKEAGCKLRI
MKPQDFEYVWQNFVEQEEGESKAFQPWEDIQENFLYYEEKLADILK
btAPOBEC-2 (Bos Taurus):
(SEQ ID NO: 20)
MAQKEEAAAAAEPASQNGEEVENLEDPEKLKELIELPPFEIVTGERLPAHYFKFQFRNVE
YSSGRNKTFLCYVVEAQSKGGQVQASRGYLEDEHATNHAEEAFFNSIMPTFDPALRYM
VTWYVSSSPCAACADRIVKTLNKTKNLRLLILVGRLFMWEEPEIQAALRKLKEAGCRLR
IMKPQDFEYIWQNFVEQEEGESKAFEPWEDIQENFLYYEEKLADILK
mAPOBEC-3-(1) (Mus musculus):
(SEQ ID NO: 21)
MQPQRLGPRAGMGPFCLGCSHRKCYSPIRNLISQETFKFHFKNLGYAKGRKDTFLCYEV
TRKDCDSPVSLHHGVFKNKDNIHAEICFLYWFHDKVLKVLSPREEFKITWYMSWSPCFE
CAEQIVRFLATHHNLSLDIFSSRLYNVQDPETQQNLCRLVQEGAQVAAMDLYEFKKCW
KKFVDNGGRRFRPWKRLLTNFRYQDSKLQEILRPCYISVPSSSSSTLSNICLTKGLPETRF
WVEGRRMDPLSEEEFYSQFYNQRVKHLCYYHRMKPYLCYQLEQFNGQAPLKGCLLSE
KGKQHAEILFLDKIRSMELSQVTITCYLTWSPCPNCAWQLAAFKRDRPDLILHIYTSRLY
FHWKRPFQKGLCSLWQSGILVDVMDLPQFTDCWTNFVNPKRPFWPWKGLEIISRRTQR
RLRRIKESWGLQDLVNDFGNLQLGPPMS
Mouse APOBEC-3-(2):
(SEQ ID NO: 22)
MGPFCLGCSHRKCYSPIRNLISQETFKFHFKNLGYAKGRKDTFLCYEVTRKDCDSPVSLH
HGVFKNKDNIHAEICFLYWFHDKVLKVLSPREEFKITWYMSWSPCFECAEQIVRFLATHHN
LSLDIFSSRLYNVQDPETQQNLCRLVQEGAQVAAMDLYEFKKCWKKFVDNGGRRFRP
WKRLLTNFRYQDSKLQEILRPCYIPVPSSSSSTLSNICLTKGLPETRFCVEGRRMDPLSEE
EFYSQFYNQRVKHLCYYHRMKPYLCYQLEQFNGQAPLKGCLLSEKGKQHAEILFLDKIR
SMELSQVTITCYLTWSPCPNCAWQLAAFKRDRPDLILHIYTSRLYFHWKRPFQKGLCSLW
QSGILVDVMDLPQFTDCWTNFVNPKRPFWPWKGLEIISRRTQRRLRRIKESWGLQDLVN
DFGNLQLGPPMS
(italic: nucleic acid editing domain)
Rat APOBEC-3:
(SEQ ID NO: 23)
MGPFCLGCSHRKCYSPIRNLISQETFKFHFKNRLRYAIDRKDTFLCYEVTRKDCDSPVSL
HHGVFKNKDNIHAEICFLYWFHDKVLKVLSPREEFKITWYMSWSPCFECAEQVLRFLATHH
NLSLDIFSSRLYNIRDPENQQNLCRLVQEGAQVAAMDLYEFKKCWKKFVDNGGRRFRP
WKKLLTNFRYQDSKLQEILRPCYIPVPSSSSSTLSNICLTKGLPETRFCVERRRVHLLSEEE
FYSQFYNQRVKHLCYYHGVKPYLCYQLEQFNGQAPLKGCLLSEKGKQHAEILFLDKIRS
MELSQVIITCYLTWSPCPNCAWQLAAFKRDRPDLILHIYTSRLYFHWKRPFQKGLCSLWQ
SGILVDVMDLPQFTDCWTNFVNPKRPFWPWKGLEIISRRTQRRLHRIKESWGLQDLVND
FGNLQLGPPMS
(italic: nucleic acid editing domain)
hAPOBEC-3A (Homo sapiens):
(SEQ ID NO: 24)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQEN
THVRLRIFAARIYDYDPLYKEALQMLRDAGAQVSIMTYDEFKHCWDTFVDHQGCPFQP
WDGLDEHSQALSGRLRAILQNQGN
hAPOBEC-3F (Homo sapiens):
(SEQ ID NO: 25)
MKPHFRNTVERMYRDTFSYNFYNRPILSRRNTVWLCYEVKTKGPSRPRLDAKIFRGQV
YSQPEHHAEMCFLSWFCGNQLPAYKCFQITWFVSWTPCPDCVAKLAEFLAEHPNVTLTI
SAARLYYYWERDYRRALCRLSQAGARVKIMDDEEFAYCWENFVYSEGQPFMPWYKFD
DNYAFLHRTLKEILRNPMEAMYPHIFYFHFKNLRKAYGRNESWLCFTMEVVKHHSPVS
WKRGVFRNQVDPETHCHAERCFLSWFCDDILSPNTNYEVTWYTSWSPCPECAGEVAEF
LARHSNVNLTIFTARLYYFWDTDYQEGLRSLSQEGASVEIMGYKDFKYCWENFVYNDD
EPFKPWKGLKYNFLFLDSKLQEILE
Rhesus macaque APOBEC-3G:
(SEQ ID NO: 26)
MVEPMDPRTFVSNFNNRPILSGLNTVWLCCEVKTKDPSGPPLDAKIFQGKVYSKAKYHP
EMRFLRWFHKWRQLHHDQEYKVTWYVSWSPCTRCANSVATFLAKDPKVTLTIFVARL
YYFWKPDYQQALRILCQKRGGPHATMKIMNYNEFQDCWNKFVDGRGKPFKPRNNLPK
HYTLLQATLGELLRHLMDPGTFTSNFNNKPWVSGQHETYLCYKVERLHNDTWVPLNQ
HRGFLRNQAPNIHGFPKGRHAELCFLDLIPFWKLDGQQYRVTCFTSWSPCFSCAQEMAK
FISNNEHVSLCIFAARIYDDQGRYQEGLRALHRDGAKIAMMNYSEFEYCWDTFVDRQG
RPFQPWDGLDEHSQALSGRLRAI
(italic: nucleic acid editing domain; underline:
cytoplasmic localization signal)
Chimpanzee APOBEC-3G:
(SEQ ID NO: 27)
MKPHFRNPVERMYQDTFSDNFYNRPILSHRNTVWLCYEVKTKGPSRPPLDAKIFRGQVY
SKLKYHPEMRFFHWFSKWRKLHRDQEYEVTWYISWSPCTKCTRDVATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTSNFNNELWVRGRHETYLCYEVERLHNDTWVLL
NQRRGFLCNQAPHKHGFLEGRHAELCFLDVIPFWKLDLHQDYRVTCFTSWSPCFSCAQEM
AKFISNNKHVSLCIFAARIYDDQGRCQEGLRTLAKAGAKISIMTYSEFKHCWDTFVDHQ
GCPFQPWDGLEEHSQALSGRLRAILQNQGN
(italic: nucleic acid editing domain; underline:
cytoplasmic localization signal)
Green monkey APOBEC-3G:
(SEQ ID NO: 28)
MNPQIRNMVEQMEPDIFVYYFNNRPILSGRNTVWLCYEVKTKDPSGPPLDANIFQGKLY
PEAKDHPEMKFLHWFRKWRQLHRDQEYEVTWYVSWSPCTRCANSVATFLAEDPKVTLTIF
VARLYYFWKPDYQQALRILCQERGGPHATMKIMNYNEFQHCWNEFVDGQGKPFKPRK
NLPKHYTLLHATLGELLRHVMDPGTFTSNFNNKPWVSGQRETYLCYKVERSHNDTWV
LLNQHRGFLRNQAPDRHGFPKGRHAELCFLDLIPFWKLDDQQYRVTCFTSWSPCFSCAQK
MAKFISNNKHVSLCIFAARIYDDQGRCQEGLRTLHRDGAKIAVMNYSEFEYCWDTFVD
RQGRPFQPWDGLDEHSQALSGRLRAI
(italic: nucleic acid editing domain; underline:
cytoplasmic localization signal)
Human APOBEC-3G:
(SEQ ID NO: 29)
MKPHFRNTVERMYRDTFSYNFYNRPILSRRNTVWLCYEVKTKGPSRPPLDAKIFRGQVY
SELKYHPEMRFFHWFSKWRKLHRDQEYEVTWYISWSPCTKCTRDMATFLAEDPKVTLTIF
VARLYYFWDPDYQEALRSLCQKRDGPRATMKIMNYDEFQHCWSKFVYSQRELFEPWN
NLPKYYILLHIMLGEILRHSMDPPTFTFNFNNEPWVRGRHETYLCYEVERMHNDTWVLL
NQRRGFLCNQAPHKHGFLEGRHAELCFLDVIPFWKLDLDQDYRVTCFTSWSPCFSCAQEM
AKFISKNKHVSLCIFTARIYDDQGRCQEGLRTLAEAGAKISIMTYSEFKHCWDTFVDHQ
GCPFQPWDGLDEHSQDLSGRLRAILQNQEN
(italic: nucleic acid editing domain; underline:
cytoplasmic localization signal)
Human APOBEC-3F:
(SEQ ID NO: 30)
MKPHFRNTVERMYRDTFSYNFYNRPILSRRNTVWLCYEVKTKGPSRPRLDAKIFRGQV
YSQPEHHAEMCFLSWFCGNQLPAYKCFQITWFVSWTPCPDCVAKLAEFLAEHPNVTLTIS
AARLYYYWERDYRRALCRLSQAGARVKIMDDEEFAYCWENFVYSEGQPFMPWYKFD
DNYAFLHRTLKEILRNPMEAMYPHIFYFHFKNLRKAYGRNESWLCFTMEVVKHHSPVS
WKRGVFRNQVDPETHCHAERCFLSWFCDDILSPNTNYEVTWYTSWSPCPECAGEVAEFLA
RHSNVNLTIFTARLYYFWDTDYQEGLRSLSQEGASVEIMGYKDFKYCWENFVYNDDEP
FKPWKGLKYNFLFLDSKLQEILE
(italic: nucleic acid editing domain)
Human APOBEC-3B:
(SEQ ID NO: 31)
MNPQIRNPMERMYRDTFYDNFENEPILYGRSYTWLCYEVKIKRGRSNLLWDTGVFRGQ
VYFKPQYHAEMCFLSWFCGNQLPAYKCFQITWFVSWTPCPDCVAKLAEFLSEHPNVTLTI
SAARLYYYWERDYRRALCRLSQAGARVTIMDYEEFAYCWENFVYNEGQQFMPWYKF
DENYAFLHRTLKEILRYLMDPDTFTFNFNNDPLVLRRRQTYLCYEVERLDNGTWVLMD
QHMGFLCNEAKNLLCGFYGRHAELRFLDLVPSLQLDPAQIYRVTWFISWSPCFSWGCAGE
VRAFLQENTHVRLRIFAARIYDYDPLYKEALQMLRDAGAQVSIMTYDEFEYCWDTFVY
RQGCPFQPWDGLEEHSQALSGRLRAILQNQGN
(italic: nucleic acid editing domain)
Rat APOBEC-3B:
(SEQ ID NO: 32)
MQPQGLGPNAGMGPVCLGCSHRRPYSPIRNPLKKLYQQTFYFHFKNVRYAWGRKNNF
LCYEVNGMDCALPVPLRQGVFRKQGHIHAELCFIYWFHDKVLRVLSPMEEFKVTWYM
SWSPCSKCAEQVARFLAAHRNLSLAIFSSRLYYYLRNPNYQQKLCRLIQEGVHVAAMD
LPEFKKCWNKFVDNDGQPFRPWMRLRINFSFYDCKLQEIFSRMNLLREDVFYLQFNNSH
RVKPVQNRYYRRKSYLCYQLERANGQEPLKGYLLYKKGEQHVEILFLEKMRSMELSQV
RITCYLTWSPCPNCARQLAAFKKDHPDLILRIYTSRLYFWRKKFQKGLCTLWRSGIHVD
VMDLPQFADCWTNFVNPQRPFRPWNELEKNSWRIQRRLRRIKESWGL
Bovine APOBEC-3B:
(SEQ ID NO: 33)
MDGWEVAFRSGTVLKAGVLGVSMTEGWAGSGHPGQGACVWTPGTRNTMNLLREVLF
KQQFGNQPRVPAPYYRRKTYLCYQLKQRNDLTLDRGCFRNKKQRHAERFIDKINSLDL
NPSQSYKIICYITWSPCPNCANELVNFITRNNHLKLEIFASRLYFHWIKSFKMGLQDLQNA
GISVAVMTHTEFEDCWEQFVDNQSRPFQPWDKLEQYSASIRRRLQRILTAPI
Chimpanzee APOBEC-3B:
(SEQ ID NO: 34)
MNPQIRNPMEWMYQRTFYYNFENEPILYGRSYTWLCYEVKIRRGHSNLLWDTGVFRGQ
MYSQPEHHAEMCFLSWFCGNQLSAYKCFQITWFVSWTPCPDCVAKLAKFLAEHPNVTL
TISAARLYYYWERDYRRALCRLSQAGARVKIMDDEEFAYCWENFVYNEGQPFMPWYK
FDDNYAFLHRTLKEIIRHLMDPDTFTFNFNNDPLVLRRHQTYLCYEVERLDNGTWVLM
DQHMGFLCNEAKNLLCGFYGRHAELRFLDLVPSLQLDPAQIYRVTWFISWSPCFSWGC
AGQVRAFLQENTHVRLRIFAARIYDYDPLYKEALQMLRDAGAQVSIMTYDEFEYCWDT
FVYRQGCPFQPWDGLEEHSQALSGRLRAILQVRASSLCMVPHRPPPPPQSPGPCLPLCSE
PPLGSLLPTGRPAPSLPFLLTASFSFPPPASLPPLPSLSLSPGHLPVPSFHSLTSCSIQPPCSSR
IRETEGWASVSKEGRDLG
Human APOBEC-3C:
(SEQ ID NO: 35)
MNPQIRNPMKAMYPGTFYFQFKNLWEANDRNETWLCFTVEGIKRRSVVSWKTGVFRN
QVDSETHCHAERCFLSWFCDDILSPNTKYQVTWYTSWSPCPDCAGEVAEFLARHSNVNLTI
FTARLYYFQYPCYQEGLRSLSQEGVAVEIMDYEDFKYCWENFVYNDNEPFKPWKGLKT
NFRLLKRRLRESLQ
(italic: nucleic acid editing domain)
Gorilla APOBEC-3C
(SEQ ID NO: 36)
MNPQIRNPMKAMYPGTFYFQFKNLWEANDRNETWLCFTVEGIKRRSVVSWKTGVFRN
QVDSETHCHAERCFLSWECDDILSPNTNYQVTWYTSWSPCPECAGEVAEFLARHSNVNLTI
FTARLYYFQDTDYQEGLRSLSQEGVAVKIMDYKDFKYCWENFVYNDDEPFKPWKGLK
YNFRFLKRRLQEILE
(italic: nucleic acid editing domain)
Human APOBEC-3A:
(SEQ ID NO: 37)
MEASPASGPRHLMDPHIFTSNFNNGIGRHKTYLCYEVERLDNGTSVKMDQHRGFLHNQ
AKNLLCGFYGRHAELRFLDLVPSLQLDPAQIYRVTWFISWSPCFSWGCAGEVRAFLQENTH
VRLRIFAARIYDYDPLYKEALQMLRDAGAQVSIMTYDEFKHCWDTFVDHQGCPFQPWD
GLDEHSQALSGRLRAILQNQGN
(italic: nucleic acid editing domain)
Rhesus macaque APOBEC-3A:
(SEQ ID NO: 38)
MDGSPASRPRHLMDPNTFTFNFNNDLSVRGRHQTYLCYEVERLDNGTWVPMDERRGF
LCNKAKNVPCGDYGCHVELRFLCEVPSWQLDPAQTYRVTWFISWSPCFRRGCAGQVRVFL
QENKHVRLRIFAARIYDYDPLYQEALRTLRDAGAQVSIMTYEEFKHCWDTFVDRQGRP
FQPWDGLDEHSQALSGRLRAILQNQGN
(italic: nucleic acid editing domain)
Bovine APOBEC-3A:
(SEQ ID NO: 39)
MDEYTFTENFNNQGWPSKTYLCYEMERLDGDATIPLDEYKGFVRNKGLDQPEKPCHAE
LYFLGKIHSWNLDRNQHYRLTCFISWSPCYDCAQKLTTFLKENHHISLHILASRIYTHNRFG
CHQSGLCELQAAGARITIMTFEDFKHCWETFVDHKGKPFQPWEGLNVKSQALCTELQAI
LKTQQN
(italic: nucleic acid editing domain)
Human APOBEC-3H:
(SEQ ID NO: 40)
MALLTAETFRLQFNNKRRLRRPYYPRKALLCYQLTPQNGSTPTRGYFENKKKCHAEICFI
NEIKSMGLDETQCYQVTCYLTWSPCSSCAWELVDFIKAHDHLNLGIFASRLYYHWCKPQQ
KGLRLLCGSQVPVEVMGFPKFADCWENFVDHEKPLSFNPYKMLEELDKNSRAIKRRLE
RIKIPGVRAQGRYMDILCDAEV
(italic: nucleic acid editing domain)
Rhesus macaque APOBEC-3H:
(SEQ ID NO: 41)
MALLTAKTFSLQFNNKRRVNKPYYPRKALLCYQLTPQNGSTPTRGHLKNKKKDHAEIR
FINKIKSMGLDETQCYQVTCYLTWSPCPSCAGELVDFIKAHRHLNLRIFASRLYYHWRP
NYQEGLLLLCGSQVPVEVMGLPEFTDCWENFVDHKEPPSFNPSEKLEELDKNSQAIKRR
LERIKSRSVDVLENGLRSLQLGPVTPSSSIRNSR
Human APOBEC-3D:
(SEQ ID NO: 42)
MNPQIRNPMERMYRDTFYDNFENEPILYGRSYTWLCYEVKIKRGRSNLLWDTGVFRGP
VLPKRQSNHRQEVYFRFENHAEMCFLSWFCGNRLPANRRFQITWFVSWNPCLPCVVKVT
KFLAEHPNVTLTISAARLYYYRDRDWRWVLLRLHKAGARVKIMDYEDFAYCWENFVC
NEGQPFMPWYKFDDNYASLHRTLKEILRNPMEAMYPHIFYFHFKNLLKACGRNESWLC
FTMEVTKHHSAVFRKRGVFRNQVDPETHCHAERCFLSWFCDDILSPNTNYEVTWYTSWSP
CPECAGEVAEFLARHSNVNLTIFTARLCYFWDTDYQEGLCSLSQEGASVKIMGYKDFVS
CWKNFVYSDDEPFKPWKGLQTNFRLLKRRLREILQ
(italic: nucleic acid editing domain)
Human APOBEC-1:
(SEQ ID NO: 43)
MTSEKGPSTGDPTLRRRIEPWEFDVFYDPRELRKEACLLYEIKWGMSRKIWRSSGKNTT
NHVEVNFIKKFTSERDFHPSMSCSITWFLSWSPCWECSQAIREFLSRHPGVTLVIYVARLF
WHMDQQNRQGLRDLVNSGVTIQIMRASEYYHCWRNFVNYPPGDEAHWPQYPPLWMM
LYALELHCIILSLPPCLKISRRWQNHLTFFRLHLQNCHYQTIPPHILLATGLIHPSVAWR
Mouse APOBEC-1:
(SEQ ID NO: 44)
MSSETGPVAVDPTLRRRIEPHEFEVFFDPRELRKETCLLYEINWGGRHSVWRHTSQNTSN
HVEVNFLEKFTTERYFRPNTRCSITWFLSWSPCGECSRAITEFLSRHPYVTLFIYIARLYH
HTDQRNRQGLRDLISSGVTIQIMTEQEYCYCWRNFVNYPPSNEAYWPRYPHLWVKLYV
LELYCIILGLPPCLKILRRKQPQLTFFTITLQTCHYQRIPPHLLWATGLK
Rat APOBEC-1:
(SEQ ID NO: 45)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVL
ELYCIILGLPPCLNILRRKQPQLTFFTIALQSCHYQRLPPHILWATGLK
Human APOBEC-2:
(SEQ ID NO: 46)
MAQKEEAAVATEAASQNGEDLENLDDPEKLKELIELPPFEIVTGERLPANFFKFQFRNVE
YSSGRNKTFLCYVVEAQGKGGQVQASRGYLEDEHAAAHAEEAFFNTILPAFDPALRYN
VTWYVSSSPCAACADRIIKTLSKTKNLRLLILVGRLFMWEEPEIQAALKKLKEAGCKLRI
MKPQDFEYVWQNFVEQEEGESKAFQPWEDIQENFLYYEEKLADILK
Mouse APOBEC-2:
(SEQ ID NO: 47)
MAQKEEAAEAAAPASQNGDDLENLEDPEKLKELIDLPPFEIVTGVRLPVNFFKFQFRNV
EYSSGRNKTFLCYVVEVQSKGGQAQATQGYLEDEHAGAHAEEAFFNTILPAFDPALKY
NVTWYVSSSPCAACADRILKTLSKTKNLRLLILVSRLFMWEEPEVQAALKKLKEAGCKL
RIMKPQDFEYIWQNFVEQEEGESKAFEPWEDIQENFLYYEEKLADILK
Rat APOBEC-2:
(SEQ ID NO: 48)
MAQKEEAAEAAAPASQNGDDLENLEDPEKLKELIDLPPFEIVTGVRLPVNFFKFQFRNV
EYSSGRNKTFLCYVVEAQSKGGQVQATQGYLEDEHAGAHAEEAFFNTILPAFDPALKY
NVTWYVSSSPCAACADRILKTLSKTKNLRLLILVSRLFMWEEPEVQAALKKLKEAGCKL
RIMKPQDFEYLWQNFVEQEEGESKAFEPWEDIQENFLYYEEKLADILK
Bovine APOBEC-2:
(SEQ ID NO: 49)
MAQKEEAAAAAEPASQNGEEVENLEDPEKLKELIELPPFEIVTGERLPAHYFKFQFRNVE
YSSGRNKTFLCYVVEAQSKGGQVQASRGYLEDEHATNHAEEAFFNSIMPTFDPALRYM
VTWYVSSSPCAACADRIVKTLNKTKNLRLLILVGRLFMWEEPEIQAALRKLKEAGCRLR
IMKPQDFEYIWQNFVEQEEGESKAFEPWEDIQENFLYYEEKLADILK
Petromyzon marinus CDA1 (pmCDA1):
(SEQ ID NO: 50)
MTDAEYVRIHEKLDIYTFKKQFFNNKKSVSHRCYVLFELKRRGERRACFWGYAVNKPQ
SGTERGIHAEIFSIRKVEEYLRDNPGQFTINWYSSWSPCADCAEKILEWYNQELRGNGHT
LKIWACKLYYEKNARNQIGLWNLRDNGVGLNVMVSEHYQCCRKIFIQSSHNQ
LNENRWLEKTLKRAEKRRSELSFMIQVKILHTTKSPAV
Human APOBEC3G D316R D317R:
(SEQ ID NO: 51)
MKPHFRNTVERMYRDTFSYNFYNRPILSRRNTVWLCYEVKTKGPSRPPLDAKIFRGQVY
SELKYHPEMRFFHWFSKWRKLHRDQEYEVTWYISWSPCTKCTRDMATFLAEDPKVTLT
IFVARLYYFWDPDYQEALRSLCQKRDGPRATMKFNYDEFQHCWSKFVYSQRELFEPWN
NLPKYYILLHFMLGEILRHSMDPPTFTFNFNNEPWVRGRHETYLCYEVERMHNDTWVL
LNQRRGFLCNQAPHKHGFLEGRHAELCFLDVIPFWKLDLDQDYRVTCFTSWSPCFSCAQ
EMAKFISKKHVSLCIFTARIYRRQGRCQEGLRTLAEAGAKISFTYSEFKHCWDTFVDHQ
GCPFQPWDGLDEHSQDLSGRLRAILQNQEN
Human APOBEC3G chain A:
(SEQ ID NO: 52)
MDPPTFTFNFNNEPWWGRHETYLCYEVERMHNDTWVLLNQRRGFLCNQAPHKHGFLE
GRHAELCFLDVIPFWKLDLDQDYRVTCFTSWSPCFSCAQEMAKFISKNKHVSLCIFTARI
YDDQGRCQEGLRTLAEAGAKISFTYSEFKHCWDTFVDHQGCPFQPWDGLD
EHSQDLSGRLRAILQ
Human APOBEC3G chain A D120R D121R:
(SEQ ID NO: 53)
MDPPTFTFNFNNEPWVRGRHETYLCYEVERMHNDTWVLLNQRRGFLCNQAPHKHGFL
EGRHAELCFLDVIPFWKLDLDQDYRVTCFTSWSPCFSCAQEMAKFISKNKHVSLCIFTAR
IYRRQGRCQEGLRTLAEAGAKISFMTYSEFKHCWDTFVDHQGCPFQPWDGLDEHSQDL
SGRLRAILQ
hAPOBEC-4 (Homo sapiens):
(SEQ ID NO: 54)
MEPIYEEYLANHGTIVKPYYWLSFSLDCSNCPYHIRTGEEARVSLTEFCQIFGFPYGTTFP
QTKHLTFYELKTSSGSLVQKGHASSCTGNYIHPESMLFEMNGYLDSAIYNNDSIRHIILYS
NNSPCNEANHCCISKMYNFLITYPGITLSIYFSQLYHTEMDFPASAWNREALRSLASLWP
RVVLSPISGGIWHSVLHSFISGVSGSHVFQPILTGRALADRHNAYEINAITGVKPYFTDVL
LQTKRNPNTKAQEALESYPLNNAFPGQFFQMPSGQLQPNLPPDLRAPVVFVLVPLRDLP
PMHMGQNPNKPRNIVRHLNMPQMSFQETKDLGRLPTGRSVEIVEITEQFASSKEADEKK
KKKGKK
mAPOBEC-4 (Mus musculus):
(SEQ ID NO: 55)
MDSLLMKQKKFLYHFKNVRWAKGRHETYLCYVVKRRDSATSCSLDFGHLRNKSGCHV
ELLFLRYISDWDLDPGRCYRVTWFTSWSPCYDCARHVAEFLRWNPNLSLRIFTARLYFC
EDRKAEPEGLRRLHRAGVQIGIMTFKDYFYCWNTFVENRERTFKAWEGLHENSVRLTR
QLRRILLPLYEVDDLRDAFRMLGF
rAPOBEC-4 (Rattus norvegicus):
(SEQ ID NO: 56)
MEPLYEEYLTHSGTIVKPYYWLSVSLNCTNCPYHIRTGEEARVPYTEFHQTFGFPWSTYP
QTKHLTFYELRSSSGNLIQKGLASNCTGSHTHPESMLFERDGYLDSLIFHDSNIRHIILYSN
NSPCDEANHCCISKMYNFLMNYPEVTLSVFFSQLYHTENQFPTSAWNREALRGLASLWP
QVTLSAISGGIWQSILETFVSGISEGLTAVRPFTAGRTLTDRYNAYEINCITEVKPYFTDAL
HSWQKENQDQKVWAASENQPLHNTTPAQWQPDMSQDCRTPAVFMLVPYRDLPPIHVN
PSPQKPRTVVRHLNTLQLSASKVKALRKSPSGRPVKKEEARKGSTRSQEANETNKSKW
KKQTLFIKSNICHLLEREQKKIGILSSWSV
mfAPOBEC-4 (Macaca fascicularis):
(SEQ ID NO: 57)
MEPTYEEYLANHGTIVKPYYWLSFSLDCSNCPYHIRTGEEARVSLTEFCQIFGFPYGTTY
PQTKHLTFYELKTSSGSLVQKGHASSCTGNYIHPESMLFEMNGYLDSAIYNNDSIRHIILY
CNNSPCNEANHCCISKVYNFLITYPGITLSIYFSQLYHTEMDFPASAWNREALRSLASLW
PRVVLSPISGGIWHSVLHSFVSGVSGSHVFQPILTGRALTDRYNAYEINAITGVKPFFTDV
LLHTKRNPNTKAQMALESYPLNNAFPGQSFQMTSGIPPDLRAPVVFVLLPLRDLPPMHM
GQDPNKPRNIIRHLNMPQMSFQETKDLERLPTRRSVETVEITERFASSKQAEEKTKKKKG
KK
pmCDA-1 (Petromyzon marinus):
(SEQ ID NO: 58)
MAGYECVRVSEKLDFDTFEFQFENLHYATERHRTYVIFDVKPQSAGGRSRRLWGYIINN
PNVCHAELILMSMIDRHLESNPGVYAMTWYMSWSPCANCSSKLNPWLKNLLEEQGHT
LTMHFSRIYDRDREGDHRGLRGLKHVSNSFRMGVVGRAEVKECLAEYVEASRRTLTWL
DTTESMAAKMRRKLFCILVRCAGMRESGIPLHLFTLQTPLLSGRVVWWRV
pmCDA-2 (Petromyzon marinus):
(SEQ ID NO: 59)
MELREVVDCALASCVRHEPLSRVAFLRCFAAPSQKPRGTVILFYVEGAGRGVTGGHAV
NYNKQGTSIHAEVLLLSAVRAALLRRRRCEDGEEATRGCTLHCYSTYSPCRDCVEYIQE
FGASTGVRVVIHCCRLYELDVNRRRSEAEGVLRSLSRLGRDFRLMGPRDAIALLLGGRL
ANTADGESGASGNAWVTETNVVEPLVDMTGFGDEDLHAQVQRNKQIREAYANYASAV
SLMLGELHVDPDKFPFLAEFLAQTSVEPSGTPRETRGRPRGASSRGPEIGRQRPADFERA
LGAYGLFLHPRIVSREADREEIKRDLIVVMRKHNYQGP
pmCDA-5 (Petromyzon marinus):
(SEQ ID NO: 60)
MAGDENVRVSEKLDFDTFEFQFENLHYATERHRTYVIFDVKPQSAGGRSRRLWGYIINN
PNVCHAELILMSMIDRHLESNPGVYAMTWYMSWSPCANCSSKLNPWLKNLLEEQGHT
LMMHFSRIYDRDREGDHRGLRGLKHVSNSFRMGVVGRAEVKECLAEYVEASRRTLTW
LDTTESMAAKMRRKLFCILVRCAGMRESGMPLHLFT
yCD (Saccharomyces cerevisiae):
(SEQ ID NO: 61)
MVTGGMASKWDQKGMDIAYEEAALGYKEGGVPIGGCLINNKDGSVLGRGHNMRFQK
GSATLHGEISTLENCGRLEGKVYKDTTLYTTLSPCDMCTGAIIMYGIPRCVVGENVNFKS
KGEKYLQTRGHEVVVVDDERCKKIMKQFIDERPQDWFEDIGE
rAPOBEC-1 (delta 177-186):
(SEQ ID NO: 62)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRGLPPC
LNILRRKQPQLTFFTIALQSCHYQRLPPHILWATGLK
rAPOBEC-1 (delta 202-213):
(SEQ ID NO: 63)
MSSETGPVAVDPTLRRRIEPHEFEVFFDPRELRKETCLLYEINWGGRHSIWRHTSQNTNK
HVEVNFIEKFTTERYFCPNTRCSITWFLSWSPCGECSRAITEFLSRYPHVTLFIYIARLYHH
ADPRNRQGLRDLISSGVTIQIMTEQESGYCWRNFVNYSPSNEAHWPRYPHLWVRLYVL
ELYCIILGLPPCLNILRRKQPQHYQRLPPHILWATGLK
Mouse APOBEC-3:
(SEQ ID NO: 64)
MGPFCLGCSHRKCYSPIRNLISQETFKFHFKNLGYAKGRKDTFLCYEVTRKDCDSPVSLH
HGVFKNKDNIHAEICFLYWFHDKVLKVLSPREEFKITWYMSWSPCFECAEQIVRFLATHHN
LSLDIFSSRLYNVQDPETQQNLCRLVQEGAQVAAMDLYEFKKCWKKFVDNGGRRFRP
WKRLLTNFRYQDSKLQEILRPCYIPVPSSSSSTLSNICLTKGLPETRFCVEGRRMDPLSEE
EFYSQFYNQRVKHLCYYHRMKPYLCYQLEQFNGQAPLKGCLLSEKGKQHAEILFLDKIR
SMELSQVTITCYLTWSPCPNCAWQLAAFKRDRPDLILHIYTSRLYFHWKRPFQKGLCSLW
QSGILVDVMDLPQFTDCWTNFVNPKRPFWPWKGLEIISRRTQRRLRRIKESWGLQDLVN
DFGNLQLGPPMS
(italic: nucleic acid editing domain)

In some embodiments, an adenosine deaminase can comprise all or a portion of an adenosine deaminase ADAR (e.g., ADAR1 or ADAR2). In another embodiment, an adenosine deaminase can comprise all or a portion of an adenosine deaminase ADAT. In some embodiments, an adenosine deaminase can comprise all or a portion of an ADAT from Escherichia coli (EcTadA) comprising one or more of the following mutations: D108N, A106V, D147Y, E155V, L84F, H123Y, I157F, or a corresponding mutation in another adenosine deaminase. The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by, e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly. In particular embodiments, the TadA is any one of the TadA described in PCT/US2017/045381 (WO 2018/027078), which is incorporated herein by reference in its entirety. Mutations were identified through rounds of evolution and selection (e.g., TadA*7.10=variant 10 from seventh round of evolution) having desirable adenosine deaminase activity on single stranded DNA as shown in Table 7.

TABLE 7
Genotypes of TadA Variants

| TadA | 23 | 26 | 36 | 37 | 48 | 49 | 51 | 72 | 84 | 87 | 105 | 108 | 123 | 125 | 142 | 145 | 147 | 152 | 155 | 156 | 157 | 16 |
|---|---|---|---|---|---|---|---|---|---|---|---|---|---|---|---|---|---|---|---|---|---|---|
| 0.1 | W | R | H | N | P | | R | N | L | S | A | D | H | G | A | S | D | R | E | I | K | K |
| 0.2 | W | R | H | N | P | | R | N | L | S | A | D | H | G | A | S | D | R | E | I | K | K |
| 1.1 | W | R | H | N | P | | R | N | L | S | A | N | H | G | A | S | D | R | E | I | K | K |
| 1.2 | W | R | H | N | P | | R | N | L | S | V | N | H | G | A | S | D | R | E | I | K | K |
| 2.1 | W | R | H | N | P | | R | N | L | S | V | N | H | G | A | S | Y | R | V | I | K | K |
| 2.2 | W | R | H | N | P | | R | N | L | S | V | N | H | G | A | S | Y | R | V | I | K | K |
| 2.3 | W | R | H | N | P | | R | N | L | S | V | N | H | G | A | S | Y | R | V | I | K | K |
| 2.4 | W | R | H | N | P | | R | N | L | S | V | N | H | G | A | S | Y | R | V | I | K | K |
| 2.5 | W | R | H | N | P | | R | N | L | S | V | N | H | G | A | S | Y | R | V | I | K | K |
| 2.6 | W | R | H | N | P | | R | N | L | S | V | N | H | G | A | S | Y | R | V | I | K | K |
| 2.7 | W | R | H | N | P | | R | N | L | S | V | N | H | G | A | S | Y | R | V | I | K | K |
| 2.8 | W | R | H | N | P | | R | N | L | S | V | N | H | G | A | S | Y | R | V | I | K | K |
| 2.9 | W | R | H | N | P | | R | N | L | S | V | N | H | G | A | S | Y | R | V | I | K | K |
| 2.10 | W | R | H | N | P | | R | N | L | S | V | N | H | G | A | S | Y | R | V | I | K | K |
| 2.11 | W | R | H | N | P | | R | N | L | S | V | N | H | G | A | S | Y | R | V | I | K | K |
| 2.12 | W | R | H | N | P | | R | N | L | S | V | N | H | G | A | S | Y | R | V | I | K | K |
| 3.1 | W | R | H | N | P | | R | N | F | S | V | N | Y | G | A | S | Y | R | V | F | K | K |
| 3.2 | W | R | H | N | P | | R | N | F | S | V | N | Y | G | A | S | Y | R | V | F | K | K |
| 3.3 | W | R | H | N | P | | R | N | F | S | V | N | Y | G | A | S | Y | R | V | F | K | K |
| 3.4 | W | R | H | N | P | | R | N | F | S | V | N | Y | G | A | S | Y | R | V | F | K | K |
| 3.5 | W | R | H | N | P | | R | N | F | S | V | N | Y | G | A | S | Y | R | V | F | K | K |
| 3.6 | W | R | H | N | P | | R | N | F | S | V | N | Y | G | A | S | Y | R | V | F | K | K |
| 3.7 | W | R | H | N | P | | R | N | F | S | V | N | Y | G | A | S | Y | R | V | F | K | K |
| 3.8 | W | R | H | N | P | | R | N | F | S | V | N | Y | G | A | S | Y | R | V | F | K | K |
| 4.1 | W | R | H | N | P | | R | N | L | S | V | N | H | G | N | S | Y | R | V | I | K | K |
| 4.2 | W | G | H | N | P | | R | N | L | S | V | N | H | G | N | S | Y | R | V | I | K | K |
| 4.3 | W | R | H | N | P | | R | N | F | S | V | N | Y | G | N | S | Y | R | V | F | K | K |
| 5.1 | W | R | L | N | P | | L | N | F | S | V | N | Y | G | A | C | Y | R | V | F | N | K |
| 5.2 | W | R | H | S | P | | R | N | F | S | V | N | Y | G | A | S | Y | R | V | F | K | T |
| 5.3 | W | R | L | N | P | | L | N | I | S | V | N | Y | G | A | C | Y | R | V | I | N | K |
| 5.4 | W | R | H | S | P | | R | N | F | S | V | N | Y | G | A | S | Y | R | V | F | K | T |
| 5.5 | W | R | L | N | P | | L | N | F | S | V | N | Y | G | A | C | Y | R | V | F | N | K |
| 5.6 | W | R | L | N | P | | L | N | F | S | V | N | Y | G | A | C | Y | R | V | F | N | K |
| 5.7 | W | R | L | N | P | | L | N | F | S | V | N | Y | G | A | C | Y | R | V | F | N | K |
| 5.8 | W | R | L | N | P | | L | N | F | S | V | N | Y | G | A | C | Y | R | V | F | N | K |
| 5.9 | W | R | L | N | P | | L | N | F | S | V | N | Y | G | A | C | Y | R | V | F | N | K |
| 5.10 | W | R | L | N | P | | L | N | F | S | V | N | Y | G | A | C | Y | R | V | F | N | K |
| 5.11 | W | R | L | N | P | | L | N | F | S | V | N | Y | G | A | C | Y | R | V | F | N | K |
| 5.12 | W | R | L | N | P | | L | N | F | S | V | N | Y | G | A | C | Y | R | V | F | N | K |
| 5.13 | W | R | H | N | P | | L | D | F | S | V | N | Y | A | A | S | Y | R | V | F | K | K |
| 5.14 | W | R | H | N | S | | L | N | F | C | V | N | Y | G | A | S | Y | R | V | F | K | K |
| 6.1 | W | R | H | N | S | | L | N | F | S | V | N | Y | G | N | S | Y | R | V | F | K | K |
| 6.2 | W | R | H | N | T | V | L | N | F | S | V | N | Y | G | N | S | Y | R | V | F | N | K |
| 6.3 | W | R | L | N | S | | L | N | F | S | V | N | Y | G | A | C | Y | R | V | F | N | K |
| 6.4 | W | R | L | N | S | | L | N | F | S | V | N | Y | G | N | C | Y | R | V | F | N | K |
| 6.5 | W | R | L | N | I | V | L | N | F | S | V | N | Y | G | A | C | Y | R | V | F | N | K |
| 6.6 | W | R | L | N | T | V | L | N | F | S | V | N | Y | G | N | C | Y | R | V | F | N | K |
| 7.1 | W | R | L | N | A | | L | N | F | S | V | N | Y | G | A | C | Y | R | V | F | N | K |
| 7.2 | W | R | L | N | A | | L | N | F | S | V | N | Y | G | N | C | Y | R | V | F | N | K |
| 7.3 | I | R | L | N | A | | L | N | F | S | V | N | Y | G | A | C | Y | R | V | F | N | K |
| 7.4 | R | R | L | N | A | | L | N | F | S | V | N | Y | G | A | C | Y | R | V | F | N | K |
| 7.5 | W | R | L | N | A | | L | N | F | S | V | N | Y | G | A | C | Y | H | V | F | N | K |
| 7.6 | W | R | L | N | A | | L | N | I | S | V | N | Y | G | A | C | Y | P | V | I | N | K |
| 7.7 | L | R | L | N | A | | L | N | F | S | V | N | Y | G | A | C | Y | P | V | F | N | K |
| 7.8 | I | R | L | N | A | | L | N | F | S | V | N | Y | G | N | C | Y | R | V | F | N | K |
| 7.9 | L | R | L | N | A | | L | N | F | S | V | N | Y | G | N | C | Y | P | V | F | N | K |
| 7.10 | R | R | L | N | A | | L | N | F | S | V | N | Y | G | A | C | Y | P | V | F | N | K |

In some embodiments, the TadA is provided as a monomer or dimer (e.g., a heterodimer of wild-type E. coli TadA and an engineered TadA variant). In some embodiments, the adenosine deaminase is an eighth generation TadA*8 variant as shown in Table 8 below.

TABLE 8
TadA8* Adenosine Deaminase Variants

| Adenosine | |
|---|---|
| Deaminase | Adenosine Deaminase Description |
| TadA*8.1 | Monomer_TadA*7.10 + Y147T |
| TadA*8.2 | Monomer_TadA*7.10 + Y147R |
| TadA*8.3 | Monomer_TadA*7.10 + Q154S |
| TadA*8.4 | Monomer_TadA*7.10 + Y123H |
| TadA*8.5 | Monomer_TadA*7.10 + V82S |
| TadA*8.6 | Monomer_TadA*7.10 + T166R |
| TadA*8.7 | Monomer_TadA*7.10 + Q154R |
| TadA*8.8 | Monomer_TadA*7.10 + Y147R_Q154R_Y123H |
| TadA*8.9 | Monomer_TadA*7.10 + Y147R_Q154R_I76Y |
| TadA*8.10 | Monomer_TadA*7.10 + Y147R_Q154R_T166R |
| TadA*8.11 | Monomer_TadA*7.10 + Y147T_Q154R |
| TadA*8.12 | Monomer_TadA*7.10 + Y147T_Q154S |
| TadA*8.13 | Monomer_TadA*7.10 + H123H_ Y147R_Q154R_I76Y |
| TadA*8.14 | Heterodimer_(WT) + (TadA*7.10 + Y147T) |
| TadA*8.15 | Heterodimer_(WT) + (TadA*7.10 + Y147R) |
| TadA*8.16 | Heterodimer_(WT) + (TadA*7.10 + Q154S) |
| TadA*8.17 | Heterodimer_(WT) + (TadA*7.10 + Y123H) |
| TadA*8.18 | Heterodimer_(WT) + (TadA*7.10 + V82S) |
| TadA*8.19 | Heterodimer_(WT) + (TadA*7.10 + T166R) |
| TadA*8.20 | Heterodimer_(WT) + (TadA*7.10 + Q154R) |
| TadA*8.21 | Heterodimer_(WT) + (TadA*7.10 + |
| | Y147R_Q154R_Y123H) |
| TadA*8.22 | Heterodimer_(WT) + (TadA*7.10 + Y147R_Q154R_I76Y) |
| TadA*8.23 | Heterodimer_(WT) + (TadA*7.10 + |
| | Y147R_Q154R_T166R) |
| TadA*8.24 | Heterodimer_(WT) + (TadA*7.10 + Y147T_Q154R) |
| TadA*8.25 | Heterodimer_(WT) + (TadA*7.10 + Y147T_Q154S) |
| TadA*8.26 | Heterodimer_(WT) + (TadA*7.10 + |
| | H123H_Y147T_Q154R_I76Y) |

In some embodiments, the adenosine deaminase is a ninth generation TadA*9 variant containing an alteration at an amino acid position selected from the following: 21, 23, 25, 38, 51, 54, 70, 71, 72, 72, 94, 124, 133, 138, 139, 146, and 158 of a TadA variant as shown in the reference sequence below:

```
(SEQ ID NO: 65)
                10 20 30 40
MSEVEFSHEY WMRHALTLAK RARDEREVPV GAVLVLNNRV
                50 60 70 80
IGEGWNRAIG LHDPTAHAEI MALRQGGLVM QNYRLIDATL
                90 100 110 120
YVTFEPCVMC AGAMIHSRIG RVVFGVRNAK TGAAGSLMDV
              130 140 150 160
LHYPGMNHRV EITEGILADE CAALLCYFFR MPRQVFNAQK
KAQSSTD
```

In one embodiment, the adenosine deaminase variant contains alterations at two or more amino acid positions selected from the following: 21, 23, 25, 38, 51, 54, 70, 71, 72, 94, 124, 133, 138, 139, 146, and 158 of the TadA reference sequence above. In another embodiment, the adenosine deaminase variant contains one or more (e.g., 2, 3, 4) alterations selected from the following: R21N, R23H, E25F, N38G, L51W, P54C, M70V, Q71M, N72K, Y73S, M94V, P124W, T133K, D139L, D139M, C146R, and A158K of SEQ ID NO. 1. In other embodiments, the adenosine deaminase variant further contains one or more of the following alterations: Y147T, Y147R, Q154S, Y123H, and Q154R. In still other embodiments, the adenosine deaminase variant contains a combination of alterations relative to the above TadA reference sequence selected from the following:

$$E 25 F + V 82 S + Y 123 H , T 133 K + Y 147 R + Q 154 R ;$$

$$E 25 F + V 82 S + Y 123 H + Y 147 R + Q 154 R ; \text{} L 51 W + V 82 S + Y 123 H + C 146 R + Y 147 R + Q 154 R ;$$

$$Y 73 S + V 82 S + Y 123 H + Y 147 R + Q 154 R ;$$

$$P 54 C + V 82 S + Y 123 H + Y 147 R + Q 154 R ;$$

$$N 38 G + V 82 T + Y 123 H + Y 147 R + Q 154 R ;$$

$$N 72 K + V 82 S + Y 123 H + D 139 L + Y 147 R + Q 154 R ;$$

$$E 25 F + V 82 S + Y 123 H + D 139 M + Y 147 R + Q 154 R ;$$

$$Q 71 M + V 82 S + Y 123 H + Y 147 R + Q 154 R ;$$

$$E 25 F + V 82 S + Y 123 H + T 133 K + Y 147 R + Q 154 R ;$$

$$E 25 F + V 82 S + Y 123 H + Y 147 R + Q 154 R ;$$

$$V 82 S + Y 123 H + P 124 W + Y 147 R + Q 154 R ;$$

$$L 51 W + V 82 S + Y 123 H + C 146 R + Y 147 R + Q 154 R ;$$

$$P 54 C + V 82 S + Y 123 H + Y 147 R + Q 154 R ;$$

$$Y 73 S + V 82 S + Y 123 H + Y 147 R + Q 154 R ;$$

$$N 38 G + V 82 T + Y 123 H + Y 147 R + Q 154 R ;$$

$$R 23 H + V 82 S + Y 123 H + Y 147 R + Q 154 R ;$$

$$R 21 N + V 82 S + Y 123 H + Y 147 R + Q 154 R ;$$

$$V 82 S + Y 123 H + Y 147 R + Q 154 R + A 158 K ;$$

$$N 72 K + V 82 S + Y 123 H + D 139 L + Y 147 R + Q 154 R ;$$

$$E 25 F + V 82 S + Y 123 H + D 139 M + Y 147 R + Q 154 R ;$$

$$M 70 V + V 82 S + M 94 V + Y 123 H + Y 147 R + Q 154 R ;$$

$$Q 71 M + V 82 S + Y 123 H + Y 147 R + Q 154 R ; \text{} E 25 F + I 76 Y + V 82 S + Y 123 H + Y 147 R + Q 154 R ; \text{} I 76 Y + V 82 T + Y 123 H + Y 147 R + Q 154 R ; \text{} N 38 G + I 76 Y + V 82 S + Y 123 H + Y 147 R + Q 154 R ;$$

$$R 23 H + I 76 Y + V 82 S + Y 123 H + Y 147 R + Q 154 R ;$$

$$P 54 C + I 76 Y + V 82 S + Y 123 H + Y 147 R + Q 154 R ;$$

$$R 21 N + I 76 Y + V 82 S + Y 123 H + Y 147 R + Q 154 R ;$$

$$I 76 Y + V 82 S + Y 123 H + D 138 M + Y 147 R + Q 154 R ;$$

$$Y 72 S + I 76 Y + V 82 S + Y 123 H + Y 147 R + Q 154 R ; \text{} E 25 F + I 76 Y + V 82 S + Y 123 H + Y 147 R + Q 154 ;$$

$$I 76 Y + V 82 T + Y 123 H + Y 147 R + Q 154 R ;$$

$$N 38 G + I 76 Y + V 82 S + Y 123 H + Y 147 R + Q 154 R ;$$

$$R 23 H + I 76 Y + V 82 S + Y 123 H + Y 147 R + Q 154 R ;$$

$$P 54 C + I 76 Y + V 82 S + Y 123 H + Y 147 R + Q 154 R ;$$

$$R 21 N + I 76 Y + V 82 S + Y 123 H + Y 147 R + Q 154 R ;$$

$$I 76 Y + V 82 S + Y 123 H + D 138 M + Y 147 R + Q 154 R ;$$

$$Y 72 S + I 76 Y + V 82 S + Y 123 H + Y 147 R + Q 154 R ; and$$

$$V 82 S + Q 154 ;$$

$$N 72 K + V 82 S + Y 123 H + Y 147 R + Q 154 R ;$$

$$Q 71 M + V 82 S + Y 123 H + Y 147 R + Q 154 R ;$$

$$V 82 S + Y 123 H + T 133 K + Y 147 R + Q 154 R ;$$

$$V 82 S + Y 123 H + T 133 K + Y 147 R + Q 154 R + A 158 K ;$$

$$M 70 V + Q 71 M + N 72 K + V 82 S + Y 123 H + Y 147 R + Q 154 R ;$$

$$N 72 K\_V 82 S + Y 123 H + Y 147 R + Q 154 R ;$$

$$Q 71 M\_V 82 S + Y 123 H + Y 147 R + Q 154 R ; \text{} M 70 V + V 82 S + M 94 V + Y 123 H + Y 147 R + Q 154 R ;$$

$$V 82 S + Y 123 H + T 133 K + Y 147 R + Q 154 R ;$$

$$V 82 S + Y 123 H + T 133 K + Y 147 R + Q 154 R + A 158 K ; and$$

$$M 70 V + Q 71 M + N 72 K + V 82 S + Y 123 H + Y 147 R + Q 154 R .$$

In some embodiments, the deaminase or other polypeptide sequence lacks a methionine, for example when included as a component of a fusion protein. This can alter the numbering of positions. However, the skilled person will understand that such corresponding mutations refer to the same mutation, e.g., Y73S and Y72S and D139M and D138M.

In some embodiments, Cas9 or Cas12 is fused to nuclear localization sequences, including an NLS of the SV40 large T antigen, nucleoplasmin, c-myc, hRNPA1 M9, IBB domain from importin-alpha, NLS of myoma T protein, human p53, c-abl IV, influenza virus NS1, hepatitis virus delta antigen, mouse Mx1, human poly(ADP-ribose) polymerase, steroid hormone receptor (human) glucocorticoid.

In some embodiments, a Cas9 or Cas12 protein is fused to epitope tags including, but not limited to hemagglutinin (HA) tags, histidine (His) tags, FLAG tags, Myc tags, V5 tags, VSV-G tags, SNAP tags, thioredoxin (Trx) tags.

In some embodiments, Cas9 or Cas12 is fused to reporter genes including, but not limited to glutathione-S-transferase (GST), horseradish peroxidase (HRP), chloramphenicol transferase (CAT), HcRed, DsRed, cyan fluorescent protein, yellow fluorescent protein and blue fluorescent protein, green fluorescent protein (GFP), including enhanced versions or superfolded GFP, as well as other modified versions of reporter genes.

In some embodiments, serum half-life of an engineered Cas9 or Cas12 protein is increased by fusion with heterologous proteins such as a human serum albumin protein, transferrin protein, human IgG and/or sialylated peptide, such as the carboxy-terminal peptide (CTP, of chorionic gonadotropin β chain).

In some embodiments, serum half-life of an engineered Cas9 or Cas12 protein is decreased by fusion with destabilizing domains, including but not limited to geminin, ubiquitin, FKBP12-L106P, and/or dihydrofolate reductase.

Suitable fusion partners that provide for increased or decreased stability include, but are not limited to degron sequences. Degrons are readily understood by one of ordinary skill in the art to be amino acid sequences that control the stability of the protein of which they are part. For example, the stability of a protein comprising a degron sequence is controlled at least in part by the degron sequence. In some cases, a suitable degron is constitutive such that the degron exerts its influence on protein stability independent of experimental control (i.e., the degron is not drug inducible, temperature inducible, etc.) In some cases, the degron provides the variant Cas9 polypeptide with controllable stability such that the variant Cas9 polypeptide can be turned “on” (i.e., stable) or “off (i.e., unstable, degraded) depending on the desired conditions. For example, if the degron is a temperature sensitive degron, the variant Cas9 polypeptide may be functional (i.e., “on”, stable) below a threshold temperature (e.g., 42° C., 41° C., 40° C., 39° C., 38° C., 37° C., 36° C., 35° C., 34° C., 33° C., 32° C., 31° C., 30° C., etc.) but non-functional (i.e., “off, degraded) above the threshold temperature. As another example, if the degron is a drug inducible degron, the presence or absence of drug can switch the protein from an “off (i.e., unstable) state to an “on” (i.e., stable) state or vice versa. An exemplary drug inducible degron is derived from the FKBP12 protein. The stability of the degron is controlled by the presence or absence of a small molecule that binds to the degron.

Examples of suitable degrons include, but are not limited to those degrons controlled by Shield-1, DHFR, auxins, and/or temperature. Non-limiting examples of suitable degrons are known in the art (e.g., Dohmen et al., Science, 1994. 263(5151): p. 1273-1276: Heat-inducible degron: a method for constructing temperature-sensitive mutants; Schoeber et al., Am J Physiol Renal Physiol. 2009 January; 296(1):F204-11: Conditional fast expression and function of multimeric TRPV5 channels using Shield-1; Chu et al., Bioorg Med Chem Lett. 2008 Nov. 15; 18(22):5941-4: Recent progress with FKBP-derived destabilizing domains; Kanemaki, Pflugers Arch. 2012 Dec. 28: Frontiers of protein expression control with conditional degrons; Yang et al., Mol Cell. 2012 Nov. 30; 48(4):487-8: Titivated for destruction: the methyl degron; Barbour et al., Biosci Rep. 2013 Jan. 18; 33(1): Characterization of the bipartite degron that regulates ubiquitin-independent degradation of thymidylate synthase; and Greussing et al., J Vis Exp. 2012 Nov. 10; (69): Monitoring of ubiquitin-proteasome activity in living cells using a Degron (dgn)-destabilized green fluorescent protein (GFP)-based reporter protein; all of which are hereby incorporated in their entirety by reference).

Exemplary degron sequences have been well-characterized and tested in both cells and animals. Thus, fusing dead Cas9 or Cas12 to a degron sequence produces a “tunable” and “inducible” dead Cas9 or Cas12 polypeptide.

Any of the fusion partners described herein can be used in any desirable combination. As one non-limiting example to illustrate this point, a Cas9 or Cas12 fusion protein can comprise a YFP sequence for detection, a degron sequence for stability, and transcription activator sequence to increase transcription of the target DNA. Furthermore, the number of fusion partners that can be used in a dCas9 fusion protein is unlimited. In some cases, a Cas9 fusion protein comprises one or more (e.g., two or more, three or more, four or more, or five or more) heterologous sequences.

### Recombinant Gene Technology

In accordance with the present disclosure, there may be employed conventional molecular biology, microbiology, and recombinant DNA techniques within the skill of the art. Such techniques are described in the literature (see, e.g., Sambrook, Fritsch & Maniatis, Molecular Cloning: A Laboratory Manual, Second Edition (1989) Cold Spring Harbor Laboratory Press, Cold Spring Harbor, N.Y.; DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells and Enzymes (IRL Press, (1986)); B. Perbal, A Practical Guide To Molecular Cloning (1984); F. M. Ausubel et al. (eds.), Current Protocols in Molecular Biology, John Wiley & Sons, Inc. (1994).

Recombinant expression of a gene, such as a nucleic acid encoding a polypeptide, such as an engineered Cas9 or Cas12 enzyme described herein, can include construction of an expression vector containing a nucleic acid that encodes the polypeptide. Once a polynucleotide has been obtained, a vector for the production of the polypeptide can be produced by recombinant DNA technology using techniques known in the art. Known methods can be used to construct expression vectors containing polypeptide coding sequences and appropriate transcriptional and translational control signals. These methods include, for example, in vitro recombinant DNA techniques, synthetic techniques, and in vivo genetic recombination.

An expression vector can be transferred to a host cell by conventional techniques, and the transfected cells can then be cultured by conventional techniques to produce polypeptides.

In some embodiments, a nucleotide sequence encoding a DNA-targeting RNA and/or Cas9 or Cas12 protein is operably linked to a control element, e.g., a transcriptional control element, such as a promoter. The transcriptional control element may be functional in either a eukaryotic cell, e.g., a mammalian cell; or a prokaryotic cell (e.g., bacterial or archaeal cell). In some embodiments, the eukaryotic cell is a human cell. In some embodiments, a nucleotide sequence encoding a DNA-targeting RNA and/or a Cas9 or Cas12 protein is operably linked to multiple control elements that allow expression of the encoded nucleotide sequence in both prokaryotic and eukaryotic cells.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a Rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1; 31(17)), and/or a human HI promoter (HI).

Examples of inducible promoters include, but are not limited to T7 RNA polymerase promoter, T3 RNA polymerase promoter, Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline, RNA polymerase, e.g., T7 RNA polymerase, an estrogen receptor and/or an estrogen receptor fusion.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism. Thus, a spatially restricted promoter can be used to regulate the expression of a nucleic acid encoding a subject site-directed polypeptide in a wide variety of different tissues and cell types, depending on the organism. Some spatially restricted promoters are also temporally restricted such that the promoter is in the “ON” state or “OFF” state during specific stages of embryonic development or during specific stages of a biological process (e.g., hair follicle cycle).

### Nucleobase Editors

In some embodiments, any of base editors provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, any of the base editors provided herein are capable of generating at least 0.01% of intended mutations (i.e. at least 0.01% base editing efficiency). In some embodiments, any of the base editors provided herein are capable of generating at least 0.01%, 1%, 2%, 3%, 4%, 5%, 10%, 15%, 20%, 25%, 30%, 40%, 45%, 50%, 60%, 70%, 80%, 90%, 95%, or 99% of intended mutations.

In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

### Therapeutic Applications

The methods and compositions described herein can have various therapeutic applications, for example in the treatment of liver diseases.

In some embodiments, the CRISPR methods or systems described herein can be used to edit a target nucleic acid to modify the target nucleic acid (e.g., by inserting, deleting, or mutating one or more nucleic acid residues). For example, in some embodiments the CRISPR systems described herein comprise an exogenous donor template nucleic acid (e.g., a DNA molecule or a RNA molecule), which comprises a desirable nucleic acid sequence. Upon resolution of a cleavage event induced with the CRISPR system described herein, the molecular machinery of the cell will utilize the exogenous donor template nucleic acid in repairing and/or resolving the cleavage event. Alternatively, the molecular machinery of the cell can utilize an endogenous template in repairing and/or resolving the cleavage event. In some embodiments, the CRISPR systems described herein may be used to alter a target nucleic acid resulting in an insertion, a deletion, and/or a point mutation). In some embodiments, the insertion is a scarless insertion (i.e., the insertion of an intended nucleic acid sequence into a target nucleic acid resulting in no additional unintended nucleic acid sequence upon resolution of the cleavage event). Donor template nucleic acids may be double stranded or single stranded nucleic acid molecules (e.g., DNA or RNA). In some embodiments, the CRISPR methods or systems described herein comprise a nucleobase editor.

In applications in which it is desirable to insert a polynucleotide sequence into a target DNA sequence, a polynucleotide comprising a donor sequence to be inserted is also provided to the cell. By a “donor sequence” or “donor polynucleotide” it is meant a nucleic acid sequence to be inserted at the cleavage site induced by a site-directed modifying polypeptide. The donor polynucleotide will contain sufficient homology to a genomic sequence at the cleavage site, e.g., 70%, 80%, 85%, 90%, 95%, or 100% homology with the nucleotide sequences flanking the cleavage site, e.g., within about 50 bases or less of the cleavage site, e.g., within about 30 bases, within about 15 bases, within about 10 bases, within about 5 bases, or immediately flanking the cleavage site, to support homology-directed repair between it and the genomic sequence to which it bears homology. Approximately 25, 50, 100, or 200 nucleotides, or more than 200 nucleotides, of sequence homology between a donor and a genomic sequence (or any integral value between 10 and 200 nucleotides, or more) will support homology-directed repair. Donor sequences can be of any length, e.g., 10 nucleotides or more, 50 nucleotides or more, 100 nucleotides or more, 250 nucleotides or more, 500 nucleotides or more, 1000 nucleotides or more, 5000 nucleotides or more, etc.

The donor sequence is typically not identical to the genomic sequence that it replaces. Rather, the donor sequence may contain at least one or more single base changes, insertions, deletions, inversions or rearrangements with respect to the genomic sequence, so long as sufficient homology is present to support homology-directed repair. In some embodiments, the donor sequence comprises a non-homologous sequence flanked by two regions of homology, such that homology-directed repair between the target DNA region and the two flanking sequences results in insertion of the non-homologous sequence at the target region. Donor sequences may also comprise a vector backbone containing sequences that are not homologous to the DNA region of interest and that are not intended for insertion into the DNA region of interest. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the donor polynucleotide.

The donor sequence may comprise certain sequence differences as compared to the genomic sequence, e.g., restriction sites, nucleotide polymorphisms, selectable markers (e.g., drug resistance genes, fluorescent proteins, enzymes etc.), etc., which may be used to assess for successful insertion of the donor sequence at the cleavage site or in some cases may be used for other purposes (e.g., to signify expression at the targeted genomic locus). In some cases, if located in a coding region, such nucleotide sequence differences will not change the amino acid sequence, or will make silent amino acid changes (i.e., changes which do not affect the structure or function of the protein). Alternatively, these sequences differences may include flanking recombination sequences such as FLPs, loxP sequences, or the like, that can be activated at a later time for removal of the marker sequence.

The donor sequence may be provided to the cell as single-stranded DNA, single-stranded RNA, double-stranded DNA, or double-stranded RNA. It may be introduced into a cell in linear or circular form. If introduced in linear form, the ends of the donor sequence may be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. Additional methods for protecting exogenous polynucleotides from degradation include, but are not limited to, addition of terminal amino group(s) and the use of modified internucleotide linkages such as, for example, phosphorothioates, phosphor amidates, and O-methyl ribose or deoxyribose residues. As an alternative to protecting the termini of a linear donor sequence, additional lengths of sequence may be included outside of the regions of homology that can be degraded without impacting recombination. A donor sequence can be introduced into a cell as part of a vector molecule having additional sequences such as, for example, replication origins, promoters and genes encoding antibiotic resistance. Moreover, donor sequences can be introduced as naked nucleic acid, as nucleic acid complexed with an agent such as a liposome or poloxamer, or can be delivered by viruses (e.g., adenovirus, AAV), as described above for nucleic acids encoding a DNA-targeting RNA and/or site-directed modifying polypeptide and/or donor polynucleotide.

Following the methods described above, a DNA region of interest may be cleaved and modified, i.e. “genetically modified”, ex vivo. In some embodiments, as when a selectable marker has been inserted into the DNA region of interest, the population of cells may be enriched for those comprising the genetic modification by separating the genetically modified cells from the remaining population. Prior to enriching, the “genetically modified” cells may make up only about 1% or more (e.g., 2% or more, 3% or more, 4% or more, 5% or more, 6% or more, 7% or more, 8% or more, 9% or more, 10% or more, 15% or more, or 20% or more) of the cellular population. Separation of “genetically modified” cells may be achieved by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g., magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the genetically modified cells. Cell compositions that are highly enriched for cells comprising modified DNA are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells (e.g., the genetically modified human hepatocytes) produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g., containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g., penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g., to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects. Mammalian species that may be treated with the present methods include canines and felines; equines; bovines; ovines; etc. and primates, particularly humans. Animal models, particularly small mammals (e.g., mouse, rat, guinea pig, hamster, lagomorpha (e.g., rabbit), etc.) may be used for experimental investigations.

Cells may be provided to the subject alone or with a suitable substrate or matrix, e.g., to support their growth and/or organization in the tissue to which they are being transplanted. In some embodiments, the cells may be introduced to the subject via any of the following routes: parenteral, subcutaneous, intravenous, intracranial, intraspinal, intraocular, or into spinal fluid. The cells may be introduced by injection, catheter, or the like.

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

Pharmaceutical preparations are compositions that include one or more of the base editor or base editor systems described herein in a pharmaceutically acceptable vehicle. “Pharmaceutically acceptable vehicles” may be vehicles approved by a regulatory agency of the Federal or a state government or listed in the U.S.

Pharmacopeia or other generally recognized pharmacopeia for use in mammals, such as humans. The term “vehicle” refers to a diluent, adjuvant, excipient, or carrier with which a compound of the invention is formulated for administration to a mammal. Such pharmaceutical vehicles can be lipids, e.g., liposomes, e.g., liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semisolid, liquid or gaseous forms, such as tablets, capsules, powders, granules, ointments, solutions, suppositories, injections, inhalants, gels, microspheres, and aerosols. As such, administration of a DNA-targeting RNA and/or site-directed modifying polypeptide and/or donor polynucleotide can be achieved in various ways, including oral, buccal, rectal, parenteral, intraperitoneal, intradermal, transdermal, intratracheal, intraocular, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

The effective amount given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, and enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

### Delivery Systems

The base editor or base editor system described herein, or components thereof, nucleic acid molecules thereof, and/or nucleic acid molecules encoding or providing components thereof, CRISPR-associated proteins, or RNA guides, can be delivered by various delivery systems such as vectors, e.g., plasmids and delivery vectors. Exemplary embodiments are described below. The base editor or base editor system (e.g., including the Cas9 or Cas12, and optionally comprising a nucleobase editor described herein) can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, the CRISPR system of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas9 or Cas12) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing base editors described herein can be useful for generating base editors capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a CRISPR system (e.g., including the Cas9 disclosed herein) of the present disclosure is of sufficient size so as to enable efficient packaging and delivery into a target cell via a retroviral vector. In some cases, a Cas9 is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A base editor or base editor system (e.g., including the Cas9 or Cas12 disclosed herein) can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for base editors and base editor systems are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g., lipid and/or polymer) nanoparticles can be suitable for use as delivery vehicles in certain embodiments of this disclosure. Exemplary lipids for use in nanoparticle formulations, and/or gene transfer are shown in Table 9 (below).

TABLE 9
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidyl- | DOPE | Helper |
| ethanolamine Cholesterol | | Helper |
| N-[1-(2,3-Dioleyloxy)prophyl]N,N,N- | DOTMA | Cationic |
| trimethylammonium chloride | | |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3- | GAP- | Cationic |
| bis(dodecyloxy)-1-propanaminium bromide | DLRIE | |
| Cetyltrimethylammonium bromide | CTAB | Cationic |
| 6-Lauroxyhexyl ornithinate | LHON | Cationic |
| 1-(2,3-Dioleoyloxypropyl)-2,4,6- | 2Oc | Cationic |
| trimethylpyridinium | | |
| 2,3-Dioleyloxy-N-[2(sperminecarboxamido- | DOSPA | Cationic |
| ethyl]-N,N-dimethyl-1-propanaminium | | |
| trifluoroacetate | | |
| 1,2-Dioleyl-3-trimethylammonium-propane | DOPA | Cationic |
| N-(2-Hydroxyethyl)-N,N-dimethyl-2,3- | MDRIE | Cationic |
| bis(tetradecyloxy)-1-propanaminium bromide | | |
| Dimyristooxypropyl dimethyl hydroxyethyl | DMRI | Cationic |
| ammonium bromide | | |
| 3B-[N-(N′,N′-Dimethylaminoethane)- | DC-Chol | Cationic |
| carbamoyl]cholesterol | | |
| Bis-guanidium-tren-cholesterol | BGTC | Cationic |
| 1,3-Diodeoxy-2-(6-carboxy-spermyl)- | DOSPER | Cationic |
| propylamide | | |
| Dimethyloctadecylammonium bromide | DDAB | Cationic |
| Dioctadecylamidoglicylspermidin | DSL | Cationic |
| rac-[(2,3-Dioctadecyloxypropyl)(2- | CLIP-1 | Cationic |
| hydroxyethyl)]-dimethylammonium chloride | | |
| rac-[2(2,3-Dihexadecyloxypropyl- | CLIP-6 | Cationic |
| oxymethyloxy)ethyl]trimethylammoniun | | |
| bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho | DSEPC | Cationic |
| sphocholine | | |
| N-Palmitoyl D-erythro-sphingosyl carbamoyl- | CCS | Cationic |
| spermine | | |
| N-t-Butyl-NO-tetradecyl-3- | diC14- | Cationic |
| tetradecylaminopropionamidine | amidine | |
| Octadecenolyoxy[ethyl-2-heptadecenyl-3 | DOTIM | Cationic |
| hydroxyethyl] imidazolinium chloride | | |
| N1-Cholesteryloxycarbonyl-3,7-diazanonane- | CDAN | Cationic |
| 1,9-diamine | | |
| 2-(3-[Bis(3-amino-propyl)-amino]propylamino)- | RPR209120 | Cationic |
| N-ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl- | DLin-KC2- | Cationic |
| [1,3]-dioxolane | DMA | |
| dilinoleyl-methyl-4-dimethylaminobutyrate | DLin-MC3- | Cationic |
| | DMA | |

Table 10 lists exemplary polymers for use in gene transfer and/or nanoparticle formulations.

TABLE 10
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 11 summarizes delivery methods for a polynucleotide encoding a Cas9 described herein.

TABLE 11

| | | Delivery | | | |
|---|---|---|---|---|---|
| | | into | Duration | | |
| | | Non- | of | | Type of |
| | Vector/ | Dividing | Ex- | Genome | Molecule |
| Delivery | Mode | Cells | pression | Integration | Delivered |
| Physical | (e.g., | YES | Transient | NO | Nucleic |
| | electro- | | | | Acids and |
| | poration, | | | | Proteins |
| | particle | | | | |
| | gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO | RNA |
| | | | | with | |
| | | | | modi- | |
| | | | | fication | |
| | Adenovirus | YES | Transient | NO | DNA |
| | Adeno- | YES | Stable | NO | DNA |
| | Associated | | | | |
| | Virus | | | | |
| | (AAV) | | | | |
| | Vaccinia | YES | Very | NO | DNA |
| | Virus | | Transient | | |
| | Herpes | YES | Stable | NO | DNA |
| | Simplex | | | | |
| | Virus | | | | |
| Non- | Cationic | YES | Transient | Depends | Nucleic |
| Viral | Liposomes | | | on what is | Acids and |
| | | | | delivered | Proteins |
| | Polymeric | YES | Transient | Depends | Nucleic |
| | Nano- | | | on what is | Acids and |
| | particles | | | delivered | Proteins |
| Biological | Attenuated | YES | Transient | NO | Nucleic |
| Non-Viral | Bacteria | | | | Acids |
| Delivery | Engineered | YES | Transient | NO | Nucleic |
| Vehicles | Bacterio- | | | | Acids |
| | phages | | | | |
| | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, or Cas12 or variants thereof, optionally fused to a polypeptide having biological activity (e.g., a nucleobase editor), and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants or Cas12 variants) and to direct homology directed repair (HDR).

A promoter used to drive the base editor or base editor system (e.g., including the Cas9 or Cas12 described herein) can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the Cas9 and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For osteoblasts, suitable promoters can include OG-2.

In some cases, a Cas9 of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

A promoter used to drive expression of a guide nucleic acid includes: Pol III promoters such as U6 or H1 or use of a Pol II promoter and intronic cassettes to express gRNA Adeno Associated Virus (AAV).

A Cas9 or Cas12 described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed Cas9 which is shorter in length than conventional Cas9.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 m low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA, can be delivered in the form of RNA. Cas9 or Cas12 encoding mRNA can be generated using in vitro transcription. For example, Cas9 or Cas12 mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the Cas9 sequence and/or the guide nucleic acid can be modified to include one or more modified nucleoside, e.g., using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the Cas9 or Cas12 is codon optimized for expression in the desired cell type, preferentially a eukaryotic cell, preferably a mammalian cell or a human cell. In some embodiments, the cell is a human hepatocyte.

In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g., 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Pharmaceutical Compositions

Other aspects of the present disclosure relate to pharmaceutical compositions comprising a base editor or base editor system (e.g., including Cas9 or Cas12 disclosed herein). The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249: 1527-1533; Sefton, 1989, CRC Crit. Ref Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228: 190; During et al., 1989, Ann. Neurol. 25:351; Howard et ah, 1989, J. Neurosurg. 71: 105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, the base editor or base editor systems (e.g., including the Cas9 or Cas12 described herein) are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Kits

In one aspect, the invention provides kits containing any one or more of the elements disclosed in the above methods and compositions. In some embodiments, the kit comprises a vector system and instructions for using the kit. In some embodiments, the vector system comprises one or more insertion sites for inserting a guide sequence, wherein when expressed, the guide sequence directs sequence-specific binding of a CRISPR complex to a target sequence in a eukaryotic cell, wherein the CRISPR complex comprises a CRISPR enzyme complexed with (1) the guide sequence that is hybridized to the target sequence, and (2) a sequence that is hybridized to the tracr sequence; and/or (b) a second regulatory element operably linked to an enzyme-coding sequence encoding said CRISPR enzyme comprising a nuclear localization sequence. Elements may be provide individually or in combinations, and may be provided in any suitable container, such as a vial, a bottle, or a tube. In some embodiments, the kit includes instructions in one or more languages, for example in more than one language.

In some embodiments, the kit comprises a nucleobase editor.

In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g., in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10. In some embodiments, the kit comprises one or more oligonucleotides corresponding to a guide sequence for insertion into a vector so as to operably link the guide sequence and a regulatory element. In some embodiments, the kit comprises a homologous recombination template polynucleotide.

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

### EXAMPLES

The following examples describe some of the preferred modes of making and practicing the present invention. However, it should be understood that these examples are for illustrative purposes only and are not meant to limit the scope of the invention.

### Example 1. In Vitro Base Editing Using Cas9 in Primary Human Hepatocytes for Liver Transplantation

This example illustrates in vitro Cas9 base editing targeting exemplary MHC Class I or Class II antigen genes in primary human hepatocytes.

In this example, base editing was carried out to target MHC Class I or Class II antigen genes in an effort to reduce immune rejection of an allogenic graft comprising primary hepatocytes.

Briefly, Cas9 guide RNAs targeting specific nucleotide locations within the splice site and/or the stop codon, of exemplary MHC Class I or Class II antigen genes, B2M and CIITA were designed for introduction into hepatocytes (Table 1).

Primary human hepatocytes were transfected with expression vectors containing Cas9 enzyme fused to an adenine base editor (ABE) or to a cytidine base editor (CBE) and guide RNAs (Table 1), 24 hours after plating. Cells were harvested 5 days post-transfection and total DNA was extracted.

Deep sequencing was carried out to characterize A-to-G conversion or C-to-T conversion in primary human hepatocytes. Exemplary targets were amplified using a two-round PCR to add Illumina adapters as well as unique barcodes to the target amplicons. PCR products were run on a 2% gel and gel extracted. Samples were pooled, quantified and cDNA libraries were prepared and sequenced on MiSeq. The percent A-to-G and C-to-T conversion was determined by deep sequencing, and base editing was observed.

TABLE 1
Target gene, site strategy and nucleotide
location

| | | | Protospacer | |
|---|---|---|---|---|
| | Base | Knock | Sequence | PAM |
| | Edi- | out edit | (target | Se- |
| Gene | tor | strategy | bases underlined) | quence |
| B2M | CBE | Splice | ACTCACGCTGGATAGCCTCC | AGG |
| | | site | (SEQ ID NO: 69) | |
| | | disruption | | |
| B2M | ABE | Splice | CTTACCCCACTTAACTATCT | TGG |
| | | site | (SEQ ID NO: 70) | |
| | | disruption | | |
| CIITA | CBE | Splice | CACTCACCTTAGCCTGAGCA | GGG |
| | | site | (SEQ ID NO: 71) | |
| | | disruption | | |
| CIITA | ABE | Splice | CACTCACCTTAGCCTGAGCA | GGG |
| | | site | (SEQ ID NO: 72) | |
| | | disruption | | |

The results in this example showed that guide RNAs and Cas9 achieved base editing of the B2M and CIITA immune genes in primary human hepatocytes.

Base editing was also performed in primary cultures of human hepatocytes. For these studies, base editors were evaluated for their ability to target either the B2M (HLA MHC Class I) and/or CIITA (HLA MHC Class II) in cultured primary human hepatocytes (PHHs). Both C-T (BE4) and A-G (ABE) editors were tested. A Cas9 nuclease (SpCas9) was also used as editing control. Guide RNAs designed to disrupt splice sites in the B2M and CIITA genes were tested in combination with either BE4 or ABE. These guides were also shown to generate indels when used with Cas9 nuclease. Human primary hepatocytes were plated and transfected (lipofection) with a mixture containing the RNA encoding for the base editors (or Cas9) and the guide RNAs. Cells were harvested 5 days post-transfection for genomic DNA extraction and NGS analysis.

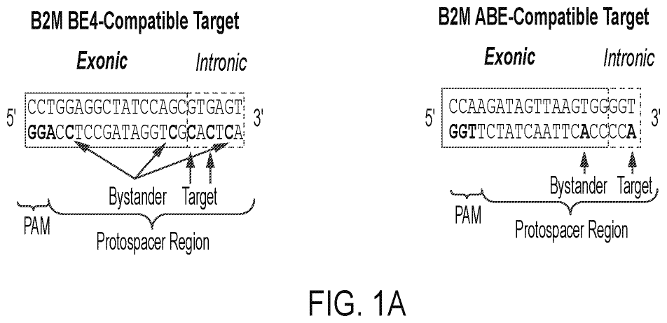

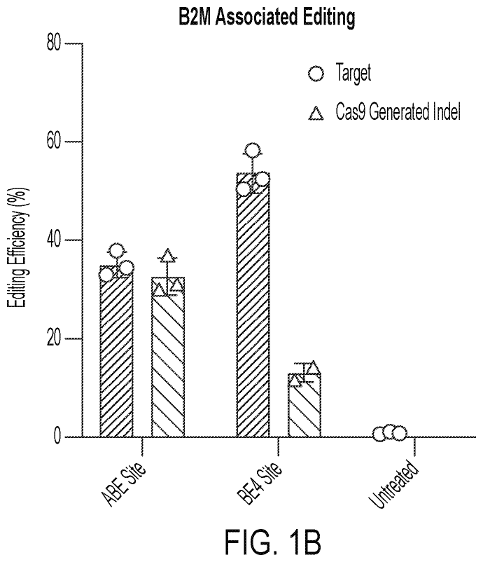

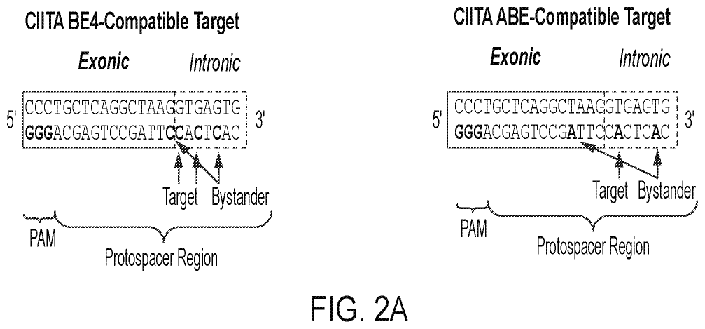

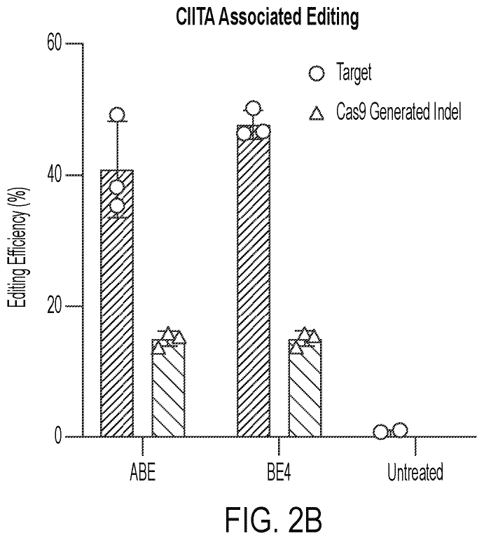

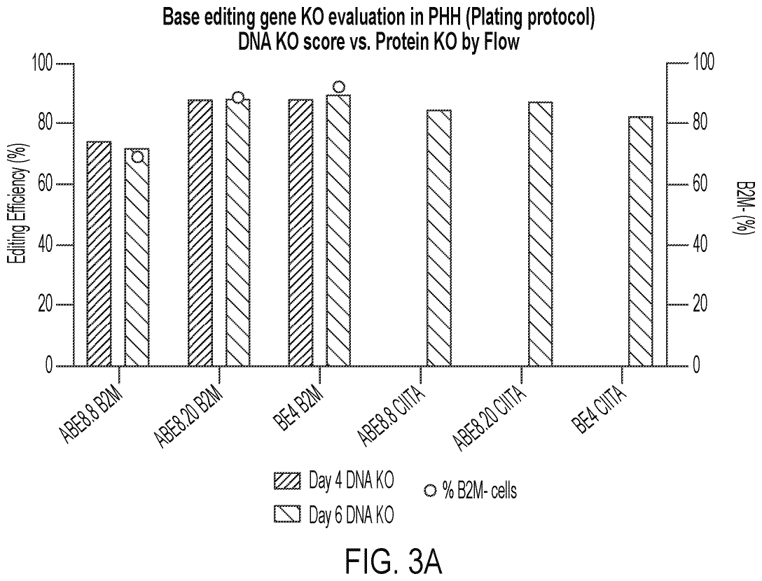

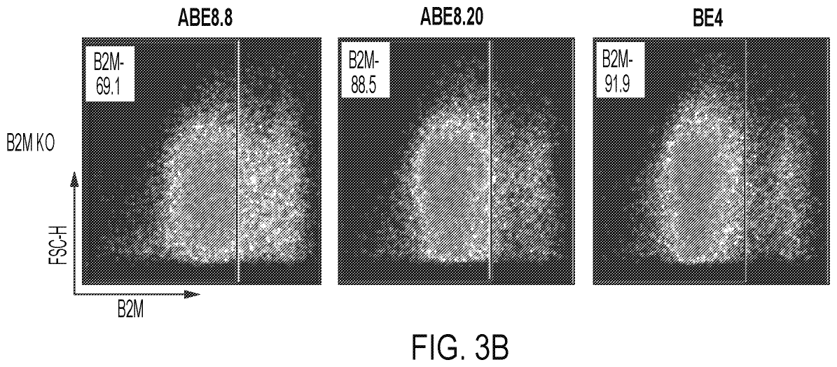

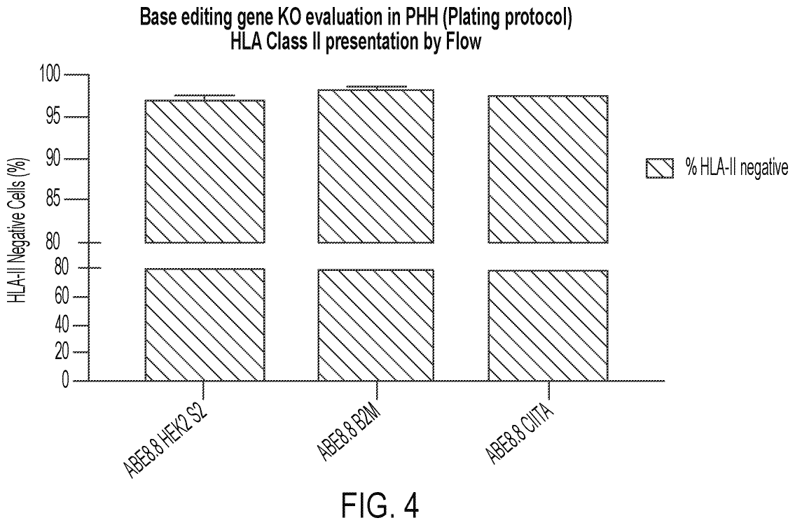

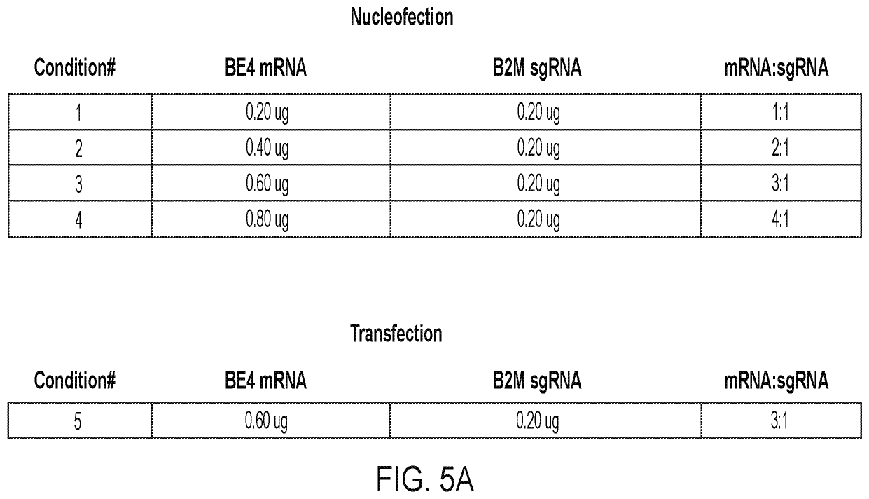

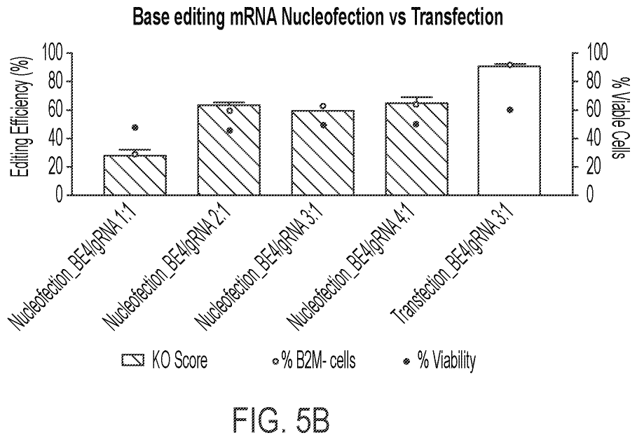

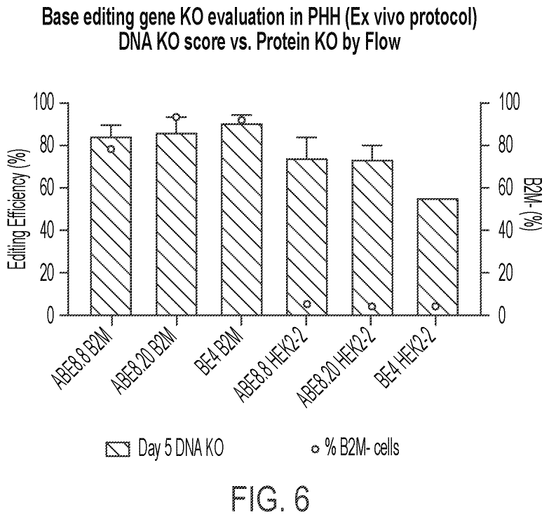

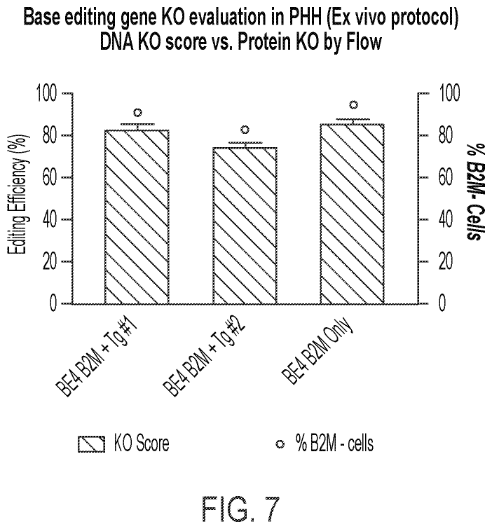

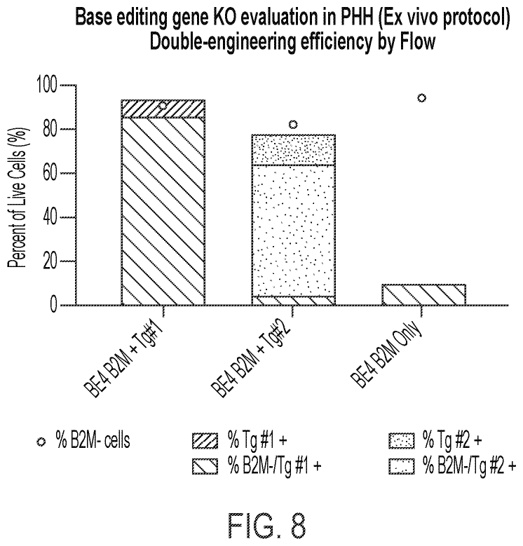

The data from these base editing experiments are shown in FIG. 1A-1B, and in FIG. 2A-2B. FIG. 1A shows the B2M base editor targets that were used to generate a potential splice site, indicated in red. Also shown in FIG. 1A are potential bystander edits outside of the intended splice site region indicated in grey. FIG. 2A shows the CIITA base editor targets that were used to generate a potential splice site, indicated in Red. FIG. 2A also shows potential bystander edits outside of the indented splice site region, indicated in grey.

The data from both of these studies showed significant base editing of B2M (FIG. 2A) and the CIITA genes (FIG. 2B). FIG. 1B shows that B2M editing efficiency was as high as 55% using the BE4 base editor. Namely, BE4 yielded 55% C to T conversion at the BE-4 associated site. FIG. 1B also shows that use of ABE site editor (ABE7.10) had the best editing efficacy at the B2M ABE-associated site, which yielded 35% A to G conversion. Cas9 was used as a direct comparison for gene disruption—base-editing shows comparable or better editing efficiency at the proposed splice site than indel generated by Cas9. FIG. 2B shows the results of base editing using an ABE editor (ABE8.2m) and BE4 to target the CIITA gene. The results showed that the ABE editor yielded significant editing, with 40% A to G conversion, and the BE4 yielded C to T conversion as high as 50%. Like in FIG. 1B, Cas9 was used as a direct comparison for gene disruption—base-editing shows comparable or better editing efficiency at the proposed splice site than indel generated by Cas9.

### Example 2. Multiplexing Guide RNAs for Base Editing of Multiple Immune Genes in Primary Human Hepatocytes

This example illustrates multiplex gene editing to target multiple immune system genes and reduce the immunogenicity of allogenic hepatocytes for liver transplantation.

Liver transplantation is subject to graft rejection due to immune responses. Gene editing by Cas9 using multiplexed guide RNAs targeting multiple immune system genes is used in this example to reduce/abolish immune responses and improve graft survival of the transplanted hepatocytes.

Guide RNAs targeting multiple gene loci in exemplary B2M, CD142 and CIITA genes will be cloned into an expression vector either expressing multiple guides from multiple promoters, or from a polycistronic transcript. These multiplexed guide RNAs will be introduced into hepatocytes along with a Cas9 enzyme.

The efficiency of base editing using multiplexed guide RNAs will be measured by determining the percentage of A-to-G and C-to-T conversion by deep sequencing.

### Example 3. Bioinformatic Screen to Identify Additional Guide RNAs for Immune System Genes

This example demonstrates the identification of additional guide RNAs targeting immune system genes using a bioinformatics screen.

A bioinformatics screen was used to search for additional guide RNAs to expand CRISPR's targeting range for immune system genes. Exemplary immune system genes targeted included the MHC Class I or Class II genes, including J2 microglobulin (B2M) and Class II Major Histocompatibility Complex Transactivator (CIITA). The screen utilized seed sequences of Cas9 from the S. pyogenes. Bioinformatics was carried out using the tblastn variant of BLAST with an e-value threshold of 1e-6 for considering BLAST hits. Additional bioinformatics screens will be performed to determine guide RNAs targeting other exemplary immune system genes including CD142, and Human Leukocyte Antigen A (HLA-A) and Human Leukocyte Antigen B (HLA-B).

Guide RNA sequences and their PAMs are shown in Tables 2, 3, 4, 5 and 6 for exemplary immune system genes, B2M, CD142, CIITA, HLA-A and HLA-B.

TABLE 2
Base editor, PAM sequences, guide RNA for B2M target gene.

| | Search | Strategy | Full sequence | |
|---|---|---|---|---|
| Editor | PAM | site | (Protospacer underlined) | PAM |
| KKH-SaCas9-ABE | NNNRRT | Splice site | TCCTCAGGTACTCCAAAGATTCAGGT | TCAGGT |
| | | | (SEQ ID NO: 73) | |
| NGA-SpCas9-BE4 | NGA | Splice site | CGATCTATGAAAAAGACAGTGGA | GGA |
| | | | (SEQ ID NO: 74) | |
| NGA-SpCas9-BE4 | NGA | Stop codon | AAAGACCAGTCCTTGCTGAAAGA | AGA |
| | | | (SEQ ID NO: 75) | |
| NGA-SpCas9-BE4 | NGA | Splice site | GGAGTACCTGAGGAATATCGGGA | GGA |
| | | | (SEQ ID NO: 76) | |
| NGC-SpCas9-ABE | NGC | Splice site | TTCATAGATCGAGACATGTAAGC | AGC |
| | | | (SEQ ID NO: 77) | |
| NGC-SpCas9-ABE | NGC | Splice site | CTCACGCTGGATAGCCTCCAGGC | GGC |
| | | | (SEQ ID NO: 78) | |
| NGC-SpCas9-AIBE | NGC | Splice site | TTCATAGATCGAGACATGTAAGC | AGC |
| | | | (SEQ ID NO: 79) | |
| NGC-SpCas9-AIBE | NGC | Splice site | AGGAGAGACTCACGCTGGATAGC | AGC |
| | | | (SEQ ID NO: 80) | |
| NGC-SpCas9-AIBE | NGC | Splice site | CTCACGCTGGATAGCCTCCAGGC | GGC |
| | | | (SEQ ID NO: 81) | |
| NGC-SpCas9-BE4 | NGC | Stop codon | ATAGATCGAGACATGTAAGCAGC | AGC |
| | | | (SEQ ID NO: 82) | |
| NGC-SpCas9-BE4 | NGC | Stop codon | TACCCCACTTAACTATCTTGGGC | GGC |
| | | | (SEQ ID NO: 83) | |
| NGC-SpCas9-BE4 | NGC | Splice site | CTCACGCTGGATAGCCTCCAGGC | GGC |
| | | | (SEQ ID NO: 84) | |
| SpCas9-ABE | NGG | Splice site | CTCAGGTACTCCAAAGATTCAGG | AGG |
| | | | (SEQ ID NO: 85) | |
| SpCas9-ABE | NGG | Splice site | CTTACCCCACTTAACTATCTTGG | TGG |
| | | | (SEQ ID NO: 86) | |
| SpCas9-ABE | NGG | Splice site | ACTCACGCTGGATAGCCTCCAGG | AGG |
| | | | (SEQ ID NO: 87) | |
| SpCas9-AIBE | NGG | Splice site | CTCAGGTACTCCAAAGATTCAGG | AGG |
| | | | (SEQ ID NO: 88) | |
| SpCas9-AIBE | NGG | Splice site | CTTACCCCACTTAACTATCTTGG | TGG |
| | | | (SEQ ID NO: 89) | |
| SpCas9-AIBE | NGG | Splice site | ACTCACGCTGGATAGCCTCCAGG | AGG |
| | | | (SEQ ID NO: 90) | |
| SpCas9-BE4 | NGG | Splice site | TCGATCTATGAAAAAGACAGTGG | TGG |
| | | | (SEQ ID NO: 91) | |
| SpCas9-BE4 | NGG | Splice site | CTTACCCCACTTAACTATCTTGG | TGG |
| | | | (SEQ ID NO: 92) | |
| SpCas9-BE4 | NGC | Stop codon | CTTACCCCACTTAACTATCTTGG | TGG |
| | | | (SEQ ID NO: 93) | |
| SpCas9-BE4 | NGC | Splice site | TTACCCCACTTAACTATCTTGGG | GGG |
| | | | (SEQ ID NO: 94) | |
| SpCas9-BE4 | NGC | Stop codon | TTACCCCACTTAACTATCTTGGG | GGG |
| | | | (SEQ ID NO: 95) | |
| SpCas9-BE4 | NGC | Splice site | ACTCACGCTGGATAGCCTCCAGG | AGG |
| | | | (SEQ ID NO: 96) | |

TABLE 2A
Spacer sequences for B2M target gene.

| | | Stra- | Full sequence |
|---|---|---|---|
| | Search | tegy | (Protospacer |
| Editor | PAM | site | underlined) |
| KKH- | NNNRRT | Splice | UCCUCAGGUACUCCAAAGAU |
| SaCas9- | | site | (SEQ ID NO: 97) |
| ABE | | | |
| NGA- | NGA | Splice | CGAUCUAUGAAAAAGACAGU |
| SpCas9- | | site | (SEQ ID NO: 98) |
| BE4 | | | |
| NGA- | NGA | Stop | AAAGACCAGUCCUUGCUGAA |
| SpCas9- | | codon | (SEQ ID NO: 99) |
| BE4 | | | |
| NGA- | NGA | Splice | GGAGUACCUGAGGAAUAUCG |
| SpCas9- | | site | (SEQ ID NO: 100) |
| BE4 | | | |
| NGC- | NGC | Splice | UUCAUAGAUCGAGACAUGUA |
| SpCas9- | | site | (SEQ ID NO: 101) |
| ABE | | | |
| NGC- | NGC | Splice | CUCACGCUGGAUAGCCUCCA |
| SpCas9- | | site | (SEQ ID NO: 102) |
| ABE | | | |
| NGC- | NGC | Splice | UUCAUAGAUCGAGACAUGUA |
| SpCas9- | | site | (SEQ ID NO: 103) |
| AIBE | | | |
| NGC- | NGC | Splice | AGGAGAGACUCACGCUGGAU |
| SpCas9- | | site | (SEQ ID NO: 104) |
| AIBE | | | |
| NGC- | NGC | Splice | CUCACGCUGGAUAGCCUCCA |
| SpCas9- | | site | (SEQ ID NO: 105) |
| AIBE | | | |
| NGC- | NGC | Stop | AUAGAUCGAGACAUGUAAGC |
| SpCas9- | | codon | (SEQ ID NO: 106) |
| BE4 | | | |
| NGC- | NGC | Stop | UACCCCACUUAACUAUCUUG |
| SpCas9- | | codon | (SEQ ID NO: 107) |
| BE4 | | | |
| NGC- | NGC | Splice | CUCACGCUGGAUAGCCUCCA |
| SpCas9- | | site | (SEQ ID NO: 108) |
| BE4 | | | |
| SpCas9- | NGG | Splice | CUCAGGUACUCCAAAGAUUC |
| ABE | | site | (SEQ ID NO: 109) |
| SpCas9- | NGG | Splice | CUUACCCCACUUAACUAUCU |
| ABE | | site | (SEQ ID NO: 110) |
| SpCas9- | NGG | Splice | ACUCACGCUGGAUAGCCUCC |
| ABE | | site | (SEQ ID NO: 111) |
| SpCas9- | NGG | Splice | CUCAGGUACUCCAAAGAUUC |
| AIBE | | site | (SEQ ID NO: 112) |
| SpCas9- | NGG | Splice | CUUACCCCACUUAACUAUCU |
| AIBE | | site | (SEQ ID NO: 113) |
| SpCas9-A | NGG | Splice | ACUCACGCUGGAUAGCCUCC |
| IBE | | site | (SEQ ID NO: 114) |
| SpCas9- | NGG | Splice | UCGAUCUAUGAAAAAGACAG |
| BE4 | | site | (SEQ ID NO: 115) |
| SpCas9- | NGG | Splice | CUUACCCCACUUAACUAUCU |
| BE4 | | site | (SEQ ID NO: 116) |
| SpCas9- | NGC | Stop | CUUACCCCACUUAACUAUCU |
| BE4 | | codon | (SEQ ID NO: 117) |
| SpCas9- | NGC | Splice | UUACCCCACUUAACUAUCUU |
| BE4 | | site | (SEQ ID NO: 118) |
| SpCas9- | NGC | Stop | UUACCCCACUUAACUAUCUU |
| BE4 | | codon | (SEQ ID NO: 119) |
| SpCas9- | NGC | Splice | ACUCACGCUGGAUAGCCUCC |
| BE4 | | site | (SEQ ID NO: 120) |

TABLE 3
Base editor, PAM sequences, guide RNA for CD142 target gene.

| | Search | Strategy | Full sequence | |
|---|---|---|---|---|
| Editor | PAM | site | (Protospacer underlined) | PAM |
| Cas12b-ABE | RTTN | Splice site | GTTGTTTAAAGGCACTACAAATAC | GTTG |
| | | | (SEQ ID NO: 121) | |
| KKH-SaCas9-ABE | NNNRRT | Splice site | TGCTCACCTTTCCTGAACTTGAAGAT | GAAGAT |
| | | | (SEQ ID NO: 122) | |
| KKH-SaCas9-ABE | NNNRRT | Splice site | CTTTCTTTAGCACTAAGTCAGGAGAT | GGAGAT |
| | | | (SEQ ID NO: 123) | |
| NGA-SpCas9-ABE | NGA | Splice site | ATGCTCACCTTTCCTGAACTTGA | TGA |
| | | | (SEQ ID NO: 124) | |
| NGA-SpCas9-ABE | NGA | Splice site | CTCACCTTTCCTGAACTTGAAGA | AGA |
| | | | (SEQ ID NO: 125) | |
| NGA-SpCas9-ABE | NGA | Splice site | CTTACTCTCCAGGTAAGGTGTGA | TGA |
| | | | (SEQ ID NO: 126) | |
| NGA-SpCas9-ABE | NGA | Splice site | TTCTTTAGCACTAAGTCAGGAGA | AGA |
| | | | (SEQ ID NO: 127) | |
| NGA-SpCas9-BE4 | NGA | Splice site | ATGCTCACCTTTCCTGAACTTGA | TGA |
| | | | (SEQ ID NO: 128) | |
| NGA-SpCas9-BE4 | NGA | Splice site | CTCACCTTTCCTGAACTTGAAGA | AGA |
| | | | (SEQ ID NO: 129) | |
| NGA-SpCas9-BE4 | NGA | Splice site | GTTTGCTGAAACAAAGGAAATGA | TGA |
| | | | (SEQ ID NO: 130) | |
| NGA-SpCas9-BE4 | NGA | Splice site | CTTACTCTCCAGGTAAGGTGTGA | TGA |
| | | | (SEQ ID NO: 131) | |
| NGA-SpCas9-BE4 | NGA | Splice site | CTTAGTGCTAAAGAAAGAAAAGA | AGA |
| | | | (SEQ ID NO: 132) | |
| NGA-SpCas9-BE4 | NGA | Splice site | GTGCTAAAGAAAGAAAAGAAGGA | GGA |
| | | | (SEQ ID NO: 133) | |
| NGA-SpCas9-BE4 | NGA | Splice site | TTACCTTATTTGAACAGTGTAGA | AGA |
| | | | (SEQ ID NO: 134) | |
| NGA-SpCas9-BE4 | NGA | Stop codon | TTCCCACTCCAAAATTGTCTTGA | TGA |
| | | | (SEQ ID NO: 135) | |
| NGA-SpCas9-BE4 | NGA | Splice site | GTAGTGCCTTTAAACAACAGAGA | AGA |
| | | | (SEQ ID NO: 136) | |
| NGA-SpCas9-BE4 | NGA | Stop codon | CCTCGGACAGCCAACAATTCAGA | AGA |
| | | | (SEQ ID NO: 137) | |
| NGA-SpCas9-BE4 | NGA | Stop codon | TGAACAGGTGGGAACAAAAGTGA | TGA |
| | | | (SEQ ID NO: 138) | |
| NGA-SpCas9-BE4 | NGA | Stop codon | TCCCTCCCGAACAGTTAACCGGA | GGA |
| | | | (SEQ ID NO: 139) | |
| NGA-SpCas9-BE4 | NGA | Stop codon | CTCCCGAACAGTTAACCGGAAGA | AGA |
| | | | (SEQ ID NO: 140) | |
| NGA-SpCas9-BE4 | NGA | Stop codon | AGTGGGGCAGAGCTGGAAGGAGA | AGA |
| | | | (SEQ ID NO: 141) | |
| NGC-SpCas9-ABE | NGC | Splice site | TGTTTCAGCAAACCTCGGACAGC | AGC |
| | | | (SEQ ID NO: 142) | |
| NGC-SpCas9-AIBE | NGC | Splice site | TGTTTCAGCAAACCTCGGACAGC | CGC |
| | | | (SEQ ID NO: 143) | |
| NGC-SpCas9-AIBE | NGC | Splice site | TGCCACTCACCTGAAGCGCCGGC | GGC |
| | | | (SEQ ID NO: 144) | |
| NGC-SpCas9-AIBE | NGC | Splice site | TGTTTCAGCAAACCTCGGACAGC | AGC |
| | | | (SEQ ID NO: 145) | |
| NGC-SpCas9-BE4 | NGC | Stop codon | TTCCAGCTCTGCCCCACTCCTGC | TGC |
| | | | (SEQ ID NO: 146) | |
| NGC-SpCas9-BE4 | NGC | Splice site | AATATTTCTGAAAAATAAAGGGC | GGC |
| | | | (SEQ ID NO: 147) | |
| NGC-SpCas9-BE4 | NGC | Splice site | TTGCTGAAACAAAGGAAATGAGC | AGC |
| | | | (SEQ ID NO: 148) | |
| NGC-SpCas9-BE4 | NGC | Stop codon | TTTCCAATCTCCTGACTTAGTGC | TGC |
| | | | (SEQ ID NO: 149) | |
| NGC-SpCas9-BE4 | NGC | Stop codon | GATTTCCAAGTTAAATTATATGC | TGC |
| | | | (SEQ ID NO: 150) | |
| NGC-SpCas9-BE4 | NGC | Stop codon | TTCCAAGTTAAATTATATGCTGC | TGC |
| | | | (SEQ ID NO: 151) | |
| NGC-SpCas9-BE4 | NGC | Stop codon | CGAAGACCCAGCCGAGCAGGAGC | AGC |
| | | | (SEQ ID NO: 152) | |
| SpCas9-ABE | NGG | Splice site | TAAAGGCACTACAAATACTGTGG | TGG |
| | | | (SEQ ID NO: 153) | |
| SpCas9-AIBE | NGG | Splice site | AGCCACTTACTCTCCAGGTAAGG | AGG |
| | | | (SEQ ID NO: 154) | |
| SpCas9-AIBE | NGG | Splice site | GTGCCACTCACCTGAAGCGCCGG | CGG |
| | | | (SEQ ID NO: 155) | |
| SpCas9-AIBE | NGG | Splice site | TAAAGGCACTACAAATACTGTGG | TGG |
| | | | (SEQ ID NO: 156) | |
| SpCas9-AIBE | NGG | Splice site | TCTTTCTTTAGCACTAAGTCAGG | AGG |
| | | | (SEQ ID NO: 157) | |
| SpCas9-AIBE | NGG | Splice site | TCCTTTGTTTCAGCAAACCTCGG | CGG |
| | | | (SEQ ID NO: 158) | |
| SpCas9-BE4 | NGG | Splice site | AGTGCTAAAGAAAGAAAAGAAGG | AGG |
| | | | (SEQ ID NO: 159) | |
| SpCas9-BE4 | NGG | Stop codon | GCCCAGGTGGCCGGCGCTTCAGG | AGG |
| | | | (SEQ ID NO: 160) | |
| SpCas9-BE4 | NGG | Stop codon | ACTGTTCAAATAAGGTAAGCTGG | TGG |
| | | | (SEQ ID NO: 161) | |
| SpCas9-BE4 | NGG | Stop codon | CTGTTCAAATAAGGTAAGCTGGG | GGG |
| | | | (SEQ ID NO: 162) | |
| SpCas9-BE4 | NGG | Stop codon | TGAAGCAGACGTACTTGGCACGG | CGG |
| | | | (SEQ ID NO: 163) | |
| SpCas9-BE4 | NGG | Stop codon | GAAGCAGACGTACTTGGCACGGG | GGG |
| | | | (SEQ ID NO: 164) | |
| SpCas9-BE4 | NGG | Stop codon | AACAATTCAGAGTTTTGAACAGG | AGG |
| | | | (SEQ ID NO: 165) | |
| SpCas9-BE4 | NGG | Stop codon | AATTCAGAGTTTTGAACAGGTGG | TGG |
| | | | (SEQ ID NO: 166) | |
| SpCas9-BE4 | NGG | Stop codon | ATTCAGAGTTTTGAACAGGTGGG | GGG |
| | | | (SEQ ID NO: 167) | |

TABLE 3A
Spacer sequences for CD142 target gene.

| | | Stra- | Full sequence |
|---|---|---|---|
| | Search | tegy | (Protospacer |
| Editor | PAM | site | underlined) |
| Cas12b-ABE | RTTN | Splice | UUUAAAGGCACUACAAAUAC |
| | | site | (SEQ ID NO: 168) |
| KKH-SaCas9- | NNNRRT | Splice | UGCUCACCUUUCCUGAACUU |
| ABE | | site | (SEQ ID NO: 169) |
| KKH-SaCas9- | NNNRRT | Splice | CUUUCUUUAGCACUAAGUCA |
| ABE | | site | (SEQ ID NO: 170) |
| NGA-SpCas9- | NGA | Splice | AUGCUCACCUUUCCUGAACU |
| ABE | | site | (SEQ ID NO: 171) |
| NGA-SpCas9- | NGA | Splice | CUCACCUUUCCUGAACUUGA |
| ABE | | site | (SEQ ID NO: 172) |
| NGA-SpCas9- | NGA | Splice | CUUACUCUCCAGGUAAGGUG |
| ABE | | site | (SEQ ID NO: 173) |
| NGA-SpCas9- | NGA | Splice | UUCUUUAGCACUAAGUCAGG |
| ABE | | site | (SEQ ID NO: 174) |
| NGA-SpCas9- | NGA | Splice | AUGCUCACCUUUCCUGAACU |
| BE4 | | site | (SEQ ID NO: 175) |
| NGA-SpCas9- | NGA | Splice | CUCACCUUUCCUGAACUUGA |
| BE4 | | site | (SEQ ID NO: 176) |
| NGA-SpCas9- | NGA | Splice | GUUUGCUGAAACAAAGGAAA |
| BE4 | | site | (SEQ ID NO: 177) |
| NGA-SpCas9- | NGA | Splice | CUUACUCUCCAGGUAAGGUG |
| BE4 | | site | (SEQ ID NO: 178) |
| NGA-SpCas9- | NGA | Splice | CUUAGUGCUAAAGAAAGAAA |
| BE4 | | site | (SEQ ID NO: 179) |
| NGA-SpCas9- | NGA | Splice | GUGCUAAAGAAAGAAAAGAA |
| BE4 | | site | (SEQ ID NO: 180) |
| NGA-SpCas9- | NGA | Splice | UUACCUUAUUUGAACAGUGU |
| BE4 | | site | (SEQ ID NO: 181) |
| NGA-SpCas9- | NGA | Stop | UUCCCACUCCAAAAUUGUCU |
| BE4 | | codon | (SEQ ID NO: 182) |
| NGA-SpCas9- | NGA | Splice | GUAGUGCCUUUAAACAACAG |
| BE4 | | site | (SEQ ID NO: 183) |
| NGA-SpCas9- | NGA | Stop | CCUCGGACAGCCAACAAUUC |
| BE4 | | codon | (SEQ ID NO: 184) |
| NGA-SpCas9- | NGA | Stop | UGAACAGGUGGGAACAAAAG |
| BE4 | | codon | (SEQ ID NO: 185) |
| NGA-SpCas9- | NGA | Stop | UCCCUCCCGAACAGUUAACC |
| BE4 | | codon | (SEQ ID NO: 186) |
| NGA-SpCas9- | NGA | Stop | CUCCCGAACAGUUAACCGGA |
| BE4 | | codon | (SEQ ID NO: 187) |
| NGA-SpCas9- | NGA | Stop | AGUGGGGCAGAGCUGGAAGG |
| BE4 | | codon | (SEQ ID NO: 188) |
| NGC-SpCas9- | NGC | Splice | UGUUUCAGCAAACCUCGGAC |
| ABE | | site | (SEQ ID NO: 189) |
| NGC-SpCas9- | NGC | Splice | UGUUUCAGCAAACCUCGGAC |
| AIBE | | site | (SEQ ID NO: 189) |
| NGC-SpCas9- | NGC | Splice | UGCCACUCACCUGAAGCGCC |
| AIBE | | site | (SEQ ID NO: 190) |
| NGC-SpCas9- | NGC | Splice | UGUUUCAGCAAACCUCGGAC |
| AIBE | | site | (SEQ ID NO: 191) |
| NGC-SpCas9- | NGC | Stop | UUCCAGCUCUGCCCCACUCC |
| BE4 | | codon | (SEQ ID NO: 192) |
| NGC-SpCas9- | NGC | Splice | AAUAUUUCUGAAAAAUAAAG |
| BE4 | | site | (SEQ ID NO: 193) |
| NGC-SpCas9- | NGC | Splice | UUGCUGAAACAAAGGAAAUG |
| BE4 | | site | (SEQ ID NO: 194) |
| NGC-SpCas9- | NGC | Stop | UUUUUAAUCUCCUGACUUAG |
| BE4 | | codon | (SEQ ID NO: 195) |
| NGC-SpCas9- | NGC | Stop | GAUUUCCAAGUUAAAUUAUA |
| BE4 | | codon | (SEQ ID NO: 197) |
| NGC-SpCas9- | NGC | Stop | UUCCAAGUUAAAUUAUAUGC |
| BE4 | | codon | (SEQ ID NO: 198) |
| NGC-SpCas9- | NGC | Stop | CGAAGACCCAGCCGAGCAGG |
| BE4 | | codon | (SEQ ID NO: 199) |
| SpCas9-ABE | NGG | Splice | UAAAGGCACUACAAAUACUG |
| | | site | (SEQ ID NO: 200) |
| SpCas9-AIBE | NGG | Splice | AGCCACUUACUCUCCAGGUA |
| | | site | (SEQ ID NO: 201) |
| SpCas9-AIBE | NGG | Splice | GUGCCACUCACCUGAAGCGC |
| | | site | (SEQ ID NO: 202) |
| SpCas9-AIBE | NGG | Splice | UAAAGGCACUACAAAUACUG |
| | | site | (SEQ ID NO: 203) |
| SpCas9-AIBE | NGG | Splice | UCUUUCUUUAGCACUAAGUC |
| | | site | (SEQ ID NO: 204) |
| SpCas9-AIBE | NGG | Splice | UCCUUUGUUUCAGCAAACCU |
| | | site | (SEQ ID NO: 205) |
| SpCas9-BE4 | NGG | Splice | AGUGCUAAAGAAAGAAAAGA |
| | | site | (SEQ ID NO: 206) |
| SpCas9-BE4 | NGG | Stop | GCCCAGGUGGCCGGCGCUUC |
| | | codon | (SEQ ID NO: 207) |
| SpCas9-BE4 | NGG | Stop | ACUGUUCAAAUAAGGUAAGC |
| | | codon | (SEQ ID NO: 208) |
| SpCas9-BE4 | NGG | Stop | CUGUUCAAAUAAGGUAAGCU |
| | | codon | (SEQ ID NO: 209) |
| SpCas9-BE4 | NGG | Stop | UGAAGCAGACGUACUUGGCA |
| | | codon | (SEQ ID NO: 210) |
| SpCas9-BE4 | NGG | Stop | GAAGCAGACGUACUUGGCAC |
| | | codon | (SEQ ID NO: 211) |
| SpCas9-BE4 | NGG | Stop | AACAAUUCAGAGUUUUGAAC |
| | | codon | (SEQ ID NO: 212) |
| SpCas9-BE4 | NGG | Stop | AAUUCAGAGUUUUGAACAGG |
| | | codon | (SEQ ID NO: 213) |
| SpCas9-BE4 | NGG | Stop | AUUCAGAGUUUUGAACAGGU |
| | | codon | (SEQ ID NO: 214) |

TABLE 4
Base editor, PAM sequences, guide RNA for CIITA target gene.

| | Search | Strategy | Full sequence | PAM |
|---|---|---|---|---|
| Editor | PAM | site | (Protospacer underlined) | |
| Cas12b-ABE | RTTN | Splice site | GTTTTCTCTGCAGCCTTCCCAGAG | GTTT |
| | | | (SEQ ID NO: 215) | |
| Cas12b-ABE | NGG | Stop codon | GTTCTCTCCAGGACGAGAAGTTCC | GTTC |
| | | | (SEQ ID NO: 216) | |
| Cas12b-ABE | NGG | Stop codon | ATTCCACCTGCAGCCTGGATGCGC | ATTC |
| | | | (SEQ ID NO: 217) | |
| Cas12b-ABE | NGG | Stop codon | GTTTCCGACAGCTTGTACAATAAC | GTTT |
| | | | (SEQ ID NO: 218) | |
| Cas12b-ABE | NGG | Stop codon | GTTTCTCTTGCCAGCGTCCAGTAC | GTTT |
| | | | (SEQ ID NO: 219) | |
| KKH-SaCas9-ABE | NNNRRT | Splice site | TGTCTGGGCAGCGGAACTGGACCAGT | ACCAGT |
| | | | (SEQ(SEQ ID NO: 220) | |
| KKH-SaCas9-ABE | NNNRRT | Splice site | TCAAAGTAGAGCACATAGGACCAGAT | CCAGAT |
| | | | (SEQ(SEQ ID NO: 221) | |
| KKH-SaCas9-ABE | NNNRRT | Splice site | CTCACAGCTGAGCCCCCCACTGTGGT | TGTGGT |
| | | | (SEQ(SEQ ID NO: 222) | |
| KKH-SaCas9-ABE | NNNRRT | Splice site | GGCCTTTGCAGAGCCGGTGGAGCAGT | AGCAGT |
| | | | (SEQ(SEQ ID NO: 223) | |
| KKH-SaCas9-ABE | NNNRRT | Splice site | CCACCTGCAGCCTGGATGCGCTGAGT | CTGAGT |
| | | | (SEQ(SEQ ID NO: 224) | |
| KKH-SaCas9-ABE | NNNRRT | Splice site | TCTTGCCAGCGTCCAGTACAACAAGT | ACAAGT |
| | | | (SEQ(SEQ ID NO: 225) | |
| KKH-SaCas9-ABE | NNNRRT | Splice site | CCACTCACCTTAGCCTGAGCAGGGAT | AGGGAT |
| | | | (SEQ(SEQ ID NO: 226) | |
| KKH-SaCas9-ABE | NNNRRT | Splice site | CCTAACATACTGGGAATCTGGTCGGT | GTCGGT |
| | | | (SEQ(SEQ ID NO: 227) | |
| KKH-SaCas9-ABE | NNNRRT | Splice site | GAGGGCCCACCTGAGTAGAGCTCAAT | CTCAAT |
| | | | (SEQ(SEQ ID NO: 228) | |
| KKH-SaCas9-ABE | NNNRRT | Splice site | CTTTTTACCTTGGGGCTCTGACAGGT | ACAGGT |
| | | | (SEQ(SEQ ID NO: 229) | |
| NGA-SpCas9-ABE | NGA | Splice site | AAAGTAGAGCACATAGGACCAGA | AGA |
| | | | (SEQ ID NO: 230) | |
| NGA-SpCas9-ABE | NGA | Splice site | CTCCACAGGGCTGCCTTGAGCGA | CGA |
| | | | (SEQ ID NO: 231) | |
| NGA-SpCas9-ABE | NGA | Splice site | TCCAGGACGAGAAGTTCCTCGGA | GGA |
| | | | (SEQ ID NO: 232) | |
| NGA-SpCas9-ABE | NGA | Splice site | CACCTGCAGCCTGGATGCGCTGA | TGA |
| | | | (SEQ ID NO: 233) | |
| NGA-SpCas9-ABE | NGA | Splice site | TGCAGCCTGGATGCGCTGAGTGA | TGA |
| | | | (SEQ ID NO: 234) | |
| NGA-SpCas9-ABE | NGA | Splice site | CTTACGCCAGCGTCTCCACATGA | TGA |
| | | | (SEQ ID NO: 235) | |
| NGA-SpCas9-ABE | NGA | Splice site | ACTCACTCCATCACCCGGAGGGA | GGA |
| | | | (SEQ ID NO: 236) | |
| NGA-SpCas9-ABE | NGA | Splice site | ACTCACCTTAGCCTGAGCAGGGA | GGA |
| | | | (SEQ ID NO: 237) | |
| NGA-SpCas9-ABE | NGA | Splice site | ACTCACTTGAGGGTTTCCAAGGA | GGA |
| | | | (SEQ ID NO: 238) | |
| NGA-SpCas9-ABE | NGA | Splice site | ACTCACTCCAGATGCTGCAGGGA | GGA |
| | | | (SEQ ID NO: 239) | |
| NGA-SpCas9-ABE | NGA | Splice site | ATCACTCACCAGGCCATTTTGGA | GGA |
| | | | (SEQ ID NO: 240) | |
| NGA-SpCas9-BE4 | NGA | Stop codon | GCCCCAAGGTAAAAAGGCCGGGA | GGA |
| | | | (SEQ ID NO: 241) | |
| NGA-SpCas9-BE4 | NGA | Stop codon | AGCTCACAGTGTGCCACCATGGA | GGA |
| | | | (SEQ ID NO: 242) | |
| NGA-SpCas9-BE4 | NGA | Stop codon | ATGACCAGATGGACCTGGCTGGA | GGA |
| | | | (SEQ ID NO: 243) | |
| NGA-SpCas9-BE4 | NGA | Stop codon | GACCAGATGGACCTGGCTGGAGA | AGA |
| | | | (SEQ ID NO: 244) | |
| NGA-SpCas9-BE4 | NGA | Stop codon | CTGGACCAGTATGTCTTCCAGGA | GGA |
| | | | (SEQ ID NO: 245) | |
| NGA-SpCas9-BE4 | NGA | Stop codon | GTCTTCCAGGACTCCCAGCTGGA | GGA |
| | | | (SEQ ID NO: 246) | |
| NGA-SpCas9-BE4 | NGA | Stop codon | GGACTCCCAGCTGGAGGGCCTGA | TGA |
| | | | (SEQ ID NO: 247) | |
| NGA-SpCas9-BE4 | NGA | Stop codon | TTGGGCAGAAAAGTCAGAAAAGA | AGA |
| | | | (SEQ ID NO: 248) | |
| NGA-SpCas9-BE4 | NGA | Stop codon | AAAGTCAGAAAAGACGTGAGTGA | TGA |
| | | | (SEQ ID NO: 249) | |
| NGA-SpCas9-BE4 | NGA | Stop codon | CTCCGGCCAGATGCGCCTGGAGA | AGA |
| | | | (SEQ ID NO: 250) | |
| NGA-SpCas9-BE4 | NGA | Stop codon | TCTGGCAAATCTCTGAGGCTGGA | GGA |
| | | | (SEQ ID NO: 251) | |
| NGA-SpCas9-BE4 | NGA | Stop codon | CCACCCAATGCCCGGCAGCTGGA | GGA |
| | | | (SEQ ID NO: 252) | |
| NGA-SpCas9-BE4 | NGA | Stop codon | ACCCAATGCCCGGCAGCTGGAGA | AGA |
| | | | (SEQ ID NO: 253) | |
| NGA-SpCas9-BE4 | NGA | Stop codon | CTGCAGGACACGTATGGTGCCGA | CGA |
| | | | (SEQ ID NO: 254) | |
| NGA-SpCas9-BE4 | NGA | Stop codon | TCTGGTGCAGGCCAGGCTGGAGA | AGA |
| | | | (SEQ ID NO: 255) | |
| NGA-SpCas9-BE4 | NGA | Stop codon | GGTGCAGGCCAGGCTGGAGAGGA | GGA |
| | | | (SEQ ID NO: 256) | |
| NGA-SpCas9-BE4 | NGA | Stop codon | CTGGCCCAAGGAGGCCTGGCTGA | TGA |
| | | | (SEQ ID NO: 257) | |
| NGA-SpCas9-BE4 | NGA | Stop codon | CCACAGCCACTCGTGGCGGCCGA | CGA |
| | | | (SEQ ID NO: 258) | |
| NGA-SpCas9-BE4 | NGA | Stop codon | TTTTCCAGAAGAAGCTGCTCCGA | CGA |
| | | | (SEQ ID NO: 259) | |
| NGA-SpCas9-BE4 | NGA | Stop codon | GTCCAGAGCCTGAGCAAGGCCGA | CGA |
| | | | (SEQ ID NO: 260) | |
| NGA-SpCas9-BE4 | NGA | Stop codon | GGAGCAGGCCCAGGCATACGTGA | TGA |
| | | | (SEQ ID NO: 261) | |
| NGA-SpCas9-BE4 | NGA | Stop codon | AGAGCACCAAGACAGAGCCCTGA | TGA |
| | | | (SEQ ID NO: 262) | |
| NGA-SpCas9-BE4 | NGA | Stop codon | AGACATCAAAGTACCCTACAGGA | GGA |
| | | | (SEQ ID NO: 263) | |
| NGA-SpCas9-BE4 | NGA | Stop codon | CATCAAAGTACCCTACAGGAGGA | GGA |
| | | | (SEQ ID NO: 264) | |
| NGA-SpCas9-BE4 | NGA | Stop codon | GAGGACCAGTTCCCATCCGCAGA | AGA |
| | | | (SEQ ID NO: 265) | |
| NGA-SpCas9-BE4 | NGA | Stop codon | GCTCCCGCAGTACCTAGCATTGA | TGA |
| | | | (SEQ ID NO: 266) | |
| NGA-SpCas9-BE4 | NGA | Stop codon | CAGGAAGCAGAAGGTGCTTGCGA | CGA |
| | | | (SEQ ID NO: 267) | |
| NGA-SpCas9-BE4 | NGA | Stop codon | ATTTGGCAGCACGTGGTACAGGA | GGA |
| | | | (SEQ ID NO: 268) | |
| NGA-SpCas9-BE4 | NGA | Stop codon | GCGGGCCAAGACTTCTCCCTGGA | GGA |
| | | | (SEQ ID NO: 269) | |
| NGA-SpCas9-BE4 | NGA | Stop codon | GTCCCTGCAGCAGCATGGGGAGA | AGA |
| | | | (SEQ ID NO: 270) | |
| NGA-SpCas9-BE4 | NGA | Stop codon | GCTACTTCAGGCAGCAGAGGAGA | AGA |
| | | | (SEQ ID NO: 271) | |
| NGA-SpCas9-BE4 | NGA | Stop codon | CTTGTGCAGACTCAGAGGTGAGA | AGA |
| | | | (SEQ ID NO: 272) | |
| NGA-SpCas9-BE4 | NGA | Stop codon | GTGCAGACTCAGAGGTGAGAGGA | GGA |
| | | | (SEQ ID NO: 273) | |
| NGA-SpCas9-BE4 | NGA | Stop codon | GCAGACTCAGAGGTGAGAGGAGA | AGA |
| | | | (SEQ ID NO: 274) | |
| NGA-SpCas9-BE4 | NGA | Stop codon | TTCCCCCAGCTGAAGTCCTTGGA | GGA |
| | | | (SEQ ID NO: 275) | |
| NGA-SpCas9-BE4 | NGA | Stop codon | CTGTCCCAGAACAACATCACTGA | TGA |
| | | | (SEQ ID NO: 276) | |
| NGA-SpCas9-BE4 | NGA | Stop codon | CCTGCAACAACAGGATTCACGGA | GGA |
| | | | (SEQ ID NO: 277) | |
| NGA-SpCas9-BE4 | NGA | Stop codon | CGTCCACATCCTGCAAGGGGGGA | GGA |
| | | | (SEQ ID NO: 278) | |
| NGA-SpCas9-BE4 | NGA | Splice site | ACATCCTGCAAGGGGGGATGGGA | GGA |
| | | | (SEQ ID NO: 279) | |
| NGA-SpCas9-BE4 | NGA | Splice site | CTTACGCCAGCGTCTCCACATGA | TGA |
| | | | (SEQ ID NO: 280) | |
| NGA-SpCas9-BE4 | NGA | Splice site | ACTCACTCCATCACCCGGAGGGA | GGA |
| | | | (SEQ ID NO: 281) | |
| NGA-SpCas9-BE4 | NGA | Splice site | ACTCACCTTAGCCTGAGCAGGGA | GGA |
| | | | (SEQ ID NO: 282) | |
| NGA-SpCas9-BE4 | NGA | Splice site | GACAGACTGCGGGGACACAGTGA | TGA |
| | | | (SEQ ID NO: 283) | |
| NGA-SpCas9-BE4 | NGA | Splice site | ACTCACTTGAGGGTTTCCAAGGA | GGA |
| | | | (SEQ ID NO: 284) | |
| NGA-SpCas9-BE4 | NGA | Splice site | TCCAGGCTGCAGGTGGAATCAGA | AGA |
| | | | (SEQ ID NO: 285) | |
| NGA-SpCas9-BE4 | NGA | Splice site | ACTCACTCCAGATGCTGCAGGGA | GGA |
| | | | (SEQ ID NO: 286) | |
| NGA-SpCas9-BE4 | NGA | Stop codon | AGCCAGCCACAGGGCCCCCAGGA | GGA |
| | | | (SEQ ID NO: 287) | |
| NGA-SpCas9-BE4 | NGA | Stop codon | GCCCAGGTCCTCACGTCTGCGGA | GGA |
| | | | (SEQ ID NO: 288) | |
| NGA-SpCas9-BE4 | NGA | Stop codon | CAGCCCAATAGCTCTTGCCCTGA | TGA |
| | | | (SEQ ID NO: 289) | |
| NGA-SpCas9-BE4 | NGA | Stop codon | GGCCATTTTGGAAGCTTGTTGGA | GGA |
| | | | (SEQ ID NO: 290) | |
| NGA-SpCas9-BE4 | NGA | Splice site | TTACCTGTCATGTTTGCTCGGGA | GGA |
| | | | (SEQ ID NO: 291) | |
| NGA-SpCas9-BE4 | NGA | Splice site | ACCTCACCTACATTGGGGGTGGA | GGA |
| | | | (SEQ ID NO: 292) | |
| NGA-SpCas9-BE4 | NGA | Splice site | CTCACCTACATTGGGGGTGGAGA | AGA |
| | | | (SEQ ID NO: 293) | |
| NGA-SpCas9-BE4 | NGA | Stop codon | TTTGCCAGAGCCCATGGGGCAGA | AGA |
| | | | (SEQ ID NO: 294) | |
| NGA-SpCas9-BE4 | NGA | Splice site | AAGGCTGCAGAGAAAACATGTGA | TGA |
| | | | (SEQ ID NO: 295) | |
| NGA-SpCas9-BE4 | NGA | Splice site | GCTCTACTTTGAGAAAAACCAGA | AGA |
| | | | (SEQ ID NO: 296) | |
| NGA-SpCas9-BE4 | NGC | Splice site | CTCCCAGGCAGCTCACAGTGTGC | TGC |
| | | | (SEQ ID NO: 297) | |
| NGA-SpCas9-BE4 | NGC | Splice site | CTCTGCAGCCTTCCCAGAGGAGC | AGC |
| | | | (SEQ ID NO: 298) | |
| NGA-SpCas9-BE4 | NGC | Splice site | AATGTAGGTGAGGTGCCCCAGGC | GGC |
| | | | (SEQ ID NO: 299) | |
| NGA-SpCas9-BE4 | NGC | Splice site | CCGACAGCTTGTACAATAACTGC | TGC |
| | | | (SEQ ID NO: 300) | |
| NGA-SpCas9-BE4 | NGC | Splice site | CTCACCTCTGAGTCTGCACAAGC | AGC |
| | | | (SEQ ID NO: 301) | |
| NGA-SpCas9-BE4 | NGC | Splice site | ACTCACCAGGCCATTTTGGAAGC | AGC |
| | | | (SEQ ID NO: 302) | |
| NGA-SpCas9-BE4 | NGC | Splice site | CCTGCACACCTGGCTTCCAGTGC | TGC |
| | | | (SEQ ID NO: 303) | |
| NGA-SpCas9-BE4 | NGC | Splice site | AAACTTACTGAAAATGTCCTTGC | TGC |
| | | | (SEQ ID NO: 304) | |
| NGA-SpCas9-BE4 | NGC | Splice site | TCCTCACCGATATTGGCATAAGC | AGC |
| | | | (SEQ ID NO: 305) | |
| NGA-SpCas9-AIBE | NGC | Splice site | CTCCCAGGCAGCTCACAGTGTGC | TGC |
| | | | (SEQ ID NO: 306) | |
| NGA-SpCas9-AIBE | NGC | Splice site | CTCTGCAGCCTTCCCAGAGGAGC | AGC |
| | | | (SEQ ID NO: 307) | |
| NGA-SpCas9-AIBE | NGC | Splice site | CACCCCCAATGTAGGTGAGGTGC | TGC |
| | | | (SEQ ID NO: 308) | |
| NGA-SpCas9-AIBE | NGC | Splice site | AATGTAGGTGAGGTGCCCCAGGC | GGC |
| | | | (SEQ ID NO: 309) | |
| NGA-SpCas9-AIBE | NGC | Splice site | GGCCTTTGCAGAGCCGGTGGAGC | AGC |
| | | | (SEQ ID NO: 310) | |
| NGA-SpCas9-AIBE | NGC | Splice site | CCCTCCACAGGGCTGCCTTGAGC | AGC |
| | | | (SEQ ID NO: 311) | |
| NGA-SpCas9-AIBE | NGC | Splice site | GATTCCACCTGCAGCCTGGATGC | TGC |
| | | | (SEQ ID NO: 312) | |
| NGA-SpCas9-AIBE | NGC | Splice site | TTCCACCTGCAGCCTGGATGCGC | CGC |
| | | | (SEQ ID NO: 313) | |
| NGA-SpCas9-AIBE | NGC | Splice site | CCGACAGCTTGTACAATAACTGC | TGC |
| | | | (SEQ ID NO: 314) | |
| NGA-SpCas9-AIBE | NGC | Splice site | CCCCCCTTGCAGGATGTGGACGC | CGC |
| | | | (SEQ ID NO: 315) | |
| NGA-SpCas9-AIBE | NGC | Splice site | GCCCCACTCACCTTAGCCTGAGC | AGC |
| | | | (SEQ ID NO: 316) | |
| NGA-SpCas9-AIBE | NGC | Splice site | GAGTCTATACTCACTCCAGATGC | TGC |
| | | | (SEQ ID NO: 317) | |
| NGA-SpCas9-AIBE | NGC | Splice site | TCTATACTCACTCCAGATGCTGC | TGC |
| | | | (SEQ ID NO: 318) | |
| NGA-SpCas9-AIBE | NGC | Splice site | TCTCCTCTCACCTCTGAGTCTGC | TGC |
| | | | (SEQ ID NO: 319) | |
| NGA-SpCas9-AIBE | NGC | Splice site | CTCACCTCTGAGTCTGCACAAGC | AGC |
| | | | (SEQ ID NO: 320) | |
| NGA-SpCas9-AIBE | NGC | Splice site | ACTCACCAGGCCATTTTGGAAGC | AGC |
| | | | (SEQ ID NO: 321) | |
| NGA-SpCas9-AIBE | NGC | Splice site | GGGTCCTTACCTGTCATGTTTGC | TGC |
| | | | (SEQ ID NO: 322) | |
| NGA-SpCas9-AIBE | NGC | Splice site | CCTGCACACCTGGCTTCCAGTGC | TGC |
| | | | (SEQ ID NO: 323) | |
| NGA-SpCas9-AIBE | NGC | Splice site | AAACTTACTGAAAATGTCCTTGC | TGC |
| | | | (SEQ ID NO: 324) | |
| NGA-SpCas9-AIBE | NGC | Splice site | GGTGCTTCCTCACCGATATTGGC | GGC |
| | | | (SEQ ID NO: 325) | |
| NGA-SpCas9-AIBE | NGC | Splice site | TCCTCACCGATATTGGCATAAGC | AGC |
| | | | (SEQ ID NO: 326) | |
| NGA-SpCas9-AIBE | NGC | Splice site | AGGAGGGCCCACCTGAGTAGAGC | AGC |
| | | | (SEQ ID NO: 327) | |
| NGC-SpCas9-BE4 | NGC | Stop codon | ACTCCCAGCTGGAGGGCCTGAGC | AGC |
| | | | (SEQ ID NO: 328) | |
| NGC-SpCas9-BE4 | NGC | Stop codon | AGTCAGAAAAGACGTGAGTGAGC | AGC |
| | | | (SEQ ID NO: 329) | |
| NGC-SpCas9-BE4 | NGC | Stop codon | TCAACCAGGAGCCAGCCTCCGGC | GGC |
| | | | (SEQ ID NO: 330) | |
| NGC-SpCas9-BE4 | NGC | Stop codon | GGAGCAGTTCTACCGCTCACTGC | TGC |
| | | | (SEQ ID NO: 331) | |
| NGC-SpCas9-BE4 | NGC | Stop codon | TCACTGCAGGACACGTATGGTGC | TGC |
| | | | (SEQ ID NO: 332) | |
| NGC-SpCas9-BE4 | NGC | Stop codon | AACGGCAGCTGGCCCAAGGAGGC | GGC |
| | | | (SEQ ID NO: 333) | |
| NGC-SpCas9-BE4 | NGC | Stop codon | TGAGACACGAGTGATTGCTGTGC | TGC |
| | | | (SEQ ID NO: 334) | |
| NGC-SpCas9-BE4 | NGC | Stop codon | GGTCAGGGCAAGAGCTATTGGGC | GGC |
| | | | (SEQ ID NO: 335) | |
| NGC-SpCas9-BE4 | NGC | Stop codon | GGCCCACAGCCACTCGTGGCGGC | GGC |
| | | | (SEQ ID NO: 336) | |
| NGC-SpCas9-BE4 | NGC | Stop codon | GGAAGCGCAAGATGGCTTCCTGC | TGC |
| | | | (SEQ ID NO: 337) | |
| NGC-SpCas9-BE4 | NGC | Stop codon | CTGGTCCAGAGCCTGAGCAAGGC | GGC |
| | | | (SEQ ID NO: 338) | |
| NGC-SpCas9-BE4 | NGC | Stop codon | GCAGGCCCAGGCATACGTGATGC | TGC |
| | | | (SEQ ID NO: 339) | |
| NGC-SpCas9-BE4 | NGC | Stop codon | AGGCCCAGGCATACGTGATGCGC | CGC |
| | | | (SEQ ID NO: 340) | |
| NGC-SpCas9-BE4 | NGC | Stop codon | GCACCAAGACAGAGCCCTGACGC | CGC |
| | | | (SEQ ID NO: 341) | |
| NGC-SpCas9-BE4 | NGC | Stop codon | GTGCCAGCTCTCAGAGGCCCTGC | TGC |
| | | | (SEQ ID NO: 342) | |
| NGC-SpCas9-BE4 | NGC | Stop codon | TTAGTCCAACACCCACCGCGGGC | GGC |
| | | | (SEQ ID NO: 343) | |
| NGC-SpCas9-BE4 | NGC | Stop codon | GTCCAACACCCACCGCGGGCCGC | CGC |
| | | | (SEQ ID NO: 344) | |
| NGC-SpCas9-BE4 | NGC | Stop codon | CTCCTGCAATGCTTCCTGGGGGC | GGC |
| | | | (SEQ ID NO: 345) | |
| NGC-SpCas9-BE4 | NGC | Stop codon | TCTTCCAGCCTCCCGCCCGCTGC | TGC |
| | | | (SEQ ID NO: 346) | |
| NGC-SpCas9-BE4 | NGC | Stop codon | GCGGCTGCAGCCGGGGACACTGC | TGC |
| | | | (SEQ ID NO: 347) | |
| NGC-SpCas9-BE4 | NGC | Stop codon | CTGCAGCCGGGGACACTGCGGGC | GGC |
| | | | (SEQ ID NO: 348) | |
| NGC-SpCas9-BE4 | NGC | Stop codon | GGCGCGGCAGCTGCTGGAGCTGC | TGC |
| | | | (SEQ ID NO: 349) | |
| NGC-SpCas9-BE4 | NGC | Stop codon | GCGGCAGCTGCTGGAGCTGCTGC | TGC |
| | | | (SEQ ID NO: 350) | |
| NGC-SpCas9-BE4 | NGC | Stop codon | TTGGCAGCACGTGGTACAGGAGC | AGC |
| | | | (SEQ ID NO: 351) | |
| NGC-SpCas9-BE4 | NGC | Stop codon | TGGTACAGGAGCTCCCCGGCCGC | CGC |
| | | | (SEQ ID NO: 352) | |
| NGC-SpCas9-BE4 | NGC | Stop codon | GCAGCAGCATGGGGAGACCAAGC | AGC |
| | | | (SEQ ID NO: 353) | |
| NGC-SpCas9-BE4 | NGC | Stop codon | GACTCAGAGGTGAGAGGAGAGGC | GGC |
| | | | (SEQ ID NO: 354) | |
| NGC-SpCas9-BE4 | NGC | Stop codon | GTCCAGTACAACAAGTTCACGGC | GGC |
| | | | (SEQ ID NO: 355) | |
| NGC-SpCas9-BE4 | NGC | Stop codon | GGGCCCAGCAGCTCGCTGCCAGC | AGC |
| | | | (SEQ ID NO: 356) | |
| NGC-SpCas9-BE4 | NGC | Stop codon | AACAACAGGATTCACGGATCAGC | AGC |
| | | | (SEQ ID NO: 357) | |
| NGC-SpCas9-BE4 | NGC | Splice site | TACAAGCTGTCGGAAACAGAGGC | GGC |
| | | | (SEQ ID NO: 358) | |
| NGC-SpCas9-BE4 | NGC | Splice site | GCCCAGCCTAGGAGGCAAAGAGC | AGC |
| | | | (SEQ ID NO: 359) | |
| NGC-SpCas9-BE4 | NGC | Splice site | CTCACCTCTGAGTCTGCACAAGC | AGC |
| | | | (SEQ ID NO: 360) | |
| NGC-SpCas9-BE4 | NGC | Stop codon | CTCCCACAGCGCCACCGTGTCGC | CGC |
| | | | (SEQ ID NO: 361) | |
| NGC-SpCas9-BE4 | NGC | Splice site | CCACCTGAAACGGGTGACACAGC | AGC |
| | | | (SEQ ID NO: 362) | |
| NGC-SpCas9-BE4 | NGC | Stop codon | TGCCAAATTCCAGCCTCCTCGGC | GGC |
| | | | (SEQ ID NO: 363) | |
| NGC-SpCas9-BE4 | NGC | Stop codon | CCTCCAGCCAGTTGTCATAGGGC | GGC |
| | | | (SEQ ID NO: 364) | |
| NGC-SpCas9-BE4 | NGC | Stop codon | CAGCCACAGGGCCCCCAGGAAGC | AGC |
| | | | (SEQ ID NO: 365) | |
| NGC-SpCas9-BE4 | NGC | Stop codon | ATCGCCCAGGTCCTCACGTCTGC | TGC |
| | | | (SEQ ID NO: 366) | |
| NGC-SpCas9-BE4 | NGC | Stop codon | GCTCCCAGGCCAGCTTGGCCAGC | AGC |
| | | | (SEQ ID NO: 367) | |
| NGC-SpCas9-BE4 | NGC | Stop codon | CAAGCCCAGGCCCGGCTCACTGC | TGC |
| | | | (SEQ ID NO: 368) | |
| NGC-SpCas9-BE4 | NGC | Splice site | CCGGCTCTGCAAAGGCCAGGGGC | GGC |
| | | | (SEQ ID NO: 369) | |
| NGC-SpCas9-BE4 | NGC | Splice site | ACTCACCAGGCCATTTTGGAAGC | AGC |
| | | | (SEQ ID NO: 370) | |
| NGC-SpCas9-BE4 | NGC | Splice site | TTGTGCTCTGGAGATGGAGAAGC | AGC |
| | | | (SEQ ID NO: 371) | |
| NGC-SpCas9-BE4 | NGC | Stop codon | AGATTTGCCAGAGCCCATGGGGC | GGC |
| | | | (SEQ ID NO: 372) | |
| NGC-SpCas9-BE4 | NGC | Splice site | AAGGCACTGCAAGAGACAAAGGC | GGC |
| | | | (SEQ ID NO: 373) | |
| NGC-SpCas9-BE4 | NGC | Splice site | GGCTCAGCTGTGAGGAAGTGGGC | GGC |
| | | | (SEQ ID NO: 374) | |
| NGC-SpCas9-BE4 | NGC | Stop codon | GGCTTCCAGTGCTTCAGGTCTGC | TGC |
| | | | (SEQ ID NO: 375) | |
| NGC-SpCas9-BE4 | NGC | Splice site | AAACTTACTGAAAATGTCCTTGC | TGC |
| | | | (SEQ ID NO: 376) | |
| NGC-SpCas9-BE4 | NGC | Splice site | CCGCTGCCCAGACAAGGAAAAGC | AGC |
| | | | (SEQ ID NO: 377) | |
| NGC-SpCas9-BE4 | NGC | Splice site | TCCTCACCGATATTGGCATAAGC | AGC |
| | | | (SEQ ID NO: 378) | |
| NGC-SpCas9-BE4 | NGC | Splice site | CTGCCTGGGAGGGAAGACAATGC | TGC |
| | | | (SEQ ID NO: 379) | |
| SaCas9-ABE | NNGRRT | Splice site | CCACCTGCAGCCTGGATGCGCTGAGT | CTGAGT |
| | | | (SEQ(SEQ ID NO: 380) | |
| SaCas9-ABE | NNGRRT | Splice site | CCACTCACCTTAGCCTGAGCAGGGAT | AGGGAT |
| | | | (SEQ(SEQ ID NO: 381) | |
| SaCas9-ABE | NGG | Splice site | CACAGCTGAGCCCCCCACTGTGG | TGG |
| | | | (SEQ ID NO: 382) | |
| SaCas9-ABE | NGG | Splice site | CAATGTAGGTGAGGTGCCCCAGG | AGG |
| | | | (SEQ ID NO: 383) | |
| SaCas9-ABE | NGG | Splice site | CTCCAGGACGAGAAGTTCCTCGG | CGG |
| | | | (SEQ ID NO: 384) | |
| SaCas9-ABE | NGG | Splice site | CCTAGGCTGGGCCCTGTCTCAGG | AGG |
| | | | (SEQ ID NO: 385) | |
| SaCas9-ABE | NGG | Splice site | ACACTCACTCCATCACCCGGAGG | AGG |
| | | | (SEQ ID NO: 386) | |
| SaCas9-ABE | NGG | Splice site | CACTCACTCCATCACCCGGAGGG | GGG |
| | | | (SEQ ID NO: 387) | |
| SaCas9-ABE | NGG | Splice site | CCACTCACCTTAGCCTGAGCAGG | AGG |
| | | | (SEQ ID NO: 388) | |
| SaCas9-ABE | NGG | Splice site | CACTCACCTTAGCCTGAGCAGGG | GGG |
| | | | (SEQ ID NO: 389) | |
| SaCas9-ABE | NGG | Splice site | CACTCACTTGAGGGTTTCCAAGG | AGG |
| | | | (SEQ ID NO: 390) | |
| SaCas9-ABE | NGG | Splice site | ATACTCACTCCAGATGCTGCAGG | AGG |
| | | | (SEQ ID NO: 391) | |
| SaCas9-ABE | NGG | Splice site | TACTCACTCCAGATGCTGCAGGG | GGG |
| | | | (SEQ ID NO: 392) | |
| SaCas9-ABE | NGG | Splice site | CCTTACCTGTCATGTTTGCTCGG | CGG |
| | | | (SEQ ID NO: 393) | |
| SaCas9-ABE | NGG | Splice site | CTTACCTGTCATGTTTGCTCGGG | GGG |
| | | | (SEQ ID NO: 394) | |
| SaCas9-ABE | NGG | Splice site | TAACATACTGGGAATCTGGTCGG | CGG |
| | | | (SEQ ID NO: 395) | |
| SaCas9-ABE | NGG | Splice site | TTTTACCTTGGGGCTCTGACAGG | AGG |
| | | | (SEQ ID NO: 396) | |
| SaCas9-AIBE | NGG | Splice site | CCTTGTCTGGGCAGCGGAACTGG | TGG |
| | | | (SEQ ID NO: 397) | |
| SaCas9-AIBE | NGG | Splice site | TTTCTCAAAGTAGAGCACATAGG | AGG |
| | | | (SEQ ID NO: 398) | |
| SaCas9-AIBE | NGG | Splice site | TTTCTCTGCAGCCTTCCCAGAGG | AGG |
| | | | (SEQ ID NO: 399) | |
| SaCas9-AIBE | NGG | Splice site | CACAGCTGAGCCCCCCACTGTGG | TGG |
| | | | (SEQ ID NO: 400) | |
| SaCas9-AIBE | NGG | Splice site | CAATGTAGGTGAGGTGCCCCAGG | AGG |
| | | | (SEQ ID NO: 401) | |
| SaCas9-AIBE | NGG | Splice site | CCTGGCCTTTGCAGAGCCGGTGG | TGG |
| | | | (SEQ ID NO: 402) | |
| SaCas9-AIBE | NGG | Splice site | CTCCAGGACGAGAAGTTCCTCGG | CGG |
| | | | (SEQ ID NO: 403) | |
| SaCas9-AIBE | NGG | Splice site | CCTAGGCTGGGCCCTGTCTCAGG | AGG |
| | | | (SEQ ID NO: 404) | |
| SaCas9-AIBE | NGG | Splice site | CCCACACTCACTCCATCACCCGG | CGG |
| | | | (SEQ ID NO: 405) | |
| SaCas9-AIBE | NGG | Splice site | ACACTCACTCCATCACCCGGAGG | AGG |
| | | | (SEQ ID NO: 406) | |
| SaCas9-AIBE | NGG | Splice site | CACTCACTCCATCACCCGGAGGG | GGG |
| | | | (SEQ ID NO: 407) | |
| SaCas9-AIBE | NGG | Splice site | CCACTCACCTTAGCCTGAGCAGG | AGG |
| | | | (SEQ ID NO: 408) | |
| SaCas9-AIBE | NGG | Splice site | CACTCACCTTAGCCTGAGCAGGG | GGG |
| | | | (SEQ ID NO: 409) | |
| SaCas9-AIBE | NGG | Splice site | CACTCACTTGAGGGTTTCCAAGG | AGG |
| | | | (SEQ ID NO: 410) | |
| SaCas9-AIBE | NGG | Splice site | ATACTCACTCCAGATGCTGCAGG | AGG |
| | | | (SEQ ID NO: 411) | |
| SaCas9-AIBE | NGG | Splice site | TACTCACTCCAGATGCTGCAGGG | GGG |
| | | | (SEQ ID NO: 412) | |
| SaCas9-AIBE | NGG | Splice site | GCCCCTCACCCCACCTGAAACGG | CGG |
| | | | (SEQ ID NO: 413) | |
| SaCas9-AIBE | NGG | Splice site | CCCCTCACCCCACCTGAAACGGG | GGG |
| | | | (SEQ ID NO: 414) | |
| SaCas9-AIBE | NGG | Splice site | CATCACTCACCAGGCCATTTTGG | TGG |
| | | | (SEQ ID NO: 415) | |
| SaCas9-AIBE | NGG | Splice site | CCTTACCTGTCATGTTTGCTCGG | CGG |
| | | | (SEQ ID NO: 416) | |
| SaCas9-AIBE | NGG | Splice site | CTTACCTGTCATGTTTGCTCGGG | GGG |
| | | | (SEQ ID NO: 417) | |
| SaCas9-AIBE | NGG | Splice site | CCCCTAACATACTGGGAATCTGG | TGG |
| | | | (SEQ ID NO: 418) | |
| SaCas9-AIBE | NGG | Splice site | TAACATACTGGGAATCTGGTCGG | CGG |
| | | | (SEQ ID NO: 419) | |
| SaCas9-AIBE | NGG | Splice site | AGGTGCTTCCTCACCGATATTGG | TGG |
| | | | (SEQ ID NO: 420) | |
| SaCas9-AIBE | NGG | Splice site | TTTTACCTTGGGGCTCTGACAGG | AGG |
| | | | (SEQ ID NO: 421) | |
| SpCas9-BE4 | NGG | Stop codon | GAGCCCCAAGGTAAAAAGGCCGG | CGG |
| | | | (SEQ ID NO: 422) | |
| SpCas9-BE4 | NGG | Stop codon | AGCCCCAAGGTAAAAAGGCCGGG | GGG |
| | | | (SEQ ID NO: 423) | |
| SpCas9-BE4 | NGG | Stop codon | CAGCTCACAGTGTGCCACCATGG | TGG |
| | | | (SEQ ID NO: 424) | |
| SpCas9-BE4 | NGG | Stop codon | TATGACCAGATGGACCTGGCTGG | TGG |
| | | | (SEQ ID NO: 425) | |
| SpCas9-BE4 | NGG | Stop codon | ACTGGACCAGTATGTCTTCCAGG | AGG |
| | | | (SEQ ID NO: 426) | |
| SpCas9-BE4 | NGG | Stop codon | TGTCTTCCAGGACTCCCAGCTGG | TGG |
| | | | (SEQ ID NO: 427) | |
| SpCas9-BE4 | NGG | Stop codon | CTTCCAGGACTCCCAGCTGGAGG | AGG |
| | | | (SEQ ID NO: 428) | |
| SpCas9-BE4 | NGG | Stop codon | TTCCAGGACTCCCAGCTGGAGGG | GGG |
| | | | (SEQ ID NO: 429) | |
| SpCas9-BE4 | NGG | Stop codon | TTCAACCAGGAGCCAGCCTCCGG | CGG |
| | | | (SEQ ID NO: 430) | |
| SpCas9-BE4 | NGG | Stop codon | GACCAGATTCCCAGTATGTTAGG | AGG |
| | | | (SEQ ID NO: 431) | |
| SpCas9-BE4 | NGG | Stop codon | CTCTGGCAAATCTCTGAGGCTGG | TGG |
| | | | (SEQ ID NO: 432) | |
| SpCas9-BE4 | NGG | Stop codon | AGCCAAGTACCCCCTCCCAGTGG | TGG |
| | | | (SEQ ID NO: 433) | |
| SpCas9-BE4 | NGG | Stop codon | ACCTCCCGAGCAAACATGACAGG | AGG |
| | | | (SEQ ID NO: 434) | |
| SpCas9-BE4 | NGG | Stop codon | CCCACCCAATGCCCGGCAGCTGG | TGG |
| | | | (SEQ ID NO: 435) | |
| SpCas9-BE4 | NGG | Stop codon | TGGTGCAGGCCAGGCTGGAGAGG | AGG |
| | | | (SEQ ID NO: 436) | |
| SpCas9-BE4 | NGG | Stop codon | GAACGGCAGCTGGCCCAAGGAGG | AGG |
| | | | (SEQ ID NO: 437) | |
| SpCas9-BE4 | NGG | Stop codon | GGCCCAAGGAGGCCTGGCTGAGG | AGG |
| | | | (SEQ ID NO: 438) | |
| SpCas9-BE4 | NGG | Stop codon | GACACGAGTGATTGCTGTGCTGG | TGG |
| | | | (SEQ ID NO: 439) | |
| SpCas9-BE4 | NGG | Stop codon | ACACGAGTGATTGCTGTGCTGGG | GGG |
| | | | (SEQ ID NO: 440) | |
| SpCas9-BE4 | NGG | Stop codon | CTGGTCAGGGCAAGAGCTATTGG | TGG |
| | | | (SEQ ID NO: 441) | |
| SpCas9-BE4 | NGG | Stop codon | TGGTCAGGGCAAGAGCTATTGGG | GGG |
| | | | (SEQ ID NO: 442) | |
| SpCas9-BE4 | NGG | Stop codon | GGGCCCACAGCCACTCGTGGCGG | CGG |
| | | | (SEQ ID NO: 443) | |
| SpCas9-BE4 | NGG | Stop codon | TTCCAGAAGAAGCTGCTCCGAGG | AGG |
| | | | (SEQ ID NO: 444) | |
| SpCas9-BE4 | NGG | Stop codon | CCTGGTCCAGAGCCTGAGCAAGG | AGG |
| | | | (SEQ ID NO: 445) | |
| SpCas9-BE4 | NGG | Stop codon | CAGACATCAAAGTACCCTACAGG | AGG |
| | | | (SEQ ID NO: 446) | |
| SpCas9-BE4 | NGG | Stop codon | ACATCAAAGTACCCTACAGGAGG | AGG |
| | | | (SEQ ID NO: 447) | |
| SpCas9-BE4 | NGG | Stop codon | CTTAGTCCAACACCCACCGCGGG | GGG |
| | | | (SEQ ID NO: 448) | |
| SpCas9-BE4 | NGG | Stop codon | CCTCCTGCAATGCTTCCTGGGGG | GGG |
| | | | (SEQ ID NO: 449) | |
| SpCas9-BE4 | NGG | Stop codon | GGAAGCAGAAGGTGCTTGCGAGG | AGG |
| | | | (SEQ ID NO: 450) | |
| SpCas9-BE4 | NGG | Stop codon | GGCTGCAGCCGGGGACACTGCGG | CGG |
| | | | (SEQ ID NO: 451) | |
| SpCas9-BE4 | NGG | Stop codon | GCTGCAGCCGGGGACACTGCGGG | GGG |
| | | | (SEQ ID NO: 452) | |
| SpCas9-BE4 | NGG | Stop codon | AATTTGGCAGCACGTGGTACAGG | AGG |
| | | | (SEQ ID NO: 453) | |
| SpCas9-BE4 | NGG | Stop codon | GGCGGGCCAAGACTTCTCCCTGG | TGG |
| | | | (SEQ ID NO: 454) | |
| SpCas9-BE4 | NGG | Stop codon | TGTGCAGACTCAGAGGTGAGAGG | AGG |
| | | | (SEQ ID NO: 455) | |
| SpCas9-BE4 | NGG | Stop codon | AGACTCAGAGGTGAGAGGAGAGG | AGG |
| | | | (SEQ ID NO: 456) | |
| SpCas9-BE4 | NGG | Stop codon | CCCCCAGGCTTTCCCCAAACTGG | TGG |
| | | | (SEQ ID NO: 457) | |
| SpCas9-BE4 | NGG | Stop codon | CTTCCCCCAGCTGAAGTCCTTGG | TGG |
| | | | (SEQ ID NO: 458) | |
| SpCas9-BE4 | NGG | Stop codon | CGTCCAGTACAACAAGTTCACGG | CGG |
| | | | (SEQ ID NO: 459) | |
| SpCas9-BE4 | NGG | Stop codon | ACCTGCAACAACAGGATTCACGG | CGG |
| | | | (SEQ ID NO: 460) | |
| SpCas9-BE4 | NGG | Stop codon | TGGGCGTCCACATCCTGCAAGGG | GGG |
| | | | (SEQ ID NO: 461) | |
| SpCas9-BE4 | NGG | Stop codon | GGGCGTCCACATCCTGCAAGGGG | GGG |
| | | | (SEQ ID NO: 462) | |
| SpCas9-BE4 | NGG | Stop codon | GGCGTCCACATCCTGCAAGGGGG | GGG |
| | | | (SEQ ID NO: 463) | |
| SpCas9-BE4 | NGG | Stop codon | GCGTCCACATCCTGCAAGGGGGG | GGG |
| | | | (SEQ ID NO: 464) | |
| SpCas9-BE4 | NGG | Splice site | CCACATCCTGCAAGGGGGGATGG | TGG |
| | | | (SEQ ID NO: 465) | |
| SpCas9-BE4 | NGG | Splice site | CACATCCTGCAAGGGGGGATGGG | GGG |
| | | | (SEQ ID NO: 466) | |
| SpCas9-BE4 | NGG | Splice site | ACACTCACTCCATCACCCGGAGG | AGG |
| | | | (SEQ ID NO: 467) | |
| SpCas9-BE4 | NGG | Splice site | CACTCACTCCATCACCCGGAGGG | GGG |
| | | | (SEQ ID NO: 468) | |
| SpCas9-BE4 | NGG | Splice site | GTACAAGCTGTCGGAAACAGAGG | AGG |
| | | | (SEQ ID NO: 469) | |
| SpCas9-BE4 | NGG | Splice site | CCACTCACCTTAGCCTGAGCAGG | AGG |
| | | | (SEQ ID NO: 470) | |
| SpCas9-BE4 | NGG | Splice site | CACTCACCTTAGCCTGAGCAGGG | GGG |
| | | | (SEQ ID NO: 471) | |
| SpCas9-BE4 | NGG | Splice site | CAGACTGCGGGGACACAGTGAGG | AGG |
| | | | (SEQ ID NO: 472) | |
| SpCas9-BE4 | NGG | Splice site | AGACTGCGGGGACACAGTGAGGG | GGG |
| | | | (SEQ ID NO: 473) | |
| SpCas9-BE4 | NGG | Splice site | CACTCACTTGAGGGTTTCCAAGG | AGG |
| | | | (SEQ ID NO: 474) | |
| SpCas9-BE4 | NGG | Splice site | AGGCTGCAGGTGGAATCAGATGG | TGG |
| | | | (SEQ ID NO: 475) | |
| SpCas9-BE4 | NGG | Splice site | ATACTCACTCCAGATGCTGCAGG | AGG |
| | | | (SEQ ID NO: 476) | |
| SpCas9-BE4 | NGG | Splice site | TACTCACTCCAGATGCTGCAGGG | GGG |
| | | | (SEQ ID NO: 477) | |
| SpCas9-BE4 | NGG | Splice site | TCACTCCAGATGCTGCAGGGAGG | AGG |
| | | | (SEQ ID NO: 478) | |
| SpCas9-BE4 | NGG | Splice site | AGCCTAGGAGGCAAAGAGCAAGG | AGG |
| | | | (SEQ ID NO: 479) | |
| SpCas9-BE4 | NGG | Stop codon | CTGCCAAATTCCAGCCTCCTCGG | CGG |
| | | | (SEQ ID NO: 480) | |
| SpCas9-BE4 | NGG | Stop codon | GAGCCAGCCACAGGGCCCCCAGG | AGG |
| | | | (SEQ ID NO: 481) | |
| SpCas9-BE4 | NGG | Stop codon | CGCCCAGGTCCTCACGTCTGCGG | CGG |
| | | | (SEQ ID NO: 482) | |
| SpCas9-BE4 | NGG | Splice site | ACCGGCTCTGCAAAGGCCAGGGG | GGG |
| | | | (SEQ ID NO: 483) | |
| SpCas9-BE4 | NGG | Stop codon | AGGCCATTTTGGAAGCTTGTTGG | TGG |
| | | | (SEQ ID NO: 484) | |
| SpCas9-BE4 | NGG | Splice site | TGCTCTGGAGATGGAGAAGCAGG | AGG |
| | | | (SEQ ID NO: 485) | |
| SpCas9-BE4 | NGG | Splice site | CCTTACCTGTCATGTTTGCTCGG | CGG |
| | | | (SEQ ID NO: 486) | |
| SpCas9-BE4 | NGG | Splice site | CTTACCTGTCATGTTTGCTCGGG | GGG |
| | | | (SEQ ID NO: 487) | |
| SpCas9-BE4 | NGG | Splice site | AAAGGCACTGCAAGAGACAAAGG | AGG |
| | | | (SEQ ID NO: 488) | |
| SpCas9-BE4 | NGG | Splice site | TAACATACTGGGAATCTGGTCGG | CGG |
| | | | (SEQ ID NO: 489) | |
| SpCas9-BE4 | NGG | Stop codon | TTCCAGTGCTTCAGGTCTGCCGG | CGG |
| | | | (SEQ ID NO: 490) | |
| SpCas9-BE4 | NGG | Splice site | TTTTACCTTGGGGCTCTGACAGG | AGG |
| | | | (SEQ ID NO: 491) | |

TABLE 4A
Spacer sequences for RNA for CIITA target gene

| | Search | Strategy | Full sequence |
|---|---|---|---|
| Editor | PAM | site | (Protospacer underlined) |
| Cas12b-ABE | RTTN | Splice site | UCUCUGCAGCCUUCCCAGAG |
| | | | (SEQ ID NO: 492) |
| Cas12b-ABE | NGG | Stop codon | UCUCCAGGACGAGAAGUUCC |
| | | | (SEQ ID NO: 493) |
| Cas12b-ABE | NGG | Stop codon | CACCUGCAGCCUGGAUGCGC |
| | | | (SEQ ID NO: 494) |
| Cas12b-ABE | NGG | Stop codon | CCGACAGCUUGUACAAUAAC |
| | | | (SEQ ID NO: 495) |
| Cas12b-ABE | NGG | Stop codon | CUCUUGCCAGCGUCCAGUAC |
| | | | (SEQ ID NO: 496) |
| KKH-SaCas9-ABE | NNNRRT | Splice site | UGUCUGGGCAGCGGAACUGG |
| | | | (SEQ ID NO: 497) |
| KKH-SaCas9-ABE | NNNRRT | Splice site | UCAAAGUAGAGCACAUAGGA |
| | | | (SEQ ID NO: 498) |
| KKH-SaCas9-ABE | NNNRRT | Splice site | CUCACAGCUGAGCCCCCCAC |
| | | | (SEQ ID NO: 499) |
| KKH-SaCas9-ABE | NNNRRT | Splice site | GGCCUUUGCAGAGCCGGUGG |
| | | | (SEQ ID NO: 500) |
| KKH-SaCas9-ABE | NNNRRT | Splice site | CCACCUGCAGCCUGGAUGCG |
| | | | (SEQ ID NO: 501) |
| KKH-SaCas9-ABE | NNNRRT | Splice site | UCUUGCCAGCGUCCAGUACA |
| | | | (SEQ ID NO: 502) |
| KKH-SaCas9-ABE | NNNRRT | Splice site | CCACUCACCUUAGCCUGAGC |
| | | | (SEQ ID NO: 503) |
| KKH-SaCas9-ABE | NNNRRT | Splice site | CCUAACAUACUGGGAAUCUG |
| | | | (SEQ ID NO: 504) |
| KKH-SaCas9-ABE | NNNRRT | Splice site | GAGGGCCCACCUGAGUAGAG |
| | | | (SEQ ID NO: 505) |
| KKH-SaCas9-ABE | NNNRRT | Splice site | CUUUUUACCUUGGGGCUCUG |
| | | | (SEQ ID NO: 506) |
| NGA-SpCas9-ABE | NGA | Splice site | AAAGUAGAGCACAUAGGACC |
| | | | (SEQ ID NO: 507) |
| NGA-SpCas9-ABE | NGA | Splice site | CUCCACAGGGCUGCCUUGAG |
| | | | (SEQ ID NO: 508) |
| NGA-SpCas9-ABE | NGA | Splice site | UCCAGGACGAGAAGUUCCUC |
| | | | (SEQ ID NO: 509) |
| NGA-SpCas9-ABE | NGA | Splice site | CACCUGCAGCCUGGAUGCGC |
| | | | (SEQ ID NO: 510) |
| NGA-SpCas9-ABE | NGA | Splice site | UGCAGCCUGGAUGCGCUGAG |
| | | | (SEQ ID NO: 511) |
| NGA-SpCas9-ABE | NGA | Splice site | CUUACGCCAGCGUCUCCACA |
| | | | (SEQ ID NO: 512) |
| NGA-SpCas9-ABE | NGA | Splice site | ACUCACUCCAUCACCCGGAG |
| | | | (SEQ ID NO: 513) |
| NGA-SpCas9-ABE | NGA | Splice site | ACUCACCUUAGCCUGAGCAG |
| | | | (SEQ ID NO: 514) |
| NGA-SpCas9-ABE | NGA | Splice site | ACUCACUUGAGGGUUUCCAA |
| | | | (SEQ ID NO: 515) |
| NGA-SpCas9-ABE | NGA | Splice site | ACUCACUCCAGAUGCUGCAG |
| | | | (SEQ ID NO: 516) |
| NGA-SpCas9-ABE | NGA | Splice site | AUCACUCACCAGGCCAUUUU |
| | | | (SEQ ID NO: 517) |
| NGA-SpCas9-BE4 | NGA | Stop codon | GCCCCAAGGUAAAAAGGCCG |
| | | | (SEQ ID NO: 518) |
| NGA-SpCas9-BE4 | NGA | Stop codon | AGCUCACAGUGUGCCACCAU |
| | | | (SEQ ID NO: 519) |
| NGA-SpCas9-BE4 | NGA | Stop codon | AUGACCAGAUGGACCUGGCU |
| | | | (SEQ ID NO: 520) |
| NGA-SpCas9-BE4 | NGA | Stop codon | GACCAGAUGGACCUGGCUGG |
| | | | (SEQ ID NO: 521) |
| NGA-SpCas9-BE4 | NGA | Stop codon | CUGGACCAGUAUGUCUUCCA |
| | | | (SEQ ID NO: 522) |
| NGA-SpCas9-BE4 | NGA | Stop codon | GUCUUCCAGGACUCCCAGCU |
| | | | (SEQ ID NO: 523) |
| NGA-SpCas9-BE4 | NGA | Stop codon | GGACUCCCAGCUGGAGGGCC |
| | | | (SEQ ID NO: 524) |
| NGA-SpCas9-BE4 | NGA | Stop codon | UUGGGCAGAAAAGUCAGAAA |
| | | | (SEQ ID NO: 525) |
| NGA-SpCas9-BE4 | NGA | Stop codon | AAAGUCAGAAAAGACGUGAG |
| | | | (SEQ ID NO: 526) |
| NGA-SpCas9-BE4 | NGA | Stop codon | CUCCGGCCAGAUGCGCCUGG |
| | | | (SEQ ID NO: 527) |
| NGA-SpCas9-BE4 | NGA | Stop codon | UCUGGCAAAUCUCUGAGGCU |
| | | | (SEQ ID NO: 528) |
| NGA-SpCas9-BE4 | NGA | Stop codon | CCACCCAAUGCCCGGCAGCU |
| | | | (SEQ ID NO: 529) |
| NGA-SpCas9-BE4 | NGA | Stop codon | ACCCAAUGCCCGGCAGCUGG |
| | | | (SEQ ID NO: 530) |
| NGA-SpCas9-BE4 | NGA | Stop codon | CUGCAGGACACGUAUGGUGC |
| | | | (SEQ ID NO: 531) |
| NGA-SpCas9-BE4 | NGA | Stop codon | UCUGGUGCAGGCCAGGCUGG |
| | | | (SEQ ID NO: 532) |
| NGA-SpCas9-BE4 | NGA | Stop codon | GGUGCAGGCCAGGCUGGAGA |
| | | | (SEQ ID NO: 533) |
| NGA-SpCas9-BE4 | NGA | Stop codon | CUGGCCCAAGGAGGCCUGGC |
| | | | (SEQ ID NO: 534) |
| NGA-SpCas9-BE4 | NGA | Stop codon | CCACAGCCACUCGUGGCGGC |
| | | | (SEQ ID NO: 535) |
| NGA-SpCas9-BE4 | NGA | Stop codon | UUUUCCAGAAGAAGCUGCUC |
| | | | (SEQ ID NO: 536) |
| NGA-SpCas9-BE4 | NGA | Stop codon | GUCCAGAGCCUGAGCAAGGC |
| | | | (SEQ ID NO: 537) |
| NGA-SpCas9-BE4 | NGA | Stop codon | GGAGCAGGCCCAGGCAUACG |
| | | | (SEQ ID NO: 538) |
| NGA-SpCas9-BE4 | NGA | Stop codon | AGAGCACCAAGACAGAGCCC |
| | | | (SEQ ID NO: 539) |
| NGA-SpCas9-BE4 | NGA | Stop codon | AGACAUCAAAGUACCCUACA |
| | | | (SEQ ID NO: 540) |
| NGA-SpCas9-BE4 | NGA | Stop codon | CAUCAAAGUACCCUACAGGA |
| | | | (SEQ ID NO: 541) |
| NGA-SpCas9-BE4 | NGA | Stop codon | GAGGACCAGUUCCCAUCCGC |
| | | | (SEQ ID NO: 542) |
| NGA-SpCas9-BE4 | NGA | Stop codon | GCUCCCGCAGUACCUAGCAU |
| | | | (SEQ ID NO: 543) |
| NGA-SpCas9-BE4 | NGA | Stop codon | CAGGAAGCAGAAGGUGCUUG |
| | | | (SEQ ID NO: 544) |
| NGA-SpCas9-BE4 | NGA | Stop codon | AUUUGGCAGCACGUGGUACA |
| | | | (SEQ ID NO: 545) |
| NGA-SpCas9-BE4 | NGA | Stop codon | GCGGGCCAAGACUUCUCCCU |
| | | | (SEQ ID NO: 546) |
| NGA-SpCas9-BE4 | NGA | Stop codon | GUCCCUGCAGCAGCAUGGGG |
| | | | (SEQ ID NO: 547) |
| NGA-SpCas9-BE4 | NGA | Stop codon | GCUACUUCAGGCAGCAGAGG |
| | | | (SEQ ID NO: 548) |
| NGA-SpCas9-BE4 | NGA | Stop codon | CUUGUGCAGACUCAGAGGUG |
| | | | (SEQ ID NO: 549) |
| NGA-SpCas9-BE4 | NGA | Stop codon | GUGCAGACUCAGAGGUGAGA |
| | | | (SEQ ID NO: 550) |
| NGA-SpCas9-BE4 | NGA | Stop codon | GCAGACUCAGAGGUGAGAGG |
| | | | (SEQ ID NO: 551) |
| NGA-SpCas9-BE4 | NGA | Stop codon | UUCCCCCAGCUGAAGUCCUU |
| | | | (SEQ ID NO: 552) |
| NGA-SpCas9-BE4 | NGA | Stop codon | CUGUCCCAGAACAACAUCAC |
| | | | (SEQ ID NO: 553) |
| NGA-SpCas9-BE4 | NGA | Stop codon | CCUGCAACAACAGGAUUCAC |
| | | | (SEQ ID NO: 554) |
| NGA-SpCas9-BE4 | NGA | Stop codon | CGUCCACAUCCUGCAAGGGG |
| | | | (SEQ ID NO: 555) |
| NGA-SpCas9-BE4 | NGA | Splice site | ACAUCCUGCAAGGGGGGAUG |
| | | | (SEQ ID NO: 556) |
| NGA-SpCas9-BE4 | NGA | Splice site | CUUACGCCAGCGUCUCCACA |
| | | | (SEQ ID NO: 557) |
| NGA-SpCas9-BE4 | NGA | Splice site | ACUCACUCCAUCACCCGGAG |
| | | | (SEQ ID NO: 558) |
| NGA-SpCas9-BE4 | NGA | Splice site | ACUCACCUUAGCCUGAGCAG |
| | | | (SEQ ID NO: 559) |
| NGA-SpCas9-BE4 | NGA | Splice site | GACAGACUGCGGGGACACAG |
| | | | (SEQ ID NO: 560) |
| NGA-SpCas9-BE4 | NGA | Splice site | ACUCACUUGAGGGUUUCCAA |
| | | | (SEQ ID NO: 561) |
| NGA-SpCas9-BE4 | NGA | Splice site | UCCAGGCUGCAGGUGGAAUC |
| | | | (SEQ ID NO: 562) |
| NGA-SpCas9-BE4 | NGA | Splice site | ACUCACUCCAGAUGCUGCAG |
| | | | (SEQ ID NO: 563) |
| NGA-SpCas9-BE4 | NGA | Stop codon | AGCCAGCCACAGGGCCCCCA |
| | | | (SEQ ID NO: 564) |
| NGA-SpCas9-BE4 | NGA | Stop codon | GCCCAGGUCCUCACGUCUGC |
| | | | (SEQ ID NO: 565) |
| NGA-SpCas9-BE4 | NGA | Stop codon | CAGCCCAAUAGCUCUUGCCC |
| | | | (SEQ ID NO: 566) |
| NGA-SpCas9-BE4 | NGA | Stop codon | GGCCAUUUUGGAAGCUUGUU |
| | | | (SEQ ID NO: 567) |
| NGA-SpCas9-BE4 | NGA | Splice site | UUACCUGUCAUGUUUGCUCG |
| | | | (SEQ ID NO: 568) |
| NGA-SpCas9-BE4 | NGA | Splice site | ACCUCACCUACAUUGGGGGU |
| | | | (SEQ ID NO: 569) |
| NGA-SpCas9-BE4 | NGA | Splice site | CUCACCUACAUUGGGGGUGG |
| | | | (SEQ ID NO: 570) |
| NGA-SpCas9-BE4 | NGA | Stop codon | UUUGCCAGAGCCCAUGGGGC |
| | | | (SEQ ID NO: 571) |
| NGA-SpCas9-BE4 | NGA | Splice site | AAGGCUGCAGAGAAAACAUG |
| | | | (SEQ ID NO: 572) |
| NGA-SpCas9-BE4 | NGA | Splice site | GCUCUACUUUGAGAAAAACC |
| | | | (SEQ ID NO: 573) |
| NGA-SpCas9-BE4 | NGC | Splice site | CUCCCAGGCAGCUCACAGUG |
| | | | (SEQ ID NO: 574) |
| NGA-SpCas9-BE4 | NGC | Splice site | CUCUGCAGCCUUCCCAGAGG |
| | | | (SEQ ID NO: 575) |
| NGA-SpCas9-BE4 | NGC | Splice site | AAUGUAGGUGAGGUGCCCCA |
| | | | (SEQ ID NO: 576) |
| NGA-SpCas9-BE4 | NGC | Splice site | CCGACAGCUUGUACAAUAAC |
| | | | (SEQ ID NO: 577) |
| NGA-SpCas9-BE4 | NGC | Splice site | CUCACCUCUGAGUCUGCACA |
| | | | (SEQ ID NO: 578) |
| NGA-SpCas9-BE4 | NGC | Splice site | ACUCACCAGGCCAUUUUGGA |
| | | | (SEQ ID NO: 579) |
| NGA-SpCas9-BE4 | NGC | Splice site | CCUGCACACCUGGCUUCCAG |
| | | | (SEQ ID NO: 580) |
| NGA-SpCas9-BE4 | NGC | Splice site | AAACUUACUGAAAAUGUCCU |
| | | | (SEQ ID NO: 581) |
| NGA-SpCas9-BE4 | NGC | Splice site | UCCUCACCGAUAUUGGCAUA |
| | | | (SEQ ID NO: 582) |
| NGA-SpCas9-AIBE | NGC | Splice site | CUCCCAGGCAGCUCACAGUG |
| | | | (SEQ ID NO: 583) |
| NGA-SpCas9-AIBE | NGC | Splice site | CUCUGCAGCCUUCCCAGAGG |
| | | | (SEQ ID NO: 584) |
| NGA-SpCas9-AIBE | NGC | Splice site | CACCCCCAAUGUAGGUGAGG |
| | | | (SEQ ID NO: 585) |
| NGA-SpCas9-AIBE | NGC | Splice site | AAUGUAGGUGAGGUGCCCCA |
| | | | (SEQ ID NO: 586) |
| NGA-SpCas9-AIBE | NGC | Splice site | GGCCUUUGCAGAGCCGGUGG |
| | | | (SEQ ID NO: 587) |
| NGA-SpCas9-AIBE | NGC | Splice site | CCCUCCACAGGGCUGCCUUG |
| | | | (SEQ ID NO: 588) |
| NGA-SpCas9-AIBE | NGC | Splice site | GAUUCCACCUGCAGCCUGGA |
| | | | (SEQ ID NO: 589) |
| NGA-SpCas9-AIBE | NGC | Splice site | UUCCACCUGCAGCCUGGAUG |
| | | | (SEQ ID NO: 590) |
| NGA-SpCas9-AIBE | NGC | Splice site | CCGACAGCUUGUACAAUAAC |
| | | | (SEQ ID NO: 591) |
| NGA-SpCas9-AIBE | NGC | Splice site | CCCCCCUUGCAGGAUGUGGA |
| | | | (SEQ ID NO: 592) |
| NGA-SpCas9-AIBE | NGC | Splice site | GCCCCACUCACCUUAGCCUG |
| | | | (SEQ ID NO: 593) |
| NGA-SpCas9-AIBE | NGC | Splice site | GAGUCUAUACUCACUCCAGA |
| | | | (SEQ ID NO: 594) |
| NGA-SpCas9-AIBE | NGC | Splice site | UCUAUACUCACUCCAGAUGC |
| | | | (SEQ ID NO: 595) |
| NGA-SpCas9-AIBE | NGC | Splice site | UCUCCUCUCACCUCUGAGUC |
| | | | (SEQ ID NO: 596) |
| NGA-SpCas9-AIBE | NGC | Splice site | CUCACCUCUGAGUCUGCACA |
| | | | (SEQ ID NO: 597) |
| NGA-SpCas9-AIBE | NGC | Splice site | ACUCACCAGGCCAUUUUGGA |
| | | | (SEQ ID NO: 598) |
| NGA-SpCas9-AIBE | NGC | Splice site | GGGUCCUUACCUGUCAUGUU |
| | | | (SEQ ID NO: 599) |
| NGA-SpCas9-AIBE | NGC | Splice site | CCUGCACACCUGGCUUCCAG |
| | | | (SEQ ID NO: 600) |
| NGA-SpCas9-AIBE | NGC | Splice site | AAACUUACUGAAAAUGUCCU |
| | | | (SEQ ID NO: 601) |
| NGA-SpCas9-AIBE | NGC | Splice site | GGUGCUUCCUCACCGAUAUU |
| | | | (SEQ ID NO: 602) |
| NGA-SpCas9-AIBE | NGC | Splice site | UCCUCACCGAUAUUGGCAUA |
| | | | (SEQ ID NO: 603) |
| NGA-SpCas9-AIBE | NGC | Splice site | AGGAGGGCCCACCUGAGUAG |
| | | | (SEQ ID NO: 604) |
| NGC-SpCas9-BE4 | NGC | Stop codon | ACUCCCAGCUGGAGGGCCUG |
| | | | (SEQ ID NO: 605) |
| NGC-SpCas9-BE4 | NGC | Stop codon | AGUCAGAAAAGACGUGAGUG |
| | | | (SEQ ID NO: 606) |
| NGC-SpCas9-BE4 | NGC | Stop codon | UCAACCAGGAGCCAGCCUCC |
| | | | (SEQ ID NO: 607) |
| NGC-SpCas9-BE4 | NGC | Stop codon | GGAGCAGUUCUACCGCUCAC |
| | | | (SEQ ID NO: 608) |
| NGC-SpCas9-BE4 | NGC | Stop codon | UCACUGCAGGACACGUAUGG |
| | | | (SEQ ID NO: 609) |
| NGC-SpCas9-BE4 | NGC | Stop codon | AACGGCAGCUGGCCCAAGGA |
| | | | (SEQ ID NO: 610) |
| NGC-SpCas9-BE4 | NGC | Stop codon | UGAGACACGAGUGAUUGCUG |
| | | | (SEQ ID NO: 611) |
| NGC-SpCas9-BE4 | NGC | Stop codon | GGUCAGGGCAAGAGCUAUUG |
| | | | (SEQ ID NO: 612) |
| NGC-SpCas9-BE4 | NGC | Stop codon | GGCCCACAGCCACUCGUGGC |
| | | | (SEQ ID NO: 613) |
| NGC-SpCas9-BE4 | NGC | Stop codon | GGAAGCGCAAGAUGGCUUCC |
| | | | (SEQ ID NO: 614) |
| NGC-SpCas9-BE4 | NGC | Stop codon | CUGGUCCAGAGCCUGAGCAA |
| | | | (SEQ ID NO: 615) |
| NGC-SpCas9-BE4 | NGC | Stop codon | GCAGGCCCAGGCAUACGUGA |
| | | | (SEQ ID NO: 616) |
| NGC-SpCas9-BE4 | NGC | Stop codon | AGGCCCAGGCAUACGUGAUG |
| | | | (SEQ ID NO: 617) |
| NGC-SpCas9-BE4 | NGC | Stop codon | GCACCAAGACAGAGCCCUGA |
| | | | (SEQ ID NO: 618) |
| NGC-SpCas9-BE4 | NGC | Stop codon | GUGCCAGCUCUCAGAGGCCC |
| | | | (SEQ ID NO: 619) |
| NGC-SpCas9-BE4 | NGC | Stop codon | UUAGUCCAACACCCACCGCG |
| | | | (SEQ ID NO: 620) |
| NGC-SpCas9-BE4 | NGC | Stop codon | GUCCAACACCCACCGCGGGC |
| | | | (SEQ ID NO: 621) |
| NGC-SpCas9-BE4 | NGC | Stop codon | CUCCUGCAAUGCUUCCUGGG |
| | | | (SEQ ID NO: 622) |
| NGC-SpCas9-BE4 | NGC | Stop codon | UCUUCCAGCCUCCCGCCCGC |
| | | | (SEQ ID NO: 623) |
| NGC-SpCas9-BE4 | NGC | Stop codon | GCGGCUGCAGCCGGGGACAC |
| | | | (SEQ ID NO: 624) |
| NGC-SpCas9-BE4 | NGC | Stop codon | CUGCAGCCGGGGACACUGCG |
| | | | (SEQ ID NO: 625) |
| NGC-SpCas9-BE4 | NGC | Stop codon | GGCGCGGCAGCUGCUGGAGC |
| | | | (SEQ ID NO: 626) |
| NGC-SpCas9-BE4 | NGC | Stop codon | GCGGCAGCUGCUGGAGCUGC |
| | | | (SEQ ID NO: 627) |
| NGC-SpCas9-BE4 | NGC | Stop codon | UUGGCAGCACGUGGUACAGG |
| | | | (SEQ ID NO: 628) |
| NGC-SpCas9-BE4 | NGC | Stop codon | UGGUACAGGAGCUCCCCGGC |
| | | | (SEQ ID NO: 629) |
| NGC-SpCas9-BE4 | NGC | Stop codon | GCAGCAGCAUGGGGAGACCA |
| | | | (SEQ ID NO: 630) |
| NGC-SpCas9-BE4 | NGC | Stop codon | GACUCAGAGGUGAGAGGAGA |
| | | | (SEQ ID NO: 631) |
| NGC-SpCas9-BE4 | NGC | Stop codon | GUCCAGUACAACAAGUUCAC |
| | | | (SEQ ID NO: 632) |
| NGC-SpCas9-BE4 | NGC | Stop codon | GGGCCCAGCAGCUCGCUGCC |
| | | | (SEQ ID NO: 633) |
| NGC-SpCas9-BE4 | NGC | Stop codon | AACAACAGGAUUCACGGAUC |
| | | | (SEQ ID NO: 634) |
| NGC-SpCas9-BE4 | NGC | Splice site | UACAAGCUGUCGGAAACAGA |
| | | | (SEQ ID NO: 635) |
| NGC-SpCas9-BE4 | NGC | Splice site | GCCCAGCCUAGGAGGCAAAG |
| | | | (SEQ ID NO: 636) |
| NGC-SpCas9-BE4 | NGC | Splice site | CUCACCUCUGAGUCUGCACA |
| | | | (SEQ ID NO: 637) |
| NGC-SpCas9-BE4 | NGC | Stop codon | CUCCCACAGCGCCACCGUGU |
| | | | (SEQ ID NO: 638) |
| NGC-SpCas9-BE4 | NGC | Splice site | CCACCUGAAACGGGUGACAC |
| | | | (SEQ ID NO: 639) |
| NGC-SpCas9-BE4 | NGC | Stop codon | UGCCAAAUUCCAGCCUCCUC |
| | | | (SEQ ID NO: 640) |
| NGC-SpCas9-BE4 | NGC | Stop codon | CCUCCAGCCAGUUGUCAUAG |
| | | | (SEQ ID NO: 641) |
| NGC-SpCas9-BE4 | NGC | Stop codon | CAGCCACAGGGCCCCCAGGA |
| | | | (SEQ ID NO: 642) |
| NGC-SpCas9-BE4 | NGC | Stop codon | AUCGCCCAGGUCCUCACGUC |
| | | | (SEQ ID NO: 643) |
| NGC-SpCas9-BE4 | NGC | Stop codon | GCUCCCAGGCCAGCUUGGCC |
| | | | (SEQ ID NO: 644) |
| NGC-SpCas9-BE4 | NGC | Stop codon | CAAGCCCAGGCCCGGCUCAC |
| | | | (SEQ ID NO: 645) |
| NGC-SpCas9-BE4 | NGC | Splice site | CCGGCUCUGCAAAGGCCAGG |
| | | | (SEQ ID NO: 646) |
| NGC-SpCas9-BE4 | NGC | Splice site | ACUCACCAGGCCAUUUUGGA |
| | | | (SEQ ID NO: 647) |
| NGC-SpCas9-BE4 | NGC | Splice site | UUGUGCUCUGGAGAUGGAGA |
| | | | (SEQ ID NO: 648) |
| NGC-SpCas9-BE4 | NGC | Stop codon | AGAUUUGCCAGAGCCCAUGG |
| | | | (SEQ ID NO: 649) |
| NGC-SpCas9-BE4 | NGC | Splice site | AAGGCACUGCAAGAGACAAA |
| | | | (SEQ ID NO: 650) |
| NGC-SpCas9-BE4 | NGC | Splice site | GGCUCAGCUGUGAGGAAGUG |
| | | | (SEQ ID NO: 651) |
| NGC-SpCas9-BE4 | NGC | Stop codon | GGCUUCCAGUGCUUCAGGUC |
| | | | (SEQ ID NO: 652) |
| NGC-SpCas9-BE4 | NGC | Splice site | AAACUUACUGAAAAUGUCCU |
| | | | (SEQ ID NO: 653) |
| NGC-SpCas9-BE4 | NGC | Splice site | CCGCUGCCCAGACAAGGAAA |
| | | | (SEQ ID NO: 654) |
| NGC-SpCas9-BE4 | NGC | Splice site | UCCUCACCGAUAUUGGCAUA |
| | | | (SEQ ID NO: 655) |
| NGC-SpCas9-BE4 | NGC | Splice site | CTGCCUGGGAGGGAAGACAA |
| | | | (SEQ ID NO: 656) |
| SaCas9-ABE | NNGRRT | Splice site | CCACCUGCAGCCUGGAUGCG |
| | | | (SEQ ID NO: 657) |
| SaCas9-ABE | NNGRRT | Splice site | CCACUCACCUUAGCCUGAGC |
| | | | (SEQ ID NO: 658) |
| SaCas9-ABE | NGG | Splice site | CACAGCUGAGCCCCCCACUG |
| | | | (SEQ ID NO: 659) |
| SaCas9-ABE | NGG | Splice site | CAAUGUAGGUGAGGUGCCCC |
| | | | (SEQ ID NO: 660) |
| SaCas9-ABE | NGG | Splice site | CUCCAGGACGAGAAGUUCCU |
| | | | (SEQ ID NO: 661) |
| SaCas9-ABE | NGG | Splice site | CCUAGGCUGGGCCCUGUCUC |
| | | | (SEQ ID NO: 662) |
| SaCas9-ABE | NGG | Splice site | ACACUCACUCCAUCACCCGG |
| | | | (SEQ ID NO: 663) |
| SaCas9-ABE | NGG | Splice site | CACUCACUCCAUCACCCGGA |
| | | | (SEQ ID NO: 664) |
| SaCas9-ABE | NGG | Splice site | CCACUCACCUUAGCCUGAGC |
| | | | (SEQ ID NO: 665) |
| SaCas9-ABE | NGG | Splice site | CACUCACCUUAGCCUGAGCA |
| | | | (SEQ ID NO: 666) |
| SaCas9-ABE | NGG | Splice site | CACUCACUUGAGGGUUUCCA |
| | | | (SEQ ID NO: 667) |
| SaCas9-ABE | NGG | Splice site | AUACUCACUCCAGAUGCUGC |
| | | | (SEQ ID NO: 668) |
| SaCas9-ABE | NGG | Splice site | UACUCACUCCAGAUGCUGCA |
| | | | (SEQ ID NO: 669) |
| SaCas9-ABE | NGG | Splice site | CCUUACCUGUCAUGUUUGCU |
| | | | (SEQ ID NO: 670) |
| SaCas9-ABE | NGG | Splice site | CUUACCUGUCAUGUUUGCUC |
| | | | (SEQ ID NO: 671) |
| SaCas9-ABE | NGG | Splice site | UAACAUACUGGGAAUCUGGU |
| | | | (SEQ ID NO: 672) |
| SaCas9-ABE | NGG | Splice site | UUUUACCUUGGGGCUCUGAC |
| | | | (SEQ ID NO: 673) |
| SaCas9-AIBE | NGG | Splice site | CCUUGUCUGGGCAGCGGAAC |
| | | | (SEQ ID NO: 674) |
| SaCas9-AIBE | NGG | Splice site | UUUCUCAAAGUAGAGCACAU |
| | | | (SEQ ID NO: 675) |
| SaCas9-AIBE | NGG | Splice site | UUUCUCUGCAGCCUUCCCAG |
| | | | (SEQ ID NO: 676) |
| SaCas9-AIBE | NGG | Splice site | CACAGCUGAGCCCCCCACUG |
| | | | (SEQ ID NO: 677) |
| SaCas9-AIBE | NGG | Splice site | CAAUGUAGGUGAGGUGCCCC |
| | | | (SEQ ID NO: 678) |
| SaCas9-AIBE | NGG | Splice site | CCUGGCCUUUGCAGAGCCGG |
| | | | (SEQ ID NO: 679) |
| SaCas9-AIBE | NGG | Splice site | CUCCAGGACGAGAAGUUCCU |
| | | | (SEQ ID NO: 680) |
| SaCas9-AIBE | NGG | Splice site | CCUAGGCUGGGCCCUGUCUC |
| | | | (SEQ ID NO: 681) |
| SaCas9-AIBE | NGG | Splice site | CCCACACUCACUCCAUCACC |
| | | | (SEQ ID NO: 682) |
| SaCas9-AIBE | NGG | Splice site | ACACUCACUCCAUCACCCGG |
| | | | (SEQ ID NO: 683) |
| SaCas9-AIBE | NGG | Splice site | CACUCACUCCAUCACCCGGA |
| | | | (SEQ ID NO: 684) |
| SaCas9-AIBE | NGG | Splice site | CCACUCACCUUAGCCUGAGC |
| | | | (SEQ ID NO: 685) |
| SaCas9-AIBE | NGG | Splice site | CACUCACCUUAGCCUGAGCA |
| | | | (SEQ ID NO: 686) |
| SaCas9-AIBE | NGG | Splice site | CACUCACUUGAGGGUUUCCA |
| | | | (SEQ ID NO: 687) |
| SaCas9-AIBE | NGG | Splice site | AUACUCACUCCAGAUGCUGC |
| | | | (SEQ ID NO: 688) |
| SaCas9-AIBE | NGG | Splice site | UACUCACUCCAGAUGCUGCA |
| | | | (SEQ ID NO: 689) |
| SaCas9-AIBE | NGG | Splice site | GCCCCUCACCCCACCUGAAA |
| | | | (SEQ ID NO: 690) |
| SaCas9-AIBE | NGG | Splice site | CCCCUCACCCCACCUGAAAC |
| | | | (SEQ ID NO: 691) |
| SaCas9-AIBE | NGG | Splice site | CAUCACUCACCAGGCCAUUU |
| | | | (SEQ ID NO: 692) |
| SaCas9-AIBE | NGG | Splice site | CCUUACCUGUCAUGUUUGCU |
| | | | (SEQ ID NO: 693) |
| SaCas9-AIBE | NGG | Splice site | CUUACCUGUCAUGUUUGCUC |
| | | | (SEQ ID NO: 694) |
| SaCas9-AIBE | NGG | Splice site | CCCCUAACAUACUGGGAAUC |
| | | | (SEQ ID NO: 695) |
| SaCas9-AIBE | NGG | Splice site | UAACAUACUGGGAAUCUGGU |
| | | | (SEQ ID NO: 696) |
| SaCas9-AIBE | NGG | Splice site | AGGUGCUUCCUCACCGAUAU |
| | | | (SEQ ID NO: 697) |
| SaCas9-AIBE | NGG | Splice site | UUUUACCUUGGGGCUCUGAC |
| | | | (SEQ ID NO: 698) |
| SpCas9-BE4 | NGG | Stop codon | GAGCCCCAAGGUAAAAAGGC |
| | | | (SEQ ID NO: 699) |
| SpCas9-BE4 | NGG | Stop codon | AGCCCCAAGGUAAAAAGGCC |
| | | | (SEQ ID NO: 700) |
| SpCas9-BE4 | NGG | Stop codon | CAGCUCACAGUGUGCCACCA |
| | | | (SEQ ID NO: 701) |
| SpCas9-BE4 | NGG | Stop codon | UAUGACCAGAUGGACCUGGC |
| | | | (SEQ ID NO: 702) |
| SpCas9-BE4 | NGG | Stop codon | ACUGGACCAGUATGTCUUCC |
| | | | (SEQ ID NO: 703) |
| SpCas9-BE4 | NGG | Stop codon | UGUCUUCCAGGACUCCCAGC |
| | | | (SEQ ID NO: 704) |
| SpCas9-BE4 | NGG | Stop codon | CUUCCAGGACUCCCAGCUGG |
| | | | (SEQ ID NO: 705) |
| SpCas9-BE4 | NGG | Stop codon | UUCCAGGACUCCCAGCUGGA |
| | | | (SEQ ID NO: 706) |
| SpCas9-BE4 | NGG | Stop codon | UUCAACCAGGAGCCAGCCUC |
| | | | (SEQ ID NO: 707) |
| SpCas9-BE4 | NGG | Stop codon | GACCAGAUUCCCAGUAUGUU |
| | | | (SEQ ID NO: 708) |
| SpCas9-BE4 | NGG | Stop codon | CUCUGGCAAAUCUCUGAGGC |
| | | | (SEQ ID NO: 709) |
| SpCas9-BE4 | NGG | Stop codon | AGCCAAGUACCCCCUCCCAG |
| | | | (SEQ ID NO: 710) |
| SpCas9-BE4 | NGG | Stop codon | ACCUCCCGAGCAAACAUGAC |
| | | | (SEQ ID NO: 711) |
| SpCas9-BE4 | NGG | Stop codon | CCCACCCAAUGCCCGGCAGC |
| | | | (SEQ ID NO: 712) |
| SpCas9-BE4 | NGG | Stop codon | UGGUGCAGGCCAGGCUGGAG |
| | | | (SEQ ID NO: 713) |
| SpCas9-BE4 | NGG | Stop codon | GAACGGCAGCUGGCCCAAGG |
| | | | (SEQ ID NO: 714) |
| SpCas9-BE4 | NGG | Stop codon | GGCCCAAGGAGGCCUGGCUG |
| | | | (SEQ ID NO: 715) |
| SpCas9-BE4 | NGG | Stop codon | GACACGAGUGAUUGCUGUGC |
| | | | (SEQ ID NO: 716) |
| SpCas9-BE4 | NGG | Stop codon | ACACGAGUGAUUGCUGUGCU |
| | | | (SEQ ID NO: 717) |
| SpCas9-BE4 | NGG | Stop codon | CUGGUCAGGGCAAGAGCUAU |
| | | | (SEQ ID NO: 718) |
| SpCas9-BE4 | NGG | Stop codon | UGGUCAGGGCAAGAGCUAUU |
| | | | (SEQ ID NO: 719) |
| SpCas9-BE4 | NGG | Stop codon | GGGCCCACAGCCACUCGUGG |
| | | | (SEQ ID NO: 720) |
| SpCas9-BE4 | NGG | Stop codon | UUCCAGAAGAAGCUGCUCCG |
| | | | (SEQ ID NO: 721) |
| SpCas9-BE4 | NGG | Stop codon | CCUGGUCCAGAGCCUGAGCA |
| | | | (SEQ ID NO: 722) |
| SpCas9-BE4 | NGG | Stop codon | CAGACAUCAAAGUACCCUAC |
| | | | (SEQ ID NO: 723) |
| SpCas9-BE4 | NGG | Stop codon | ACAUCAAAGUACCCUACAGG |
| | | | (SEQ ID NO: 724) |
| SpCas9-BE4 | NGG | Stop codon | CUUAGUCCAACACCCACCGC |
| | | | (SEQ ID NO: 725) |
| SpCas9-BE4 | NGG | Stop codon | CCUCCUGCAAUGCUUCCUGG |
| | | | (SEQ ID NO: 726) |
| SpCas9-BE4 | NGG | Stop codon | GGAAGCAGAAGGUGCUUGCG |
| | | | (SEQ ID NO: 727) |
| SpCas9-BE4 | NGG | Stop codon | GGCUGCAGCCGGGGACACUGCGG |
| | | | (SEQ ID NO: 728) |
| SpCas9-BE4 | NGG | Stop codon | GCUGCAGCCGGGGACACUGC |
| | | | (SEQ ID NO: 729) |
| SpCas9-BE4 | NGG | Stop codon | AAUUUGGCAGCACGUGGUAC |
| | | | (SEQ ID NO: 730) |
| SpCas9-BE4 | NGG | Stop codon | GGCGGGCCAAGACUUCUCCC |
| | | | (SEQ ID NO: 731) |
| SpCas9-BE4 | NGG | Stop codon | UGUGCAGACUCAGAGGUGAG |
| | | | (SEQ ID NO: 732) |
| SpCas9-BE4 | NGG | Stop codon | AGACUCAGAGGUGAGAGGAG |
| | | | (SEQ ID NO: 733) |
| SpCas9-BE4 | NGG | Stop codon | CCCCCAGGCUUUCCCCAAAC |
| | | | (SEQ ID NO: 734) |
| SpCas9-BE4 | NGG | Stop codon | CUUCCCCCAGCUGAAGUCCU |
| | | | (SEQ ID NO: 735) |
| SpCas9-BE4 | NGG | Stop codon | CGUCCAGUACAACAAGUUCA |
| | | | (SEQ ID NO: 736) |
| SpCas9-BE4 | NGG | Stop codon | ACCUGCAACAACAGGAUUCA |
| | | | (SEQ ID NO: 737) |
| SpCas9-BE4 | NGG | Stop codon | UGGGCGUCCACAUCCUGCAA |
| | | | (SEQ ID NO: 738) |
| SpCas9-BE4 | NGG | Stop codon | GGGCGUCCACAUCCUGCAAG |
| | | | (SEQ ID NO: 739) |
| SpCas9-BE4 | NGG | Stop codon | GGCGUCCACAUCCUGCAAGG |
| | | | (SEQ ID NO: 740) |
| SpCas9-BE4 | NGG | Stop codon | GCGUCCACAUCCUGCAAGGG |
| | | | (SEQ ID NO: 741) |
| SpCas9-BE4 | NGG | Splice site | CCACAUCCUGCAAGGGGGGA |
| | | | (SEQ ID NO: 742) |
| SpCas9-BE4 | NGG | Splice site | CACAUCCUGCAAGGGGGGAU |
| | | | (SEQ ID NO: 743) |
| SpCas9-BE4 | NGG | Splice site | ACACUCACUCCAUCACCCGG |
| | | | (SEQ ID NO: 744) |
| SpCas9-BE4 | NGG | Splice site | CACUCACUCCAUCACCCGGA |
| | | | (SEQ ID NO: 745) |
| SpCas9-BE4 | NGG | Splice site | GUACAAGCUGUCGGAAACAG |
| | | | (SEQ ID NO: 746) |
| SpCas9-BE4 | NGG | Splice site | CCACUCACCUUAGCCUGAGC |
| | | | (SEQ ID NO: 747) |
| SpCas9-BE4 | NGG | Splice site | CACUCACCUUAGCCUGAGCA |
| | | | (SEQ ID NO: 748) |
| SpCas9-BE4 | NGG | Splice site | CAGACUGCGGGGACACAGUG |
| | | | (SEQ ID NO: 749) |
| SpCas9-BE4 | NGG | Splice site | AGACUGCGGGGACACAGUGA |
| | | | (SEQ ID NO: 750) |
| SpCas9-BE4 | NGG | Splice site | CACUCACUUGAGGGUUUCCA |
| | | | (SEQ ID NO: 751) |
| SpCas9-BE4 | NGG | Splice site | AGGCUGCAGGUGGAAUCAGA |
| | | | (SEQ ID NO: 752) |
| SpCas9-BE4 | NGG | Splice site | AUACUCACUCCAGAUGCUGC |
| | | | (SEQ ID NO: 753) |
| SpCas9-BE4 | NGG | Splice site | UACUCACUCCAGAUGCUGCA |
| | | | (SEQ ID NO: 754) |
| SpCas9-BE4 | NGG | Splice site | UCACUCCAGAUGCUGCAGGG |
| | | | (SEQ ID NO: 755) |
| SpCas9-BE4 | NGG | Splice site | AGCCUAGGAGGCAAAGAGCA |
| | | | (SEQ ID NO: 756) |
| SpCas9-BE4 | NGG | Stop codon | CUGCCAAAUUCCAGCCUCCU |
| | | | (SEQ ID NO: 757) |
| SpCas9-BE4 | NGG | Stop codon | GAGCCAGCCACAGGGCCCCC |
| | | | (SEQ ID NO: 758) |
| SpCas9-BE4 | NGG | Stop codon | CGCCCAGGUCCUCACGUCUG |
| | | | (SEQ ID NO: 759) |
| SpCas9-BE4 | NGG | Splice site | ACCGGCUCUGCAAAGGCCAG |
| | | | (SEQ ID NO: 760) |
| SpCas9-BE4 | NGG | Stop codon | AGGCCAUUUUGGAAGCUUGU |
| | | | (SEQ ID NO: 761) |
| SpCas9-BE4 | NGG | Splice site | UGCUCUGGAGAUGGAGAAGC |
| | | | (SEQ ID NO: 762) |
| SpCas9-BE4 | NGG | Splice site | CCUUACCUGUCAUGUUUGCU |
| | | | (SEQ ID NO: 763) |
| SpCas9-BE4 | NGG | Splice site | CUUACCUGUCAUGUUUGCUC |
| | | | (SEQ ID NO: 764) |
| SpCas9-BE4 | NGG | Splice site | AAAGGCACUGCAAGAGACAA |
| | | | (SEQ ID NO: 765) |
| SpCas9-BE4 | NGG | Splice site | UAACAUACUGGGAAUCUGGU |
| | | | (SEQ ID NO: 766) |
| SpCas9-BE4 | NGG | Stop codon | UUCCAGUGCUUCAGGUCUGC |
| | | | (SEQ ID NO: 767) |
| SpCas9-BE4 | NGG | Splice site | UUUUACCUUGGGGCUCUGAC |
| | | | (SEQ ID NO: 768) |

TABLE 5
Base editor, PAM sequences, guide RNA for HLA-A target gene.

| | Search | Strategy | Full sequence | |
|---|---|---|---|---|
| Editor | PAM | site | (Protospacer underlined) | PAM |
| BE4 | NGG | Splice site | CCTTACCCCATCTCAGGGTGAGG (SEQ ID | AGG |
| | | | NO: 769) | |
| BE4 | NGG | Stop codon | CCTTACCCCATCTCAGGGTGAGG (SEQ ID | AGG |
| | | | NO: 770) | |
| BE4 | NGG | Splice site | CTTACCCCATCTCAGGGTGAGGG (SEQ ID | GGG |
| | | | NO: 771) | |
| BE4 | NGG | Stop codon | CTTACCCCATCTCAGGGTGAGGG (SEQ ID | GGG |
| | | | NO: 772) | |
| BE4 | NGG | Splice site | TTACCCCATCTCAGGGTGAGGGG (SEQ | GGG |
| | | | ID NO: 773) | |
| BE4 | NGG | Stop codon | TTACCCCATCTCAGGGTGAGGGG (SEQ | GGG |
| | | | ID NO: 774) | |
| BE4 | NGG | Stop codon | CAGGGCCCAGCACCTCAGGGTGG (SEQ | TGG |
| | | | ID NO: 775) | |
| SpCas9-ABE | NGG | Splice site | CCCCAGGCTCCCACTCCATGAGG (SEQ ID | AGG |
| | | | NO: 776) | |
| SpCas9-ABE | NGG | Splice site | CCTTACCCCATCTCAGGGTGAGG (SEQ ID | AGG |
| | | | NO: 777) | |
| SpCas9-ABE | NGG | Splice site | CTTACCCCATCTCAGGGTGAGGG (SEQ ID | GGG |
| | | | NO: 778) | |
| SpCas9-ABE | NGG | Splice site | GTCACTCACCGGCCTCGCTCTGG (SEQ ID | TGG |
| | | | NO: 779) | |
| NGA-SpCas9-ABE | NGA | Splice site | CTCCTTACCCCATCTCAGGGTGA (SEQ ID | TGA |
| | | | NO: 780) | |
| SpCas9-AIBE | NGG | Splice site | CCCCAGGCTCCCACTCCATGAGG (SEQ ID | AGG |
| | | | NO: 781) | |
| SpCas9-AIBE | NGG | Splice site | CCTTACCCCATCTCAGGGTGAGG (SEQ ID | AGG |
| | | | NO: 782) | |
| SpCas9-AIBE | NGG | Splice site | CTTACCCCATCTCAGGGTGAGGG (SEQ ID | GGG |
| | | | NO: 783) | |
| SpCas9-AIBE | NGG | Splice site | GTCACTCACCGGCCTCGCTCTGG (SEQ ID | TGG |
| | | | NO: 784) | |
| NGC-SpCas9-AIBE | NGC | Splice site | CCGGGGTCACTCACCGGCCTCGC (SEQ ID | CGC |
| | | | NO: 785) | |
| SaCas9-CBE | NNGRRT | Stop codon | CTACAACCAGAGCGAGGCCGGTGAGT | GTGAGT |
| | | | (SEQ ID NO: 786) | |
| KKH-SaCas9-ABE | NNNRRT | Splice site | GGGTCACTCACCGGCCTCGCTCTGGT | TCTGGT |
| | | | (SEQ ID NO: 787) | |

TABLE 5A
Spacer sequences for HLA-A target gene.

| | Search | Strategy | Full sequence |
|---|---|---|---|
| Editor | PAM | site | (Protospacer underlined) |
| BE4 | NGG | Splice site | CCUUACCCCAUCUCAGGGUG (SEQ ID |
| | | | NO: 788) |
| BE4 | NGG | Stop codon | CCUUACCCCAUCUCAGGGUG (SEQ ID |
| | | | NO: 789) |
| BE4 | NGG | Splice site | CUUACCCCAUCUCAGGGUGA (SEQ ID |
| | | | NO: 790) |
| BE4 | NGG | Stop codon | CUUACCCCAUCUCAGGGUGA (SEQ ID |
| | | | NO: 791) |
| BE4 | NGG | Splice site | UUACCCCAUCUCAGGGUGAG (SEQ ID |
| | | | NO: 792) |
| BE4 | NGG | Stop codon | UUACCCCAUCUCAGGGUGAG (SEQ ID |
| | | | NO: 793) |
| BE4 | NGG | Stop codon | CAGGGCCCAGCACCUCAGGG (SEQ ID |
| | | | NO: 794) |
| SpCas9-ABE | NGG | Splice site | CCCCAGGCUCCCACUCCAUG (SEQ ID |
| | | | NO: 795) |
| SpCas9-ABE | NGG | Splice site | CCUUACCCCAUCUCAGGGUG (SEQ ID |
| | | | NO: 796) |
| SpCas9-ABE | NGG | Splice site | CUUACCCCAUCUCAGGGUGA (SEQ ID |
| | | | NO: 797) |
| SpCas9-ABE | NGG | Splice site | GUCACUCACCGGCCUCGCUC (SEQ ID |
| | | | NO: 798) |
| NGA-SpCas9-ABE | NGA | Splice site | CUCCUUACCCCAUCUCAGGG (SEQ ID |
| | | | NO: 799) |
| SpCas9-AIBE | NGG | Splice site | CCCCAGGCUCCCACUCCAUG (SEQ ID |
| | | | NO: 800) |
| SpCas9-AIBE | NGG | Splice site | CCUUACCCCAUCUCAGGGUG (SEQ ID |
| | | | NO: 801) |
| SpCas9-AIBE | NGG | Splice site | CUUACCCCAUCUCAGGGUGA (SEQ ID |
| | | | NO: 802) |
| SpCas9-AIBE | NGG | Splice site | GUCACUCACCGGCCUCGCUC (SEQ ID |
| | | | NO: 803) |
| NGC-SpCas9-AIBE | NGC | Splice site | CCGGGGUCACUCACCGGCCU (SEQ ID |
| | | | NO: 804) |
| SaCas9-CBE | NNGRRT | Stop codon | CUACAACCAGAGCGAGGCCG (SEQ ID |
| | | | NO: 805) |
| KKH-SaCas9-ABE | NNNRRT | Splice site | GGGUCACUCACCGGCCUCGC (SEQ ID |
| | | | NO: 806) |

TABLE 6
Base editor, PAM sequences, guide RNA for HLA-B target gene.

| | Search | Strategy | Full sequence | |
|---|---|---|---|---|
| Editor | PAM | site | (Protospacer underlined) | PAM |
| BE4 | NGG | Splice site | CCTTACCCCATCTCAGGGTGAGG (SEQ ID | AGG |
| | | | NO: 807) | |
| BE4 | NGG | Stop codon | CCTTACCCCATCTCAGGGTGAGG (SEQ ID | AGG |
| | | | NO: 808) | |
| BE4 | NGG | Splice site | CTTACCCCATCTCAGGGTGAGGG (SEQ ID | GGG |
| | | | NO: 809) | |
| BE4 | NGG | Stop codon | CTTACCCCATCTCAGGGTGAGGG (SEQ ID | GGG |
| | | | NO: 810) | |
| BE4 | NGG | Splice site | TTACCCCATCTCAGGGTGAGGGG (SEQ | GGG |
| | | | ID NO: 811) | |
| BE4 | NGG | Stop codon | TTACCCCATCTCAGGGTGAGGGG (SEQ | GGG |
| | | | ID NO: 812) | |
| BE4 | NGG | Stop codon | CAGGGCCCAGCACCTCAGGGTGG (SEQ | TGG |
| | | | ID NO: 813) | |
| SpCas9-ABE | NGG | Splice site | CCCCAGGCTCCCACTCCATGAGG (SEQ ID | AGG |
| | | | NO: 814) | |
| SpCas9-ABE | NGG | Splice site | CCTTACCCCATCTCAGGGTGAGG (SEQ ID | GGG |
| | | | NO: 815) | |
| SpCas9-ABE | NGG | Splice site | CTTACCCCATCTCAGGGTGAGGG (SEQ ID | TGG |
| | | | NO: 816) | |
| SpCas9-ABE | NGG | Splice site | GTCACTCACCGGCCTCGCTCTGG (SEQ ID | AGG |
| | | | NO: 817) | |
| NGA-SpCas9-ABE | NGA | Splice site | CTCCTTACCCCATCTCAGGGTGA (SEQ ID | TGA |
| | | | NO: 818) | |
| SpCas9-AIBE | NGG | Splice site | CCCCAGGCTCCCACTCCATGAGG (SEQ ID | AGG |
| | | | NO: 819) | |
| SpCas9-AIBE | NGG | Splice site | CTTACCCCATCTCAGGGTGAGGG (SEQ ID | GGG |
| | | | NO: 820) | |
| SpCas9-AIBE | NGG | Splice site | GTCACTCACCGGCCTCGCTCTGG (SEQ ID | TGG |
| | | | NO: 821) | |
| SpCas9-AIBE | NGG | Splice site | CCCCAGGCTCCCACTCCATGAGG (SEQ ID | AGG |
| | | | NO: 822) | |
| NGC-SpCas9-AIBE | NGC | Splice site | CCGGGGTCACTCACCGGCCTCGC (SEQ ID | CGC |
| | | | NO: 823) | |
| SaCas9-CBE | NNGRRT | Stop codon | CTACAACCAGAGCGAGGCCGGTGAGT | GTGAGT |
| | | | (SEQ ID NO: 824) | |
| KKH-SaCas9-ABE | NNNRRT | Splice site | GGGTCACTCACCGGCCTCGCTCTGGT | TCTGGT |
| | | | (SEQ ID NO: 825) | |

TABLE 6A
Spacer sequences for HLA-B target gene.

| | Search | Strategy | Full sequence |
|---|---|---|---|
| Editor | PAM | site | (Protospacer underlined) |
| BE4 | NGG | Splice site | CCUUACCCCAUCUCAGGGUG (SEQ ID |
| | | | NO: 826) |
| BE4 | NGG | Stop codon | CCUUACCCCAUCUCAGGGUG (SEQ ID |
| | | | NO: 827) |
| BE4 | NGG | Splice site | CUUACCCCAUCUCAGGGUGA (SEQ ID |
| | | | NO: 828) |
| BE4 | NGG | Stop codon | CUUACCCCAUCUCAGGGUGA (SEQ ID |
| | | | NO: 829) |
| BE4 | NGG | Splice site | UUACCCCAUCUCAGGGUGAG (SEQ ID |
| | | | NO: 830) |
| BE4 | NGG | Stop codon | UUACCCCAUCUCAGGGUGAG (SEQ ID |
| | | | NO: 831) |
| BE4 | NGG | Stop codon | CAGGGCCCAGCACCUCAGGG (SEQ ID |
| | | | NO: 832) |
| SpCas9-ABE | NGG | Splice site | CCCCAGGCUCCCACUCCAUG (SEQ ID |
| | | | NO: 833) |
| SpCas9-ABE | NGG | Splice site | CCUUACCCCAUCUCAGGGUG (SEQ ID |
| | | | NO: 834) |
| SpCas9-ABE | NGG | Splice site | CUUACCCCAUCUCAGGGUGA (SEQ ID |
| | | | NO: 835) |
| SpCas9-ABE | NGG | Splice site | GUCACUCACCGGCCUCGCUC (SEQ ID |
| | | | NO: 836) |
| NGA-SpCas9-ABE | NGA | Splice site | CUCCUUACCCCAUCUCAGGG (SEQ ID |
| | | | NO: 837) |
| SpCas9-AIBE | NGG | Splice site | CCCCAGGCUCCCACUCCAUG (SEQ ID |
| | | | NO: 838) |
| SpCas9-AIBE | NGG | Splice site | CUUACCCCAUCUCAGGGUGA (SEQ ID |
| | | | NO: 839) |
| SpCas9-AIBE | NGG | Splice site | GUCACUCACCGGCCUCGCUC (SEQ ID |
| | | | NO: 840) |
| SpCas9-AIBE | NGG | Splice site | CCCCAGGCUCCCACUCCAUG (SEQ ID |
| | | | NO: 841) |
| NGC-SpCas9-AIBE | NGC | Splice site | CCGGGGUCACUCACCGGCCU (SEQ ID |
| | | | NO: 842) |
| SaCas9-CBE | NNGRRT | Stop codon | CUACAACCAGAGCGAGGCCG (SEQ ID |
| | | | NO: 843) |
| KKH-SaCas9-ABE | NNNRRT | Splice site | GGGUCACUCACCGGCCUCGC (SEQ ID |
| | | | NO: 844) |

### Example 4. Large-Scale Production of Base-Edited Human Hepatocytes

This example illustrates large-scale production of base-edited human hepatocytes.

Cryopreserved primary hepatocytes or plateable/engraftable primary hepatocytes will be obtained. Multiplexed gene editing will be carried out on hepatocytes as described in Examples 1 and 2.

Modified human hepatocytes produced will be validated by measuring A-to-G and C-to-T base conversion.

Modified human hepatocytes will be introduced in FRG mice and expanded for large scale production.

About 200-500 million cells will be engrafted in FRG pigs, either directly from primary human hepatocyte culture or from FRG mice.

The results of this example will produce large scale base-edited human hepatocytes that abolish or reduce host immune reaction for liver transplantation.

### Example 5. Evaluating Engraftment of Base-Edited Hepatocytes in a FRG Mouse Model of Liver Failure and Metabolic Disease

This example illustrates engraftment and base-edited hepatocyte retention in Fah^{-/-}/Rag2^{-/-}/Il2rg^{-/-} (FRG) mice, an animal model of liver failure and metabolic disease.

FRG mice will be pre-treated by intravenous administration of urokinase-expressing adenovirus (uPA virus) at a dose about 5×10⁹ plaque forming units (pfu/mouse).

About a million base-edited hepatocytes will be injected intrasplenically 24-48 hours after uPA administration and NTBC will be withdrawn. Liver disease in fumarylacetoacetate hydrolase (Fah) mutant mice is only developed when the drug 2-(2-nitro-4-trifluoromethylbenzoyl)-1,3-cyclohexanedione (NTBC) is withdrawn. NTBC withdrawal in FRG mice results in gradual hepatocellular injury and unless corrected, eventual death after 4-8 weeks.

FAH enzyme activity will be measured to determine hepatocytic function of engrafted cells. In addition, human albumin levels will be measured to confirm the presence of human edited cells. Histological/IHC analysis will be performed to confirm engraftment.

The results of this example will determine in vivo efficiency of engraftment and retention of transplanted base-edited hepatocytes in a mouse model.

### Example 6. Evaluating Engraftment of Hepatocytes in a FRG Pig Bioreactor

This example illustrates engraftment of base-edited cells in a FRG pig bioreactor for large-scale production of hepatocytes.

Obtaining and expanding hepatocytes in a FRG pig bioreactor overcomes the problem of limited supply of high-quality hepatocytes due to the limited supply of donor livers for organ transplantation.

In order to evaluate engraftment and expansion of edited hepatocytes, WT and base-edited hepatocytes will be engrafted in a FRG pig model by portal vein infusion.

After transplantation, the protective drug 2-(2-nitro-4-trifluoromethylbenzyol)-1,3 cyclohexanedione (NTBC) will be withheld from recipient pigs to provide a selective advantage for expansion of fumarylacetoacetate hydrolase (Fah+) cells.

Human albumin levels will be evaluated after 1, 3 and 6 months post-engraftment to confirm presence of human edited cells in FRG pig. Small amounts of blood will be collected with a heparinized blood capillary. After dilution with Tris-buffered saline, human albumin concentration will be measured using a human albumin ELISA quantitation kit. The degree of humanization of the liver generally correlates with human albumin blood levels such that 1 mg/mL corresponds to about 20% human hepatocytes.

Immunohistochemistry analysis of mouse liver tissue will also be performed at 4 or 6 months to confirm sufficient engraftment. Immunohistochemistry will be carried out for FAH or human albumin or cytokeratin expression.

At the end of about 12 months, the expanded human hepatocytes will be isolated, sorted and characterized by flow cytometry for presence/absence of Class I and II markers and Next Generation Sequencing will be used to assess editing retention post-engraftment (FIG. 1).

The results of this example will demonstrate the use of a FRG pig bioreactor for large scale production of modified hepatocytes following base editing that are suitable for liver transplantation.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method of producing genetically modified human hepatocytes suitable for hepatocyte transplantation comprising: disrupting one or more major histocompatibility complex (MHC) Class I or Class II genes in isolated human hepatocytes or in a hepatocyte progenitor cell by introducing a base editor and one or more gRNAs that hybridize with a target sequence in the one or more Class I or Class II genes, thereby producing genetically modified human hepatocytes.

2. The method of claim 1, wherein:
(i) the base editor comprises a CRISPR protein fused to a deaminase;
(ii) the genetically modified human hepatocytes have one or more nucleobase edits in a target sequence;
(iii) the genetically modified human hepatocytes have a disrupted target sequence;
(iv) the genetically modified human hepatocytes have reduced or abolished alloreactivity;
(v) the CRISPR protein is Cas9 or Cas12;
(vi) the isolated human hepatocytes have been previously cryopreserved and subsequently thawed; and/or
(vii) the genetically modified human hepatocytes overexpress CD47 and/or CD142 in comparison to a non-genetically modified human hepatocyte.

3.-5. (canceled)

6. The method of claim 1, wherein the Class I or Class II genes are selected from one or more of B2M, CD142, CIITA, HLA-A or HLA-B genes, wherein a stop codon or a splice site is introduced into one or more of the B2M, CD142, CIITA, HLA-A or HLA-B genes, and further wherein:
(i) a splice site is introduced at nucleotide position 19 of the B2M gene;
(ii) a stop codon is introduced at nucleotide position 5 of the B2M gene;
(iii) a splice site is introduced at nucleotide position 28 of the CD142 gene;
(iv) a stop codon is introduced at nucleotide position 19 of the CD142 gene;
(v) a splice site is introduced at nucleotide position 147 of the CIITA gene; or
(vi) a stop codon is introduced at nucleotide position 130 of the CIITA gene.

7-14. (canceled)

15. The method of claim 1, wherein the Cas9 (i) is from Streptococcus pyogenes (SpCas9) or Staphylococcus aureus (SaCas9), (ii) is a hyper-accurate Cas9, (iii) comprises mutations corresponding to N692A, M694A, Q695A and/or H698A with reference to SpyCas9 (SEQ ID NO: 68), (iv) is a high-fidelity Cas9, (v) comprises mutations corresponding to N467A, R661A, Q695A and/or Q926A with reference to SpyCas9 (SEQ ID NO: 68), (vi) is a SuperFi-Cas9, and/or (vi) wherein Y1016, R1019, Y1010, Y1013, K1031, Q1027 and/or V1018 residues corresponding to SpyCas9 (SEQ ID NO: 68) are mutated to aspartic acid.

15b-15g. (canceled)

16. The method of claim 2, wherein the CRISPR protein (i) is fused to an adenine base editor (ABE), a cytidine base editor (CBE), or an inosine base editor (IBE); (ii) is fused to a base editor comprising an adenine or adenosine deaminase domain or a cytidine or cytosine deaminase domain; (iii) is fused to a base editor comprising an adenine or adenosine deaminase domain and a cytidine or cytosine deaminase domain; (iv) comprises a nuclear localization sequence (NLS) and/or a FLAG, HIS or HA tag; and/or (v) comprises at least one mutation in SEQ ID NO: 1 (SpCas9), SEQ ID NO: 2 (SaCas9), or SEQ ID NO: 3 (Cas12).

16b-21. (canceled)

22. The method of claim 16 wherein the at least one mutation is an aspartic acid-to-alanine substitution at amino acid 10 (D10A), a histidine-to-alanine substitution at amino acid 840 (H840A) of SpCas9, or a corresponding mutation thereof in a RuvC domain and/or HNH domain of a Cas9 protein.

23. (canceled)

24. The method of claim 16, wherein the Cas9 protein has nickase activity.

25. The method of claim 1, wherein (i) the CRISPR protein is fused to an adenosine deaminase and has an amino acid sequence at least 80% identical to SEQ ID NO: 65, and/or (ii) the CRISPR protein is fused to a cytosine deaminase and has an amino acid sequence at least 80% identical to SEQ ID NO: 4-64.

26. (canceled)

27. The method of claim 1, wherein the SpCas9 protein recognizes a PAM sequence comprising 5′-NGG-3′,5′-NGA-3′, or 5′-NGC-3′; wherein the SaCas9 protein recognizes a PAM sequence comprising 5′-NNNRRT-3′, or 5′-NNGRRT-3′ or wherein the Cas12 protein recognizes a PAM sequence comprising 5′-RTTN-3′.

28.-30. (canceled)

31. The method of claim 1, wherein the genetically modified human hepatocytes overexpress CD47 and/or CD142 in comparison to a non-genetically modified human hepatocyte.

32. The method of claim 1, wherein the genetically modified human hepatocytes are engrafted into a humanized animal model for expansion, wherein (i) the humanized animal model is the FRG pig, the FRG mouse, or the FRG rat, (ii) the genetically modified human hepatocytes are first engrafted into the FRG mouse or FRG rat for an initial cell expansion, (iii) the initially expanded cells or the further expanded cells are isolated from an animal, and/or (iv) the initially expanded cells or the further expanded cells are isolated by fluorescence-activated cell sorting, immunomagnetic cell separation, density gradient centrifugation, and/or immunodensity cell separation.

33.-37. (canceled)

38. The method of claim 1, wherein the genetically modified human hepatocytes have one, two, three or more nucleobase edits, and wherein (i) a single base editor in combination with more than one guide produces the two, three or more nucleobase edits, or (ii) wherein more than one base editor produces the one, two, three or more nucleobase edits.

39.-40. (canceled)

41. The method of claim 1, wherein (i) the method comprises a base editor and one or more guide RNAs that target the B2M gene, wherein the base editor and corresponding one or more guide RNAs comprising any one of the protospacer sequences listed in Table 2 are selected; (ii) the method comprises a base editor and one or more guide RNAs that target the CD142 gene, wherein the base editor and corresponding one or more guide RNAs comprising any one of the protospacer sequences listed in Table 3 are selected; (iii) the method comprises a base editor and one or more guide RNAs that target the CIITA gene, wherein the base editor and corresponding one or more guide RNAs comprising any one of the protospacer sequences listed in Table 4 are selected; (iv) the method comprises a base editor and one or more guide RNAs that target the HLA-A gene, wherein the base editor and corresponding one or more guide RNAs comprising any one of the protospacer sequences listed in Table 5 are selected; (v) the method comprises a base editor and one or more guide RNAs that target the HLA-B gene, wherein the base editor and corresponding one or more guide RNAs comprising any one of the protospacer sequences listed in Table 6 are selected; (vi) the method comprises a base editor and one or more guide RNAs that target the B2M gene, wherein the base editor and corresponding one or more guide RNAs comprising any one of the sequences listed in Table 2A are selected; (vii) the method comprises a base editor and one or more guide RNAs that target the CD142 gene, wherein the base editor and corresponding one or more guide RNAs comprising any one of the sequences listed in Table 3A are selected; (viii) the method comprises a base editor and one or more guide RNAs that target the CIITA gene, wherein the base editor and corresponding one or more guide RNAs comprising any one of the sequences listed in Table 4A are selected; (ix) the method comprises a base editor and one or more guide RNAs that target the HLA-A gene, wherein the base editor and corresponding one or more guide RNAs comprising any one of the sequences listed in Table 5A are selected; and/or (x) the method comprises a base editor and one or more guide RNAs that target the HLA-B gene, wherein the base editor and corresponding one or more guide RNAs comprising any one of the sequences listed in Table 6A are selected.

41b.-41k (canceled)

42. A nucleic acid encoding the base editor and one or more gRNAs that hybridize with a sequence of claim 41, wherein the nucleic acid is codon-optimized for expression in human cell.

43.-44. (canceled)

45. A vector encoding the nucleic acid of claim 42.

46. A eukaryotic cell comprising the base editor and one or more gRNAs comprising any one of the sequences listed in Tables 2A-6A or an RNA version of any one of the protospacer sequences listed in Table 2-6 that hybridize with a target sequence, wherein the cell is a human hepatocyte.

47-48. (canceled)

49. A method of treating a liver disease, the method comprising administering to a subject in need thereof, genetically modified human hepatocytes of claim 46, wherein about 10-15 billion genetically modified human hepatocytes are injected into the portal vein of a subject in need thereof.

50.-56. (canceled)

57. A guide RNA comprising any one of the sequences listed in Tables 2A-6A or an RNA version of any one of the protospacer sequences listed in Table 2-6, wherein one, two, three, or more than three edits are made to the target gene.

57b.-57p (canceled)

58. A cell comprising a base editor and one or more guide RNAs of claim 57.

59. A cell of claim 58, wherein the cell is a genetically modified human hepatocyte that has one or more edits in an MHC gene, wherein the MHC gene is selected from B2M, CD142, CIITA, HLA-A and/or HLA-B, and wherein edits to one or more of B2M, CD142, CIITA, HLA-A and/or HLA-B genes results in increased expression of the B2M, CD142, CIITA, HLA-A and/or HLA-B genes in comparison to a non-genetically modified human hepatocyte.

60.-61. (canceled)
